(19) United States
(12) Patent Application Publication
(10) Pub. No.: US 2023/0348351 A1
(43) Pub. Date: Nov. 2, 2023

(54) COMPOUND, POLYMER, COMPOSITION, COMPOSITION FOR FILM FORMATION, PATTERN FORMATION METHOD, INSULATING FILM FORMATION METHOD, AND METHOD FOR PRODUCING COMPOUND

(71) Applicant: Mitsubishi Gas Chemical Company, Inc., Tokyo (JP)

(72) Inventors: Tadashi OMATSU, Hiratsuka-shi, Kanagawa (JP); Yu OKADA, Hiratsuka-shi, Kanagawa (JP); Takeru KOGUMA, Hiratsuka-shi, Kanagawa (JP); Masahiro MATSUMOTO, Niigata-shi, Niigata (JP); Yushi NIIMI, Niigata-shi, Niigata (JP); Masatoshi ECHIGO, Tokyo (JP)

(21) Appl. No.: 18/268,940

(22) PCT Filed: Dec. 21, 2021
(86) PCT No.: PCT/JP2021/047416

§ 371 Date: Jun. 21, 2023

(30) Foreign Application Priority Data
Dec. 21, 2020 (JP) 2020-211688
Feb. 5, 2021 (JP) 2021-017621

(51) Int. Cl. C07C 37/20; C07C 39/373; C07C 41/22; C07C 41/26; C07C 41/30; C07C 43/225; C07C 43/23; C07C 45/63; C07C 49/84; C07C 59/70; C07C 67/08; C07C 69/63; C07C 69/712; C07C 69/96; C07D 309/12; C08F 12/16; C08F 212/14; C08F 246/00; G03F 7/038; G03F 7/039; G03F 7/20

(52) U.S. Cl. CPC C07C 37/20; C07C 39/373; C07C 41/22; C07C 41/26; C07C 41/30; C07C 43/225; C07C 43/23; C07C 45/63; C07C 49/84; C07C 59/70; C07C 67/08; C07C 69/63; C07C 69/712; C07C 69/96; C07D 309/12; C08F 12/16; C08F 212/14; C08F 246/00; G03F 7/038; G03F 7/039; G03F 7/20

(57) ABSTRACT

Provided is a compound, a polymer, a composition, a composition for film formation, a pattern formation method, an insulating film formation method, and a method for producing a compound, by which a resist having excellent exposure sensitivity can be obtained.

A compound represented by the following formula (1):

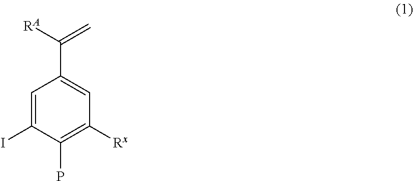

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group.

### TECHNICAL FIELD

The present invention relates to a compound, a polymer, a composition, a composition for film formation, a pattern formation method, an insulating film formation method, and a method for producing a compound.

### BACKGROUND ART

In recent years, in the production of semiconductor elements and liquid crystal display elements, semiconductors (patterns) and pixels have been rapidly miniaturized due to the advance in lithography technology. For pixel miniaturization, the exposure light source has been shifted to have a shorter wavelength, in general. Specifically, ultraviolet rays typified by g-ray and i-ray have been used conventionally, but nowadays, an exposure method for using far ultraviolet such as KrF excimer laser (248 nm) and ArF excimer laser (193 nm) is being the center of mass production. Furthermore, the introduction of extreme ultraviolet (EUV) lithography (13.5 nm) is progressing. In addition, electron beam (EB) is also used for forming a fine pattern.

Up to now, typical resist materials are polymer based resist materials capable of forming an amorphous film. Examples include polymer based resist compositions such as polymethyl methacrylate, polyhydroxy styrene with an acid dissociation group, and polyalkyl methacrylate (see, for example, Non Patent Literature 1). Conventionally, a line pattern of about 10 to 100 nm is formed by irradiating a resist thin film made by coating a substrate with a solution of these resist compositions with ultraviolet, far ultraviolet, electron beam, extreme ultraviolet or the like.

In addition, lithography using electron beam or extreme ultraviolet (EUV) has a reaction mechanism different from that of normal photolithography (see Non Patent Literature 2, Non Patent Literature 3). Furthermore, lithography with electron beam or extreme ultraviolet aims at forming fine patterns of several nm to ten-odd nm. Accordingly, there is a demand for a resist composition having higher sensitivity to an exposure light source when the dimension of the resist pattern is reduced. In particular, lithography with extreme ultraviolet (EUV) is required to further increase sensitivity in terms of throughput. There is not necessarily a correlation between the sensitivity in extreme ultraviolet (EUV) and the sensitivity in electron beam (EB), and it is required to exhibit particularly high sensitivity to extreme ultraviolet (EUV).

As a resist material that solves the problems as mentioned above, a resist composition having a metallic complex such as titanium, tin, hafnium and zirconium has been proposed (see, for example, Patent Literature 1).

There is a demand for a resist composition having higher sensitivity to an exposure light source when the dimension of the resist pattern is reduced, and 4-hydroxystyrene containing iodine is proposed as a raw material monomer of the resist composition, (see, for example, Patent Literatures 2 to 3), but there is room for improvement in the effects thereof.

### CITATION LIST

### Patent Literature

Patent Literature 1: Japanese Patent Laid-Open No. 2015-108781
Patent Literature 2: U.S. Patent No. 2019/0187342
Patent Literature 3: International Publication No. WO 2019/187881

### Non Patent Literature

Non Patent Literature 1: Shinji Okazaki et al., and eight others “Development of Lithography Technology in These 40 Years”, S&T Publishing Inc., Dec. 9, 2016
Non Patent Literature 2: H. Yamamoto, et al., Jpn. J. Appl. Phys. 46, L142 (2007)
Non Patent Literature 3: H. Yamamoto, et al., J. Vac. Sci. Technol. b 23, 2728 (2005)

### SUMMARY OF INVENTION

### Technical Problem

However, conventionally developed compositions for film formation have a problem of insufficient sensitivity to an exposure light source in the formation of a further thinned pattern.

To solve these problems, an object of the present invention is to provide a compound, a polymer, a composition, a composition for film formation, a pattern formation method, an insulating film formation method, and a method for producing a compound, by which a resist having further excellent exposure sensitivity can be obtained.

### Solution to Problem

The inventors have, as a result of devoted examinations to solve the aforementioned problems, found out that the exposure sensitivity of a resist formed by using a compound having a specific structure or a polymer including the compound as a constitutional unit can be increased, and reached the present invention.

More specifically, the present invention is as follows.

[1]

A compound represented by the following formula (1):

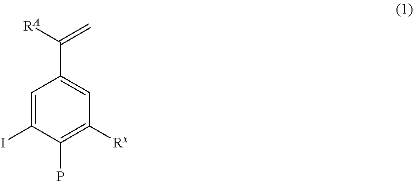

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group.
[2]

The compound according to [1], wherein R^{A} is a hydrogen atom or a methyl group.

[3]

The compound according to [1] or [2], wherein R^{B} is an alkyl group having 1 to 4 carbon atoms.

[4]

The compound according to any one of [1] to [3], wherein P is a hydroxy group, an ester group, an acetal group, a carbonate ester group, or a carboxyalkoxy group.

[5]

The compound according to any one of [1] to [4], wherein P is an ester group, an acetal group, or a carbonate ester group.

[6]

A composition comprising a compound represented by the following formula (1A) in an amount of 1 ppm by mass or more and 10% by mass or less, based on the total compound according to any one of [1] to [5]:

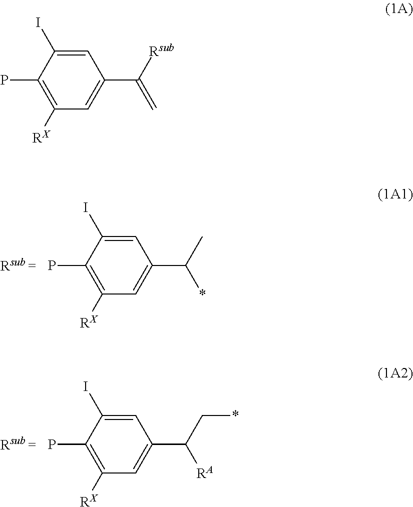

wherein R^{A}, R^{X}, R^{B}, and P have the same meanings as in the formula (1); R^{sub} represents the formula (1A1) or formula (1A2); and * is a site for binding with an adjacent constitutional unit.
[7]

A composition comprising: the compound according to any one of [1] to [5]; and based on the total compound, 1 ppm by mass or more and 10% by mass or less of a compound represented by the following formula (1B):

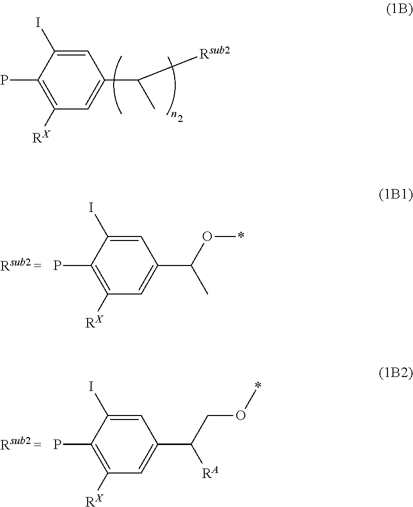

wherein R^{A}, R^{X}, R^{B}, and P have the same meanings as in the formula (1); n₂ is an integer of 0 or more and 4 or less; R^{sub2} represents the formula (1B1) or the formula (1B2); and * is a site for binding with an adjacent constitutional unit.
[8]

A composition comprising a compound represented by the following formula (1C) in an amount of 1 ppm by mass or more and 10% by mass or less, based on the total compound according to any one of [1] to [5]:

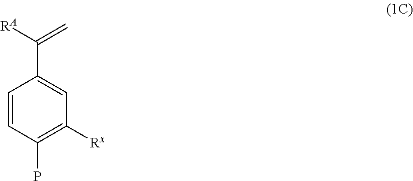

wherein R^{A}, R^{X}, R^{B}, and P have the same meanings as in the formula (1); provided that neither R^{B} nor P contains I.
[9]

A composition comprising the compound according to any one of [1] to [5],
wherein a content of impurities containing K is 1 ppm by mass or less, in terms of element, based on the total compound.
   [10]

The composition according to [9], wherein a content of peroxide is 10 ppm by mass or less, based on the total compound.

[11]

The composition according to [9] or [10], wherein a content of impurities containing one or more elements selected from the group consisting of Mn, Al, Si, and Li is 1 ppm by mass or less, in terms of element, based on the total compound.

[12]

The composition according to any one of [9] to [11], wherein a content of a phosphorus-containing compound is 10 ppm by mass or less, based on the total compound.

[13]

The composition according to any one of [9] to [12], wherein a content of maleic acid is 10 ppm by mass or less, based on the total compound.

[14]

A polymer comprising a constitutional unit represented by the following formula (1-A), comprising a constitutional unit derived from the compound according to any one of [1] to [5]:

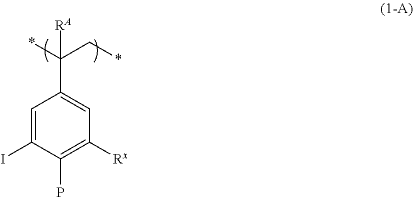

wherein R^{A}, R^{X}, R^{B}, and P have the same meanings as in the formula (1); and * is a site for binding with an adjacent constitutional unit.
[15]

The polymer according to [14], further comprising a constitutional unit represented by the following formula (C0), the following formula (C1), or the following formula (C2):

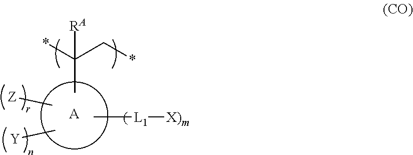

wherein
each X is independently I, F, Cl, Br, or an organic group having 1 to 30 carbon atoms and having 1 or more and 5 or less substituents selected from the group consisting of I, F, Cl, and Br;
each L¹ is independently a single bond, an ether group, an ester group, a thioether group, an amino group, a thioester group, an acetal group, a phosphine group, a phosphone group, a urethane group, a urea group, an amide group, an imide group, or a phosphate group, and the ether group, the ester group, the thioether group, the amino group, the thioester group, the acetal group, the phosphine group, the phosphone group, the urethane group, the urea group, the amide group, the imide group, or the phosphate group of L¹ optionally has a substituent;
each Y is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;
R^{A} is as defined in the formula (1);
A is an organic group having 1 to 30 carbon atoms;
each Z is independently an alkoxy group, an ester group, an acetal group, a carboxyalkoxy group, or a carbonate ester group, and the alkoxy group, the ester group, the acetal group, the carboxyalkoxy group, or the carbonate ester group of Z optionally has a substituent; and
m is an integer of 0 or more, n is an integer of 1 or more, and r is an integer of 0 or more;

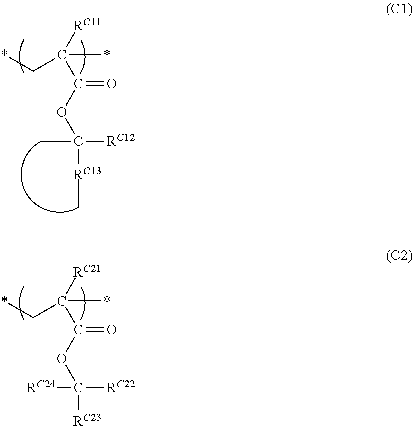

wherein, in the formula (C1),
R^{C11} is a hydrogen atom, a methyl group, or a trifluoromethyl group;
R^{C12} is a hydrogen atom, or an alkyl group having 1 to 4 carbon atoms;
R^{C13} is a cycloalkyl group or heterocycloalkyl group having 4 to 20 carbon atoms formed by taking R^{C13} together with a carbon atom bonded thereto; and
* is a site for binding with an adjacent constitutional unit;
wherein, in the formula (C2),
R^{C21} is a hydrogen atom, a methyl group, or a trifluoromethyl group;
each of R^{C22} and R^{C23} is independently an alkyl group having 1 to 4 carbon atoms;
R^{C24} is an alkyl group having 1 to 4 carbon atoms or a cycloalkyl group having 5 to 20 carbon atoms;
two or three selected from R^{C22}, R^{C23}, and R^{C24} are optionally taken together with a carbon atom bonded to the two or three selected from R^{C22}, R^{C23}, and R^{C24} to form an alicyclic structure having 3 to 20 carbon atoms; and
* is a site for binding with an adjacent constitutional unit.
   [16]

A composition for film formation comprising the compound according to any one of [1] to [5], the composition according to any one of [6] to [13], or the polymer according to [14] or [15].

[17]

The composition for film formation according to [16], further comprising an acid generating agent, a base generating agent, or a basic compound.

[18]

A resist pattern formation method comprising:
a step of forming a resist film on a substrate using the composition for film formation according to [16] or [17];
a step of exposing a pattern on the resist film; and
a step of subjecting the resist film after exposure to a development treatment.
   [19]

An insulating film formation method comprising:
a step of forming a resist film on a substrate using the composition for film formation according to [16] or [17];
a step of exposing a pattern on the resist film; and
a step of subjecting the resist film after exposure to a development treatment.
   [20]

A method for producing an iodine-containing vinyl monomer represented by the following formula (1), comprising:
a) a step of providing an iodine-containing alcohol substrate having a general structure represented by the following formula (1-1):

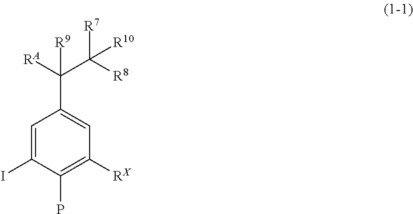

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
b) a dehydration step of subjecting the iodine-containing alcohol substrate to a dehydration treatment:

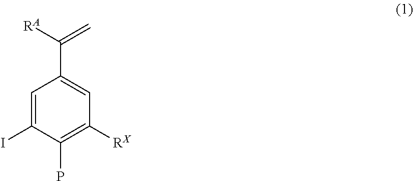

wherein R^{A} is a hydrogen atom, a methyl group, or a trifluoromethyl group;
R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group.
[21]

The method for producing the iodine-containing vinyl monomer represented by the formula (1) according to [20], further comprising:
c) a step of providing an iodine-containing ketone substrate having a general structure represented by the following formula (1-2):

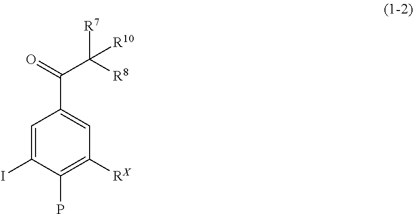

wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
d) a reduction step of subjecting the iodine-containing ketone substrate to a reduction treatment.
   [22]

The method for producing the iodine-containing vinyl monomer represented by the formula (1) according to [20], further comprising:
e) a step of providing an alcohol substrate having a general structure represented by the following formula (1-3):

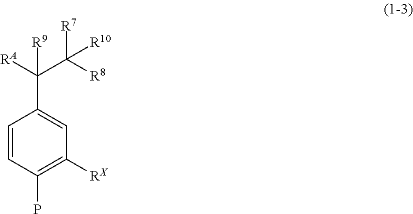

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
f) an iodine introduction step of introducing an iodine atom into the alcohol substrate.
   [23]

The method for producing the iodine-containing vinyl monomer represented by the formula (1) according to [20], further comprising:
g) a step of providing a ketone substrate having a general structure represented by the following formula (1-4):

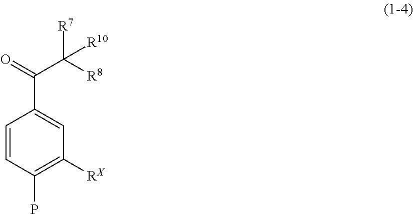

wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
h) an iodine introduction step of introducing an iodine atom into the ketone substrate.
   [24]

The method for producing the iodine-containing vinyl monomer represented by the formula (1) according to [20], further comprising:
i) a step of providing a ketone substrate having a general structure represented by the following formula (1-4):

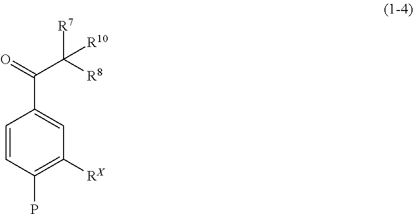

wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
j) a reduction step of subjecting the ketone substrate to a reduction treatment.
   [25]
   A method for producing an iodine-containing vinyl monomer represented by the following formula (2), comprising:
k) a step of providing an iodine-containing vinyl monomer having a general structure represented by the following formula (1):

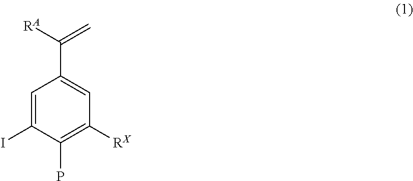

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and
l) an acylation step of subjecting the iodine-containing vinyl monomer to an acylation treatment;

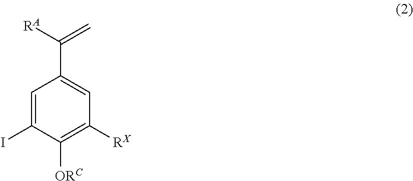

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and R^{C} is a substituted or unsubstituted acyl group having 1 to 30 carbon atoms.
[26]

A method for producing an iodine-containing alcohol compound represented by the following formula (1-1), comprising:
c) a step of providing an iodine-containing ketone substrate having a general structure represented by the following formula (1-2):

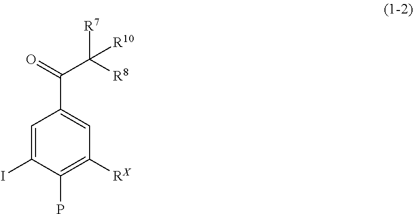

wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
d) a reduction step of subjecting the iodine-containing ketone substrate to a reduction treatment;

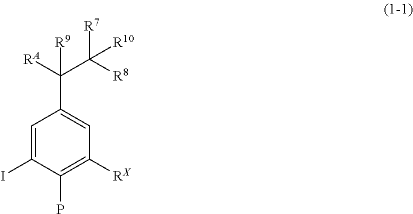

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group.
[27]

A method for producing an iodine-containing alcohol compound represented by the following formula (1-1), comprising:
e) a step of providing an alcohol substrate having a general structure represented by the following formula (1-3):

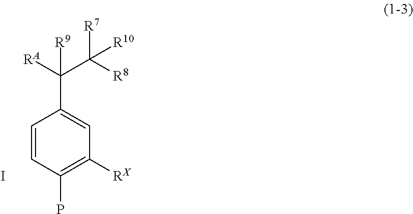

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
f) an iodine introduction step of introducing an iodine atom into the alcohol substrate;

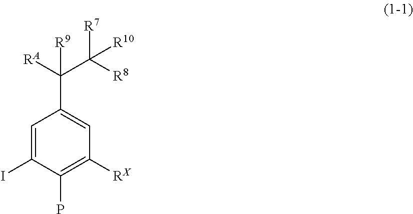

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group.
[28]

A method for producing an iodine-containing ketone compound represented by the following formula (1-2), comprising:
g) a step of providing a ketone substrate having a general structure represented by the following formula (1-4):

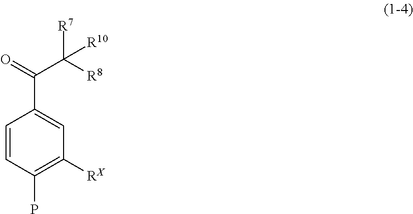

wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
h) an iodine introduction step of introducing an iodine atom into the ketone substrate;

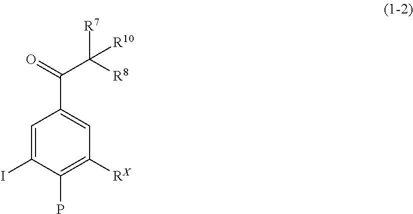

wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group.
[29]

A method for producing an alcohol compound represented by the following formula (1-3), comprising:
i) a step of providing a ketone substrate having a general structure represented by the following formula (1-4):

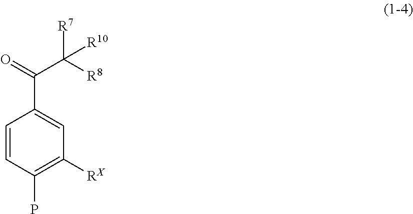

wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
j) a reduction step of subjecting the ketone substrate to a reduction treatment;

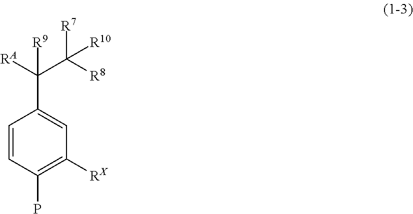

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group.
[30]

A method for producing an iodine-containing vinyl monomer represented by the following formula (1), comprising:
a) a step of providing an iodine-containing aldehyde substrate or iodine-containing ketone substrate having a general structure represented by the following formula (1-5):

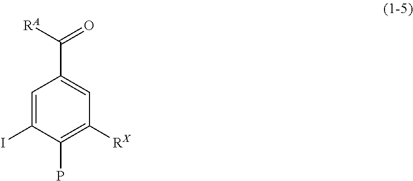

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and
b) a Wittig reaction step of forming an alkene from a carbonyl moiety of the iodine-containing aldehyde substrate or the iodine-containing ketone substrate by a Wittig reaction;

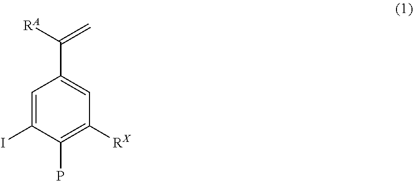

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group.
[31]

A method for producing an iodine-containing vinyl monomer represented by the following formula (1), comprising:
a) a step of providing an iodine-containing aldehyde substrate or iodine-containing ketone substrate having a general structure represented by the following formula (1-5):

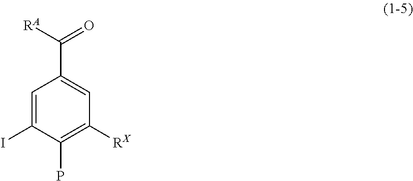

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group;
b) a malonic acid addition step of adding malonic acid to the iodine-containing aldehyde substrate or the iodine-containing ketone substrate;
c) a hydrolysis step of subjecting the iodine-containing aldehyde substrate or iodine-containing ketone substrate to which the malonic acid is added to a hydrolysis treatment to produce an iodine-containing carboxylic acid substrate; and
d) a decarbonation step of subjecting the iodine-containing carboxylic acid substrate to a decarbonation treatment;

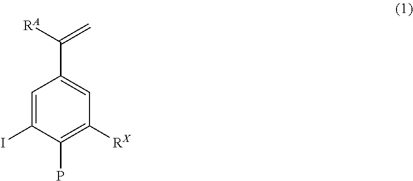

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group.
[32]

The compound, polymer, composition, composition for film formation, pattern formation method, insulating film formation method, and method for producing a compound according to any one of [1] to [31] applied to extreme ultraviolet applications.

### Advantageous Effects of Invention

The present invention can provide a compound, a polymer, a composition, a composition for film formation, a pattern formation method, an insulating film formation method, and a method for producing a compound, by which a resist having further excellent exposure sensitivity can be obtained.

### DESCRIPTION OF EMBODIMENTS

### First Embodiment

Hereinafter, the first embodiment of the present invention will be described (hereinafter, may be referred to as the “present embodiment”). The present embodiment is given in order to illustrate the present invention. The present invention is not limited to only the present embodiment.

As used herein, the meaning of each term is as follows.

The term “(meth)acrylate” means at least one selected from acrylate, haloacrylate, and methacrylate. The term haloacrylate means an acrylate in which the position of the methyl group in methacrylate is substituted with a halogen. Other terms having the expression (meth) should be similarly interpreted as (meth)acrylate.

The term “(co)polymer” means at least one selected from a homopolymer and a copolymer.

[Compound (A)]

The compound according to the first present embodiment (hereinafter, also referred to as “compound (A)” in the first present embodiment) is represented by the following formula (1).

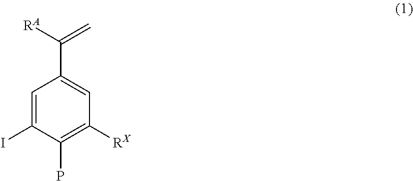

(In the formula (1), R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group.)

The compound (A) according to the present embodiment can provide a compound for obtaining a resist having further excellent exposure sensitivity.

In the present embodiment, unless otherwise defined, the term “substituted” means that one or more hydrogen atoms in a functional group are substituted with a substituent. Examples of the “substituent” include, but are not particularly limited to, a halogen atom, a hydroxyl group, a carboxyl group, a cyano group, a nitro group, a thiol group, a heterocyclic ring group, an alkyl group having 1 to 30 carbon atoms, an aryl group having 6 to 30 carbon atoms, an alkoxyl group having 1 to 30 carbon atoms, an alkenyl group having 2 to 30 carbon atoms, an alkynyl group having 2 to 30 carbon atoms, an acyl group having 1 to 30 carbon atoms, and an amino group having 0 to 30 carbon atoms. The alkyl group may be any of a linear aliphatic hydrocarbon group, a branched aliphatic hydrocarbon group, and a cyclic aliphatic hydrocarbon group.

From the viewpoint of increasing the hydrophilicity to achieve high sensitivity, in the formula (1), R^{A} is preferably a hydrogen atom or a methyl group.

R^{B} is preferably an alkyl group having 1 to 4 carbon atoms.

From the viewpoint of high sensitivity, P is preferably a hydroxy group, an ester group, an acetal group, a carbonate ester group, or a carboxyalkoxy group, and more preferably an ester group, an acetal group, or a carbonate ester group.

The compound (A) described above is preferably used in combination with a compound represented by the following formula (1A). That is, the composition according to the present embodiment preferably contains the compound (A) and the compound represented by the formula (1A). The composition is preferably prepared such that the compound represented by the formula (1A) is contained within a range of 1 ppm by mass or more and 10% by mass or less, more preferably within a range of 1 ppm by mass or more and 5% by mass or less, further preferably within a range of 1 ppm by mass or more and 3% by mass or less, and particularly preferably within a range of 1 ppm by mass or more and 1% by mass or less, based on the total compound (A), from the viewpoint of improving the exposure sensitivity and reducing the residue defect.

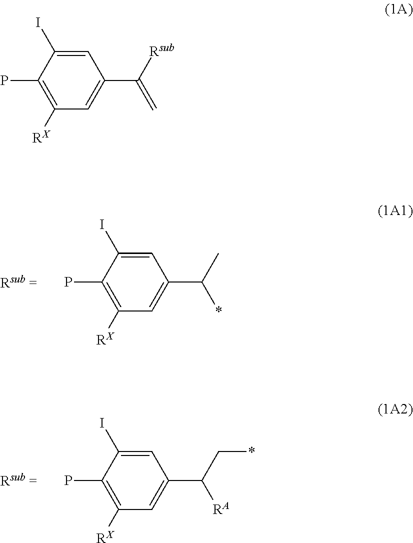

(In the formula (1A), the formula (1A1), and the formula (1A2), R^{A}, R^{X}, R^{B}, and P have the same meanings as in the formula (1); R^{sub} represents the formula (1A1) or formula (1A2); and * is a site for binding with an adjacent constitutional unit.)

The compound (A) described above is preferably used in combination with a compound represented by the following formula (1B). That is, the composition according to the present embodiment preferably contains the compound (A) and the compound represented by the formula (1B). The composition is preferably prepared such that the compound represented by the formula (1B) is contained within a range of 1 ppm by mass or more and 10% by mass or less, more preferably within a range of 1 ppm by mass or more and 5% by mass or less, further preferably within a range of 1 ppm by mass or more and 3% by mass or less, and particularly preferably within a range of 1 ppm by mass or more and 1% by mass or less, based on the total compound (A), from the viewpoint of improving the exposure sensitivity and reducing the residue defect.

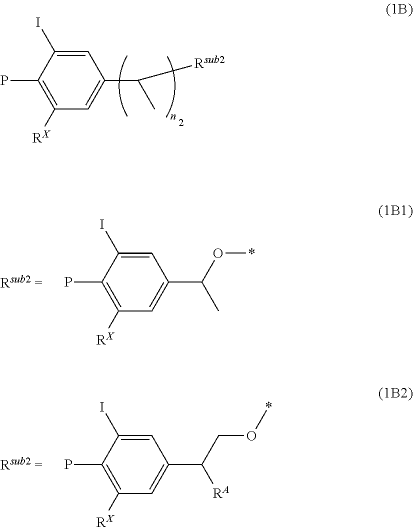

(In the formula (1B), the formula (1B1), or the formula (1B2), R^{A}, R^{X}, R^{B}, and P have the same meanings as in the formula (1); n₂ is an integer of 0 or more and 4 or less; R^{sub2} represents the formula (1B1) or the formula (1B2); and * is a site for binding with an adjacent constitutional unit.)

The compound (A) described above is preferably used in combination with a compound represented by the following formula (1C). That is, the composition according to the present embodiment preferably contains the compound (A) and the compound represented by the formula (1C). The composition preferably contains the compound represented by the formula (1C) within a range of 1 ppm by mass or more and 10% by mass or less, more preferably within a range of 1 ppm by mass or more and 5% by mass or less, further preferably within a range of 1 ppm by mass or more and 3% by mass or less, and particularly preferably within a range of 1 ppm by mass or more and 1% by mass or less, based on the total compound (A), from the viewpoint of stability and reducing the residue defect.

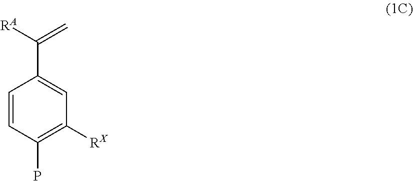

(In the formula (1C), R^{A}, R^{X}, R^{B}, and P have the same meanings as in the formula (1), provided that neither R^{B} nor P contains I.)

The composition of the present embodiment contains the compound (A). The composition may contain K (potassium). In the composition, the content of impurities containing K is preferably 1 ppm by mass or less, more preferably 0.5 ppm by mass or less, further preferably 0.1 ppm by mass or less, and still more preferably 0.005 ppm by mass or less, in terms of element, based on the total compound (A).

In the composition of the present embodiment, the content of peroxide is preferably 10 ppm or less, more preferably 1 ppm or less, and further preferably 0.1 ppm or less, based on the total compound (A).

In the composition of the present embodiment, the content of one or more elemental impurities selected from the group consisting of Mn (manganese), Al (aluminum), Si (silicon), and Li (lithium) (preferably, one or more elemental impurities selected from the group consisting of Mn and Al) is preferably 1 ppm or less, more preferably 0.5 ppm or less, further preferably 0.1 ppm or less, in terms of element, based on the total compound (A).

In the composition of the present embodiment, the content of the phosphorus-containing compound is preferably 10 ppm or less, more preferably 8 ppm or less, and further preferably 5 ppm or less, based on the total compound (A).

In the composition of the present embodiment, the content of maleic acid is preferably 10 ppm or less, more preferably 8 ppm or less, and further preferably 5 ppm or less, based on the total compound (A).

### [Polymer (A)]

The polymer (A) of the present embodiment contains a constitutional unit derived from the aforementioned compound (A). By containing the constitutional unit derived from the compound (A), the polymer (A) can increase the sensitivity to an exposure light source when being blended in a resist composition. In particular, the polymer (A) can exhibit sufficient sensitivity and can form good thin line patterns having a narrow line width even in the case of using an extreme ultraviolet ray as the exposure light source.

According to the polymer (A) of the present embodiment, the stability of the resist composition is improved, and the reduction in the sensitivity to an exposure light source is suppressed even in the case of long-term storage.

The polymer (A) of the present embodiment contains the constitutional unit derived from the compound (A). The constitutional unit derived from the compound (A) contained in the polymer (A) includes, for example, a constitutional unit represented by the following formula (1-A).

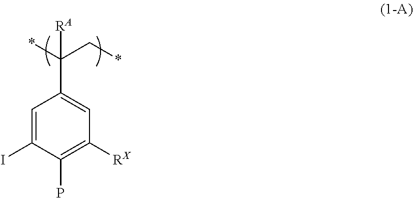

(In the formula (1-A), R^{A}, R^{X}, R^{B}, and P have the same meanings as in the formula (1); and * is a site for binding with an adjacent constitutional unit.)

Other monomers to be copolymerized with the compound (A) in the polymer (A) preferably contains a constitutional unit represented by the following formula (C0). That is, the polymer (A) preferably further contains the constitutional unit represented by the following formula (C0), a constitutional unit represented by the following formula (C1), or a constitutional unit represented by the following formula (C2), in addition to the constitutional unit represented by the formula (1-A).

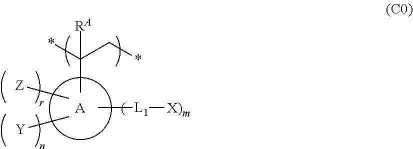

(In the formula (C0),
each X is independently I, F, Cl, Br, or an organic group having 1 to 30 carbon atoms and having 1 or more and 5 or less substituents selected from the group consisting of I, F, Cl, and Br;
each L¹ is independently a single bond, an ether group, an ester group, a thioether group, an amino group, a thioester group, an acetal group, a phosphine group, a phosphone group, a urethane group, a urea group, an amide group, an imide group, or a phosphate group, and the ether group, the ester group, the thioether group, the amino group, the thioester group, the acetal group, the phosphine group, the phosphone group, the urethane group, the urea group, the amide group, the imide group, or the phosphate group of L¹ optionally has a substituent;
each Y is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;
R^{A} is as defined in the formula (1);
A is an organic group having 1 to 30 carbon atoms;
each Z is independently an alkoxy group, an ester group, an acetal group, a carboxyalkoxy group, or a carbonate ester group, and the alkoxy group, the ester group, the acetal group, the carboxyalkoxy group, or the carbonate ester group of Z optionally has a substituent; and
m is an integer of 0 or more, n is an integer of 1 or more, and r is an integer of 0 or more.)

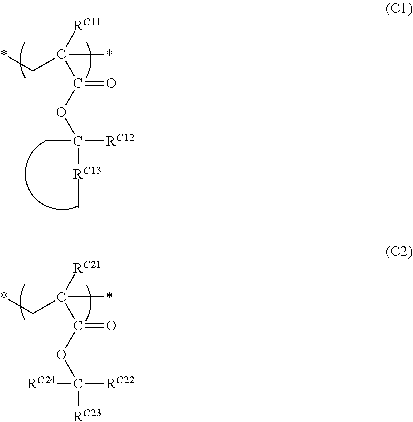

(In the formula (C1),
R^{C11} is a hydrogen atom, a methyl group, or a trifluoromethyl group;
R^{C12} is a hydrogen atom, or an alkyl group having 1 to 4 carbon atoms;
R^{C13} is a cycloalkyl group or heterocycloalkyl group having 4 to 20 carbon atoms formed by taking R^{C13} together with a carbon atom bonded thereto; and
* is a site for binding with an adjacent constitutional unit.

In the formula (C2),
R^{C21} is a hydrogen atom, a methyl group, or a trifluoromethyl group;
each of R^{C22} and R^{C23} is independently an alkyl group having 1 to 4 carbon atoms;
R^{C24} is an alkyl group having 1 to 4 carbon atoms or a cycloalkyl group having 5 to 20 carbon atoms;
two or three selected from R^{C22}, R^{C23}, and R^{C24} are optionally taken together with a carbon atom bonded to the two or three selected from R^{C22}, R^{C23}, and R^{C24} to form an alicyclic structure having 3 to 20 carbon atoms; and
* is a site for binding with an adjacent constitutional unit.)

### [Composition for Film Formation]

The composition for film formation of the present embodiment can also be used as an optical component forming composition applying lithography technology. The optical component is used in the form of a film or a sheet and is also useful as a plastic lens (a prism lens, a lenticular lens, a microlens, a Fresnel lens, a viewing angle control lens, a contrast improving lens, etc.), a phase difference film, a film for electromagnetic wave shielding, a prism, an optical fiber, a solder resist for flexible printed wiring, a plating resist, an interlayer insulating film for multilayer printed circuit boards, a photosensitive optical waveguide, a liquid crystal display, an organic electroluminescent (EL) display, an optical semiconductor (LED) element, a solid state image sensing element, an organic thin film solar cell, a dye sensitized solar cell, and an organic thin film transistor (TFT). The composition can be suitably utilized as an embedded film and a smoothed film on a photodiode, a smoothed film in front of or behind a color filter, a microlens, and a smoothed film and a conformal film on a microlens, all of which are components of a solid state image sensing element, to which high refractive index is particularly demanded.

The composition for film formation of the present embodiment may contain the compound (A), the composition of the present embodiment, or the polymer (A). The composition for film formation of the present embodiment may further contain an acid generating agent (C), a base generating agent (G), or an acid diffusion controlling agent (E) (basic compound).

### [Method for Forming Resist Pattern and Insulating Film]

The resist pattern formation method of the present embodiment may comprise:
a step of forming a resist film on a substrate using the composition for film formation of the present embodiment;
a step of exposing a pattern on the resist film; and
a step of subjecting the resist film after exposure to a development treatment.

The insulating film formation method of the present embodiment may comprise the resist pattern formation method of the present embodiment. That is, the insulating film formation method of the present embodiment may comprise:
a step of forming a resist film on a substrate using the composition for film formation of the present embodiment;
a step of exposing a pattern on the resist film; and
a step of subjecting the resist film after exposure to a development treatment.

In the present embodiment, the method for producing the iodine-containing vinyl monomer represented by the formula (1) may comprise:
a) a step of providing an iodine-containing alcohol substrate having a general structure represented by the following formula (1-1):

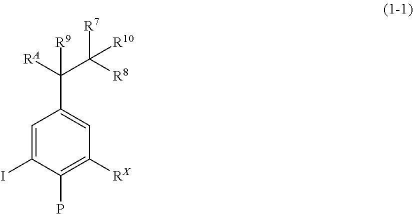

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
b) a dehydration step of subjecting the iodine-containing alcohol substrate to a dehydration treatment:

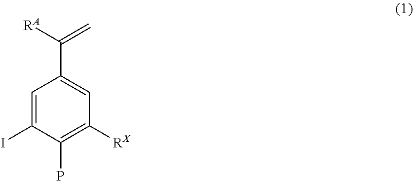

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group.

In the present embodiment, the method for producing the iodine-containing vinyl monomer represented by the formula (1) may further comprise:
c) a step of providing an iodine-containing ketone substrate having a general structure represented by the following formula (1-2):

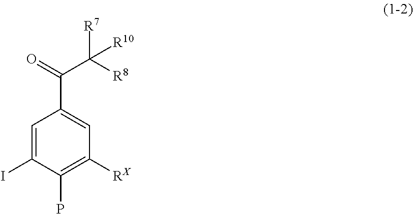

wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
d) a reduction step of subjecting the iodine-containing ketone substrate to a reduction treatment.

In the present embodiment, the method for producing the iodine-containing vinyl monomer represented by the formula (1) may further comprise:
e) a step of providing an alcohol substrate having a general structure represented by the following formula (1-3):

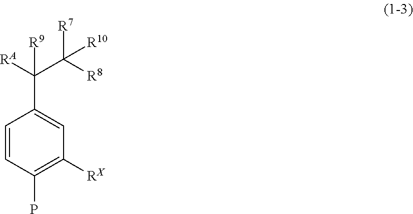

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
f) an iodine introduction step of introducing an iodine atom into the alcohol substrate.

In the present embodiment, the method for producing the iodine-containing vinyl monomer represented by the formula (1) may further comprise:
g) a step of providing a ketone substrate having a general structure represented by the following formula (1-4):

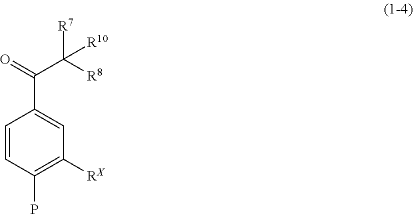

wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
h) an iodine introduction step of introducing an iodine atom into the ketone substrate.

In the present embodiment, the method for producing the iodine-containing vinyl monomer represented by the formula (1) may further comprise:
i) a step of providing a ketone substrate having a general structure represented by the following formula (1-4):

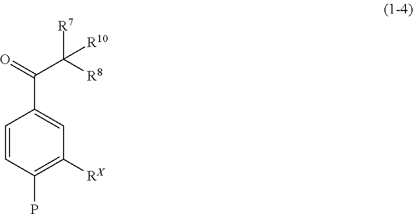

wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
j) a reduction step of subjecting the ketone substrate to a reduction treatment.

In the present embodiment, the method for producing the iodine-containing vinyl monomer represented by the following formula (2) may comprise:
k) a step of providing an iodine-containing vinyl monomer having a general structure represented by the following formula (1):

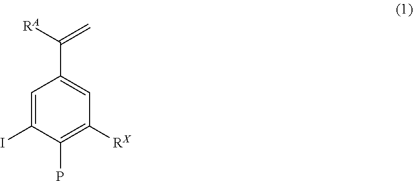

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and
l) an acylation step of subjecting the iodine-containing vinyl monomer to an acylation treatment;

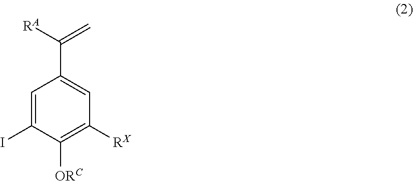

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and R^{C} is a substituted or unsubstituted acyl group having 1 to 30 carbon atoms.

In the present embodiment, the method for producing the iodine-containing alcohol compound represented by the following formula (1-1) may comprise:
c) a step of providing an iodine-containing ketone substrate having a general structure represented by the following formula (1-2):

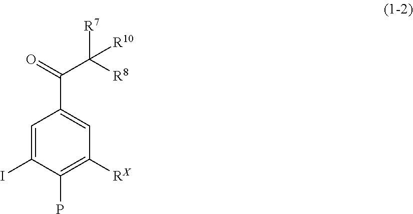

wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
d) a reduction step of subjecting the iodine-containing ketone substrate to a reduction treatment;

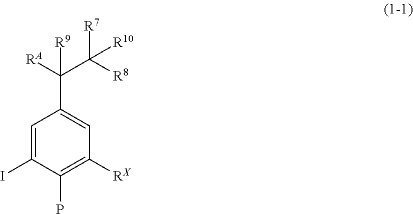

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group.

In the present embodiment, the method for producing the iodine-containing alcohol compound represented by the following formula (1-1) may comprise:
e) a step of providing an alcohol substrate having a general structure represented by the following formula (1-3):

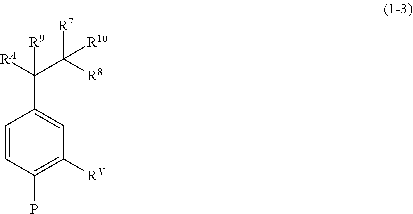

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
f) an iodine introduction step of introducing an iodine atom into the alcohol substrate;

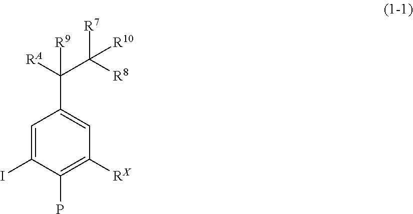

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group.

In the present embodiment, the method for producing the iodine-containing ketone compound represented by the following formula (1-2) may comprise:
g) a step of providing a ketone substrate having a general structure represented by the following formula (1-4):

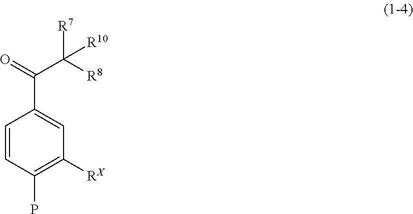

wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
h) an iodine introduction step of introducing an iodine atom into the ketone substrate;

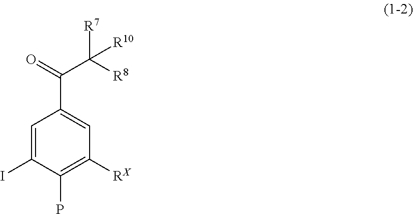

wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group.

In the present embodiment, the method for producing the iodine-containing vinyl monomer represented by the following formula (1) may comprise:
i) a step of providing a ketone substrate having a general structure represented by the following formula (1-4):

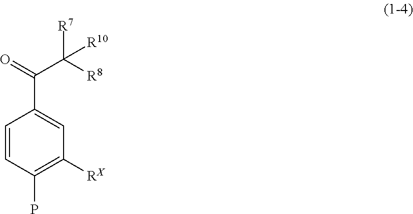

wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
j) a reduction step of subjecting the ketone substrate to a reduction treatment;

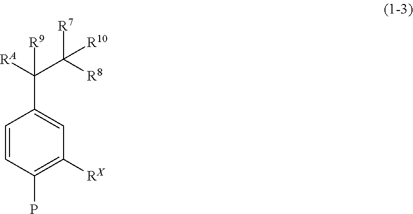

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group.

In the present embodiment, the method for producing the iodine-containing vinyl monomer represented by the following formula (1) may comprise:
a) a step of providing an iodine-containing aldehyde substrate or iodine-containing ketone substrate having a general structure represented by the following formula (1-5):

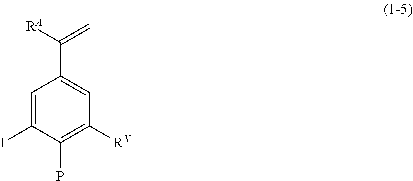

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and
b) a Wittig reaction step of forming an alkene from a carbonyl moiety of the iodine-containing aldehyde substrate or the iodine-containing ketone substrate by a Wittig reaction;

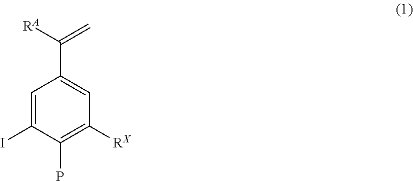

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group.

In the present embodiment, the method for producing the iodine-containing vinyl monomer represented by the following formula (1) may comprise:
a) a step of providing an iodine-containing aldehyde substrate or iodine-containing ketone substrate having a general structure represented by the following formula (1-5):

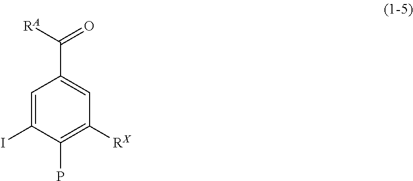

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group;
b) a malonic acid addition step of adding malonic acid to the iodine-containing aldehyde substrate or the iodine-containing ketone substrate;
c) a hydrolysis step of subjecting the iodine-containing aldehyde substrate or iodine-containing ketone substrate to which the malonic acid is added to a hydrolysis treatment to produce an iodine-containing carboxylic acid substrate; and
d) a decarbonation step of subjecting the iodine-containing carboxylic acid substrate to a decarbonation treatment;

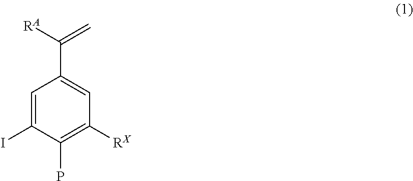

wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group.

The compound, polymer, composition, composition for film formation, pattern formation method, insulating film formation method, and method for producing a compound described above in the present embodiment may be applied to extreme ultraviolet applications.

The description of the first embodiment is as above.

### Second Embodiment

Hereinafter, the second embodiment of the present invention will be described. The second embodiment is an embodiment in the case where R^{X} in the compound (A) in the first embodiment is OR^{B}. The second embodiment is given in order to illustrate the present invention. The present invention is not limited to only the second embodiment.

### [Compound (A)]

The compound according to the second embodiment (hereinafter, also referred to as “compound (A)”) is represented by the following formula (1).

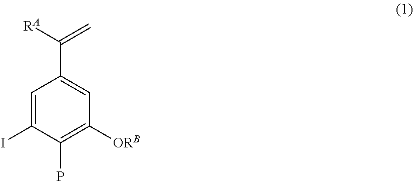

In the formula (1), R^{A} is a hydrogen atom, a methyl group, or a trifluoromethyl group; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group.

In the formula (1), R^{A} is a hydrogen atom, a methyl group, or a trifluoromethyl group. From the viewpoint of increasing the hydrophilicity to achieve high sensitivity, R^{A} is preferably a hydrogen atom or a methyl group.

From the viewpoint of increasing the absorption to EUV to achieve high sensitivity, R^{A} is preferably a trifluoromethyl group.

In the formula (1), R^{B} is a substituted or unsubstituted alkyl group having 1 to 30 carbon atoms. From the viewpoint of raw material availability for industrial production, or from the viewpoint of increasing the hydrophilicity to achieve high sensitivity, R^{B} is preferably an alkyl group having 1 to 4 carbon atoms, and more preferably an alkyl group having 1 to 2 carbon atoms.

In the second embodiment, unless otherwise defined, the term “substituted” means that one or more hydrogen atoms in a functional group are substituted with a substituent. Examples of the “substituent” include, but are not particularly limited to, a halogen atom, a hydroxyl group, a carboxyl group, a cyano group, a nitro group, a thiol group, a heterocyclic ring group, an alkyl group having 1 to 30 carbon atoms, an aryl group having 6 to 30 carbon atoms, an alkoxyl group having 1 to 30 carbon atoms, an alkenyl group having 2 to 30 carbon atoms, an alkynyl group having 2 to 30 carbon atoms, an acyl group having 1 to 30 carbon atoms, and an amino group having 0 to 30 carbon atoms.

The alkyl group may be any of a linear aliphatic hydrocarbon group, a branched aliphatic hydrocarbon group, and a cyclic aliphatic hydrocarbon group.

Examples of the alkyl group having 1 to 30 carbon atoms include, but are not limited to, a methyl group, an ethyl group, an n-propyl group, an i-propyl group, an n-butyl group, an i-butyl group, a t-butyl group, an n-pentyl group, an n-hexyl group, an n-dodecyl group, and a valeryl group.

Examples of the aryl group having 6 to 30 carbon atoms include, but are not limited to, a phenyl group, a naphthalene group, a biphenyl group, an anthracyl group, a pyrenyl group, and a perylene group.

Examples of the alkenyl group having 2 to 30 carbon atoms include, but are not limited to, an ethynyl group, a propenyl group, a butynyl group, and a pentynyl group.

Examples of the alkynyl group having 2 to 30 carbon atoms include, but are not limited to, an acetylene group and an ethynyl group.

Examples of an alkoxy group having 1 to 30 carbon atoms include, but are not limited to, a methoxy group, an ethoxy group, a propoxy group, a butoxy group, and pentoxy.

In the formula (1), each P is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of P optionally have a substituent.

Examples of P include at least one group selected from the group consisting of an alkoxy group [^{*3}—O—R²], an ester group [^{*3}—O—(C═O)—R² or ^{*3}—(C═O)—O—R²], an acetal group [^{*3}—O—(C(R²¹)₂)—O—R² (wherein each R²¹ is independently H or a hydrocarbon group having 1 to 10 carbon atoms; and R² and R²¹ are optionally bonded to form a cyclic ether)], a carboxyalkoxy group [^{*3}—O—R²²—(C═O)—O—R² (wherein R²² is a divalent hydrocarbon group having 1 to 10 carbon atoms)], and a carbonate ester group [^{*3}—O—(C═O)—O—R²]. The ester group is preferably a tertiary ester group, from the viewpoint of achieving high sensitivity. In the formula, ^{*3} is a site for binding with A.

Among them, P is preferably a hydroxy group, an ester group, an acetal group, a carbonate ester group, or a carboxyalkoxy group, more preferably an acetal group, a carbonate ester group, or a carboxyalkoxy group, and further preferably an acetal group or a carboxyalkoxy group, from the viewpoint of high sensitivity. From the viewpoint of producing a polymer having a stable quality by radical polymerization, an ester group, a carboxyalkoxy group, and a carbonate ester group are preferable. Further, from the viewpoint of increasing the difference in the dissolution rate between before and after exposure to increase the resolution, a tertiary ester group, an acetal group, a carbonate ester group, or a carboxyalkoxy group is preferable.

Each P is preferably independently a group represented by the following formula (P-1).

-L²-R² (P-1)

In the formula (P-1),

L² is a group which is cleaved by the action of an acid or a base. Examples of the group which is cleaved by the action of an acid or a base include at least one divalent linking group selected from the group consisting of an ester group [^{*1}—O—(C═O)—^{*2} or ^{*1}—(C═O)—O—^{*2}], an acetal group [^{*1}—O—(C(R²¹)₂)—O—^{*2} (each R²¹ is independently H or a hydrocarbon group having 1 to 10 carbon atoms)], a carboxyalkoxy group [^{*1}—O—R²²—(C═O)—O—^{*2} (R²² is a divalent hydrocarbon group having 1 to 10 carbon atoms)], and a carbonate ester group [^{*1}—O—(C═O)—O—^{*2}]. The ester group is preferably a tertiary ester group, from the viewpoint of achieving high sensitivity. In the formula, ^{*1} is a site for binding with a benzene ring, and ^{*2} is a site for binding with R². Among them, L² is preferably a tertiary ester group, an acetal group, a carbonate ester group, or a carboxyalkoxy group, more preferably an acetal group, a carbonate ester group, or a carboxyalkoxy group, and further preferably an acetal group or a carboxyalkoxy group, from the viewpoint of high sensitivity. From the viewpoint of producing a polymer having a stable quality by radical polymerization, an ester group, a carboxyalkoxy group, and a carbonate ester group are preferable.

As another effect, P is preferably a group represented by the formula (P-1) to control the polymerization properties of resin and the degree of polymerization in a desired range, when the compound (A) of the second embodiment is used as a polymerization unit of a copolymer. Since the compound (A) has a large influence on activity species in the polymer formation reaction due to having iodine and thus the desired control is difficult, variation of copolymer formation derived from the hydrophilic group and polymerization inhibition can be suppressed by having a group represented by the formula (P-1) in the hydrophilic group in the compound (A) as a protecting group.

R² is a linear, branched, or cyclic aliphatic group having 1 to 30 carbon atoms, an aromatic group having 6 to 30 carbon atoms, a linear, branched, or cyclic aliphatic group containing a heteroatom and having 1 to 30 carbon atoms, or a linear, branched, or cyclic aromatic group containing a heteroatom and having 1 to 30 carbon atoms, and the aliphatic group, the aromatic group, the aliphatic group containing a heteroatom, and the aromatic group containing a heteroatom of R² optionally further have or may not have a substituent. As the substituent here, the aforementioned substituents are used, but a linear, branched, or cyclic aliphatic group having 1 to 20 carbon atoms or an aromatic group having 6 to 20 carbon atoms are preferable. Among them, R² is preferably an aliphatic group. The aliphatic group in R² is preferably a branched or cyclic aliphatic group. The number of carbon atoms of the aliphatic group is preferably 1 or more and 20 or less, more preferably 3 or more and 10 or less, and further preferably 4 or more and 8 or less. Examples of the aliphatic group include, but are not particularly limited to, a methyl group, an isopropyl group, a sec-butyl group, a tert-butyl group, an isobutyl group, a cyclohexyl group, a methylcyclohexyl group, and an adamantyl group. Among them, a tert-butyl group, a cyclohexyl group, or an adamantyl group is preferable.

L² is preferably ^{*1}—(C═O)—O—^{*2} or a carboxyalkoxy group, because, when L² is cleaved by the action of an acid or a base, a carboxylic acid group is formed and the difference in the solubility and the difference in the dissolution rate between a cleaved portion and an uncleaved portion are increased in the development treatment, so that the resolution is improved and, in particular, residues at the pattern bottom in thin line patterns are suppressed.

Specific examples of P include the followings. Each P is independently a group represented by any of the following formulas.

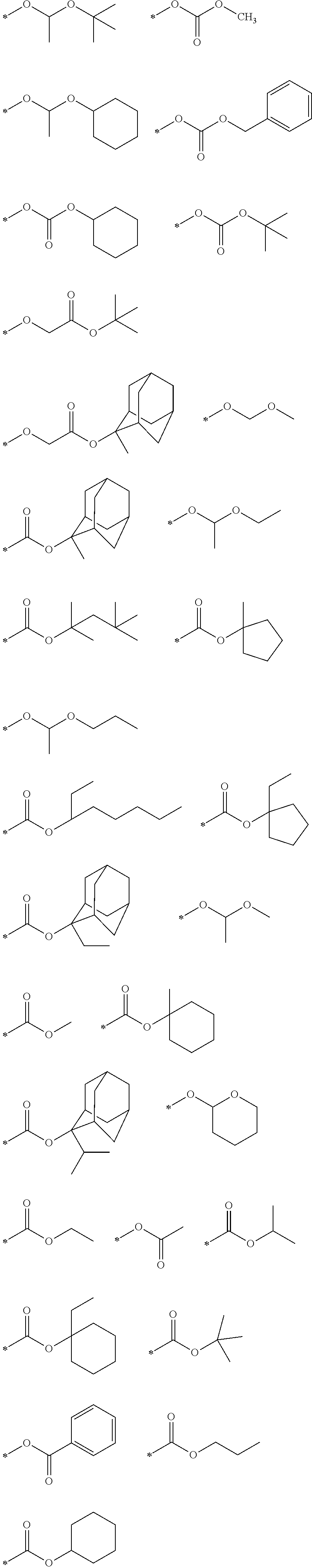

Examples of the alkoxy group that can be used as P include an alkoxy group having 1 or more carbon atoms, and from the viewpoint of the solubility of a resin after the compound is combined with other monomers to form the resin, an alkoxy group having 2 or more carbon atoms is preferable, and an alkoxy group having 3 or more carbon atoms or having a cyclic structure is preferable.

Specific examples of the alkoxy group that can be used as P include, but are not limited to, the followings.

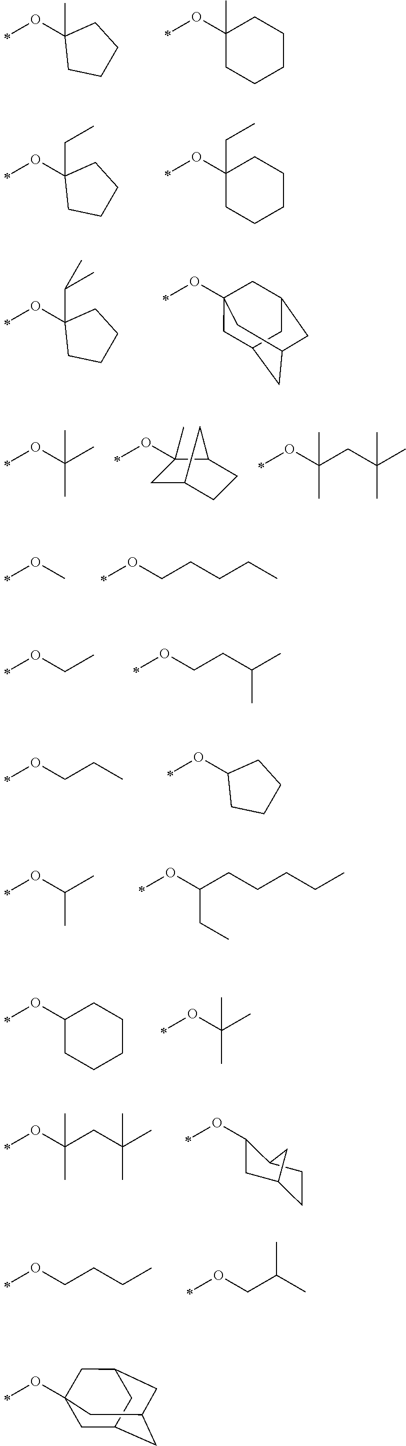

As the amino group and amide group that can be used as P, a primary amino group, a secondary amino group, a tertiary amino group, a group having a quaternary ammonium salt structure, an amide having a substituent, or the like can be arbitrarily used. Specific examples of the amino group or amide group that can be used include, but are not limited to, the followings.

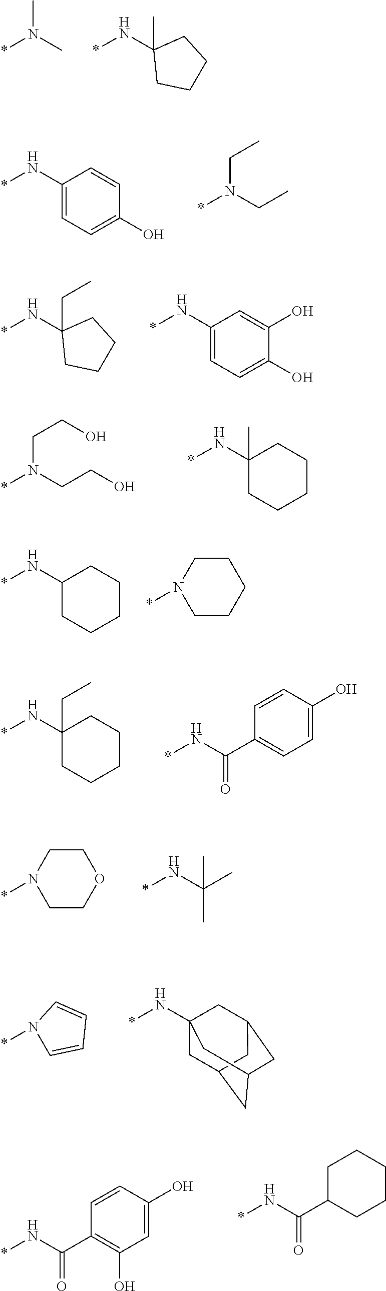

It is presumed that the compound (A) according to the second embodiment contains an iodine group and OR^{B} in the molecule, and thus, when a polymer using the compound (A) is applied to a resist composition and pattern formation is carried out by lithography processing comprising film formation, exposure, and development, the iodine group and the OR^{B} group improve the solubility in a developing solution, allowing both development defects such as development residues, roughness, and bridges to be reduced, and other lithography performances such as sensitivity and resolution to be achieved, and consequently allowing the pattern quality in finer pattern formation to be improved.

As a result, it is considered to be effective for the improvement of the pattern quality of a pattern that has a problem of the defect due to the solubility in a developing solution, such as, in particular, a line and space pattern.

Examples of the compound (A) according to the second embodiment include compounds having the structures given below.

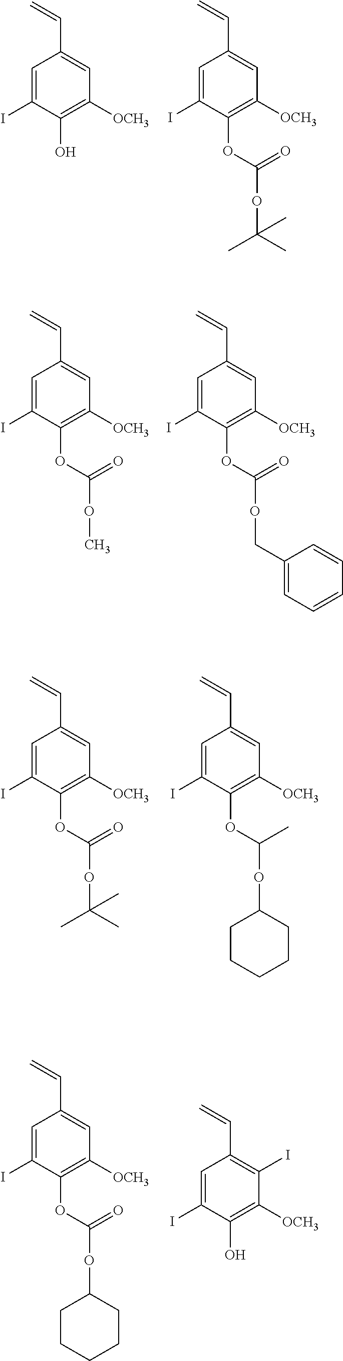

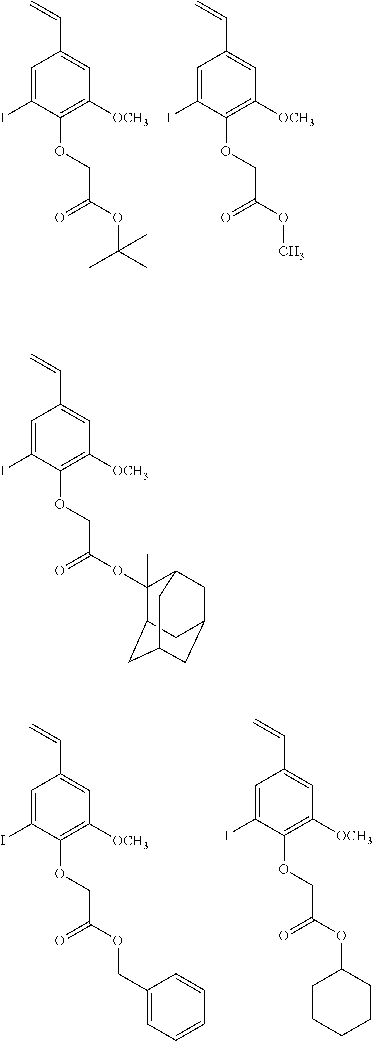 [SMILES: C=Cc1cc(I)c(OCC(=O)OC(C)(C)C)c(OC)c1 ; C=Cc1cc(I)c(OCC(=O)OC)c(OC)c1 ; C=Cc1cc(I)c(OCC(=O)OC2(C)C3CC4CC(C3)CC2C4)c(OC)c1 ; C=Cc1cc(I)c(OCC(=O)OCc2ccccc2)c(OC)c1 ; C=Cc1cc(I)c(OCC(=O)OC2CCCCC2)c(OC)c1]

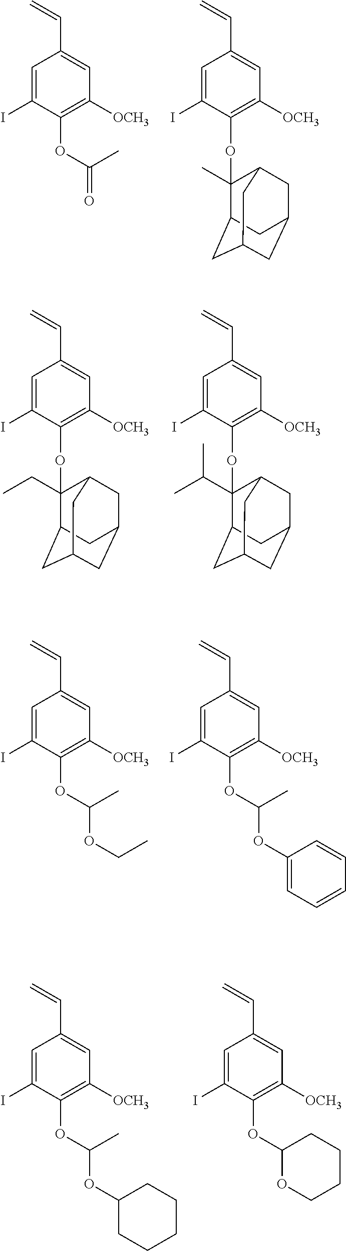

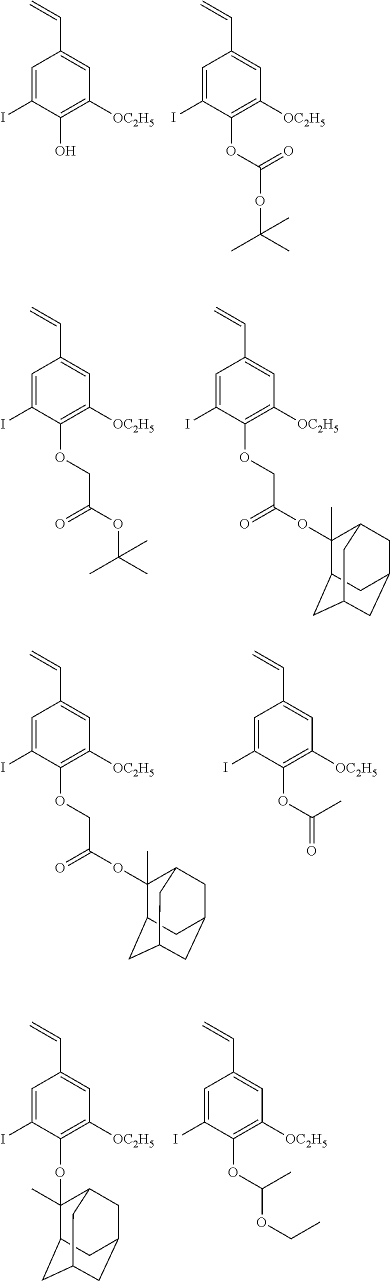

The compound (A) described above is preferably used in combination with a compound represented by the following formula (1A). That is, the composition according to the second embodiment preferably contains the compound (A) and the compound represented by the formula (1A).

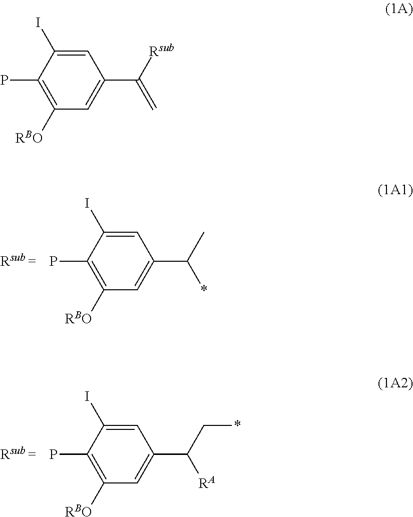

(In the formula (1A), the formula (1A1), and the formula (1A2), R^{A}, R^{B}, and P have the same meanings as in the formula (1); R^{sub} represents the formula (1A1) or formula (1A2); and * is a site for binding with an adjacent constitutional unit.)

The composition is preferably prepared such that the compound represented by the formula (1A) is contained within a range of 1 ppm by mass or more and 10% by mass or less, more preferably within a range of 1 ppm by mass or more and 5% by mass or less, further preferably within a range of 1 ppm by mass or more and 3% by mass or less, and particularly preferably within a range of 1 ppm by mass or more and 1% by mass or less, based on the total compound (A), from the viewpoint of improving the exposure sensitivity and reducing the residue defect. In the resin form after the formation of a resin made of starting materials including the composition thus prepared, the presence of a moiety containing iodine and a moiety consisting of P at a high density in the proximity area becomes the starting point for improving the exposure sensitivity. Further, a local increase in the solubility of the resin leads to a reduction in the residue defect after development in a lithography process.

Examples of the compound (1A) according to the second embodiment include compounds having the structures given below.

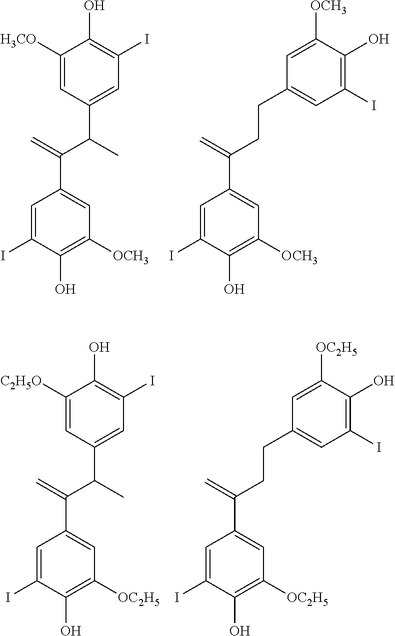

The compound (A) described above is preferably used in combination with a compound represented by the following formula (1B). That is, the composition according to the second embodiment preferably contains the compound (A) and the compound represented by the formula (1B).

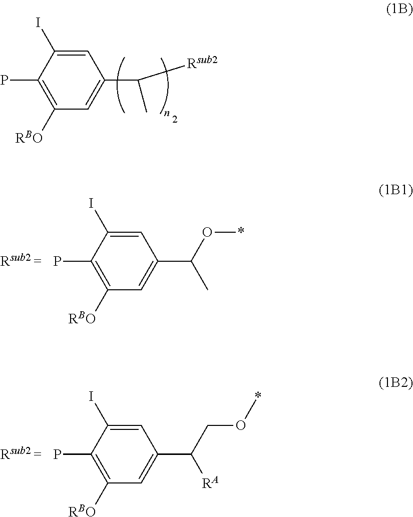

(In the formula (1B), the formula (1B1), or the formula (1B2), R^{A}, R^{B}, and P have the same meanings as in the formula (1); n₂ is an integer of 0 to 4; R^{sub2} represents the formula (1B1) or the formula (1B2); and * is a site for binding with an adjacent constitutional unit.)

The composition is preferably prepared such that the compound represented by the formula (1B) is contained within a range of 1 ppm by mass or more and 10% by mass or less, more preferably within a range of 1 ppm by mass or more and 5% by mass or less, further preferably within a range of 1 ppm by mass or more and 3% by mass or less, and particularly preferably within a range of 1 ppm by mass or more and 1% by mass or less, based on the total compound (A), from the viewpoint of improving the exposure sensitivity and reducing the residue defect. In the resin form after the formation of a resin made of starting materials including the composition thus prepared, the presence of a moiety containing iodine and a moiety consisting of P at a high density in the proximity area becomes the starting point for improving the exposure sensitivity. Further, a local increase in the solubility of the resin leads to a reduction in the residue defect after development in a lithography process.

Examples of the compound (1B) according to the second embodiment include compounds having the structures given below.

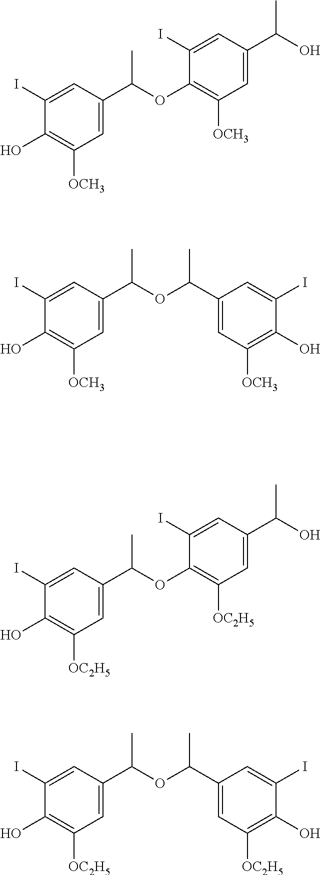 [SMILES: COc1cc(C(C)Oc2c(I)cc(C(C)O)cc2OC)cc(I)c1O ; COc1cc(C(C)OC(C)c2cc(I)c(O)c(OC)c2)cc(I)c1O ; CCOc1cc(C(C)Oc2c(I)cc(C(C)O)cc2OCC)cc(I)c1O ; CCOc1cc(C(C)OC(C)c2cc(I)c(O)c(OCC)c2)cc(I)c1O]

The compound (A) described above is preferably used in combination with a compound represented by the following formula (1C). That is, the composition according to the second embodiment preferably contains the compound (A) and the compound represented by the formula (1C).

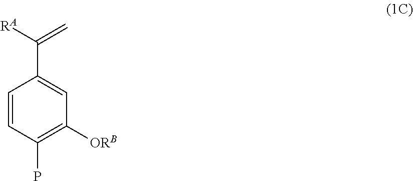

In the formula (1C), R^{A}, R^{B}, and P have the same meanings as in the formula (1). Provided that neither R^{B} nor P contains I.

The composition preferably contains the compound represented by the formula (1C) within a range of 1 ppm by mass or more and 10% by mass or less, more preferably within a range of 1 ppm by mass or more and 5% by mass or less, further preferably within a range of 1 ppm by mass or more and 3% by mass or less, and particularly preferably within a range of 1 ppm by mass or more and 1% by mass or less, based on the total compound (A), from the viewpoint of stability and reducing the residue defect.

In the composition thus prepared, its stability tends to increase. The reason is not clear, but can be presumed that the equilibrium reaction of iodine atoms occurs between the compound (A) containing iodine and the compound (1C) containing no iodine and thus the composition is stabilized.

In this case, the composition preferably contains, as the compound (1C), a compound having a structure in which iodine atoms are eliminated from a compound exemplified as the compound (A) mentioned above, in combination.

In the composition thus prepared, its stability increases, leading to not only an increase in the storage stability, but also formation of a resin having stable properties, providing stable resist performance, and further, a reduction in the residue defect after development in a lithography process.

Examples of the method for using the compound represented by the formula (1C) in a range of 1 ppm by mass or more and 10% by mass or less based on the total compound (A) in the composition containing the compound (A) include, but are not particularly limited to, a method for adding the compound (1C) to the compound (A) and a method for producing the compound (1C) as a by-product during production of the compound (A).

Examples of the compound (1C) according to the second embodiment include compounds having the structures given below.

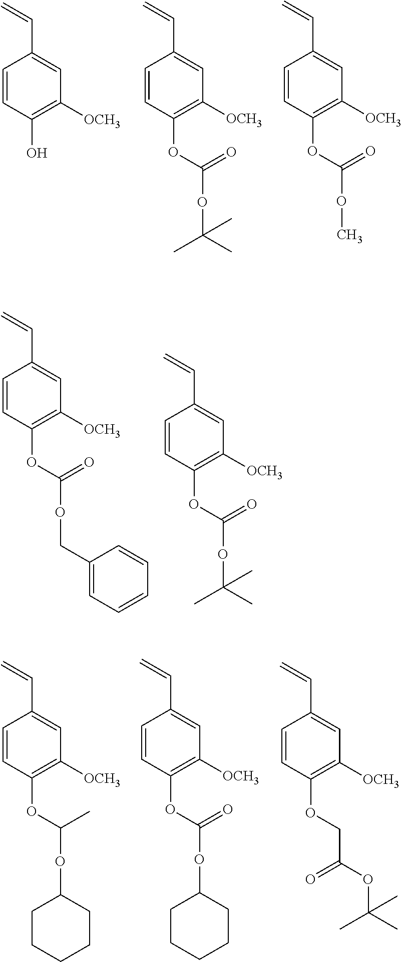

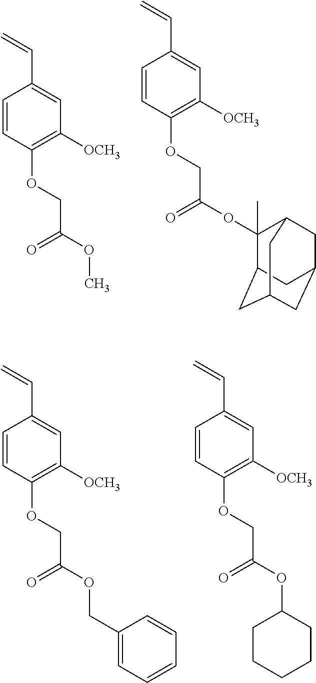 [SMILES: C=Cc1ccc(OCC(=O)OC)c(OC)c1 ; C=Cc1ccc(OCC(=O)OC2(C)C3CC4CC(C3)CC2C4)c(OC)c1 ; C=Cc1ccc(OCC(=O)OCc2ccccc2)c(OC)c1 ; C=Cc1ccc(OCC(=O)OC2CCCCC2)c(OC)c1]

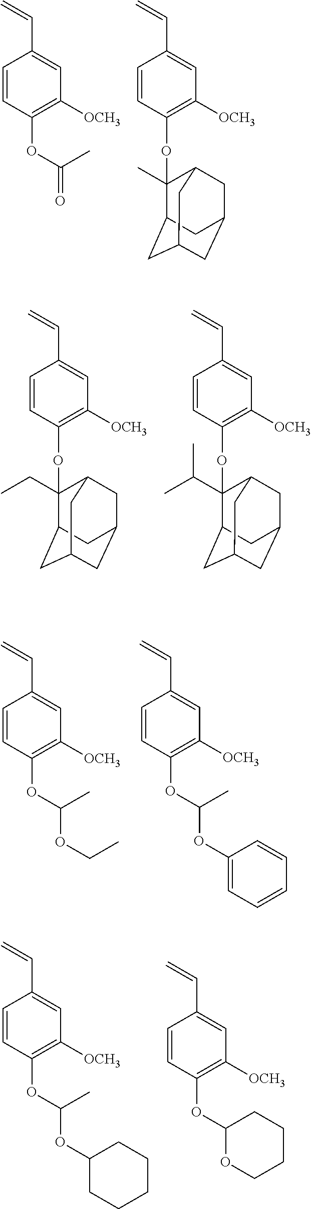

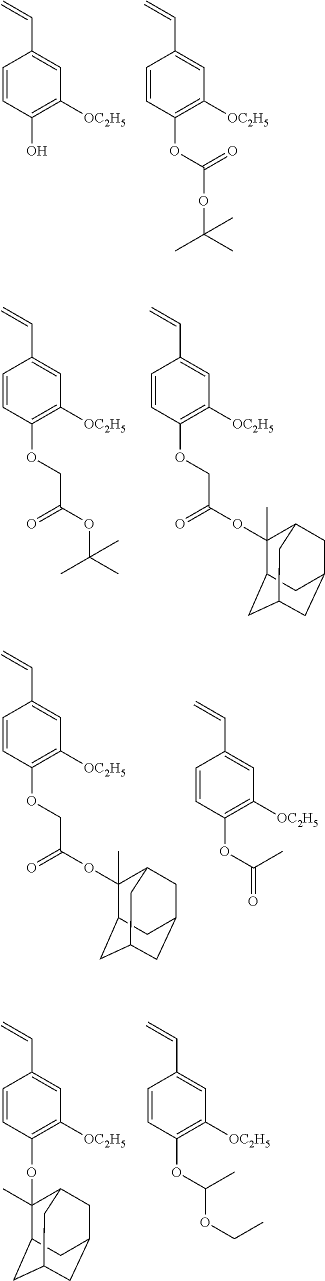

### [Method for Producing Compound (A)]

The compound represented by the formula (1) can be produced by various known synthetic methods.

As an example of the synthetic method for the compound represented by the formula (1) in which P is a hydroxy group, the synthesis can be carried out by introducing a halogen group, I, F, Cl, or Br, into a hydroxy group-containing aromatic aldehyde derivative, and then converting the aldehyde group into a vinyl group, but it is not particularly limited thereto. As another example of the synthetic method, a method for reacting iodine chloride in an organic solvent by carrying out iodination reaction on a hydroxybenzaldehyde derivative (e.g., see Japanese Patent Laid-Open No. 2012-180326), a method for dropping iodine in an aqueous alkaline solution of phenol under alkaline conditions in the presence of βcyclodextrin (Japanese Patent Laid-Open No. 63-101342, Japanese Patent Laid-Open No. 2003-64012), or the like can be arbitrarily selected.

In the second embodiment, the iodination reaction through iodine chloride in an organic solvent is preferably used. The compound (A) of the second embodiment can be synthesized by converting the aldehyde moiety of the synthesized iodine-introduced hydroxybenzaldehyde derivative into a vinyl group. As the method for converting the aldehyde moiety into a vinyl group, a Wittig reaction (e.g., the methods described in Synthetic Communications; Vol. 22; nb4; 1992 p 513, Synthesis; Vol. 49; nb. 23; 2017; p 5217) can be arbitrarily used.

That is, the method for producing the compound (A) represented by the formula (1) (iodine-containing vinyl monomer) comprises:
a) a step of providing an iodine-containing aldehyde substrate or iodine-containing ketone substrate having a general structure represented

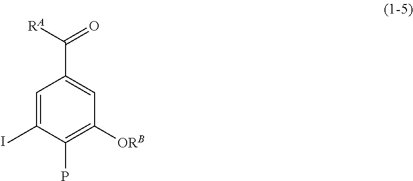

wherein R^{A} is a hydrogen atom, a methyl group, or a trifluoromethyl group; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and
b) a Wittig reaction step of forming an alkene from a carbonyl moiety of the iodine-containing aldehyde substrate or the iodine-containing ketone substrate by a Wittig reaction.

Examples of the iodine-containing aldehyde substrate or iodine-containing ketone substrate having a general structure represented by the formula (1-5) include 4-hydroxy-3-iodo-5-methoxybenzaldehyde and 3-ethoxy4-hydroxy-5-iodo-benzaldehyde.

The Wittig reaction step is a step of forming alkene by a Witting reaction, and is a step of forming alkene from a carbonyl moiety having aldehyde or ketone using phosphorus ylide, although it is not limited. As the phosphorus ylide, for example, triphenyl alkyl phosphine bromide such as triphenyl methyl phosphine bromide that can form a stable phosphorus ylide can be used. Also, as the phosphorus ylide, a phosphonium salt can be reacted with a base to form a phosphorus ylide in the reaction system, which can be used in the aforementioned reaction. As the base, a conventionally known one can be used, and for example, an alkali metal salt of alkoxide can be arbitrarily used.

As another method for converting the aldehyde moiety into a vinyl group, a method for reacting malonic acid in the presence of a base (e.g., the methods described in Tetrahedron; Vol. 46; nb. 40; 2005; p 6893, Tetrahedron; Vol. 63; nb. 4; 2007; p 900, US2004/118673), or the like can be arbitrarily used.

In the second embodiment, the method for producing the compound (A) represented by the formula (1) (iodine-containing vinyl monomer) comprises:
a) a step of providing an iodine-containing aldehyde substrate or iodine-containing ketone substrate having a general structure represented by the formula (1-5);
b) a malonic acid addition step of adding malonic acid to the iodine-containing aldehyde substrate or the iodine-containing ketone substrate;
c) a hydrolysis step of subjecting the iodine-containing aldehyde substrate or iodine-containing ketone substrate to which the malonic acid is added to a hydrolysis treatment to produce an iodine-containing carboxylic acid substrate; and
d) a decarbonation step of subjecting the iodine-containing carboxylic acid substrate subjected to the hydrolysis treatment to a decarbonation treatment.

The malonic acid addition step in the second embodiment is a step of forming a malonic acid derivative, and is a reaction of aldehyde with malonic acid, a malonic acid ester, or a malonic acid anhydride, although it is not limited.

The hydrolysis step in the second embodiment is a step of forming a carboxylic acid substrate by hydrolysis, and is a reaction of hydrolyzing an ester by an action of an acid or water, although it is not limited.

The decarbonation step in the second embodiment is a step of carrying out decarboxylation of the carboxylic acid substrate to obtain a vinyl monomer, and is preferably carried out at a low temperature of 100° C. or less, and more preferably uses a fluoride-based catalyst, although it is not limited.

As the synthetic method of the compound (A) of the second embodiment, for example, the method described in the above references can be arbitrarily used, but is not limited thereto.

As an example of the synthetic method for the compound represented by the formula (1) in which P is an alkoxy group, an ester group, an acetal group, a carboxyalkoxy group, or a carbonate ester group, the compound can be obtained by reacting the compound represented by the formula (1) in which P is a hydroxy group with, for example, an active carboxylic acid derivative compound such as acid chloride, acid anhydride, or dicarbonate, an alkyl halide, a vinyl alkyl ether, dihydropyran, or a halocarboxylic acid alkyl ester, but it is not particularly limited thereto.

For example, the compound represented by the formula (1) in which P is a hydroxy group is dissolved or suspended in an aprotic solvent such as acetone, tetrahydrofuran, or propylene glycol monomethyl ether acetate. Subsequently, a vinyl alkyl ether such as ethyl vinyl ether, or dihydropyran is added to the solution or the suspension, and the mixture is reacted at 20 to 60° C. at normal pressure for 6 to 72 hours in the presence of an acid catalyst such as pyridinium p-toluenesulfonate. The reaction solution is neutralized with an alkali compound, added to distilled water to precipitate a white solid, and then, the separated white solid is washed with distilled water and dried, thereby allowing the compound represented by the formula (1) in which P is an alkoxy group, an ester group, an acetal group, a carboxyalkoxy group, or a carbonate ester group to be obtained.

Moreover, the compound represented by the formula (1) in which P is a hydroxy group is dissolved or suspended in an aprotic solvent such as acetone, THF, or propylene glycol monomethyl ether acetate. Subsequently, an alkyl halide such as ethyl chloromethyl ether or a halocarboxylic acid alkyl ester such as methyladamantyl bromoacetate is added to the solution or the suspension, and the mixture is reacted at 20 to 110° C. at normal pressure for 6 to 72 hours in the presence of an alkali catalyst such as potassium carbonate. The reaction solution is neutralized with an acid such as hydrochloric acid, added to distilled water to precipitate a white solid, and then, the separated white solid is washed with distilled water and dried, thereby allowing the compound represented by the formula (1) in which P is an alkoxy group, an ester group, an acetal group, a carboxyalkoxy group, or a carbonate ester group to be obtained.

The synthetic method of the compound (A) of the second embodiment more preferably comprises the synthetic methods given below, from the viewpoint of suppressing the yield and the amount of waste.

### [Iodine-Containing Alcohol Substrate]

The iodine-containing alcohol substrate used in the second embodiment may be, for example, an iodine-containing alcohol substrate having a general structure represented by the following formula (1-1).

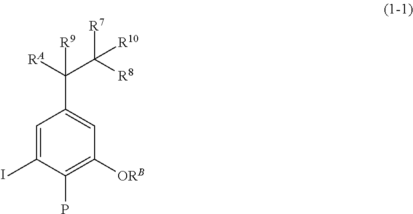

(In the formula (1-1), R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently hydrogen, a hydroxy group, a methoxy group, a halogen, or a cyano group, provided that one of R⁷ to R¹⁰ is a hydroxy group or a methoxy group.)

Examples of suitable iodine-containing alcohol substrates include, but are not limited to, 1-(4-hydroxy-3-methoxy-5-iodophenyl)ethanol, 1-(3-ethoxy-4-hydroxy-5-iodophenyl)ethanol, 4-(1-hydroxyethyl)-3-methoxy-5-iodophenol, and 3-ethoxy-4-(1-hydroxyethyl)-5-iodophenol. At least one iodine atom is introduced, and two or more iodine atoms are preferably introduced.

These iodine-containing alcohol substrates can be obtained by many methods, but is preferably obtained by the method described later, from the viewpoint of the availability and yield of the raw material.

The method for producing the iodine-containing vinyl monomer represented by the formula (1) comprises:
a) a step of providing an iodine-containing alcohol substrate having a general structure represented by the formula (1-1); and
b) a dehydration step of subjecting the iodine-containing alcohol substrate to a dehydration treatment.

As the organic solvent, a wide variety of organic solvents including a polar aprotic organic solvent and protic polar organic solvent are used. A single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used, and a polar aprotic solvent or a mixture thereof is preferable. The solvent is effective, but is not an essential component. Examples of suitable polar aprotic solvents include, but are not limited to, an alcohol solvent such as methanol and ethanol, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, hexamethylphosphorous triamide; and dimethylsulfoxide. Dimethylsulfoxide is preferable. Examples of suitable protic polar solvents include, but are not limited to, di(propylene glycol) methyl ether, di(ethylene glycol) methyl ether, 2-butoxyethanol, ethylene glycol, 2-methoxyethanol, propyleneglycol methylether, n-hexanol, and n-butanol.

The amount of the solvent used can be arbitrarily set according to, for example, the substrate, catalyst, and reaction conditions to be used, without particular limitation. In general, it is suitably 0 to 10000 parts by mass, and from the viewpoint of the yield, preferably 100 to 2000 parts by mass, based on 100 parts by mass of the reaction raw materials.

The dehydration step is conducted by using, for example, a catalyst. As the catalyst, a wide variety of dehydration catalysts which function under the reaction conditions of the second embodiment are used. As the dehydration catalyst, an acid catalyst is preferable. Examples of suitable acid catalysts include, but are not limited to, an inorganic acid such as hydrochloric acid, sulfuric acid, phosphoric acid, hydrobromic acid, and hydrofluoric acid; an organic acid such as oxalic acid, malonic acid, succinic acid, adipic acid, sebacic acid, citric acid, fumaric acid, maleic acid, formic acid, p-toluenesulfonic acid, methanesulfonic acid, trifluoroacetic acid, dichloroacetic acid, trichloroacetic acid, trifluoromethanesulfonic acid, benzenesulfonic acid, naphthalenesulfonic acid, and naphthalenedisulfonic acid; a Lewis acid such as zinc chloride, aluminum chloride, iron chloride, and boron trifluoride; and a solid acid such as tungstosilicic acid, tungstophosphoric acid, silicomolybdic acid, and phosphomolybdic acid. These acid catalysts are used alone as one kind or in combination of two or more kinds. Among them, organic acids and solid acids are preferable from the viewpoint of production, and it is preferable to use hydrochloric acid or sulfuric acid from the viewpoint of production such as easy availability and handleability.

The amount of the catalyst used can be arbitrarily set according to, for example, the substrate, catalyst, and reaction conditions to be used, without particular limitation. In general, it is suitably 0.0001 to 100 parts by mass, and from the viewpoint of the yield, preferably 0.001 to 10 parts by mass, based on 100 parts by mass of the reaction raw materials.

As the polymerization inhibitor, a wide variety of polymerization inhibitors which function under the reaction conditions of the second embodiment are used. The polymerization inhibitor is effective, but is not an essential component. Examples of suitable polymerization inhibitors include, but are not limited to, hydroquinone, hydroquinone monomethyl ether, 4-tert-butylcatechol, phenothiazine, N-oxyl (nitroxide) inhibitor, for example, Prostab® 5415 (bis(1-oxyl-2,2,6,6-tetramethylpiperidin-4-yl)sebacate which is commercially available from Ciba Specialty Chemicals, Tarrytown, NY, CAS #2516-92-9), 4-hydroxy-TEMPO (4-hydroxy-2,2,6,6-tetramethylpiperidin-1-yloxy, which is commercially available from TCI, CAS #2226-96-2), Uvinul® 4040P (1,6-hexamethylene-bis(N-formyl-N-(1-oxyl-2,2,6,6-tetramethylpiperidin-4-yl)amine which is commercially available from BASF Corp., Worcester, MA), ammonium-N-nitrosophenylhydroxylamine (Q1300 which is commercially available from FUJIFILM Wako Pure Chemical Corporation), and N-nitrosophenylhydroxylamine aluminium salt (Q1301 which is commercially available from FUJIFILM Wako Pure Chemical Corporation).

The amount of the polymerization inhibitor used can be arbitrarily set according to, for example, the substrate, catalyst, and reaction conditions to be used, without particular limitation. In general, it is suitably 0.0001 to 100 parts by mass, and from the viewpoint of the yield, it is preferably 0.001 to 10 parts by mass, based on 100 parts by mass of the reaction raw materials.

As the polymerization retarder, a wide variety of polymerization retarders which function under the reaction conditions of the second embodiment are used. The polymerization retarder is effective, but is not an essential component. A polymerization retardant is effectively used by combining with the polymerization inhibitor. The polymerization retardant is well-known in the art as a compound that can delay the polymerization reaction, but cannot prevent all polymerization from occurring. A typical retardant is an aromatic nitro compound such as dinitro-ortho-cresol (DNOC) and dinitro butyl phenol (DNBP). The method for producing the polymerization retardant is common and well-known in the art (e.g., see U.S. Pat. No. 6,339,177; Park et al., Polymer (Korea) (1988), 12(8), 710-19), and its use in the control of styrene polymerization is well documented (e.g., see Bushby et al., Polymer (1998), 39(22), 5567-5571).

The amount of the polymerization retarder used can be arbitrarily set according to, for example, the substrate, catalyst, and reaction conditions to be used, without particular limitation. In general, it is suitably 0.0001 to 100 parts by mass, and from the viewpoint of the yield, preferably 0.001 to 10 parts by mass, based on 100 parts by mass of the reaction raw materials.

### Reaction Conditions

The reaction mixture is formed by adding the iodine-containing alcohol substrate having the formula (1-1), the catalyst, and the organic solvent to a reactor. Any suitable reactor is used.

The reaction may be carried out by arbitrarily selecting a known method such as a batch method, a semi-batch method, or a continuous method.

The reaction temperature is not particularly limited. The preferred range varies according to the concentration of the substrate, the stability of the formed product, the selection of the catalyst, and the desired yield. In general, a temperature of 0° C. to 200° C. is suitable, and from the viewpoint of the yield, a temperature of 0° C. to 100° C. is preferable.

In the reaction using 1-(4-hydroxy-3-iodo-5-methoxyphenyl)ethanol as the iodine-containing alcohol substrate, the temperature range is preferably 0° C. to 100° C.

The reaction pressure is not particularly limited. The preferred range varies according to the concentration of the substrate, the stability of the formed product, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation.

In the reaction using 1-(4-hydroxy-3-iodo-5-methoxyphenyl)ethanol as the iodine-containing alcohol substrate, the preferred reaction pressure is reduced pressure to normal pressure, and reduced pressure is preferable.

The reaction time is not particularly limited. The preferred range varies according to the concentration of the substrate, the stability of the formed product, the selection of the catalyst, and the desired yield. However, the majority of the reactions are carried out for less than 6 hours, and the reaction time is typically 15 minutes to 600 minutes.

In the reaction using 1-(4-hydroxy-3-iodo-5-methoxyphenyl)ethanol as the substrate, the preferred reaction time range is 15 minutes to 600 minutes.

Isolation and purification can be conducted by using a conventionally known suitable method after terminating the reaction. For example, the reaction mixture is poured in ice water and extracted in an organic solvent such as ethyl acetate or diethyl ether. Then, the product is recovered by removing the solvent using evaporation at reduced pressure. The product can be isolated and purified as the desired high purity monomer by a separation and purification method by filtration, concentration, distillation, extraction, crystallization, recrystallization, column chromatography, activated carbon, or the like, which are well-known purification methods in the art, or a combined method thereof.

### [Production Method (I) of Iodine-Containing Alcohol Substrate Represented by Formula (1-1)]

The iodine-containing ketone substrate used in the production of the iodine-containing alcohol substrate represented by the formula (1-1) is, for example, an iodine-containing ketone substrate having a general structure represented by the formula (1-2).

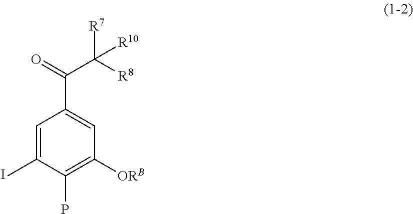

(In the formula (1-2), R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group.)

Examples of suitable iodine-containing ketone substrates include, but are not limited to, 4-hydroxy-3-iodo-5-methoxyphenylmethylketone and 5-ethoxy-4-hydroxy-3-iodophenylmethylketone.

These iodine-containing ketone substrates can be obtained by many methods, but is preferably obtained by the method described later, from the viewpoint of the availability and yield of the raw material.

The method for producing the iodine-containing alcohol substrate having a general structure represented by the formula (1-1) comprises:
c) a step of providing an iodine-containing ketone substrate having a general structure represented by the formula (1-2); and
d) a reduction step of subjecting the iodine-containing ketone substrate to a reduction treatment.

The method for producing the iodine-containing vinyl monomer having a general structure represented by the formula (1) may comprise the method for producing the iodine-containing alcohol substrate having a general structure represented by the formula (1-1). That is, the method for producing the iodine-containing vinyl monomer having a general structure represented by the formula (1) may comprise:
c) a step of providing an iodine-containing ketone substrate having a general structure represented by the formula (1-2); and
d) a reduction step of subjecting the iodine-containing ketone substrate to a reduction treatment.

As the organic solvent, a wide variety of organic solvents including a polar aprotic organic solvent and protic polar organic solvent are used. A single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used, and a polar aprotic solvent or a mixture thereof is preferable. The solvent is effective, but is not an essential component. Examples of suitable polar aprotic solvents include, but are not limited to, an alcohol solvent such as methanol and ethanol, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, hexamethylphosphorous triamide; and dimethylsulfoxide. Dimethylsulfoxide is preferable. Examples of suitable protic polar solvents include, but are not limited to, di(propylene glycol) methyl ether, di(ethylene glycol) methyl ether, 2-butoxyethanol, ethylene glycol, 2-methoxyethanol, propyleneglycol methylether, n-hexanol, and n-butanol.

The amount of the solvent used can be arbitrarily set according to, for example, the substrate, catalyst, and reaction conditions to be used, without particular limitation. In general, it is suitably 0 to 10000 parts by mass, and from the viewpoint of the yield, preferably 100 to 2000 parts by mass, based on 100 parts by mass of the reaction raw materials.

The reduction step is conducted by using, for example, a reducing agent. As the reducing agent, a wide variety of reducing agents which function under the reaction conditions of the second embodiment are used. Examples of suitable reducing agents include, but are not limited to, a metal hydride and a metal hydride complex compound, such as borane dimethylsulfide, diisobutylaluminum hydride, sodium borohydride, lithium borohydride, potassium borohydride, zinc borohydride, lithium tri-s-butylborohydride, potassium tri-s-butylborohydride, lithium triethylborohydride, lithium aluminum hydride, lithium tri-t-butoxyaluminum hydride, and sodium bis(methoxyethoxy)aluminum hydride.

The amount of the catalyst used can be arbitrarily set according to, for example, the substrate, catalyst, and reaction conditions to be used, without particular limitation. In general, it is suitably 1 to 500 parts by mass, and from the viewpoint of the yield, preferably 10 to 200 parts by mass, based on 100 parts by mass of the reaction raw materials.

As a quenching agent, a wide variety of quenching agents which function under the reaction conditions of the second embodiment are used. The quenching agent has a function of deactivating the reducing agent. The quenching agent is effective, but is not an essential component. Examples of suitable quenching agents include, but are not limited to, ethanol, aqueous ammonium chloride solution, water, hydrochloric acid, and sulfuric acid.

The amount of the quenching agent used can be arbitrarily set according to the amount of the reducing agent to be used, without particular limitation. In general, it is suitably 1 to 500 parts by mass, and from the viewpoint of the yield, it is preferably 50 to 200 parts by mass, based on 100 parts by mass of the reducing agent.

### Reaction Conditions

The reaction mixture is formed by adding the iodine-containing ketone substrate having the formula (1-2), the catalyst, and the organic solvent to a reactor. Any suitable reactor is used.

The reaction may be carried out by arbitrarily selecting a known method such as a batch method, a semi-batch method, or a continuous method.

The reaction temperature is not particularly limited. The preferred range varies according to the concentration of the iodine-containing ketone substrate, the stability of the formed product, the selection of the catalyst, and the desired yield. In general, a temperature of 0° C. to 200° C. is suitable, and from the viewpoint of the yield, a temperature of 0° C. to 100° C. is preferable.

In the reaction using 4′-hydroxy-3′-iodo-5′-methoxyacetophenone as the iodine-containing ketone substrate, the temperature range is preferably 0° C. to 100° C.

The reaction pressure is not particularly limited. The preferred range varies according to the concentration of the iodine-containing ketone substrate, the stability of the formed product, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation.

In the reaction using 4′-hydroxy-3′-iodo-5′-methoxyacetophenone as the iodine-containing ketone substrate, the preferred reaction pressure is reduced pressure to normal pressure, and reduced pressure is preferable.

The reaction time is not particularly limited. The preferred range varies according to the concentration of the iodine-containing ketone substrate, the stability of the formed product, the selection of the catalyst, and the desired yield. However, the majority of the reactions are carried out for less than 6 hours, and the reaction time is typically 15 minutes to 600 minutes.

In the reaction using 4′-hydroxy-3′-iodo-5′-methoxyacetophenone as the iodine-containing ketone substrate, the preferred reaction time range is 15 minutes to 600 minutes.

Isolation and purification can be conducted by using a conventionally known suitable method after terminating the reaction. For example, the reaction mixture is poured in ice water and extracted in an organic solvent such as ethyl acetate or diethyl ether. Then, the product is recovered by removing the solvent using evaporation at reduced pressure. The product can be isolated and purified as the desired high purity monomer by a separation and purification method by filtration, concentration, distillation, extraction, crystallization, recrystallization, column chromatography, activated carbon, or the like, which are well-known purification methods in the art, or a combined method thereof.

### [Production Method (II) of Iodine-Containing Alcohol Substrate Represented by Formula (1-1)]

The alcohol substrate used in the iodine-containing alcohol substrate represented by the formula (1-1) is, for example, an alcohol substrate having a general structure represented by the formula (1-3).

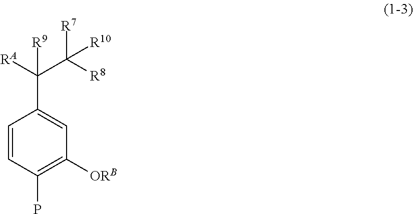

(In the formula (1-3), R^{A} is a hydrogen atom, a methyl group, or a trifluoromethyl group; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group.)

Examples of suitable alcohol substrates include, but are not limited to, 1-(4-hydroxy-3-methoxyphenyl)ethanol, 1-(3-ethoxy-4-hydroxyphenyl)ethanol, 4-(1-hydroxyethyl)-3-methoxyphenol, and 3-ethoxy-4-(1-hydroxyethyl)phenol.

These alcohol substrates can be obtained by many methods, but is preferably obtained by the method described later, from the viewpoint of the availability and yield of the raw material.

The method for producing the iodine-containing alcohol substrate having a general structure represented by the formula (1-1) comprises:
e) a step of providing an alcohol substrate having a general structure represented by the formula (1-3); and
f) an iodine introduction step of introducing an iodine atom into the alcohol substrate.

The iodine introduction step in the second embodiment is not particularly limited, but a method for reacting an iodinating agent in a solvent (e.g., Japanese Patent Laid-Open No. 2012-180326), a method for dropping iodine in an aqueous alkaline solution of phenol under alkaline conditions in the presence of βcyclodextrin (Japanese Patent Laid-Open No. 63-101342, Japanese Patent Laid-Open No. 2003-64012), or the like can be arbitrarily selected. Examples of the iodinating agent include, but are not particularly limited to, iodinating agents such as iodine chloride, iodine, and N-iodosuccinimide. Among them, iodine chloride is preferable.

In the second embodiment, the iodination reaction through iodine chloride in an organic solvent is preferably used, particularly when introduction of a plurality of iodine atoms is intended. As the synthetic method of the compound (A) of the second embodiment, for example, the method described in the above references can be arbitrarily used, but is not limited thereto.

The method for producing the iodine-containing vinyl monomer having a general structure represented by the formula (1) may comprise the method for producing the iodine-containing alcohol substrate having a general structure represented by the formula (1-1). That is, the method for producing the iodine-containing vinyl monomer having a general structure represented by the formula (1) may comprise:
e) a step of providing an alcohol substrate having a general structure represented by the formula (1-3); and
f) an iodine introduction step.

As the organic solvent, a wide variety of organic solvents including a polar aprotic organic solvent and protic polar organic solvent are used. A single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used, and a polar aprotic solvent or a mixture thereof is preferable. The solvent is effective, but is not an essential component. Examples of suitable polar aprotic solvents include, but are not limited to, an alcohol solvent such as methanol and ethanol, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, hexamethylphosphorous triamide; and dimethylsulfoxide. Dimethylsulfoxide is preferable. Examples of suitable protic polar solvents include, but are not limited to, di(propylene glycol) methyl ether, di(ethylene glycol) methyl ether, 2-butoxyethanol, ethylene glycol, 2-methoxyethanol, propyleneglycol methylether, n-hexanol, and n-butanol.

The amount of the solvent used can be arbitrarily set according to, for example, the substrate, catalyst, and reaction conditions to be used, without particular limitation. In general, it is suitably 0 to 10000 parts by mass, and from the viewpoint of the yield, preferably 100 to 2000 parts by mass, based on 100 parts by mass of the reaction raw materials.

As the catalyst, a wide variety of dehydration catalysts which function under the reaction conditions of the second embodiment are used. An acid catalyst is preferable. Examples of suitable acid catalysts include, but are not limited to, an inorganic acid such as hydrochloric acid, sulfuric acid, phosphoric acid, hydrobromic acid, and hydrofluoric acid; an organic acid such as oxalic acid, malonic acid, succinic acid, adipic acid, sebacic acid, citric acid, fumaric acid, maleic acid, formic acid, p-toluenesulfonic acid, methanesulfonic acid, trifluoroacetic acid, dichloroacetic acid, trichloroacetic acid, trifluoromethanesulfonic acid, benzenesulfonic acid, naphthalenesulfonic acid, and naphthalenedisulfonic acid; a Lewis acid such as zinc chloride, aluminum chloride, iron chloride, and boron trifluoride; and a solid acid such as tungstosilicic acid, tungstophosphoric acid, silicomolybdic acid, and phosphomolybdic acid. These acid catalysts are used alone as one kind or in combination of two or more kinds. Among them, organic acids and solid acids are preferable from the viewpoint of production, and it is preferable to use hydrochloric acid or sulfuric acid from the viewpoint of production such as easy availability and handleability.

The amount of the catalyst used can be arbitrarily set according to, for example, the substrate, catalyst, and reaction conditions to be used, without particular limitation. In general, it is suitably 0.0001 to 100 parts by mass, and from the viewpoint of the yield, preferably 0.001 to 10 parts by mass, based on 100 parts by mass of the reaction raw materials.

### Reaction Conditions

The reaction mixture is formed by adding the alcohol substrate having the formula (1-3), the catalyst, and the organic solvent to a reactor. Any suitable reactor is used.

The reaction may be carried out by arbitrarily selecting a known method such as a batch method, a semi-batch method, or a continuous method.

The reaction temperature is not particularly limited. The preferred range varies according to the concentration of the substrate, the stability of the formed product, the selection of the catalyst, and the desired yield. In general, a temperature of 0° C. to 200° C. is suitable, and from the viewpoint of the yield, a temperature of 0° C. to 100° C. is preferable.

In the reaction using 1-(4-hydroxy-3-methoxyphenyl)ethanol as the substrate, the temperature range is preferably 0° C. to 100° C.

The reaction pressure is not particularly limited. The preferred range varies according to the concentration of the substrate, the stability of the formed product, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation.

In the reaction using 1-(4-hydroxy-3-methoxyphenyl)ethanol as the substrate, the preferred reaction pressure is reduced pressure to normal pressure, and reduced pressure is preferable.

The reaction time is not particularly limited. The preferred range varies according to the concentration of the substrate, the stability of the formed product, the selection of the catalyst, and the desired yield. However, the majority of the reactions are carried out for less than 6 hours, and the reaction time is typically 15 minutes to 600 minutes.

In the reaction using 1-(4-hydroxy-3-methoxyphenyl)ethanol as the substrate, the preferred reaction time range is 15 minutes to 600 minutes.

Isolation and purification can be conducted by using a conventionally known suitable method after terminating the reaction. For example, the reaction mixture is poured in ice water and extracted in an organic solvent such as ethyl acetate or diethyl ether. Then, the product is recovered by removing the solvent using evaporation at reduced pressure. The product can be isolated and purified as the desired high purity monomer by a separation and purification method by filtration, concentration, distillation, extraction, crystallization, recrystallization, column chromatography, activated carbon, or the like, which are well-known purification methods in the art, or a combined method thereof.

### [Production Method of Iodine-Containing Ketone Substrate Represented by Formula (1-2)]

The ketone substrate used in the production of the iodine-containing ketone substrate represented by the formula (1-2) is, for example, a ketone substrate having a general structure represented by the formula (1-4).

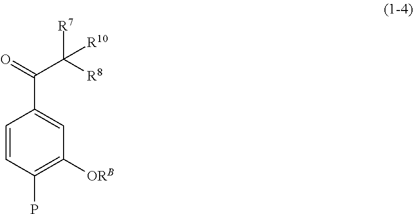

(In the formula (1-4), R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group.)

Examples of suitable ketone substrates include, but are not limited to, 4-hydroxy-5-methoxyphenylmethylketone and 5-ethoxy-4-hydroxyphenylmethylketone.

These ketone substrates can be obtained by many methods.

The method for producing the iodine-containing ketone substrate having a general structure represented by the formula (1-2) may comprise:
g) a step of providing a ketone substrate having a general structure represented by the formula (1-4); and
h) an iodine introduction step of introducing an iodine atom into the ketone substrate.

The method for producing the iodine-containing vinyl monomer having a general structure represented by the formula (1) may comprise the method for producing the iodine-containing ketone substrate having a general structure represented by the formula (1-2). That is, the method for producing the iodine-containing vinyl monomer having a general structure represented by the formula (1) may comprise:
g) a step of providing a ketone substrate having a general structure represented by the formula (1-4); and
h) an iodine introduction step of introducing an iodine atom into the ketone substrate.

As the organic solvent, a wide variety of organic solvents including a polar aprotic organic solvent and protic polar organic solvent are used. A single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used, and a polar aprotic solvent or a mixture thereof is preferable. The solvent is effective, but is not an essential component. Examples of suitable polar aprotic solvents include, but are not limited to, an alcohol solvent such as methanol and ethanol, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, hexamethylphosphorous triamide; and dimethylsulfoxide. Dimethylsulfoxide is preferable. Examples of suitable protic polar solvents include, but are not limited to, di(propylene glycol) methyl ether, di(ethylene glycol) methyl ether, 2-butoxyethanol, ethylene glycol, 2-methoxyethanol, propyleneglycol methylether, n-hexanol, and n-butanol.

The amount of the solvent used can be arbitrarily set according to, for example, the substrate, catalyst, and reaction conditions to be used, without particular limitation. In general, it is suitably 0 to 10000 parts by mass, and from the viewpoint of the yield, preferably 100 to 2000 parts by mass, based on 100 parts by mass of the reaction raw materials.

As the catalyst, a wide variety of dehydration catalysts which function under the reaction conditions of the second embodiment are used. An acid catalyst is preferable. Examples of suitable acid catalysts include, but are not limited to, an inorganic acid such as hydrochloric acid, sulfuric acid, phosphoric acid, hydrobromic acid, and hydrofluoric acid; an organic acid such as oxalic acid, malonic acid, succinic acid, adipic acid, sebacic acid, citric acid, fumaric acid, maleic acid, formic acid, p-toluenesulfonic acid, methanesulfonic acid, trifluoroacetic acid, dichloroacetic acid, trichloroacetic acid, trifluoromethanesulfonic acid, benzenesulfonic acid, naphthalenesulfonic acid, and naphthalenedisulfonic acid; a Lewis acid such as zinc chloride, aluminum chloride, iron chloride, and boron trifluoride; and a solid acid such as tungstosilicic acid, tungstophosphoric acid, silicomolybdic acid, and phosphomolybdic acid. These acid catalysts are used alone as one kind or in combination of two or more kinds. Among them, organic acids and solid acids are preferable from the viewpoint of production, and it is preferable to use hydrochloric acid or sulfuric acid from the viewpoint of production such as easy availability and handleability.

The amount of the catalyst used can be arbitrarily set according to, for example, the substrate, catalyst, and reaction conditions to be used, without particular limitation. In general, it is suitably 0.0001 to 100 parts by mass, and from the viewpoint of the yield, preferably 0.001 to 10 parts by mass, based on 100 parts by mass of the reaction raw materials.

### Reaction Conditions

The reaction mixture is formed by adding the ketone substrate having the formula (1-4), the catalyst, and the organic solvent to a reactor. Any suitable reactor is used.

The reaction may be carried out by arbitrarily selecting a known method such as a batch method, a semi-batch method, or a continuous method.

The reaction temperature is not particularly limited. The preferred range varies according to the concentration of the substrate, the stability of the formed product, the selection of the catalyst, and the desired yield. In general, a temperature of 0° C. to 200° C. is suitable, and from the viewpoint of the yield, a temperature of 0° C. to 100° C. is preferable.

In the reaction using 4′-hydroxy-3′-methoxyacetophenone as the substrate, the temperature range is preferably 0° C. to 100° C.

The reaction pressure is not particularly limited. The preferred range varies according to the concentration of the substrate, the stability of the formed product, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation.

In the reaction using 4′-hydroxy-3′-methoxyacetophenone as the substrate, the preferred reaction pressure is reduced pressure to normal pressure, and reduced pressure is preferable.

The reaction time is not particularly limited. The preferred range varies according to the concentration of the substrate, the stability of the formed product, the selection of the catalyst, and the desired yield. However, the majority of the reactions are carried out for less than 6 hours, and the reaction time is typically 15 minutes to 600 minutes.

In the reaction using 4′-hydroxy-3′-methoxyacetophenone as the substrate, the preferred reaction time range is 15 minutes to 600 minutes.

Isolation and purification can be conducted by using a conventionally known suitable method after terminating the reaction. For example, the reaction mixture is poured in ice water and extracted in an organic solvent such as ethyl acetate or diethyl ether. Then, the product is recovered by removing the solvent using evaporation at reduced pressure. The product can be isolated and purified as the desired high purity monomer by a separation and purification method by filtration, concentration, distillation, extraction, crystallization, recrystallization, column chromatography, activated carbon, or the like, which are well-known purification methods in the art, or a combined method thereof.

### [Production Method of Alcohol Substrate Represented by Formula (1-3)]

The ketone substrate used in the production of the alcohol substrate having a general structure represented by the formula (1-3) is, for example, the ketone substrate having a general structure represented by the aforementioned formula (1-4).

The method for producing the alcohol substrate having a general structure represented by the formula (1-3) comprises:
i) a step of providing a ketone substrate having a general structure represented by the formula (1-4); and
j) a reduction step of subjecting the ketone substrate to a reduction treatment.

The method for producing the iodine-containing vinyl monomer having a general structure represented by the formula (1) may comprise the method for producing the alcohol substrate having a general structure represented by the formula (1-3). That is, the method for producing the iodine-containing vinyl monomer having a general structure represented by the formula (1) may comprise:
i) a step of providing a ketone substrate having a general structure represented by the formula (1-4); and
j) a reduction step of subjecting the ketone substrate to a reduction treatment.

As the organic solvent, a wide variety of organic solvents including a polar aprotic organic solvent and protic polar organic solvent are used. A single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used, and a polar aprotic solvent or a mixture thereof is preferable. The solvent is effective, but is not an essential component. Examples of suitable polar aprotic solvents include, but are not limited to, an alcohol solvent such as methanol and ethanol, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, hexamethylphosphorous triamide; and dimethylsulfoxide. Dimethylsulfoxide is preferable. Examples of suitable protic polar solvents include, but are not limited to, di(propylene glycol) methyl ether, di(ethylene glycol) methyl ether, 2-butoxyethanol, ethylene glycol, 2-methoxyethanol, propyleneglycol methylether, n-hexanol, and n-butanol.

The amount of the solvent used can be arbitrarily set according to, for example, the substrate, catalyst, and reaction conditions to be used, without particular limitation. In general, it is suitably 0 to 10000 parts by mass, and from the viewpoint of the yield, preferably 100 to 2000 parts by mass, based on 100 parts by mass of the reaction raw materials.

In the reduction step, the ketone substrate is reduced by using, for example, a reducing agent. As the reducing agent, a wide variety of reducing agents which function under the reaction conditions of the second embodiment are used. Examples of suitable reducing agents include, but are not limited to, a metal hydride and a metal hydride complex compound, such as borane dimethylsulfide, diisobutylaluminum hydride, sodium borohydride, lithium borohydride, potassium borohydride, zinc borohydride, lithium tri-s-butylborohydride, potassium tri-s-butylborohydride, lithium triethylborohydride, lithium aluminum hydride, lithium tri-t-butoxyaluminum hydride, and sodium bis(methoxyethoxy)aluminum hydride. The amount of the catalyst used can be arbitrarily set according to, for example, the substrate, catalyst, and reaction conditions to be used, without particular limitation. In general, it is suitably 1 to 500 parts by mass, and from the viewpoint of the yield, preferably 10 to 200 parts by mass, based on 100 parts by mass of the reaction raw materials.

As a quenching agent, a wide variety of quenching agents which function under the reaction conditions of the second embodiment are used. The quenching agent has a function of deactivating the reducing agent. The quenching agent is effective, but is not an essential component. Examples of suitable quenching agents include, but are not limited to, ethanol, aqueous ammonium chloride solution, water, hydrochloric acid, and sulfuric acid.

The amount of the quenching agent used can be arbitrarily set according to the amount of the reducing agent to be used, without particular limitation. In general, it is suitably 1 to 500 parts by mass, and from the viewpoint of the yield, it is preferably 50 to 200 parts by mass, based on 100 parts by mass of the reaction raw materials.

### Reaction Conditions

The reaction mixture is formed by adding the ketone substrate having the formula (1-4), the catalyst, and the organic solvent to a reactor. Any suitable reactor is used.

The reaction may be carried out by arbitrarily selecting a known method such as a batch method, a semi-batch method, or a continuous method.

The reaction temperature is not particularly limited. The preferred range varies according to the concentration of the substrate, the stability of the formed product, the selection of the catalyst, and the desired yield. In general, a temperature of 0° C. to 200° C. is suitable, and from the viewpoint of the yield, a temperature of 0° C. to 100° C. is preferable.

In the reaction using 4′-hydroxy-3′-methoxyacetophenone as the substrate, the temperature range is preferably 0° C. to 100° C.

The reaction pressure is not particularly limited. The preferred range varies according to the concentration of the substrate, the stability of the formed product, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation.

In the reaction using 4′-hydroxy-3′-methoxyacetophenone as the substrate, the preferred reaction pressure is reduced pressure to normal pressure, and reduced pressure is preferable.

The reaction time is not particularly limited. The preferred range varies according to the concentration of the substrate, the stability of the formed product, the selection of the catalyst, and the desired yield. However, the majority of the reactions are carried out for less than 6 hours, and the reaction time is typically 15 minutes to 600 minutes.

In the reaction using 4′-hydroxy-3′-methoxyacetophenone as the substrate, the preferred reaction time range is 15 minutes to 600 minutes.

Isolation and purification can be conducted by using a conventionally known suitable method after terminating the reaction. For example, the reaction mixture is poured in ice water and extracted in an organic solvent such as ethyl acetate or diethyl ether. Then, the product is recovered by removing the solvent using evaporation at reduced pressure. The product can be isolated and purified as the desired high purity monomer by a separation and purification method by filtration, concentration, distillation, extraction, crystallization, recrystallization, column chromatography, activated carbon, or the like, which are well-known purification methods in the art, or a combined method thereof.

### [Method for Producing Iodine-Containing Vinyl Monomer Represented by Formula (2)]

The method for producing the iodine-containing vinyl monomer according to the second embodiment may be the method for producing the iodine-containing vinyl monomer represented by the following formula (2), and specifically may be the method for producing an iodine-containing alkoxystyrene.

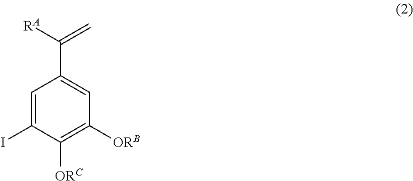

(In the formula (2), R^{A} is a hydrogen atom, a methyl group, or a trifluoromethyl group; R^{B} is a substituted or unsubstituted alkyl group having 1 to 30 carbon atoms; and R^{C} is a substituted or unsubstituted acyl group having 1 to 30 carbon atoms.)

Examples of the acetoxystyrene produced by the method of the second embodiment include, but are not limited to, 4-acetoxy-3-iodo-5-methoxystyrene and 4-acetoxy-5-ethoxy-3-iodostyrene.

### [Iodine-Containing Vinyl Monomer]

The iodine-containing vinyl monomer used in the second embodiment is, for example, the iodine-containing vinyl monomer having a general structure represented by the formula (1).

The iodine-containing vinyl monomer having a general structure represented by the formula (2) may comprise:
k) a step of providing an iodine-containing vinyl monomer having a general structure represented by the formula (1); and
l) an acylation step of subjecting the iodine-containing vinyl monomer to an acylation treatment.

As the organic solvent, a wide variety of organic solvents including a polar aprotic organic solvent and protic polar organic solvent are used. A single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used, and a polar aprotic solvent or a mixture thereof is preferable. The solvent is effective, but is not an essential component. Examples of suitable polar aprotic solvents include, but are not limited to, an alcohol solvent such as methanol and ethanol, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, hexamethylphosphorous triamide; and dimethylsulfoxide. Dimethylsulfoxide is preferable. Examples of suitable protic polar solvents include, but are not limited to, di(propylene glycol) methyl ether, di(ethylene glycol) methyl ether, 2-butoxyethanol, ethylene glycol, 2-methoxyethanol, propyleneglycol methylether, n-hexanol, and n-butanol.

The amount of the solvent used can be arbitrarily set according to, for example, the substrate, catalyst, and reaction conditions to be used, without particular limitation. In general, it is suitably 0 to 10000 parts by mass, and from the viewpoint of the yield, preferably 100 to 2000 parts by mass, based on 100 parts by mass of the reaction raw materials.

The acylation step is conducted by using, for example, a catalyst. As the catalyst, a wide variety of acylation catalysts which function under the reaction conditions of the second embodiment are used. A base catalyst is preferable. Examples of suitable base catalysts include, but are not limited to, an amine-containing catalyst such as pyridine and ethylenediamine, and non-amine basic catalyst such as a metal salt, and in particular, potassium salt or acetate is preferable. Examples of suitable catalysts include, but are not limited to, potassium acetate, potassium carbonate, potassium hydrate, sodium acetate, sodium carbonate, sodium hydroxide, and magnesium oxide.

All non-amine base catalysts of the second embodiment are commercially available from, for example, EM Science (Gibbstown) or Aldrich (Milwaukee).

The amount of the catalyst used can be arbitrarily set according to, for example, the substrate, catalyst, and reaction conditions to be used, without particular limitation. In general, it is suitably 1 to 5000 parts by mass, and from the viewpoint of the yield, preferably 50 to 3000 parts by mass, based on 100 parts by mass of the reaction raw materials.

As the polymerization inhibitor, a wide variety of polymerization inhibitors which function under the reaction conditions of the second embodiment are used. The polymerization inhibitor is effective, but is not an essential component. Examples of suitable polymerization inhibitors include, but are not limited to, hydroquinone, hydroquinone monomethyl ether, 4-tert-butylcatechol, phenothiazine, N-oxyl (nitroxide) inhibitor, for example, Prostab® 5415 (bis(1-oxyl-2,2,6,6-tetramethylpiperidin-4-yl)sebacate which is commercially available from Ciba Specialty Chemicals, Tarrytown, NY, CAS #2516-92-9), 4-hydroxy-TEMPO (4-hydroxy-2,2,6,6-tetramethylpiperidin-1-yloxy, which is commercially available from TCI, CAS #2226-96-2), Uvinul® 4040P (1,6-hexamethylene-bis(N-formyl-N-(1-oxyl-2,2,6,6-tetramethylpiperidin-4-yl)amine which is commercially available from BASF Corp., Worcester, MA), ammonium-N-nitrosophenylhydroxylamine (Q1300 which is commercially available from FUJIFILM Wako Pure Chemical Corporation), and N-nitrosophenylhydroxylamine aluminium salt (Q1301 which is commercially available from FUJIFILM Wako Pure Chemical Corporation).

The amount of the polymerization inhibitor used can be arbitrarily set according to, for example, the substrate, catalyst, and reaction conditions to be used, without particular limitation. In general, it is suitably 0.0001 to 100 parts by mass, and from the viewpoint of the yield, it is preferably 0.001 to 10 parts by mass, based on 100 parts by mass of the reaction raw materials.

As the polymerization retarder, a wide variety of polymerization retarders which function under the reaction conditions of the second embodiment are used. The polymerization retarder is effective, but is not an essential component. A polymerization retardant is effectively used by combining with the polymerization inhibitor. The polymerization retardant is well-known in the art as a compound that can delay the polymerization reaction, but cannot prevent all polymerization from occurring. A typical retardant is an aromatic nitro compound such as dinitro-ortho-cresol (DNOC) and dinitro butyl phenol (DNBP). The method for producing the polymerization retardant is common and well-known in the art (e.g., see U.S. Pat. No. 6,339,177; Park et al., Polymer (Korea) (1988), 12(8), 710-19), and its use in the control of styrene polymerization is well documented (e.g., see Bushby et al., Polymer (1998), 39(22), 5567-5571).

The amount of the polymerization retarder used can be arbitrarily set according to, for example, the substrate, catalyst, and reaction conditions to be used, without particular limitation. In general, it is suitably 0.0001 to 100 parts by mass, and from the viewpoint of the yield, preferably 0.001 to 10 parts by mass, based on 100 parts by mass of the reaction raw materials.

### Reaction Conditions

The reaction mixture is formed by adding the iodine-containing vinyl monomer having the formula (1), the catalyst, and the organic solvent to a reactor. Any suitable reactor is used.

The reaction may be carried out by arbitrarily selecting a known method such as a batch method, a semi-batch method, or a continuous method.

The reaction temperature is not particularly limited. The preferred range varies according to the concentration of the substrate, the stability of the formed product, the selection of the catalyst, and the desired yield. In general, a temperature of 0° C. to 200° C. is suitable, and from the viewpoint of the yield, a temperature of 0° C. to 100° C. is preferable.

In the reaction using 4-hydroxy-3-iodo-5-methoxystyrene as the substrate, the temperature range is preferably 0° C. to 100° C.

The reaction pressure is not particularly limited. The preferred range varies according to the concentration of the substrate, the stability of the formed product, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation.

In the reaction using 4-hydroxy-3-iodo-5-methoxystyrene as the substrate, the preferred reaction pressure is reduced pressure to normal pressure, and reduced pressure is preferable.

The reaction time is not particularly limited. The preferred range varies according to the concentration of the substrate, the stability of the formed product, the selection of the catalyst, and the desired yield. However, the majority of the reactions are carried out for less than 6 hours, and the reaction time is typically 15 minutes to 600 minutes.

In the reaction using 4-hydroxy-3-iodo-5-methoxystyrene as the substrate, the preferred reaction time range is 15 minutes to 600 minutes.

Isolation and purification can be conducted by using a conventionally known suitable method after terminating the reaction. For example, the reaction mixture is poured in ice water and extracted in an organic solvent such as ethyl acetate or diethyl ether. Then, the product is recovered by removing the solvent using evaporation at reduced pressure. The product can be isolated and purified as the desired high purity monomer by a separation and purification method by filtration, concentration, distillation, extraction, crystallization, recrystallization, column chromatography, activated carbon, or the like, which are well-known purification methods in the art, or a combined method thereof.

It is preferable that the compound in the second embodiment be obtained as a crude by the reaction described above and be then further subjected to purification, thereby removing the residual metal impurities. That is, it is preferable to avoid residual metal impurities derived from contamination by metal components which are used as a reaction aid during the production process of the compound or contaminated from a reaction vessel for production or other production equipment, from the viewpoint of preventing the deterioration of the resin with time, storage stability, and further, production yield due to processability, defects, and the like when the composition is formed into a resin and applied to a semiconductor production process.

The residual amounts of the aforementioned metal impurities are preferably less than 1 ppm, more preferably less than 100 ppb, further preferably less than 50 ppb, still more preferably less than 10 ppb, and most preferably less than 1 ppb, based on the resin. In particular, with respect to metal species such as Fe, Ni, Sb, W, and Al which are classified as transition metals, there is concern that the amount of residual metals of 1 ppm or more may cause the denaturation and deterioration of materials with time due to the interaction with the compound in the second embodiment. Further, there is also concern that the metal balance cannot be sufficiently reduced with the amount of residual metals of 1 ppm or more when a resin for semiconductor process is prepared using the compound prepared, which results in defects derived from residual metals in a semiconductor production process and reduction in yield due to performance deterioration.

The purification method is not particularly limited, but comprises a step of obtaining a solution (S) by dissolving the compound in the second embodiment in a solvent; and a step of extracting impurities in the compound in the second embodiment by bringing the obtained solution (S) into contact with an acidic aqueous solution (a first extraction step), wherein the solvent used in the step of obtaining the solution (S) contains an organic solvent that does not inadvertently mix with water.

According to the purification method, the contents of various metals that may be contained as impurities in the resin can be reduced.

More specifically, the compound in the second embodiment can be dissolved in an organic solvent that does not inadvertently mix with water to obtain the solution (S), and further, an extraction treatment can be carried out by bringing the solution (S) into contact with an acidic aqueous solution. Thereby, after the metals contained in the solution (S) is transferred to the aqueous phase, the organic phase and the aqueous phase can be separated to obtain a resin having a reduced metal content.

The solvent that does not inadvertently mix with water used in the purification method is not particularly limited, but is preferably an organic solvent that is safely applicable to a semiconductor production process, and specifically it is an organic solvent having a solubility in water at room temperature of less than 30%, and more preferably is an organic solvent having a solubility of less than 20% and particularly preferably less than 10%. The amount of the organic solvent used is preferably 1 to 100 times the total mass of the resin to be used.

Specific examples of the solvent that does not inadvertently mix with water include, but are not limited to, ethers such as diethyl ether and diisopropyl ether, esters such as ethyl acetate, n-butyl acetate, and isoamyl acetate; ketones such as methyl ethyl ketone, methyl isobutyl ketone, ethyl isobutyl ketone, cyclohexanone, cyclopentanone, 2-heptanone, and 2-pentanone; glycol ether acetates such as ethylene glycol monoethyl ether acetate, ethylene glycol monobutyl ether acetate, propylene glycol monomethyl ether acetate (PGMEA), and propylene glycol monoethyl ether acetate; aliphatic hydrocarbons such as n-hexane and n-heptane; aromatic hydrocarbons such as toluene and xylene; and halogenated hydrocarbons such as methylene chloride and chloroform. Among them, toluene, 2-heptanone, cyclohexanone, cyclopentanone, methyl isobutyl ketone, propylene glycol monomethyl ether acetate, ethyl acetate, and the like are preferable, methyl isobutyl ketone, ethyl acetate, cyclohexanone, and propylene glycol monomethyl ether acetate are more preferable, and methyl isobutyl ketone and ethyl acetate are still more preferable. Since methyl isobutyl ketone, ethyl acetate, and the like have a relatively high saturation solubility and a relatively low boiling point of the compound of the second embodiment, the load in the case of industrially distilling off a solvent and in the step of removing the solvent by drying can be reduced. These solvents can be each used alone, or can also be used as a mixture of two or more kinds.

The acidic aqueous solution used in the purification method is arbitrarily selected from among aqueous solutions in which organic compounds or inorganic compounds are dissolved in water, generally known as acidic aqueous solutions. Examples of the acidic aqueous solution include, but are not limited to, aqueous mineral acid solutions in which mineral acids such as hydrochloric acid, sulfuric acid, nitric acid, and phosphoric acid are dissolved in water, or aqueous organic acid solutions in which organic acids such as acetic acid, propionic acid, oxalic acid, malonic acid, succinic acid, fumaric acid, maleic acid, tartaric acid, citric acid, methanesulfonic acid, phenolsulfonic acid, p-toluenesulfonic acid, and trifluoroacetic acid are dissolved in water. These acidic aqueous solutions can be each used alone, and can be also used in combination of two or more kinds. Among these acidic aqueous solutions, aqueous solutions of one or more mineral acids selected from the group consisting of hydrochloric acid, sulfuric acid, nitric acid and phosphoric acid, or aqueous solutions of one or more organic acids selected from the group consisting of acetic acid, propionic acid, oxalic acid, malonic acid, succinic acid, fumaric acid, maleic acid, tartaric acid, citric acid, methanesulfonic acid, phenolsulfonic acid, p-toluenesulfonic acid and trifluoroacetic acid are preferable, aqueous solutions of sulfuric acid, nitric acid, and carboxylic acids such as acetic acid, oxalic acid, tartaric acid and citric acid are more preferable, aqueous solutions of sulfuric acid, oxalic acid, tartaric acid and citric acid are further preferable, and an aqueous solution of oxalic acid is still more preferable. It is considered that polyvalent carboxylic acids such as oxalic acid, tartaric acid and citric acid coordinate with metal ions and provide a chelating effect, and thus there is a tendency that metals can be more effectively removed. Also, as for water used herein, it is preferable to use water, the metal content of which is small, such as ion exchanged water, according to the purpose of the purification method in the second embodiment.

The pH of the acidic aqueous solution used in the purification method is not particularly limited, but it is preferable to regulate the acidity of the aqueous solution in consideration of an influence on the compound. Normally, the pH range is about 0 to 5, and is preferably about pH 0 to 3.

The amount of the acidic aqueous solution used in the purification method is not particularly limited, but it is preferable to regulate the amount from the viewpoint of reducing the number of extraction operations for removing metals and from the viewpoint of ensuring operability in consideration of the overall amount of fluid. From the above viewpoints, the amount of the acidic aqueous solution to be used is preferably 10 to 200% by mass, and more preferably 20 to 100% by mass, based on 100% by mass of the solution (S).

In the purification method, by bringing the acidic aqueous solution into contact with the solution (S), metals can be extracted from the compound in the solution (S).

In the purification method, the solution (S) can further contain an organic solvent that inadvertently mixes with water. When the solution (S) contains an organic solvent that inadvertently mixes with water, there is a tendency that the amount of the compound charged can be increased, also the fluid separability is improved, and purification can be carried out at a high reaction vessel efficiency. The method for adding the organic solvent that inadvertently mixes with water is not particularly limited. For example, any of a method involving adding it to the organic solvent-containing solution in advance, a method involving adding it to water or the acidic aqueous solution in advance, and a method involving adding it after bringing the organic solvent-containing solution into contact with water or the acidic aqueous solution may be employed. Among them, the method involving adding it to the organic solvent-containing solution in advance is preferable in terms of the workability of operations and the ease of managing the amount to be charged.

The organic solvent that inadvertently mixes with water used in the purification method is not particularly limited, but is preferably an organic solvent that is safely applicable to a semiconductor production process. The amount of the organic solvent used that inadvertently mixes with water is not particularly limited as long as the solution phase and the aqueous phase separate, but is preferably 0.1 to 100 times, more preferably 0.1 to 50 times, and further preferably 0.1 to 20 times the total mass of the compound to be used.

Specific examples of the organic solvent that inadvertently mixes with water used in the purification method include, but are not limited to, ethers such as tetrahydrofuran and 1,3-dioxolane; alcohols such as methanol, ethanol, and isopropanol; ketones such as acetone and N-methylpyrrolidone; aliphatic hydrocarbons such as glycol ethers such as ethylene glycol monoethyl ether, ethylene glycol monobutyl ether, propylene glycol monomethyl ether (PGME), and propylene glycol monoethyl ether. Among them, N-methylpyrrolidone, propylene glycol monomethyl ether and the like are preferable, and N-methylpyrrolidone and propylene glycol monomethyl ether are more preferable. These solvents can be each used alone, or can also be used as a mixture of two or more kinds.

The temperature when the extraction treatment is carried out is normally in the range of 20 to 90° C., and preferably 30 to 80° C. The extraction operation is carried out, for example, by thoroughly mixing the solution (S) and the acidic aqueous solution by stirring or the like and then leaving the obtained mixed solution to stand still. Thereby, metals contained in the solution (S) are transferred to the aqueous phase. Also, by this operation, the acidity of the solution is lowered, and the deterioration of the compound can be suppressed.

By being left to stand still, the mixed solution is separated into an aqueous phase and a solution phase containing the compound and the solvent, and thus the solution phase is recovered by decantation or the like. The time for leaving the mixed solution to stand still is not particularly limited, but it is preferable to regulate the time for leaving the mixed solution to stand still from the viewpoint of attaining good separation of the solution phase containing the solvent and the aqueous phase. Normally, the time for leaving the mixed solution to stand still is 1 minute or longer, preferably 10 minutes or longer, and more preferably 30 minutes or longer. While the extraction treatment may be carried out only once, it is effective to repeat mixing, leaving-to-stand-still, and separating operations multiple times.

It is preferable that the purification method comprise the step of extracting impurities in the resin by further bringing the solution phase containing the compound into contact with water after the first extraction step (the second extraction step). Specifically, for example, it is preferable that, after the extraction treatment is carried out using an acidic aqueous solution, the solution phase that is extracted and recovered from the aqueous solution and that contains the resin and the solvent be further subjected to an extraction treatment with water. The aforementioned extraction treatment with water is not particularly limited, and can be carried out by, for example, thoroughly mixing the solution phase and water by stirring or the like and then leaving the obtained mixed solution to stand still. The mixed solution after being left to stand still is separated into an aqueous phase and a solution phase containing the compound and the solvent, and thus the solution phase can be recovered by decantation or the like.

Water used herein is preferably water, the metal content of which is small, such as ion exchanged water, according to the purpose of the second embodiment. While the extraction treatment may be carried out only once, it is effective to repeat mixing, leaving-to-stand-still, and separating operations multiple times. The proportions of both used in the extraction treatment, and temperature, time and other conditions are not particularly limited, and may be the same as those of the previous contact treatment with the acidic aqueous solution.

Water that is possibly present in the thus-obtained solution containing the compound and the solvent can be easily removed by performing vacuum distillation or a like operation. Also, if required, the concentration of the compound can be regulated to be any concentration by adding a solvent to the solution.

In the purification method of the compound according to the second embodiment, the compound can be purified by passing a solution obtained by dissolving the resin in a solvent through a filter.

According to the purification method according to the second embodiment, the content of various metals in the resin can be effectively and significantly reduced. The amount of these metal components can be measured by the method described in Examples, which will be mentioned later.

Note that “passing” in the second embodiment means that the solution is passed from the outside of a filter through the inside of the filter, and then transferred to the outside of the filter again, and for example, an aspect in which the solution is brought into contact merely on the surface of a filter and an aspect in which the solution is transferred outside of the ion exchange resin while being brought into contact with the surface of a filter (that is, a mere contact aspect) are eliminated.

### [Filtering Purification Step (Passing Step)]

In the filter-passing step in the second embodiment, the filter used for removing the metals in a solution containing the compound and the solvent can be one which is normally commercially available as a filter for liquid filtration. The filtering precision of the filter is not particularly limited, but the nominal pore size of the filter is preferably 0.2 μm or less, more preferably less than 0.2 μm, further preferably 0.1 μm or less, still more preferably less than 0.1 μm, and even more preferably 0.05 μm or less. The lower limit value of the nominal pore size of the filter is not particularly limited, but is normally 0.005 μm. The nominal pore size herein refers to the nominal pore size indicating the separation performance of a filter and is the pore size determined by test methods defined by the manufacturer of the filter, such as a bubble point test, a mercury intrusion porosimetry test, and a standard particle capture test. In the case of using a commercial product, it is the value described in the catalog data of the manufacturer. By setting the nominal pore size to 0.2 μm or less, the content of the metals after the solution is passed through the filter once can be effectively reduced. In the second embodiment, the filter-passing step may be carried out twice or more to reduce the content of each metal in the solution.

As the form of the filter, a hollow fiber membrane filter, a membrane filter, a pleated membrane filter, and a filter packed with a filtering medium such as a non-woven fabric, cellulose, and diatomaceous earth can be used. Among the above, it is preferable that the filter be one or more selected from the group consisting of a hollow fiber membrane filter, a membrane filter, and a pleated membrane filter. In particular, in terms of highly fine filtering precision and a high filtration area as compared to other forms, a hollow fiber membrane filter is particularly preferably used.

Examples of the filter material include polyolefin such as polyethylene and polypropylene; a polyethylene resin in which a functional group having ion exchange capacity is applied by graft polymerization; a polar group-containing resin such as polyamide, polyester, and polyacrylonitrile; and a fluorine-containing resin such as fluorinated polyethylene (PTFE). Among the above, the filtering medium of the filter is preferably one or more selected from the group consisting of a filtering medium made of polyamide, a filtering medium made of polyolefin resin, and a filtering medium made of fluorine resin. From the viewpoint of an effect of reducing heavy metals such as chrome, polyamide is particularly preferable. From the viewpoint of avoiding the dissolution of metals from the filtering medium, a filter having a filter material other than sintered metals is preferably used.

Examples of the polyamide filter include (hereinafter, trademarks), but are not limited to, Polyfix nylon series manufactured by KITZ MICROFILTER CORPORATION, Ultipleat P-Nylon 66 and Ultipor N66 manufactured by Nihon Pall Ltd., and LifeASSURE PSN series and LifeASSURE EF series manufactured by 3M Japan Limited.

Examples of the polyolefin filter include, but are not limited to, Ultipleat PE-Kleen and IonKleen manufactured by Nihon Pall Ltd., and Protego series, Microgard Plus HC10, and Optimizer D manufactured by Nihon Entegris G.K.

Examples of the polyester filter include, but are not limited to, Duraflow DFE manufactured by Central Filter Mfg Co Ltd., and a pleated type, PMC manufactured by Nihon Filter Co., Ltd.

Examples of the polyacrylonitrile filter include, but are not limited to, Ultrafilter AIP-0013D, ACP-0013D, and ACP-0053D manufactured by ADVANTEC TOYO KAISHA, LTD.

Examples of the fluorine resin filter include, but are not limited to, Emflon HTPFR manufactured by Nihon Pall Ltd., and LifeASSURE FA series manufactured by 3M Japan Limited.

These filters may be each used alone, or may also be used in combination of two or more kinds.

The filter may contain an ion exchanger such as a cation exchange resin, a cation charge regulator that generates a zeta potential in an organic solvent solution to be filtered, or the like.

Examples of the filter containing an ion exchanger include, but are not limited to, Protego series manufactured by Nihon Entegris G.K. and KURANGRAFT manufactured by Kurashiki Textile Manufacturing Co., Ltd.

Examples of the filter containing a substance having a positive zeta potential, such as polyamide polyamine epichlorohydrin cationic resin, include (hereinafter, trademarks), but are not limited to, Zeta Plus 40QSH and Zeta Plus 020GN, or LifeASSURE EF series manufactured by 3M Japan Limited.

In the purification method of the compound according to the second embodiment, purification can be carried out by distilling the compound itself. The distillation method is not particularly limited, but a known method such as normal pressure distillation, vacuum distillation, molecular distillation, or steam distillation can be used.

### [Applications of Compound (A)]

The compound (A) according to the second embodiment can increase the sensitivity to an exposure light source by being added to a composition for film formation as it is or as the polymer described below. The compound (A) or a polymer thereof is preferably used for photoresists.

### [Composition]

The composition of the second embodiment contains the compound (A). The content of the compound (A) in the second embodiment is preferably 90% by mass or more, more preferably 95% by mass or more, and further preferably 99% by mass or more.

As other preferred form of the composition of the second embodiment, it is preferable that the compound (A) at least include a compound represented by the formula (1) other than the formula (1C) and a compound represented by the formula (1C). The proportion of the monomer represented by the formula (1C) contained is preferably as little as 1 ppm by mass or more and 10% by mass or less, more preferably 20 ppm by mass or more and 2% by mass or less, and 50 ppm by mass or more and 1% by mass or less, based on the total monomer represented by the formula (1).

By setting the content of the compound represented by the formula (1C) to the described range, the interaction between resins when being formed into a resin can be reduced, and by suppressing the crystalline due to the interaction between the resins after a film is formed using the resin, the locality in the solubility in the developing solution upon developing can be reduced at a molecular level from several to several tens of nanometers, the reduction in pattern quality, such as the line edge roughness and the residue defect, of a pattern which is formed in the pattern formation process in a series of lithography processes of exposure, post exposure bake, and development can be suppressed, and the resolution can be further improved.

The influence of these effects for lithography performance increases in the compound represented by the formula (1C), as a result that the hydrophilicity/hydrophobicity of the compound represented by the formula (1) and the compound represented by the formula (1C) each having the core A into which a halogen element, in particular, iodine, fluorine, or the like is introduced, is shifted to a compound having a hydroxystyrene skeleton in which no iodine and the like are introduced, and the polarization at the polar moieties is thus increased.

The composition of the second embodiment contains the compound (A). In the composition, the content of impurities containing K (potassium) is preferably 1 ppm by mass or less, more preferably 0.5 ppm by mass or less, further preferably 0.1 ppm by mass or less, and still more preferably 0.005 ppm by mass or less, in terms of element, based on the total compound (A).

In the composition of the second embodiment, the content of one or more elemental impurities selected from the group consisting of Mn (manganese), Al (aluminum), Si (silicon), and Li (lithium) (preferably, one or more elemental impurities selected from the group consisting of Mn and Al) is preferably 1 ppm or less, more preferably 0.5 ppm or less, further preferably 0.1 ppm or less, in terms of element, based on the total compound (A).

The amount of K, Mn, Al, Si, Li, and the like is measured by inorganic elemental analysis (IPC-AES/IPC-MS). Examples of the inorganic element analyzer include “AG8900” manufactured by Agilent Technologies, Inc.

In the composition of the second embodiment, the content of the phosphorus-containing compound is preferably 10 ppm or less, more preferably 8 ppm or less, and further preferably 5 ppm or less, based on the total compound (A).

In the composition of the second embodiment, the content of maleic acid is preferably 10 ppm or less, more preferably 8 ppm or less, and further preferably 5 ppm or less, based on the total compound (A).

The amount of the phosphorus-containing compound and maleic acid is calculated from the area fraction of the GC chart and the peak intensity ratio between the target peak and the reference peak by gas chromatography mass spectrometry (GC-MS).

In the composition of the second embodiment, the content of peroxide is preferably 10 ppm or less, more preferably 1 ppm or less, and further preferably 0.1 ppm or less, based on the total compound (A).

The content of peroxide is quantified by an ammonium ferrothiocyanate acid method (hereinafter, AFTA method) by adding trichloroacetic acid in a sample, then adding ammonium iron (II) sulfate and potassium thiocyanate, determining a calibration curve of peroxide which is known as a standard, and measuring the absorbance at a wavelength of 480 μm.

In the composition of the second embodiment, the moisture content is preferably 100,000 ppm or less, more preferably 20,000 ppm or less, further preferably 1,000 ppm or less, still more preferably 500 ppm or less, and still more preferably 100 ppm or less, based on the total compound (A). The moisture content is measured by a Karl Fischer method (Karl Fischer moisture content measuring apparatus).

### [Polymer (A)]

The polymer (A) of the second embodiment contains a constitutional unit derived from the aforementioned compound (A). By containing the constitutional unit derived from the compound (A), the polymer (A) can increase the sensitivity to an exposure light source when being blended in a resist composition. In particular, the polymer (A) can exhibit sufficient sensitivity and can form good thin line patterns having a narrow line width even in the case of using an extreme ultraviolet ray as the exposure light source.

The conventional resist compositions were difficult to expand for actual semiconductor production, since their sensitivity to an exposure light source may be reduced over time due to storage or the like. However, according to the polymer (A) of the second embodiment, the stability of the resist composition is improved, and the reduction in the sensitivity to an exposure light source is suppressed even in the case of long-term storage.

The polymer (A) of the second embodiment contains the constitutional unit derived from the compound (A).

The constitutional unit derived from the compound (A) contained in the polymer (A) includes, for example, a constitutional unit represented by the following formula (1-A).

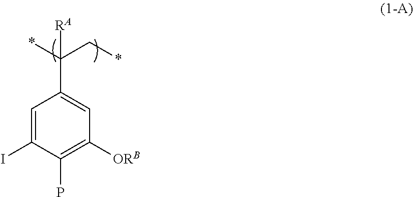

In the formula (1-A), R^{A}, R^{B}, and P have the same meanings as in the formula (1); and * is a site for binding with an adjacent constitutional unit.

R^{A} is preferably a hydrogen atom or a methyl group.

R^{B} is preferably an alkyl group having 1 to 4 carbon atoms.

Furthermore, P is a hydroxy group, a tertiary ester group, an acetal group, a carbonate ester group, or a carboxyalkoxy group.

The polymer (A) can be obtained by polymerizing the compound (A) of the second embodiment, or by copolymerizing the compound (A) and other monomers. For example, the polymer (A) can be used as a material for forming a film for lithography.

The amount of the constitutional unit derived from the compound (A) is preferably 5 mol % or more, more preferably 8 mol % or more, and further preferably 10 mol % or more, based on the total amount of the monomer components of the polymer (A). The amount of the constitutional unit derived from the compound (A) is 100 mol % or less, preferably 80 mol % or less, more preferably 50 mol % or less, and further preferably 30 mol % or less, based on the total amount of the monomer components of the polymer (A).

As a preferred form of the polymer of the second embodiment, it is preferable that, as the constitutional unit of the polymer (A), the monomer represented by the compound (A) at least include a compound represented by the formula (1) and a compound represented by the formula (1C). The proportion of the monomer represented by the formula (1C) contained is preferably as little as 10 ppm by mass or more and 10% by mass or less, more preferably 20 ppm or more and 2% by mass or less, and 50 ppm or more and 1% by mass or less, based on the total monomer represented by the formula (1).

By setting the proportion of the compound represented by the formula (1C) contained to the aforementioned range, the interaction between resins when being formed into a resin can be reduced. By suppressing the crystalline due to the interaction between the resins after a film is formed using the resin, the locality in the solubility in the developing solution upon developing can be reduced at a molecular level from several to several tens of nanometers. As a result, the reduction in pattern quality, such as the line edge roughness and the residue defect, of a pattern which is formed in the pattern formation process in a series of lithography processes of exposure, post exposure bake, and development can be suppressed, and the resolution can be further improved.

The influence of these effects for lithography performance increases in the monomer represented by the formula (1C), as a result that the hydrophilicity/hydrophobicity of the compound represented by the formula (1) and the compound represented by the formula (1C) each having the core into which a halogen element, in particular, iodine is introduced, is shifted to a compound having a hydroxystyrene skeleton in which no iodine and the like are introduced, and the polarization at the polar moieties is thus increased.

In the polymer (A), as the other monomers to be copolymerized with the compound (A), it is preferable to contain a polymerization unit that has an aromatic compound having an unsaturated double bond as a substituent, as a polymerization unit, and has a functional group for improving solubility in an alkaline developing solution by the action of an acid or a base.

In the polymer (A), examples of other monomers to be copolymerized with the compound (A) include, but are not particularly limited to, those described in International Publication No. WO 2016/125782, International Publication No. WO 2015/115613, Japanese Patent Laid-Open No. 2015/117305, International Publication No. WO 2014/175275, and Japanese Patent Laid-Open No. 2012/162498, or a compound represented by the following formula (C1) or the following formula (C2). Among them, the compound represented by the following formula (C1) or the following formula (C2) is preferable. Other monomers to be copolymerized with the compound (A) in the polymer (A) preferably contains a constitutional unit represented by the following formula (C0).

That is, the polymer (A) preferably further contains the constitutional unit represented by the following formula (C0), a constitutional unit represented by the following formula (C1), or a constitutional unit represented by the following formula (C2), in addition to the constitutional unit represented by the formula (1-A).

From the viewpoint of the quality of the pattern shape after exposure and development in a lithography process, in particular, suppressing the roughness and the pattern collapse, it is preferable that the difference between the dissolution rate Rₘᵢₙ of the resin that becomes protrusions of a pattern during alkali development on unexposed portions during exposure in the alkaline developing solution and the dissolution rate Rₘₐₓ of the resin that becomes recesses of the pattern during alkali development on exposed portions during exposure in the alkaline developing solution be 3 or more orders of magnitude larger, and it is preferable that the difference in the dissolution rate due to the presence or absence of a protective group be large and the elimination rate of the protective group in post-exposure bake (PEB) and development be large. From these viewpoints, other monomers to be copolymerized with the compound (A) in the polymer (A) preferably have a constitutional unit represented by the following formula (C1).

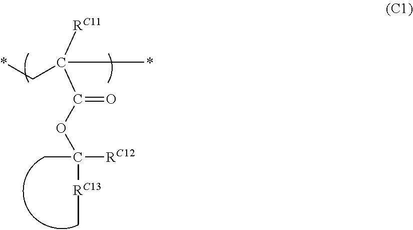

In the formula (C1),
R^{C11} is a hydrogen atom, a methyl group, or a trifluoromethyl group;
R^{C12} is a hydrogen atom, or an alkyl group having 1 to 4 carbon atoms;
R^{C13} is a cycloalkyl group or heterocycloalkyl group having 4 to 20 carbon atoms formed by being taken together with a carbon atom bonded to R^{C13}; and
* is a site for binding with an adjacent constitutional unit.

R^{C12} is preferably a hydrogen atom, or an alkyl group having 1 to 3 carbon atoms. R^{C13} is preferably a cycloalkyl group or heterocycloalkyl group having 4 to 10 carbon atoms formed by being taken together with a carbon atom bonded to R^{C13}. The cycloalkyl group or heterocycloalkyl group of R^{C13} may have a substituent (e.g., an oxo group).

The amount of the constitutional unit represented by the formula (C1) is preferably 5 mol % or more, more preferably 10 mol % or more, and further preferably 20 mol % or more, based on the total amount of the monomer components of the polymer (A). The amount of the constitutional unit represented by the formula (C1) is preferably 90 mol % or less, more preferably 80 mol % or less, further preferably 70 mol % or less, based on the total amount of the monomer components of the polymer (A).

Other monomers to be copolymerized with the compound (A) in the polymer (A) are preferably the constitutional unit represented by the following formula (C2), from the viewpoint of the quality of the pattern shape after exposure and development in a lithography process, in particular, suppressing the roughness and the pattern collapse.

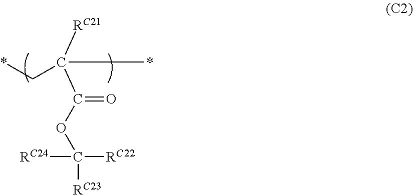

In the formula (C2),
R^{C21} is a hydrogen atom, a methyl group, or a trifluoromethyl group;
each of R^{C22} and R^{C23} is independently an alkyl group having 1 to 4 carbon atoms;
R^{C24} is an alkyl group having 1 to 4 carbon atoms or a cycloalkyl group having 5 to 20 carbon atoms;
two or three selected from R^{C22}, R^{C23}, and R^{C24} are optionally taken together with a carbon atom bonded to the two or three selected from R^{C22}, R^{C23}, and R^{C24} to form an alicyclic structure having 3 to 20 carbon atoms; and
* is a site for binding with an adjacent constitutional unit.

R^{C22} is preferably an alkyl group having 1 to 3 carbon atoms, and R^{C24} is a cycloalkyl group having 5 to 10 carbon atoms. The alicyclic structure formed by R^{C22}, R^{C23}, and R^{C24} may contain a plurality of rings such as an adamantyl group. The alicyclic structure may have a substituent (e.g., a hydroxyl group and an alkyl group).

The amount of the constitutional unit represented by the formula (C2) is preferably 5 mol % or more, more preferably 10 mol % or more, and further preferably 20 mol % or more, based on the total amount of the monomer components of the polymer (A). The amount of the constitutional unit represented by the formula (C2) is preferably 80 mol % or less, more preferably 60 mol % or less, further preferably 40 mol % or less, based on the total amount of the monomer components of the polymer (A).

Examples of the monomer raw material of the constitutional unit represented by the formula (C2) include, but are not limited to, 2-methyl-2-(meth)acryloyloxyadamantane, 2-ethyl-2-(meth)acryloyloxyadamantane, 2-isopropyl-2-(meth)acryloyloxyadamantane, 2-n-propyl-2-(meth)acryloyloxyadamantane, 2-n-butyl-2-(meth)acryloyloxyadamantane, 1-methyl-1-(meth)acryloyloxycyclopentane, 1-ethyl-1-(meth)acryloyloxycyclopentane, 1-methyl-1-(meth)acryloyloxycyclohexane, 1-ethyl-1-(meth)acryloyloxycyclohexane, 1-methyl-1-(meth)acryloyloxycycloheptane, 1-ethyl-1-(meth)acryloyloxycycloheptane, 1-methyl-1-(meth)acryloyloxycyclooctane, 1-ethyl-1-(meth)acryloyloxycyclooctane, 2-ethyl-2-(meth)acryloyloxydecahydro-1,4:5,8-dimethanonaphtalene, and 2-ethyl-2-(meth)acryloyloxynorbornane. Commercial products may be used as these monomer.

Other monomers to be copolymerized with the compound (A) in the polymer (A) are preferably the constitutional unit represented by the following formula (C0), from the viewpoint of the quality of the pattern shape after exposure and development in a lithography process, sensitization, in particular, suppressing the roughness and the pattern collapse.

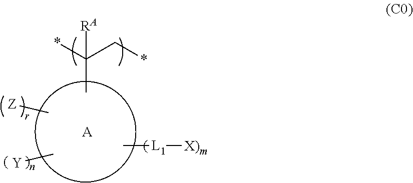

In the formula (C0),
each X is independently I, F, Cl, Br, or an organic group having 1 to 30 carbon atoms and having 1 or more and 5 or less substituents selected from the group consisting of I, F, Cl, and Br;
each L¹ is independently a single bond, an ether group, an ester group, a thioether group, an amino group, a thioester group, an acetal group, a phosphine group, a phosphone group, a urethane group, a urea group, an amide group, an imide group, or a phosphate group, and the ether group, the ester group, the thioether group, the amino group, the thioester group, the acetal group, the phosphine group, the phosphone group, the urethane group, the urea group, the amide group, the imide group, or the phosphate group of L¹ optionally has a substituent;
each Y is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;
R^{A} is as defined in the formula (1);
A is an organic group having 1 to 30 carbon atoms;
each Z is independently an alkoxy group, an ester group, an acetal group, a carboxyalkoxy group, or a carbonate ester group, and the alkoxy group, the ester group, the acetal group, the carboxyalkoxy group, or the carbonate ester group of Z optionally has a substituent; and
m is an integer of 0 or more, n is an integer of 1 or more, and r is an integer of 0 or more.)

Examples of “the organic group having 1 to 30 carbon atoms and having 1 or more and 5 or less substituents selected from the group consisting of I, F, Cl, and Br” include, but are not particularly limited to, a monoiodophenyl group, a diiodophenyl group, a triiodophenyl group, a tetraiodophenyl group, a pentaiodophenyl group, a monoiodohydroxyphenyl group, a diiodohydroxyphenyl group, a triiodohydroxyphenyl group, a monoiodoacetoxyphenyl group, a diiodoacetoxyphenyl group, a triiodoacetoxyphenyl group, a monoiodo-t-butoxycarbonylphenyl group, a diiodo-t-butoxycarbonylphenyl group, a triiodo-t-butoxycarbonylphenyl group, a monoiododihydroxyphenyl group, a diiododihydroxyphenyl group, a triiododihydroxyphenyl group, a monoiododiacetoxyphenyl group, a diiododiacetoxyphenyl group, a triiododiacetoxyphenyl group, a monoiodo-di-t-butoxycarbonylphenyl group, a diiodo-di-t-butoxycarbonylphenyl group, a triiodo-di-t-butoxycarbonylphenyl group, a monoiodotrihydroxyphenyl group, a diiodotrihydroxyphenyl group, a monoiodotriacetoxyphenyl group, a diiodotriacetoxyphenyl group, a monoiodo-tri-t-butoxycarbonylphenyl group, a diiodo-tri-t-butoxycarbonylphenyl group, a monoiodonaphthyl group, a diiodonaphthyl group, a triiodonaphthyl group, a tetraiodonaphthyl group, a pentaiodonaphthyl group, a monoiodohydroxynaphthyl group, a diiodohydroxynaphthyl group, a triiodohydroxynaphthyl group, a monoiodoacetoxynaphthyl group, a diiodoacetoxynaphthyl group, a tri iodoacetoxynaphthyl group, a monoiodo-t-butoxycarbonylnaphthyl group, a diiodo-t-butoxycarbonylnaphthyl group, a triiodo-t-butoxycarbonylnaphthyl group, a monoiododihydroxynaphthyl group, a diiododihydroxynaphthyl group, a triiododihydroxynaphthyl group, a monoiododiacetoxynaphthyl group, a diiododiacetoxynaphthyl group, a triiododiacetoxynaphthyl group, a monoiodo-di-t-butoxycarbonylnaphthyl group, a diiodo-di-t-butoxycarbonylnaphthyl group, a triiodo-di-t-butoxycarbonylnaphthyl group,
a monoiodotrihydroxynaphthyl group, a diiodotrihydroxynaphthyl group, a monoiodotriacetoxynaphthyl group, a diiodotriacetoxynaphthyl group, a monoiodo-tri-t-butoxycarbonylnaphthyl group, a diiodo-tri-t-butoxycarbonylnaphthyl group, a monoiodoadamantyl group, a diiodoadamantyl group, a triiodoadamantyl group, a monoiodohydroxyadamantyl group, a diiodohydroxynaphthyl group, a monoiodoacetoxynaphthyl group, a diiodoacetoxyadamantyl group, a monoiodo-t-butoxycarbonyladamantyl group, a diiodo-t-butoxycarbonyladamantyl group, a triiodo-t-butoxycarbonyladamantyl group, a monoiododihydroxyadamantyl group, a monoiododiacetoxyadamantyl group, a monoiodo-di-t-butoxycarbonyladamantyl group, a monoiodocyclohexyl group, a diiodocyclohexyl group, a triiodocyclohexyl group, a monoiodohydroxycyclohexyl group, a diiodohydroxynaphthyl group, a monoiodoacetoxynaphthyl group, a diiodoacetoxycyclohexyl group, a monoiodo-t-butoxycarbonylcyclohexyl group, a diiodo-t-butoxycarbonylcyclohexyl group, a triiodo-t-butoxycarbonylcyclohexyl group, a monoiododihydroxycyclohexyl group, a monoiododiacetoxycyclohexyl group, a monoiodo-di-t-butoxycarbonylcyclohexyl group,
   a monobromophenyl group, a dibromophenyl group, a tribromophenyl group, a tetrabromophenyl group, a pentabromophenyl group, a monobromohydroxyphenyl group, a dibromohydroxyphenyl group, a tribromohydroxyphenyl group, a monobromoacetoxyphenyl group, a dibromoacetoxyphenyl group, a tribromoacetoxyphenyl group, a monobromo t-butoxycarbonylphenyl group, a dibromo t-butoxycarbonylphenyl group, a tribromo t-butoxycarbonylphenyl group, a monobromodihydroxyphenyl group, a dibromodihydroxyphenyl group, a tribromodihydroxyphenyl group, a monobromodiacetoxyphenyl group, a dibromodiacetoxyphenyl group, a tribromodiacetoxyphenyl group, a monobromo di-t-butoxycarbonylphenyl group, a dibromo di-t-butoxycarbonylphenyl group, a tribromo di-t-butoxycarbonyiphenyl group,
a monobromotrihydroxyphenyl group, a dibromotrihydroxyphenyl group, a monobromotriacetoxyphenyl group, a dibromotriacetoxyphenyl group, a monobromotri-t-butoxycarbonylphenyl group, a dibromotri-t-butoxycarbonyiphenyl group, a monobromoadamantyl group, a dibromoadamantyl group, a tribromoadamantyl group, a monobromohydroxyadamantyl group, a dibromohydroxynaphthyl group, a monobromoacetoxynaphthyl group, a dibromoacetoxyadamantyl group, a monobromo t-butoxycarbonyladamantyl group, a dibromo t-butoxycarbonyladamantyl group, a tribromo t-butoxycarbonyladamantyl group, a monobromodihydroxyadamantyl group, a monobromodiacetoxyadamantyl group, a monobromo-di-t-butoxycarbonyladamantyl group,
   a monofluorophenyl group, a difluorophenyl group, a trifluorophenyl group, a tetrafluorophenyl group, a pentafluorophenyl group, a monofluorohydroxyphenyl group, a difluorohydroxyphenyl group, a trifluorohydroxyphenyl group, a monofluoroacetoxyphenyl group, a difluoroacetoxyphenyl group, a trifluoroacetoxyphenyl group, a monofluoro t-butoxycarbonyiphenyl group, a difluoro t-butoxycarbonylphenyl group, a trifluoro t-butoxycarbonylphenyl group, a monofluorodihydroxyphenyl group, a difluorodihydroxyphenyl group, a trifluorodihydroxyphenyl group, a monofluorodiacetoxyphenyl group, a difluorodiacetoxyphenyl group, a trifluorodiacetoxyphenyl group, a monofluorodi-t-butoxycarbonylphenyl group, a difluorodi-t-butoxycarbonylphenyl group, a trifluorodi-t-butoxycarbonyiphenyl group, a monofluorotrihydroxyphenyl group, a difluorotrihydroxyphenyl group, a monofluorotriacetoxyphenyl group, a difluorotriacetoxyphenyl group, a monofluorotri-t-butoxycarbonylphenyl group, a difluorotri-t-butoxycarbonylphenyl group, a monofluoroadamantyl group, a difluoroadamantyl group, a trifluoroadamantyl group, a monofluorohydroxyadamantyl group, a difluorohydroxynaphthyl group, a monofluoroacetoxynaphthyl group, a difluoroacetoxyadamantyl group, a monofluoro t-butoxycarbonyladamantyl group, a difluoro t-butoxycarbonyladamantyl group, a trifluoro t-butoxycarbonyladamantyl group, a monofluorodihydroxyadamantyl group, a monofluorodiacetoxyadamantyl group, a monofluoro-di-t-butoxycarbonyladamantyl group,
   a monochlorophenyl group, a dichlorophenyl group, a trichlorophenyl group, a tetrachlorophenyl group, a pentachlorophenyl group, a monochlorohydroxyphenyl group, a dichlorohydroxyphenyl group, a trichlorohydroxyphenyl group, a monochloroacetoxyphenyl group, a dichloroacetoxyphenyl group, a trichloroacetoxyphenyl group, a monochloro t-butoxycarbonylphenyl group, a dichloro t-butoxycarbonylphenyl group, a trichloro t-butoxycarbonylphenyl group, a monochlorodihydroxyphenyl group, a dichlorodihydroxyphenyl group, a trichlorodihydroxyphenyl group, a monochlorodiacetoxyphenyl group, a dichlorodiacetoxyphenyl group, a trichlorodiacetoxyphenyl group, a monochlorodi-t-butoxycarbonylphenyl group, a dichlorodi-t-butoxycarbonylphenyl group, a trichlorodi-t-butoxycarbonylphenyl group,
a monochlorotrihydroxyphenyl group, a dichlorotrihydroxyphenyl group, a monochlorotriacetoxyphenyl group, a dichlorotriacetoxyphenyl group, a monochlorotri-t-butoxycarbonylphenyl group, a dichlorotri-t-butoxycarbonylphenyl group, a monochloroadamantyl group, a dichloroadamantyl group, a trichloroadamantyl group, a monochlorohydroxyadamantyl group, a dichlorohydroxynaphthyl group, a monochloroacetoxynaphthyl group, a dichloroacetoxyadamantyl group, a monochloro t-butoxycarbonyladamantyl group, a dichloro t-butoxycarbonyladamantyl group, a trichloro t-butoxycarbonyladamantyl group, a monochlorodihydroxyadamantyl group, a monochlorodiacetoxyadamantyl group, and a monochlorodi-t-butoxycarbonyladamantyl group.

For example, X may be an aromatic group into which one or more F, Cl, Br, or I are introduced. Examples of such an aromatic group include a group having a benzene ring such as a phenyl group and having 1 to 5 halogens, and a group having a heteroaromatic ring such as furan, thiophene, and pyridine and having 1 to 5 halogens. Examples thereof include a phenyl group having 1 to 5 I, a phenyl group having 1 to 5 F, a phenyl group having 1 to 5 Cl, a phenyl group having 1 to 5 Br, a naphthyl group having 1 to 5 F, a naphthyl group having 1 to 5 Cl, a naphthyl group having 1 to 5 Br, a naphthyl group having 1 to 5 I, a phenol group having 1 to 4 F, a phenol group having 1 to 4 Cl, a phenol group having 1 to 4 Br, a phenol group having 1 to 4 I, a furan group having 1 to 3 F, a furan group having 1 to 3 Cl, a furan group having 1 to 3 Br, a furan group having 1 to 3 I, a thiophene group having 1 to 3 F, a thiophene group having 1 to 3 Cl, a thiophene group having 1 to 3 Br, a thiophene group having 1 to 3 I, a pyridine group having 1 to 4 F, a pyridine group having 1 to 4 Cl, a pyridine group having 1 to 4 Br, a pyridine group having 1 to 4 I, a benzodiazole group having 1 to 5 F, a benzodiazole group having 1 to 5 Cl, a benzodiazole group having 1 to 5 Br, a benzodiazole group having 1 to 5 I, a benzimidazole group having 1 to 4 F, a benzimidazole group having 1 to 4 Cl, a benzimidazole group having 1 to 4 Br, a benzimidazole group having 1 to 4 I, a benzoxazole group having 1 to 4 F, a benzoxazole group having 1 to 4 Cl, a benzoxazole group having 1 to 4 Br, a benzoxazole group having 1 to 4 I, a benzothiophene group having 1 to 4 F, a benzothiophene group having 1 to 4 Cl, a benzothiophene group having 1 to 4 Br, and a benzothiophene group having 1 to 4 I. X may be an alicyclic group into which one or more F, Cl, Br, or I are introduced. Examples of such an alicyclic group include an adamantyl group having 1 to 3 halogens, an adamantyl group having 1 to 3 F, an adamantyl group having 1 to 3 Cl, an adamantyl group having 1 to 3 Br, an adamantyl group having 1 to 3 I, a cyclopentyl group having 1 to 3 F, a cyclopentyl group having 1 to 3 Cl, a cyclopentyl group having 1 to 3 Br, a cyclopentyl group having 1 to 3 I, a bicycloundecyl group having 1 to 3 F, a bicycloundecyl group having 1 to 3 Cl, a bicycloundecyl group having 1 to 3 Br, a bicycloundecyl group having 1 to 3 I, a norbornyl group having 1 to 3 F, a norbornyl group having 1 to 3 Cl, a norbornyl group having 1 to 3 Br, and a norbornyl group having 1 to 3 I.

L¹ is a single bond, an ether group, an ester group, a thioether group, an amino group, a thioester group, an acetal group, a phosphine group, a phosphone group, a urethane group, a urea group, an amide group, an imide group, or a phosphate group. Among them, L¹ is preferably a single bond. The ether group, the ester group, the thioether group, the amino group, the thioester group, the acetal group, the phosphine group, the phosphone group, the urethane group, the urea group, the amide group, the imide group, or the phosphate group of L¹ optionally has a substituent. Examples of such a substituent are as described above.

m is an integer of 0 or more, preferably an integer of 0 or more and 5 or less, more preferably an integer of 0 or more and 2 or less, further preferably 0 or 1, and particularly preferably 0.

Each Y is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent.

Examples of Y include at least one group selected from the group consisting of an alkoxy group [^{*3}—O—R²], an ester group [^{*3}—O—(C═O)—R² or ^{*3}—(C═O)—O—R²], an acetal group) [^{*3}—O—(C(R²¹)₂)—O—R² (wherein each R²¹ is independently H or a hydrocarbon group having 1 to 10 carbon atoms)], a carboxyalkoxy group [^{*3}—O—R²² (C═O)—O—R² (wherein R²² is a divalent hydrocarbon group having 1 to 10 carbon atoms)], and a carbonate ester group [^{*3}—O—(C═O)—O—R²]. The ester group is preferably a tertiary ester group, from the viewpoint of achieving high sensitivity. In the formula, ^{*3} is a site for binding with A.

Among them, Y is preferably a tertiary ester group, an acetal group, a carbonate ester group, or a carboxyalkoxy group, more preferably an acetal group, a carbonate ester group, or a carboxyalkoxy group, and further preferably an acetal group or a carboxyalkoxy group, from the viewpoint of high sensitivity. From the viewpoint of producing a polymer having a stable quality by radical polymerization, an ester group, a carboxyalkoxy group, and a carbonate ester group are preferable.

Each Y is preferably independently a group represented by the following formula (Y-1).

-L²-R² (Y-1)

In the formula (Y-1),

L² is a group which is cleaved by the action of an acid or a base. Examples of the group which is cleaved by the action of an acid or a base include at least one divalent linking group selected from the group consisting of an ester group [^{*1}—O—(C═O)—^{*2} or ^{*1}—(C═O)—O—^{*2}], an acetal group [^{*1}—O—(C(R²¹)₂)—O—^{*2} (each R²¹ is independently H or a hydrocarbon group having 1 to 10 carbon atoms)], a carboxyalkoxy group [^{*1}—O—R²²—(C═O)—O—^{*2} (R²² is a divalent hydrocarbon group having 1 to 10 carbon atoms)], and a carbonate ester group [^{*1}—O—(C═O)—O—^{*2}]. The ester group is preferably a tertiary ester group, from the viewpoint of achieving high sensitivity. In the formula, ^{*1} is a site for binding with A, and ^{*2} is a site for binding with R². Among them, L² is preferably a tertiary ester group, an acetal group, a carbonate ester group, or a carboxyalkoxy group, more preferably an acetal group, a carbonate ester group, or a carboxyalkoxy group, and further preferably an acetal group or a carboxyalkoxy group, from the viewpoint of high sensitivity. From the viewpoint of producing a polymer having a stable quality by radical polymerization, an ester group, a carboxyalkoxy group, and a carbonate ester group are preferable.

As another effect, Y is preferably a group represented by the formula (Y-1) to control the polymerization properties of resin and the degree of polymerization in a desired range, when the compound (A) of the second embodiment is used as a polymerization unit of a copolymer. Since the compound (A) has a large influence on activity species in the polymer formation reaction due to having an X group and thus the desired control is difficult, variation of copolymer formation derived from the hydrophilic group and polymerization inhibition can be suppressed by having a group represented by the formula (Y-1) in the hydrophilic group in the compound (A) as a protecting group.

R² is a linear, branched, or cyclic aliphatic group having 1 to 30 carbon atoms, an aromatic group having 6 to 30 carbon atoms, a linear, branched, or cyclic aliphatic group containing a heteroatom and having 1 to 30 carbon atoms, or a linear, branched, or cyclic aromatic group containing a heteroatom and having 1 to 30 carbon atoms, and the aliphatic group, the aromatic group, the aliphatic group containing a heteroatom, and the aromatic group containing a heteroatom of R² optionally further have a substituent. As the substituent here, the aforementioned substituents are used, but a linear, branched, or cyclic aliphatic group having 1 to 20 carbon atoms or an aromatic group having 6 to 20 carbon atoms is preferable. Among them, R² is preferably an aliphatic group. The aliphatic group in R² is preferably a branched or cyclic aliphatic group. The number of carbon atoms of the aliphatic group is preferably 1 or more and 20 or less, more preferably 3 or more and 10 or less, and further preferably 4 or more and 8 or less. Examples of the aliphatic group include, but are not particularly limited to, a methyl group, an isopropyl group, a sec-butyl group, a tert-butyl group, an isobutyl group, a cyclohexyl group, a methylcyclohexyl group, and an adamantyl group. Among them, a tert-butyl group, a cyclohexyl group, or an adamantyl group is preferable.

L² is preferably ^{*1}—(C═O)—O—^{*2} or a carboxyalkoxy group, because, when L² is cleaved by the action of an acid or a base, a carboxylic acid group is formed and the difference in the solubility and the difference in the dissolution rate between a cleaved portion and an uncleaved portion are increased in the development treatment, so that the resolution is improved and, in particular, residues at the pattern bottom in thin line patterns are suppressed.

Specific examples of Y include the followings. Each Y is independently a group represented by any of the following formulas.

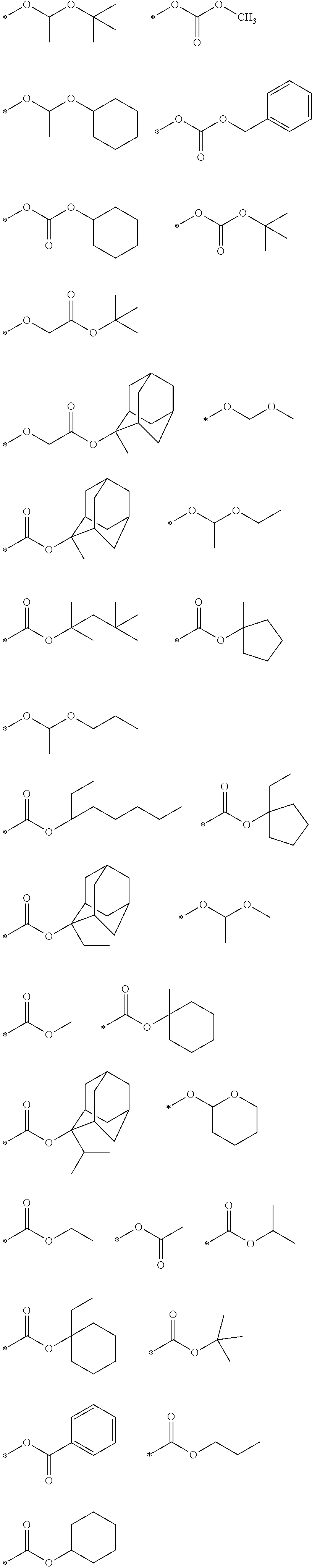

Examples of the alkoxy group that can be used as Y include an alkoxy group having 1 or more carbon atoms, and from the viewpoint of the solubility of a resin after the compound is combined with other monomers to form the resin, an alkoxy group having 2 or more carbon atoms is preferable, and an alkoxy group having 3 or more carbon atoms or having a cyclic structure is preferable.

Specific examples of the alkoxy group that can be used as Y include, but are not limited to, the followings.

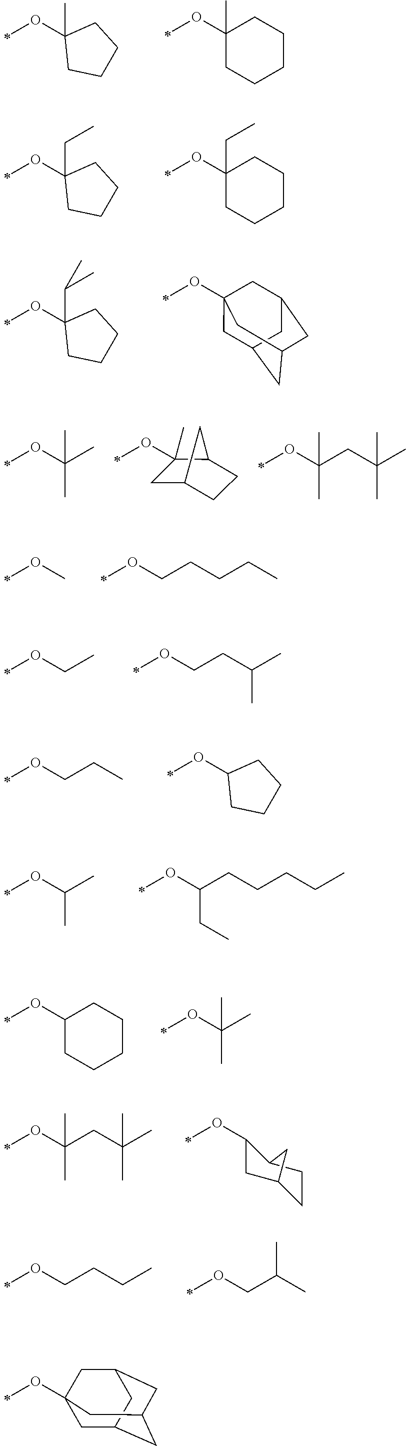

As the amino group and amide group that can be used as Y, a primary amino group, a secondary amino group, a tertiary amino group, a group having a quaternary ammonium salt structure, an amide having a substituent, or the like can be arbitrarily used. Specific examples of the amino group or amide group that can be used include, but are not limited to, the followings.

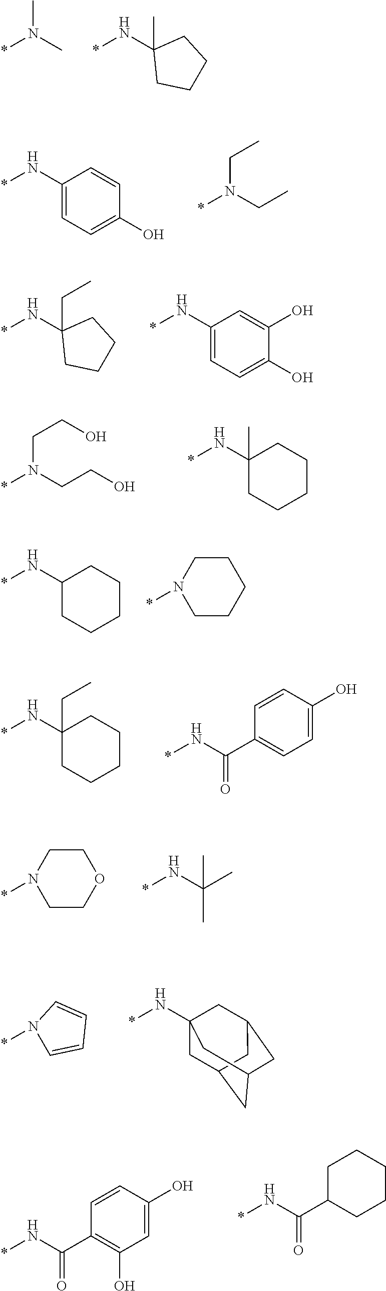

n is an integer of 1 or more, preferably an integer of 1 or more and 5 or less, more preferably an integer of 1 or more and 4 or less, further preferably an integer of 1 or more and 3 or less, still more preferably 1 or 2, and particularly preferably 1.
each R^{A} is independently H, I, F, C1, Br, or an organic group having 1 to 60 carbon atoms and optionally having a substituent. Examples of the substituent of the organic group having 1 to 60 carbon atoms include, but are not particularly limited to, I, F, Cl, Br, or other substituents. Examples of other substituents include, but are not particularly limited to, a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, and a phosphate group. Among them, the alkoxy group, the ester group, the carbonate ester group, the amino group, the ether group, the thioether group, the phosphine group, the phosphone group, the urethane group, the urea group, the amide group, the imide group, and the phosphate group optionally further have a substituent. Examples of the substituent here include a linear, branched, or cyclic aliphatic group having 1 to 20 carbon atoms and an aromatic group having 6 to 20 carbon atoms.

The number of carbon atoms of the organic group optionally having a substituent in R^{A} is preferably 1 to 30.

Examples of the organic group having 1 to 60 carbon atoms and optionally having a substituent include, but are not particularly limited to, a linear or branched aliphatic hydrocarbon group having 1 to 60 carbon atoms, a cycloaliphatic hydrocarbon group having 4 to 60 carbon atoms, and an aromatic group having 6 to 60 carbon atoms and optionally having a heteroatom.

Examples of the linear or branched aliphatic hydrocarbon group having 1 to 60 carbon atoms include, but are not particularly limited to, a methyl group, an ethyl group, an n-propyl group, an isopropyl group, an n-butyl group, an isobutyl group, a tert-butyl group, an n-pentyl group, an n-hexyl group, an n-dodecyl group, a valeryl group, and a 2-ethylhexyl group.

Examples of the cycloaliphatic hydrocarbon group include, but are not particularly limited to, a cyclohexyl group, a cyclododecyl group, a dicyclopentyl group, a tricyclodecyl group, and an adamantyl group. Further, an aromatic group optionally having a heteroatom such as a benzodiazole group, a benzotriazole group, and a benzothiadiazole group can be arbitrarily selected. A combination of these organic groups can also be selected.

Examples of the aromatic group having 6 to 60 carbon atoms and optionally having a heteroatom include, but are not particularly limited to, a phenyl group, a naphthalene group, a biphenyl group, an anthracyl group, a pyrenyl group, a benzodiazole group, a benzotriazole group, and a benzothiadiazole group.

Among the organic group having 1 to 60 carbon atoms and optionally having a substituent, a methyl group is preferable, from the viewpoint of producing a polymer having a stable quality.

A is an organic group having 1 to 30 carbon atoms. A may be a monocyclic organic group or a polycyclic organic group, or optionally has a substituent. A is preferably an aromatic ring optionally having a substituent. The number of carbon atoms of A is preferably 6 to 14, and more preferably 6 to 10.

A is preferably a group represented by any of the following formulas, more preferably a group represented by the following formulas (A-1) to (A-2), and further preferably a group represented by the following formula (A-1).

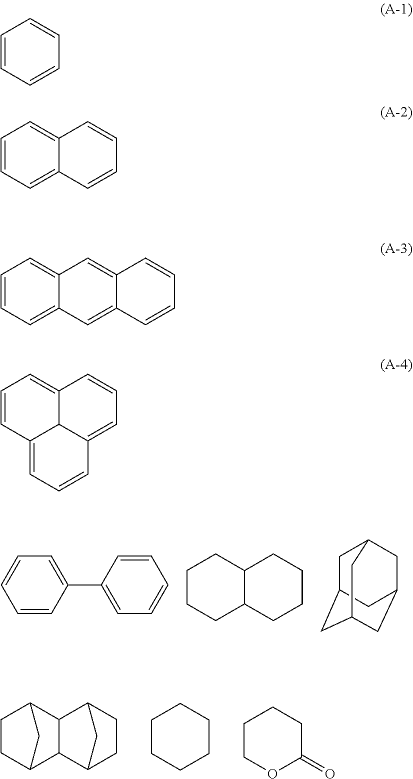

A may be an alicyclic structure optionally having a substituent. Here, the “alicyclic structure” refers to a saturated or unsaturated carbocycle having no aromatic properties. Examples of the alicyclic structure include a saturated or unsaturated carbocycle having 3 to 30 carbon atoms, and a saturated or unsaturated carbocycle having 3 to 20 carbon atoms is preferable. Examples of the alicyclic structure include a group having cyclopropyl, cyclobutyl, cyclopentyl, cyclohexyl, cycloheptyl, cyclooctyl, cyclononyl, cyclodecyl, cycloicosyl, cyclopropenyl, cyclobutenyl, cyclopentenyl, cyclohexenyl, cycloheptenyl, cyclooctenyl, cyclopentadienyl, cyclooctadienyl, adamantyl, bicycloundecyl, decahydronaphthyl, norbornyl, norbornadienyl, cubane, basketane, or housane.

A may also be a heterocyclic structure optionally having a substituent. Examples of the heterocyclic structure include, but are not particularly limited to, a nitrogen-containing cyclic structure such as pyridine, piperidine, piperidone, benzodiazole, and benzotriazole; a cyclic ether such as triazine, a cyclic urethane structure, cyclic urea, cyclic amide, cyclic imide, furan, pyran, and dioxolane; an alicyclic group having a lactone structure such as caprolactone, butyrolactone, nonalactone, decalactone, undecalactone, bicycloundecalactone, and phthalide.

Each Z is independently an alkoxy group, an ester group, an acetal group, a carboxyalkoxy group, or a carbonate ester group. These groups optionally have a substituent, and examples of the substituent include a hydrocarbon group having 1 to 60 carbon atoms and optionally further having a substituent. r is an integer of 0 or more, preferably an integer of 0 or more and 2 or less, more preferably an integer of 0 or more and 1 or less, and further preferably 0.

Examples of Z include at least one group selected from the group consisting of an alkoxy group [^{*3}—O—R²], an ester group [^{*3}—O—(C═O)—R² or ^{*3}—(C═O)—O—R²], an acetal group [^{*3}—O—(C(R²¹)₂)—O—R² (wherein each R²¹ is independently H or a hydrocarbon group having 1 to 10 carbon atoms)], a carboxyalkoxy group [^{*3}—O—R²²—(C═O)—O—R² (wherein R²² is a divalent hydrocarbon group having 1 to 10 carbon atoms)], and a carbonate ester group [^{*3}—O—(C═O)—O—R²]. The ester group is preferably a tertiary ester group, from the viewpoint of achieving high sensitivity. In the formula, ^{*3} is a site for binding with A.

Among them, Z is preferably a tertiary ester group, an acetal group, a carbonate ester group, or a carboxyalkoxy group, more preferably an acetal group, a carbonate ester group, or a carboxyalkoxy group, and further preferably an acetal group or a carboxyalkoxy group, from the viewpoint of high sensitivity. From the viewpoint of producing a polymer having a stable quality by radical polymerization, an ester group, a carboxyalkoxy group, and a carbonate ester group are preferable.

Other monomers to be copolymerized with the compound (A) in the polymer (A) preferably have a constitutional unit represented by the following formula (C3).

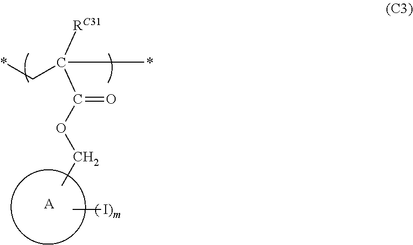

In the formula (C3), R^{C31} is a hydrogen atom, a methyl group, or a trifluoromethyl group; and m, A, and * are as defined in the formula (C0).

Next, the method for producing the polymer (A) will be described. The polymerization reaction is carried out by dissolving a monomer as a constitutional unit in a solvent and adding a polymerization initiator while heating or cooling. The reaction conditions can be inadvertently set depending on the kind of polymerization initiator, the initiation method such as heat and light, the temperature, the pressure, the concentration, the solvent, the additive, and the like. Examples of the polymerization initiator include a radical polymerization initiator such as azoisobutyronitrile and peroxide, and an anion polymerization initiator such as alkyllithium and a Grignard reagent.

As the solvent used in the polymerization reaction, a commonly available product can be used. For example, a wide variety of solvents such as alcohols, ethers, hydrocarbons, and halogenated solvents can be arbitrarily used within a range not inhibiting the reaction. A plurality of solvents may be mixed and used within a range not inhibiting the reaction.

The polymer (A) obtained in the polymerization reaction can be purified by a publicly known method. Specifically, ultrafiltration, crystallization, microfiltration, acid washing, water washing at an electrical conductivity of 10 mS/m or less, and extraction can be used in combination.

### [Composition and Composition for Film Formation]

The composition or composition for film formation of the second embodiment contains the compound (A) or the polymer (A), and is the composition particularly suitable for lithography technology. The composition or the composition for film formation can be used for, without particular limitation, film formation purposes for lithography, for example, resist film formation purposes (that is, a “resist composition”). Furthermore, the composition or the composition for film formation can be used for upper layer film formation purposes (that is, a “composition for upper layer film formation”), intermediate layer formation purposes (that is, a “composition for intermediate layer formation”), underlayer film formation purposes (that is, a “composition for underlayer film formation”), and the like. According to the composition of the second embodiment, not only a film having high sensitivity can be formed, but also a good resist pattern shape can be imparted.

The composition for film formation of the second embodiment can also be used as an optical component forming composition applying lithography technology. The optical component is used in the form of a film or a sheet and is also useful as a plastic lens (a prism lens, a lenticular lens, a microlens, a Fresnel lens, a viewing angle control lens, a contrast improving lens, etc.), a phase difference film, a film for electromagnetic wave shielding, a prism, an optical fiber, a solder resist for flexible printed wiring, a plating resist, an interlayer insulating film for multilayer printed circuit boards, a photosensitive optical waveguide, a liquid crystal display, an organic electroluminescent (EL) display, an optical semiconductor (LED) element, a solid state image sensing element, an organic thin film solar cell, a dye sensitized solar cell, and an organic thin film transistor (TFT). The composition can be suitably utilized as an embedded film and a smoothed film on a photodiode, a smoothed film in front of or behind a color filter, a microlens, and a smoothed film and a conformal film on a microlens, all of which are components of a solid state image sensing element, to which high refractive index is particularly demanded.

The composition for film formation of the second embodiment may contain the compound (A), the composition of the second embodiment, or the polymer (A). The composition for film formation of the second embodiment may further contain an acid generating agent (C), a base generating agent (G), or an acid diffusion controlling agent (E) (basic compound). The composition for film formation of the second embodiment may further contain other components such as a base material (B) and a solvent (S), if required. Hereinafter, each of these components will be described.

### [Base Material (B)]

The “base material (B)” in the second embodiment is a compound (including a resin) other than the compound (A) or the polymer (A) and means a base material applied as a resist for g-ray, i-ray, KrF excimer laser (248 nm), ArF excimer laser (193 nm), extreme ultraviolet (EUV) lithography (13.5 nm) or electron beam (EB) (for example, a base material for lithography or a base material for resist). These base materials can be used as the base material (B) in the second embodiment without particular limitation. Examples of the base material (B) include a phenol novolac resin, a cresol novolac resin, a hydroxystyrene resin, a (meth)acrylic resin, and a hydroxystyrene-(meth)acrylic copolymer, a cycloolefin-maleic anhydride copolymer, a cycloolefin, a vinyl ether-maleic anhydride copolymer, and an inorganic resist material having a metallic element such as titanium, tin, hafnium, and zirconium, and derivatives thereof. Among them, from the viewpoint of the shape of a resist pattern to be obtained, preferable are a phenol novolac resin, a cresol novolac resin, a hydroxystyrene resin, a (meth)acrylic resin, a hydroxystyrene-(meth)acrylic copolymer, and an inorganic resist material having a metallic element such as titanium, tin, hafnium, and zirconium, and derivatives thereof.

Examples of the derivative include, but are not particularly limited to, those to which a dissociation group is introduced and those to which a crosslinkable group is introduced. The above derivative to which a dissociation group or a crosslinkable group is introduced can exhibit dissociation reaction or crosslinking reaction through the effect of light, acid or the like.

The “dissociation group” refers to a characteristic group that is cleaved to generate a functional group that alters solubility, such as an alkali soluble group. Examples of the alkali soluble group include, but are not particularly limited to, a phenolic hydroxy group, a carboxyl group, a sulfonic acid group, and a hexafluoroisopropanol group, and a phenolic hydroxy group and a carboxyl group are preferable, and a phenolic hydroxy group is particularly preferable.

The “crosslinking group” refers to a group that crosslinks in the presence of a catalyst or without a catalyst. Examples of the crosslinking group include, but are not particularly limited to, an alkoxy group having 1 to 20 carbon atoms, a group having an allyl group, a group having a (meth)acryloyl group, a group having an epoxy (meth)acryloyl group, a group having a hydroxy group, a group having a urethane (meth)acryloyl group, a group having a glycidyl group, and a group having a vinyl-containing phenylmethyl group.

### [Solvent (S)]

As the solvent in the second embodiment, a known solvent can be arbitrarily used as long as it can at least dissolve the compound (A) or the polymer (A) mentioned above. Examples of the solvent include, but are not particularly limited to, ethylene glycol monoalkyl ether acetates such as ethylene glycol monomethyl ether acetate, ethylene glycol monoethyl ether acetate, ethylene glycol mono-n-propyl ether acetate, and ethylene glycol mono-n-butyl ether acetate; ethylene glycol monoalkyl ethers such as ethylene glycol monomethyl ether and ethylene glycol monoethyl ether; propylene glycol monoalkyl ether acetates such as propylene glycol monomethyl ether acetate (PGMEA), propylene glycol monoethyl ether acetate, propylene glycol mono-n-propyl ether acetate, and propylene glycol mono-n-butyl ether acetate; propylene glycol monoalkyl ethers such as propylene glycol monomethyl ether (PGME) and propylene glycol monoethyl ether; lactate esters such as methyl lactate, ethyl lactate, n-propyl lactate, n-butyl lactate, and n-amyl lactate; aliphatic carboxylic acid esters such as methyl acetate, ethyl acetate, n-propyl acetate, n-butyl acetate, n-amyl acetate, n-hexyl acetate, methyl propionate, and ethyl propionate; other esters such as methyl 3-methoxypropionate, ethyl 3-methoxypropionate, methyl 3-ethoxypropionate, ethyl 3-ethoxypropionate, methyl 3-methoxy-2-methylpropionate, 3-methoxybutylacetate, 3-methyl-3-methoxybutylacetate, butyl 3-methoxy-3-methylpropionate, butyl 3-methoxy-3-methylbutyrate, methyl acetoacetate, methyl pyruvate, and ethyl pyruvate; aromatic hydrocarbons such as toluene and xylene; ketones such as acetone, 2-butanone, 2-heptanone, 3-heptanone, 4-heptanone, cyclopentanone (CPN), and cyclohexanone (CHN); amides such as N,N-dimethylformamide, N-methylacetamide, N,N-dimethylacetamide, and N-methylpyrrolidone; and lactones such as γ-lactone. The solvent used in the second embodiment is preferably a safe solvent, more preferably at least one selected from PGMEA, PGME, CHN, CPN, 2-heptanone, anisole, butyl acetate and ethyl lactate, and further preferably at least one selected from PGMEA, PGME, CHN, CPN and ethyl lactate.

In the composition for film formation of the second embodiment, the solid component concentration is not particularly limited, but is preferably 1 to 80% by mass, more preferably 1 to 50% by mass, further preferably 2 to 40% by mass, and still more preferably 2 to 10% by mass, based on the total mass of the composition for film formation.

### [Acid Generating Agent (C)]

It is preferable that the composition for film formation of the second embodiment contain one or more acid generating agents (C) that directly or indirectly generates an acid by radiation irradiation. Radiation is at least one selected from the group consisting of visible light, ultraviolet, excimer laser, electron beam, extreme ultraviolet (EUV), X-ray, and ion beam. The acid generating agent (C) is not particularly limited, and, for example, an acid generating agent described in International Publication No. WO 2013/024778 can be used. The acid generating agent (C) can be used alone or in combination of two or more kinds.

The amount of the acid generating agent (C) blended is preferably 0.001 to 49% by mass, more preferably 1 to 40% by mass, further preferably 3 to 30% by mass, and still more preferably 10 to 25% by mass, based on the total mass of the solid components. By using the acid generating agent (C) within the above range, a pattern profile with high sensitivity and low edge roughness tends to be obtained. In the second embodiment, the acid generation method is not particularly limited, as long as an acid is generated in the system. By using excimer laser instead of ultraviolet such as g-ray and i-ray, finer processing is possible, and also by using electron beam, extreme ultraviolet, X-ray, or ion beam as a high energy ray, further finer processing is possible.

### [Base Generating Agent (G)]

The case where the base generating agent (G) is a photobase generating agent will be described.

The photobase generating agent generates a base upon exposure and does not exhibit activity under normal conditions at normal temperature and pressure, but is not particularly limited as long as the photobase generating agent generates a base (basic substance) upon irradiation with an electromagnetic wave and heating as an external stimulus.

The photobase generating agent which can be used in the second embodiment is not particularly limited, and a publicly known one can be used, and examples thereof include, for example, a carbamate derivative, an amide derivative, an imide derivative, an α-cobalt complex, an imidazole derivative, a cinnamic acid amide derivative, and an oxime derivative.

The basic substance generated from the photobase generating agent is not particularly limited, and examples thereof include compounds having an amino group, particularly monoamines, polyamines such as diamines, and amidines.

The basic substance to be generated is preferably a compound having an amino group with a higher basicity (a higher pKa value of the conjugate acid) from the viewpoint of sensitivity and resolution.

Examples of the photobase generating agent include, for example, base generating agents having a cinnamic amide structure as disclosed in Japanese Patent Laid-Open No. 2009/80452 and International Publication NO. WO 2009/123122; base generating agents having a carbamate structure as disclosed in Japanese Patent Laid-Open No. 2006/189591 and Japanese Patent Laid-Open No. 2008/247747; base generating agents having an oxime structure or a carbamoyloxime structure as disclosed in Japanese Patent Laid-Open No. 2007/249013 and Japanese Patent Laid-Open No. 2008/003581; and compounds described in Japanese Patent Laid-Open No. 2010/243773, but these are not limited thereto, and other known structures of base generating agents can be used.

The photobase generating agent can be used alone or in combination of two or more kinds.

The preferred content of the photobase generating agent in actinic ray or radiation sensitive resin composition is similar to the preferred content of the aforementioned photoacid generating agent in actinic ray or radiation sensitive resin composition.

### [Acid Diffusion Controlling Agent (E)]

The composition for film formation of the second embodiment may contain the acid diffusion controlling agent (E) as the basic compound. The acid diffusion controlling agent (E) controls diffusion of an acid generated from an acid generating agent by radiation irradiation in a resist film to inhibit any unpreferable chemical reaction in an unexposed region or the like. By using the acid diffusion controlling agent (E), there is a tendency that the storage stability of the composition of the second embodiment can be improved. Also, by using the acid diffusion controlling agent (E), there is a tendency that not only the resolution of a film formed by using the composition of the second embodiment can be improved, but also the line width change of a resist pattern due to variation in the post exposure delay time before radiation irradiation and the post exposure delay time after radiation irradiation can also be suppressed, making the composition excellent in process stability. Examples of the acid diffusion controlling agent (E) include, but are not particularly limited to, a radiation degradable basic compound such as a nitrogen atom containing basic compound, a basic sulfonium compound, and a basic iodonium compound.

The acid diffusion controlling agent (E) is not particularly limited, and, for example, an acid diffusion controlling agent described in International Publication No. WO 2013/024778 can be used. The acid diffusion controlling agent (E) can be used alone or in combination of two or more kinds.

The amount of the acid diffusion controlling agent (E) blended is preferably 0.001 to 49% by mass, more preferably 0.01 to 10% by mass, further preferably 0.01 to 5% by mass, and further preferably 0.01 to 3% by mass, based on the total mass of the solid components. When the amount of the acid diffusion controlling agent (E) blended is within the above range, there is a tendency that a decrease in resolution, and deterioration of the pattern shape and the dimension fidelity or the like can be prevented. Moreover, even though the post exposure delay time from electron beam irradiation to heating after radiation irradiation becomes longer, deterioration of the shape of the pattern upper layer portion can be suppressed. When the amount of the acid diffusion controlling agent (E) blended is 10% by mass or less, there is a tendency that a decrease in sensitivity, and developability of the unexposed portion or the like can be prevented. Also, by using such an acid diffusion controlling agent, there is a tendency that the storage stability of a resist composition is improved, also along with improvement of the resolution, the line width change of a resist pattern due to variation in the post exposure delay time before radiation irradiation and the post exposure delay time after radiation irradiation can be suppressed, making the composition excellent in process stability.

### [Other Component (F)]

To the composition for film formation of the second embodiment, if required, as the other component (F), one kind or two or more kinds of various additives such as a cross-linking agent, a dissolution promoting agent, a dissolution controlling agent, a sensitizing agent, a surfactant, and an organic carboxylic acid or an oxo acid of phosphorus or derivative thereof can be added.

### (Cross-Linking Agent)

The composition for film formation of the second embodiment may contain the cross-linking agent. The cross-linking agent may crosslink at least one of the compound (A), the polymer (A), and the base material (B). It is preferable that the crosslinking agent be an acid crosslinking agent capable of intramolecularly or intermolecularly crosslinking the base material (B) in the presence of the acid generated from the acid generating agent (C). Examples of such an acid crosslinking agent can include a compound having one or more groups capable of crosslinking the base material (B) (hereinafter, referred to as a “crosslinkable group”).

Examples of the crosslinkable group include: (i) a hydroxyalkyl group or a group derived therefrom, such as a hydroxy group, a hydroxyalkyl group (alkyl group having 1 to 6 carbon atoms), alkoxy having 1 to 6 carbon atoms (alkyl group having 1 to 6 carbon atoms) and acetoxy (alkyl group having 1 to 6 carbon atoms); (ii) a carbonyl group or a group derived therefrom, such as a formyl group and a carboxy (alkyl group having 1 to 6 carbon atoms); (iii) a nitrogenous group containing group such as a dimethylaminomethyl group, a diethylaminomethyl group, a dimethylolaminomethyl group, a diethylolaminomethyl group and a morpholinomethyl group; (iv) a glycidyl group containing group such as a glycidyl ether group, a glycidyl ester group and a glycidylamino group; (v) a group derived from an aromatic group such as an allyloxy having 1 to 6 carbon atoms (alkyl group having 1 to 6 carbon atoms) and an aralkyloxy having 1 to 6 carbon atoms (alkyl group having 1 to 6 carbon atoms) such as a benzyloxymethyl group and a benzoyloxymethyl group; and (vi) a polymerizable multiple bond containing group such as a vinyl group and an isopropenyl group. As the crosslinkable group of the crosslinking agent in the second embodiment, a hydroxyalkyl group, an alkoxyalkyl group and the like are preferable, and an alkoxymethyl group is particularly preferable.

The crosslinking agent having the crosslinkable group is not particularly limited, and, for example, an acid crosslinking agent described in International Publication No. WO 2013/024778 can be used. The crosslinking agent can be used alone or in combination of two or more kinds.

The amount of the cross-linking agent blended in the second embodiment is preferably 50% by mass or less, more preferably 40% by mass or less, further preferably 30% by mass or less, and further preferably 20% by mass or less, based on the total mass of the solid components.

### (Dissolution Promoting Agent)

The dissolution promoting agent is a component having a function of, when the solubility of a solid component is too low, increasing the solubility of the solid component in a developing solution to moderately increase the dissolution rate of the compound upon developing. As the dissolution promoting agent, those having a low molecular weight are preferable, and examples thereof include a phenolic compound having a low molecular weight. Examples of the phenolic compound having a low molecular weight include a bisphenol and a tris(hydroxyphenyl)methane. These dissolution promoting agents can be used alone or in mixture of two or more kinds.

The amount of the dissolution promoting agent blended, which is arbitrarily adjusted according to the kind of the above solid component to be used, is preferably 0 to 49% by mass of the total mass of the solid component, more preferably 0 to 5% by mass, further preferably 0 to 1% by mass, and particularly preferably 0% by mass.

### (Dissolution Controlling Agent)

The dissolution controlling agent is a component having a function of, when the solubility of a solid component is too high, controlling the solubility of the solid component in a developing solution to moderately decrease the dissolution rate upon developing. As such a dissolution controlling agent, the one which does not chemically change in steps such as calcination of resist coating, radiation irradiation, and development is preferable.

The dissolution controlling agent is not particularly limited, and examples thereof include an aromatic hydrocarbon such as phenanthrene, anthracene and acenaphthene; a ketone such as acetophenone, benzophenone and phenyl naphthyl ketone; and a sulfone such as methyl phenyl sulfone, diphenyl sulfone and dinaphthyl sulfone. These dissolution controlling agents can be used alone or in combination of two or more kinds.

The amount of the dissolution controlling agent blended, which is arbitrarily adjusted according to the kind of the above compound to be used, is preferably 0 to 49% by mass of the total mass of the solid component, more preferably 0 to 5% by mass, further preferably 0 to 1% by mass, and particularly preferably 0% by mass.

### (Sensitizing Agent)

The sensitizing agent is a component having a function of absorbing irradiated radiation energy, transmitting the energy to the acid generating agent (C), and thereby increasing the acid production amount, and improving the apparent sensitivity of a resist. Examples of such a sensitizing agent can include, but are not particularly limited to, a benzophenone, a biacetyl, a pyrene, a phenothiazine and a fluorene. These sensitizing agents can be used alone or in combination of two or more kinds.

The amount of the sensitizing agent blended, which is arbitrarily adjusted according to the kind of the above compound to be used, is preferably 0 to 49% by mass of the total mass of the solid component, more preferably 0 to 5% by mass, further preferably 0 to 1% by mass, and particularly preferably 0% by mass.

### (Surfactant)

The surfactant is a component having a function of improving coatability and striation of the composition of the second embodiment, and developability of a resist or the like. The surfactant may be any of anionic, cationic, nonionic, and amphoteric surfactants. Preferable examples of the surfactant include a nonionic surfactant. The nonionic surfactant has a good affinity with a solvent to be used in production of the composition of the second embodiment, and can further enhance the effects of the composition of the second embodiment. Examples of the nonionic surfactant include, but are not particularly limited to, a polyoxyethylene higher alkyl ether, a polyoxyethylene higher alkyl phenyl ether, and a higher fatty acid diester of polyethylene glycol. Examples of commercially available products of these surfactants include, hereinafter by trade name, EFTOP (manufactured by Jemco Inc.), MEGAFAC (manufactured by DIC Corporation), Fluorad (manufactured by Sumitomo 3M Limited), AsahiGuard, SurfIon (hereinbefore, manufactured by Asahi Glass Co., Ltd.), Pepole (manufactured by Toho Chemical Industry Co., Ltd.), KP (manufactured by Shin-Etsu Chemical Co., Ltd.), and Polyflow (manufactured by Kyoeisha Chemical Co., Ltd.).

The amount of the surfactant blended, which is arbitrarily adjusted according to the kind of the above solid component to be used, is preferably 0 to 49% by mass of the total mass of the solid component, more preferably 0 to 5% by mass, further preferably 0 to 1% by mass, and particularly preferably 0% by mass.

### (Organic Carboxylic Acid or Oxo Acid of Phosphorus or Derivative Thereof)

For the purpose of prevention of sensitivity deterioration or improvement of a resist pattern shape and post exposure delay stability or the like, and as an additional optional component, the composition of the present embodiment can contain an organic carboxylic acid or an oxo acid of phosphorus or derivative thereof. The organic carboxylic acid or the oxo acid of phosphorus or derivative thereof can be used in combination with the acid diffusion controlling agent, or may be used alone. Examples of suitable organic carboxylic acids include malonic acid, citric acid, malic acid, succinic acid, benzoic acid and salicylic acid. Examples of the oxo acid of phosphorus or derivative thereof include phosphoric acid or derivative thereof such as ester including phosphoric acid, di-n-butyl phosphate and diphenyl phosphate; phosphonic acid or derivative thereof such as ester including phosphonic acid, dimethyl phosphonate, di-n-butyl phosphonate, phenylphosphonic acid, diphenyl phosphonate and dibenzyl phosphonate; and phosphinic acid and derivative thereof such as ester including phosphinic acid and phenylphosphinic acid. Among them, phosphonic acid is particularly preferable.

The organic carboxylic acid or the oxo acid of phosphorus or derivative thereof can be used alone or in combination of two or more kinds. The amount of the organic carboxylic acid or the oxo acid of phosphorus or derivative thereof blended, which is arbitrarily adjusted according to the kind of the above compound to be used, is preferably 0 to 49% by mass of the total mass of the solid component, more preferably 0 to 5% by mass, further preferably 0 to 1% by mass, and particularly preferably 0% by mass.

### [Other Additive]

Furthermore, the composition of the second embodiment can contain one kind or two kinds or more of additives other than the components mentioned above, if required. Examples of such an additive include a dye, a pigment and an adhesion aid. For example, when the composition is blended with a dye or a pigment, a latent image of the exposed portion is visualized and influence of halation upon exposure can be alleviated, which is preferable. Also, when the composition is blended with an adhesion aid, adhesiveness to a substrate can be improved, which is preferable. Furthermore, examples of the other additive include a halation preventing agent, a storage stabilizing agent, a defoaming agent and a shape improving agent. Specific examples thereof include 4-hydroxy-4′-methylchalcone.

In the composition of the second embodiment, the total content of the optional component (F) can be 0 to 99% by mass of the total mass of the solid component, and is preferably 0 to 49% by mass, more preferably 0 to 10% by mass, further preferably 0 to 5% by mass, still more preferably 0 to 1% by mass, and particularly preferably 0% by mass.

### [Method for Forming Resist Pattern and Insulating Film]

The resist pattern formation method of the second embodiment comprises:
a step of forming a resist film on a substrate using the composition for film formation of the second embodiment;
a step of exposing a pattern on the resist film; and
a step of subjecting the resist film after exposure to a development treatment.

The insulating film formation method of the second embodiment may comprise the resist pattern formation method of the second embodiment. That is, the insulating film formation method of the second embodiment may comprise:
a step of forming a resist film on a substrate using the composition for film formation of the second embodiment;
a step of exposing a pattern on the resist film; and
a step of subjecting the resist film after exposure to a development treatment.

The composition for film formation of the second embodiment contains, for example, the compound (A), the composition of the second embodiment, or the polymer (A).

Examples of the coating method in the step of forming a resist film include, but are not particularly limited to, spin coating, dip coating, and roll coating. Examples of the substrate include, but are not particularly limited to, a silicon wafer, metal, plastic, glass, and ceramic. After formation of the resist film, a heat treatment may be carried out at a temperature of about 50° C. to 200° C. The film thickness of the resist film is not particularly limited and is for example, 50 nm to 1 μm.

In the exposure step, exposure may be carried out via a predetermined mask pattern, or shot exposure may be carried out without masking. The thickness of the coating film is, for example, 0.1 to 20 μm, and preferably about 0.3 to 2 μm. Lights of various wavelengths such as ultraviolet ray, X-ray can be utilized for exposure, and for example, far ultraviolet ray such as F2 excimer laser (wavelength: 157 nm), ArF excimer laser (wavelength: 193 nm), and KrF excimer laser (wavelength: 248 nm), extreme ultraviolet ray (wavelength: 13 n), X-ray, and electron beam can be arbitrarily selected and used, as a light source. Among them, extreme ultraviolet ray is preferable. The exposure conditions such as the exposure amount are arbitrarily selected depending on the composition of the aforementioned resin and/or compound blended, the kind of each additive, and the like.

In the second embodiment, to stably form a fine pattern with a high degree of accuracy, the resist film is preferably subjected to a heat treatment at a temperature of 50 to 200° C. for 30 seconds or more, after exposure. In this case, variation of sensitivity depending on the kind of substrate may increase at a temperature lower than 50° C. Thereafter, development is carried out with an alkaline developing solution under the conditions of normally at 10 to 50° C. for 10 to 200 seconds, and preferably 20 to 25° C. for 15 to 90 seconds, resulting in formation of a predetermined resist pattern.

The alkaline developing solution used is an alkaline aqueous solution in which an alkaline compound such as an alkali metal hydroxide, aqueous ammonia, an alkylamine, an alkanolamine, a heterocyclic amine, a tetraalkylammonium hydroxide, choline, 1,8-diazabicyclo-[5.4.0]-7-undecene, and 1,5-diazabicyclo-[4.3.0]-5-nonene is dissolved such that the concentration is normally 1 to 10% by mass and preferably 1 to 3% by mass. Into the developing solution comprising the alkaline aqueous solution, a water-soluble organic solvent and a surfactant can be arbitrarily added.

It is also possible to use a solvent as the developing solution. As the solvent used as the developing solution, a solvent having a solubility parameter (SP value) close to that of the compound or resin according to the second embodiment to be used is preferably selected. A polar solvent such as a ketone-based solvent, an ester-based solvent, an alcohol-based solvent, an amide-based solvent, and an ether-based solvent; and a hydrocarbon-based solvent, or an alkaline aqueous solution can be used. Depending on the kind of the developing solution, a positive type resist pattern and a negative type resist pattern can be individually prepared. In general, in the case of a polar solvent such as a ketone-based solvent, an ester-based solvent, an alcohol-based solvent, an amide-based solvent, and an ether-based solvent, or a hydrocarbon-based solvent, a negative type resist pattern is obtained, and in the case of an alkaline aqueous solution, a positive type resist pattern is obtained. Examples of the ketone-based solvent, the ester-based solvent, the alcohol-based solvent, the amide-based solvent, and the ether-based solvent, the hydrocarbon-based solvent, and the alkaline aqueous solution include those disclosed in International Publication No. WO 2017/033943.

A plurality of above solvents may be mixed, or the solvent may be used by mixing the solvent with a solvent other than those described above or water within the range having performance. In order to sufficiently exhibit the effect of the second embodiment, the moisture content as the whole developing solution is preferably less than 70% by mass, and furthermore, preferably less than 50% by mass, more preferably less than 30% by mass, and further preferably less than 10% by mass. Particularly preferably, the developing solution is substantially moisture free. That is, the content of the organic solvent in the developing solution is preferably 30% by mass or more and 100% by mass or less based on the total amount of the developing solution, and furthermore, preferably 50% by mass or more and 100% by mass or less, more preferably 70% by mass or more and 100% by mass or less, further preferably 90% by mass or more and 100% by mass or less, and particularly preferably 95% by mass or more and 100% by mass or less, without particular limitation.

Particularly, as the developing solution, a developing solution containing at least one kind of solvent selected from a ketone-based solvent, an ester-based solvent, an alcohol-based solvent, an amide-based solvent, and an ether-based solvent is preferable because it improves resist performance such as resolution and roughness of the resist pattern.

The vapor pressure of the developing solution is not particularly limited, and is preferably 5 kPa or less at 20° C., more preferably 3 kPa or less, and particularly preferably 2 kPa or less, for example. The evaporation of the developing solution on the substrate or in a developing cup is inhibited by setting the vapor pressure of the developing solution to 5 kPa or less, to improve temperature uniformity within a wafer surface, thereby resulting in improvement in size uniformity within the wafer surface. Examples of the developing solution having such a vapor pressure include the developing solutions disclosed in International Publication No. WO 2017/033943.

To the developing solution, a surfactant can be added in an appropriate amount, if required. The surfactant is not particularly limited, but an ionic or nonionic, fluorine-based or silicon-based surfactant or the like can be used, for example. Examples of the fluorine-based or silicon-based surfactant may include, for example, the surfactants described in Japanese Patent Laid-Open Nos. 62-36663, 61-226746, 61-226745, 62-170950, 63-34540, 7-230165, 8-62834, 9-54432, and 9-5988, and U.S. Pat. Nos. 5,405,720, 5,360,692, 5,529,881, 5,296,330, 5,436,098, 5,576,143, 5,294,511, and 5,824,451. The surfactant is preferably a nonionic surfactant. The nonionic surfactant is not particularly limited, but a fluorine-based surfactant or a silicon-based surfactant is further preferably used.

The amount of the surfactant used is usually 0.001 to 5% by mass based on the total amount of the developing solution, preferably 0.005 to 2% by mass, and further preferably 0.01 to 0.5% by mass.

For the development method, for example, a method for dipping a substrate in a bath filled with a developing solution for a fixed time (dipping method), a method for raising a developing solution on a substrate surface by the effect of a surface tension and keeping it still for a fixed time, thereby conducting the development (puddle method), a method for spraying a developing solution on a substrate surface (spraying method), and a method for continuously ejecting a developing solution on a substrate rotating at a constant speed while scanning a developing solution ejecting nozzle at a constant rate (dynamic dispense method), or the like may be applied. The time for carrying out the pattern development is not particularly limited, but is preferably 10 seconds to 90 seconds.

In addition, after the step of conducting the development, a step of stopping the development by the replacement with another solvent may be carried out.

After the development, it is preferable that a step of rinsing the resist film with a rinsing solution containing an organic solvent is included.

The rinsing solution used in the rinsing step after the development is not particularly limited as long as the rinsing solution does not dissolve the resist pattern cured by crosslinking. A solution containing a general organic solvent or water may be used as the rinsing solution. As the rinsing solution, a rinsing solution containing at least one kind of organic solvent selected from a hydrocarbon-based solvent, a ketone-based solvent, an ester-based solvent, an alcohol-based solvent, an amide-based solvent, and an ether-based solvent is preferably used. More preferably, after development, a step of rinsing the film by using a rinsing solution containing at least one kind of organic solvent selected from the group consisting of a ketone-based solvent, an ester-based solvent, an alcohol-based solvent and an amide-based solvent is conducted. Further preferably, after development, a step of rinsing the film by using a rinsing solution containing an alcohol-based solvent or an ester-based solvent is conducted. Even more preferably, after the development, a step of rinsing the film by using a rinsing solution containing a monohydric alcohol is conducted. Particularly preferably, after the development, a step of rinsing the film by using a rinsing solution containing a monohydric alcohol having 5 or more carbon atoms is carried out. The time for rinsing the pattern is not particularly limited, but is preferably 10 seconds to 90 seconds.

Here, examples of the monohydric alcohol to be used in the rinsing step after development include, but are not particularly limited to, a linear, branched or cyclic monohydric alcohol. Specifically, for example, 1-butanol, 2-butanol, 3-methyl-1-butanol, tert-butyl alcohol, 1-pentanol, 2-pentanol, 1-hexanol, 4-methyl-2-pentanol, 1-heptanol, 1-octanol, 2-hexanol, cyclopentanol, 2-heptanol, 2-octanol, 3-hexanol, 3-heptanol, 3-octanol, 4-octanol or the like can be used. As the particularly preferable monohydric alcohol having 5 or more carbon atoms, 1-hexanol, 2-hexanol, 4-methyl-2-pentanol, 1-pentanol, 3-methyl-1-butanol or the like can be used.

A plurality of these components may be mixed, or the component may be used by mixing the component with an organic solvent other than those described above.

The moisture content in the rinsing solution is not particularly limited, and is preferably 10% by mass or less, more preferably 5% by mass or less, and particularly preferably 3% by mass or less. By setting the moisture content to 10% by mass or less, better development characteristics can be obtained.

The vapor pressure at 20° C. of the rinsing solution used after development is preferably 0.05 kPa or more and 5 kPa or less, more preferably 0.1 kPa or more and 5 kPa or less, and further preferably 0.12 kPa or more and 3 kPa or less. By setting the vapor pressure of the rinsing solution to 0.05 kPa or more and 5 kPa or less, the temperature uniformity in the wafer surface is enhanced more, and furthermore, swelling due to permeation of the rinsing solution is further inhibited. As a result, the dimensional uniformity in the wafer surface is further improved.

The rinsing solution may also be used after adding an appropriate amount of a surfactant to the rinsing solution.

In the rinsing step, the wafer after the development is rinsed using the above organic solvent containing rinsing solution. The method for the rinsing treatment is not particularly limited. However, for example, a method for continuously ejecting a rinsing solution on a substrate spinning at a constant speed (spin coating method), a method for dipping a substrate in a bath filled with a rinsing solution for a fixed time (dipping method), and a method for spraying a rinsing solution on a substrate surface (spraying method), or the like can be applied. Above all, it is preferable to conduct the rinsing treatment by the spin coating method and after the rinsing, spin the substrate at a rotational speed of 2,000 rpm to 4,000 rpm, to remove the rinsing solution from the substrate surface.

The composition of the second embodiment can also be used as an optical component forming composition applying lithography technology. The optical component is used in the form of a film or a sheet and is also useful as a plastic lens (a prism lens, a lenticular lens, a microlens, a Fresnel lens, a viewing angle control lens, a contrast improving lens, etc.), a phase difference film, a film for electromagnetic wave shielding, a prism, an optical fiber, a solder resist for flexible printed wiring, a plating resist, an interlayer insulating film for multilayer printed circuit boards, a photosensitive optical waveguide, a liquid crystal display, an organic electroluminescent (EL) display, an optical semiconductor (LED) element, a solid state image sensing element, an organic thin film solar cell, a dye sensitized solar cell, and an organic thin film transistor (TFT). The composition can be suitably utilized as an embedded film and a smoothed film on a photodiode, a smoothed film in front of or behind a color filter, a microlens, and a smoothed film and a conformal film on a microlens, all of which are components of a solid state image sensing element, to which high refractive index is particularly demanded.

Also, the composition of the second embodiment can be used as a patterning material for lithography applications. For lithography process applications, the composition can be used for various applications such as a semiconductor, a liquid crystal display panel, a display panel using OLED, a power device, and CCD and other sensors. In particular, for semiconductors and integrated circuits on devices, the composition of the second embodiment can be suitably utilized in the process of forming a device element on a silicon wafer, for the purpose of constructing a semiconductor element and other devices by forming a pattern on an insulating film on the substrate side using etching based on the pattern formed on the upper surface side of the insulating layer such as a silicon oxide film and other oxide films utilizing the composition of the second embodiment, further laminating a metal film or a semiconductor material based on the formed insulating film pattern, and forming a circuit pattern.

The description of the second embodiment is as above.

### Third Embodiment

Hereinafter, the third embodiment of the present invention will be described. The third embodiment is an embodiment in the case where R^{X} in the compound (A) in the first embodiment is a hydrogen atom. In the description of the third embodiment, the description may be simplified or omitted with respect to the same contents as those of the second embodiment, in some cases. The third embodiment is given in order to illustrate the present invention. The present invention is not limited to only the second embodiment.

### [Compound (A)]

The compound according to the third embodiment (hereinafter, also referred to as “compound (A)”) is represented by the following formula (1).

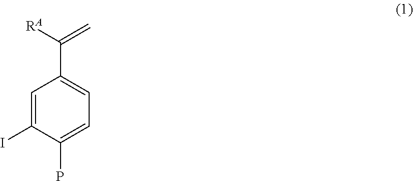

By using a compound, a polymer, a composition, or a composition for film formation containing the compound (A), a resist having significantly excellent exposure sensitivity can be obtained. In addition, a resist having significantly excellent exposure sensitivity can be obtained by a pattern formation method, insulating film formation method, or method for producing a compound using the compound (A). That is, by using the compound (A), a compound, a polymer, a composition, a composition for film formation, a pattern formation method, an insulating film formation method, and a method for producing a compound, by which a resist having significantly excellent exposure sensitivity can be obtained, can be provided.

The reason why a resist having significantly excellent exposure sensitivity is obtained by using the compound (A) is not clear, but can be presumed that the iodine atom has a high absorption effect to EUV, the absorption effect easily exerts an influence on the substituent P immediately adjacent to the iodine atom, the sensitizing effect easily generates from the substituent P, and furthermore, the sensitizing effect of P is easily exerted since the position adjacent to the substituent P and opposite to the iodine atom is unsubstituted, and thus, these interact with each other.

In the formula (1), R^{A} is a hydrogen atom, a methyl group, or a trifluoromethyl group; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group.

In the formula (1), R^{A} is a hydrogen atom, a methyl group, or a trifluoromethyl group. From the viewpoint of increasing the hydrophilicity to achieve high sensitivity, R^{A} is preferably a hydrogen atom or a methyl group.

From the viewpoint of increasing the absorption to EUV to achieve high sensitivity, R^{A} is preferably a trifluoromethyl group.

In the third embodiment, unless otherwise defined, the term “substituted” means that one or more hydrogen atoms in a functional group are substituted with a substituent. Examples of the “substituent” include, but are not particularly limited to, a halogen atom, a hydroxyl group, a carboxyl group, a cyano group, a nitro group, a thiol group, a heterocyclic ring group, an alkyl group having 1 to 30 carbon atoms, an aryl group having 6 to 30 carbon atoms, an alkoxyl group having 1 to 30 carbon atoms, an alkenyl group having 2 to 30 carbon atoms, an alkynyl group having 2 to 30 carbon atoms, an acyl group having 1 to 30 carbon atoms, and an amino group having 0 to 30 carbon atoms.

The alkyl group may be any of a linear aliphatic hydrocarbon group, a branched aliphatic hydrocarbon group, and a cyclic aliphatic hydrocarbon group.

Examples of the alkyl group having 1 to 30 carbon atoms include, but are not limited to, a methyl group, an ethyl group, an n-propyl group, an i-propyl group, an n-butyl group, an i-butyl group, a t-butyl group, an n-pentyl group, an n-hexyl group, an n-dodecyl group, and a valeryl group.

Examples of the aryl group having 6 to 30 carbon atoms include, but are not limited to, a phenyl group, a naphthalene group, a biphenyl group, an anthracyl group, a pyrenyl group, and a perylene group.

Examples of the alkenyl group having 2 to 30 carbon atoms include, but are not limited to, an ethynyl group, a propenyl group, a butynyl group, and a pentynyl group.

Examples of the alkynyl group having 2 to 30 carbon atoms include, but are not limited to, an acetylene group and an ethynyl group.

Examples of an alkoxy group having 1 to 30 carbon atoms include, but are not limited to, a methoxy group, an ethoxy group, a propoxy group, a butoxy group, and pentoxy.

In the formula (1), each P is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of P optionally have a substituent.

Examples of P include at least one group selected from the group consisting of an alkoxy group [^{*3}—O—R²], an ester group [^{*3}—O—(C═O)—R² or ^{*3}—(C═O)—O—R²], an acetal group [^{*3}—O—(C(R²¹)₂)—O—R² (wherein each R²¹ is independently H or a hydrocarbon group having 1 to 10 carbon atoms; and R² and R²¹ are optionally bonded to form a cyclic ether)], a carboxyalkoxy group [^{*3}—O—R²²—(C═O)—O—R² (wherein R²² is a divalent hydrocarbon group having 1 to 10 carbon atoms)], and a carbonate ester group [^{*3}—O—(C═O)—O—R²]. The ester group is preferably a tertiary ester group, from the viewpoint of achieving high sensitivity. In the formula, ^{*3} is a site for binding with A.

Among them, P is preferably a hydroxyl group, an alkoxy group, an ester group, an acetal group, a carboxyalkoxy group, a carbonate ester group, a nitro group, a carboxyl group, a thiol group, an ether group, a thioether group, a phosphine group, a phosphone group, a urethane group, a urea group, an amide group, an imide group, or a phosphate group, more preferably a hydroxy group, an ester group, an acetal group, a carbonate ester group, or a carboxyalkoxy group, further preferably an acetal group, a carbonate ester group, or a carboxyalkoxy group, and particularly preferably an acetal group or a carboxyalkoxy group, from the viewpoint of high sensitivity. From the viewpoint of producing a polymer having a stable quality by radical polymerization, an ester group, a carboxyalkoxy group, and a carbonate ester group are preferable. Further, from the viewpoint of increasing the difference in the dissolution rate between before and after exposure to increase the resolution, a tertiary ester group, an acetal group, a carbonate ester group, or a carboxyalkoxy group is preferable. From the viewpoint of achieving high sensitivity without adversely affecting other characteristics, P is preferably an ester group, an acetal group, or a carbonate ester group.

Each P is preferably independently a group represented by the following formula (P-1).

-L²-R² (P-1)

In the formula (P-1),

L² is a group which is cleaved by the action of an acid or a base. Examples of the group which is cleaved by the action of an acid or a base include at least one divalent linking group selected from the group consisting of an ester group [^{*1}—O—(C═O)—^{*2} or ^{*1}—(C═O)—O—^{*2}], an acetal group [^{*1}—O—(C(R²¹)₂)—O—^{*2} (each R²¹ is independently H or a hydrocarbon group having 1 to 10 carbon atoms)], a carboxyalkoxy group [^{*1}—O—R²²—(C═O)—O—^{*2} (R²² is a divalent hydrocarbon group having 1 to 10 carbon atoms)], and a carbonate ester group [^{*1}—O—(C═O)—O—^{*2}]. The ester group is preferably a tertiary ester group, from the viewpoint of achieving high sensitivity. In the formula, ^{*1} is a site for binding with a benzene ring, and ^{*2} is a site for binding with R². Among them, L² is preferably a tertiary ester group, an acetal group, a carbonate ester group, or a carboxyalkoxy group, more preferably an acetal group, a carbonate ester group, or a carboxyalkoxy group, and further preferably an acetal group or a carboxyalkoxy group, from the viewpoint of high sensitivity. From the viewpoint of producing a polymer having a stable quality by radical polymerization, an ester group, a carboxyalkoxy group, and a carbonate ester group are preferable.

As another effect, P is preferably a group represented by the formula (P-1) to control the polymerization properties of resin and the degree of polymerization in a desired range, when the compound (A) of the third embodiment is used as a polymerization unit of a copolymer. Since the compound (A) has a large influence on activity species in the polymer formation reaction due to having iodine and thus the desired control is difficult, variation of copolymer formation derived from the hydrophilic group and polymerization inhibition can be suppressed by having a group represented by the formula (P-1) in the hydrophilic group in the compound (A) as a protecting group.

R² is a linear, branched, or cyclic aliphatic group having 1 to 30 carbon atoms, an aromatic group having 6 to 30 carbon atoms, a linear, branched, or cyclic aliphatic group containing a heteroatom and having 1 to 30 carbon atoms, or a linear, branched, or cyclic aromatic group containing a heteroatom and having 1 to 30 carbon atoms, and the aliphatic group, the aromatic group, the aliphatic group containing a heteroatom, and the aromatic group containing a heteroatom of R² optionally further have or may not have a substituent. As the substituent here, the aforementioned substituents are used, but a linear, branched, or cyclic aliphatic group having 1 to 20 carbon atoms or an aromatic group having 6 to 20 carbon atoms is preferable. Among them, R² is preferably an aliphatic group. The aliphatic group in R² is preferably a branched or cyclic aliphatic group. The number of carbon atoms of the aliphatic group is preferably 1 or more and 20 or less, more preferably 3 or more and 10 or less, and further preferably 4 or more and 8 or less. Examples of the aliphatic group include, but are not particularly limited to, a methyl group, an isopropyl group, a sec-butyl group, a tert-butyl group, an isobutyl group, a cyclohexyl group, a methylcyclohexyl group, and an adamantyl group. Among them, a tert-butyl group, a cyclohexyl group, or an adamantyl group is preferable.

L² is preferably ^{*1}—(C═O)—O—^{*2} or a carboxyalkoxy group, because, when L² is cleaved by the action of an acid or a base, a carboxylic acid group is formed and the difference in the solubility and the difference in the dissolution rate between a cleaved portion and an uncleaved portion are increased in the development treatment, so that the resolution is improved and, in particular, residues at the pattern bottom in thin line patterns are suppressed.

Specific examples of P include the followings. Each P is independently a group represented by any of the following formulas.

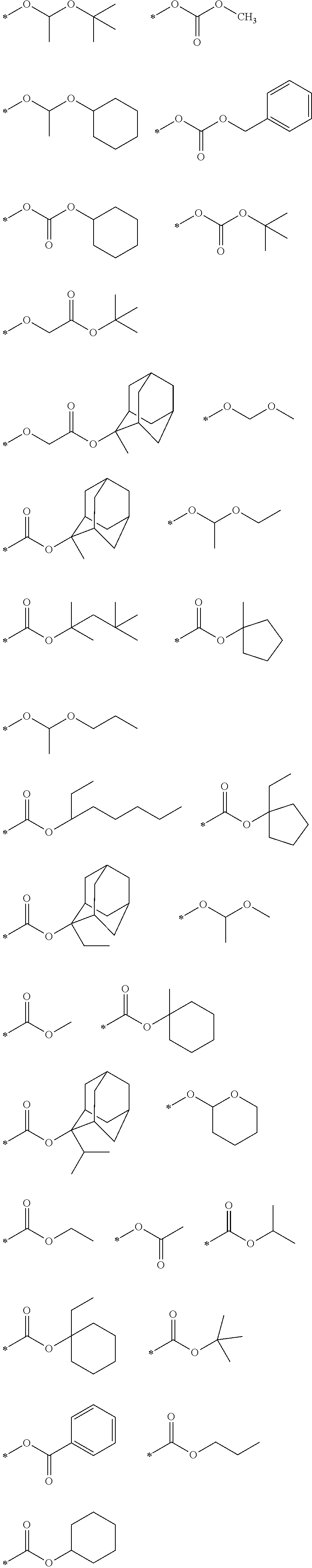

Examples of the alkoxy group that can be used as P include an alkoxy group having 1 or more carbon atoms, and from the viewpoint of the solubility of a resin after the compound is combined with other monomers to form the resin, an alkoxy group having 2 or more carbon atoms is preferable, and an alkoxy group having 3 or more carbon atoms or having a cyclic structure is preferable.

Specific examples of the alkoxy group that can be used as P include, but are not limited to, the followings.

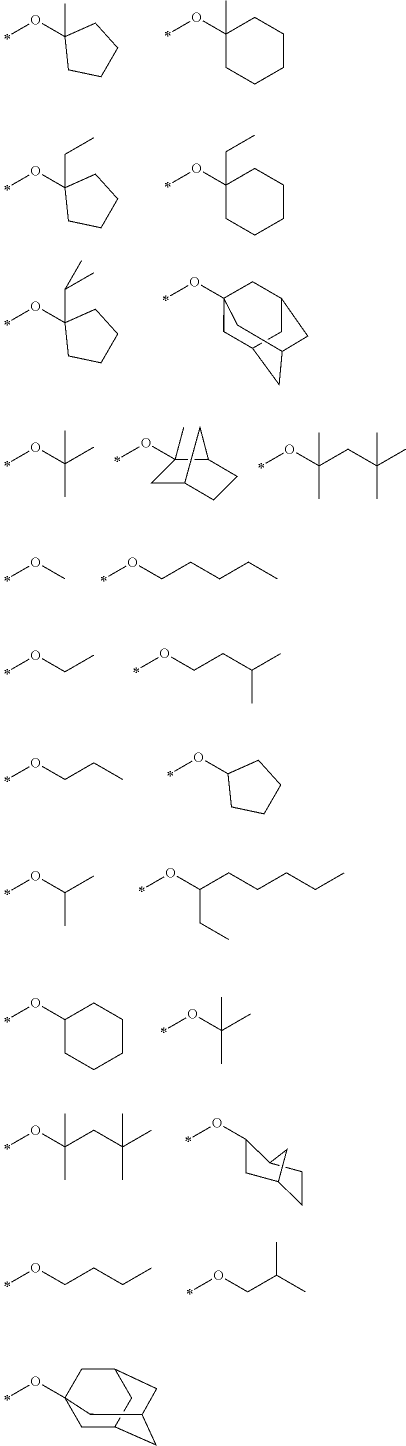

As the amino group and amide group that can be used as P, a primary amino group, a secondary amino group, a tertiary amino group, a group having a quaternary ammonium salt structure, an amide having a substituent, or the like can be arbitrarily used. Specific examples of the amino group or amide group that can be used include, but are not limited to, the followings.

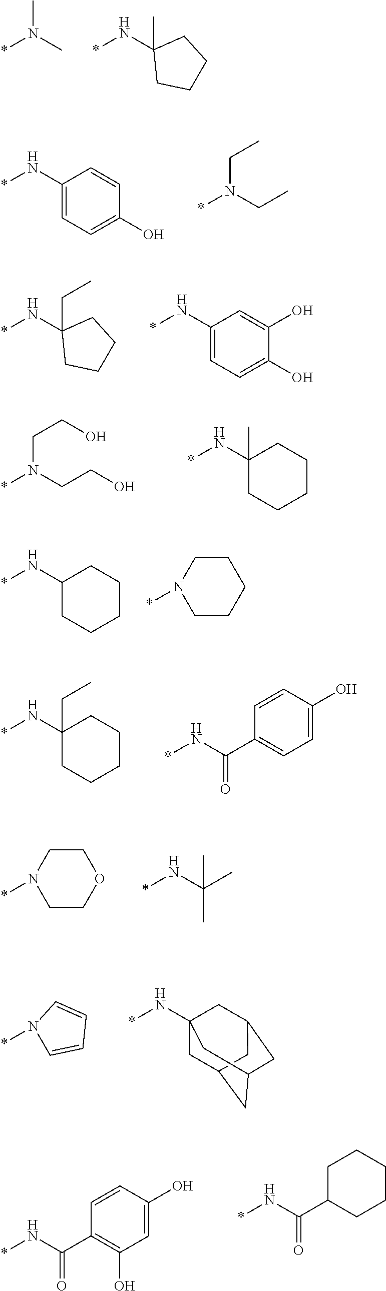

The compound (A) according to the third embodiment is considered to contribute to the efficiency of the proton generation mechanism after exposure by having a hydrogen group as a proton source in the ortho position of a phenolic hydroxy group. When a polymer using the compound (A) is applied to a resist composition and pattern formation is carried out by lithography processing comprising film formation, exposure, and development, the improvement of the proton generation efficiency after exposure compensates for a lack of protons generated which causes development residues, roughness, bridges, and the like, allowing both development defects to be reduced and other lithography performances such as sensitivity and resolution to be achieved. It is presumed that the pattern quality in finer pattern formation can be consequently improved.

As a result, the compound (A) is considered to be effective for the improvement of the pattern quality of a pattern having a low aperture ratio, such as, in particular, a hole pattern.

Examples of the compound (A) according to the third embodiment include compounds having the structures given below.

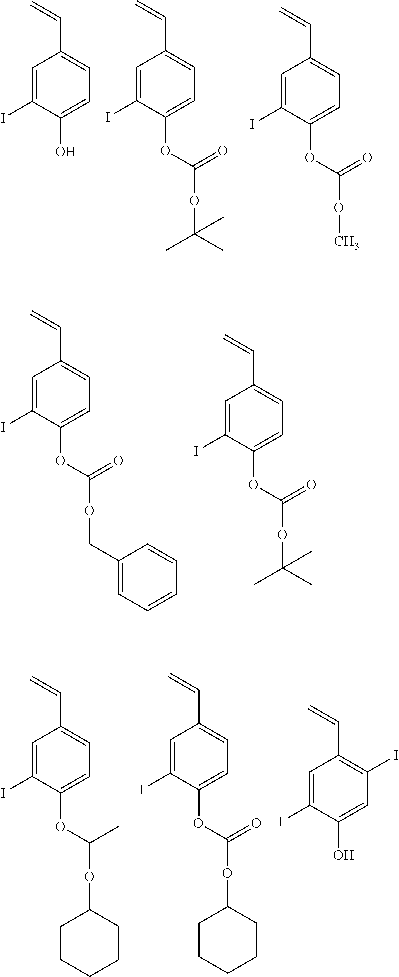

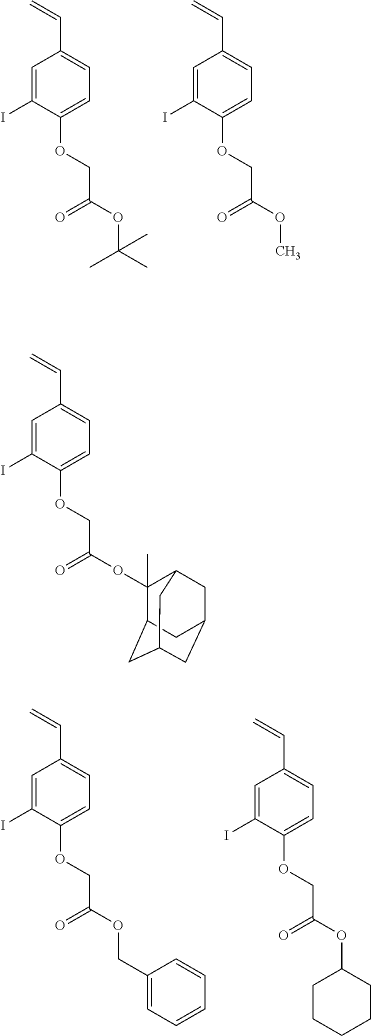 [SMILES: C=Cc1ccc(OCC(=O)OC(C)(C)C)c(I)c1 ; C=Cc1ccc(OCC(=O)OC)c(I)c1 ; C=Cc1ccc(OCC(=O)OC2(C)C3CC4CC(C3)CC2C4)c(I)c1 ; C=Cc1ccc(OCC(=O)OCc2ccccc2)c(I)c1 ; C=Cc1ccc(OCC(=O)OC2CCCCC2)c(I)c1]

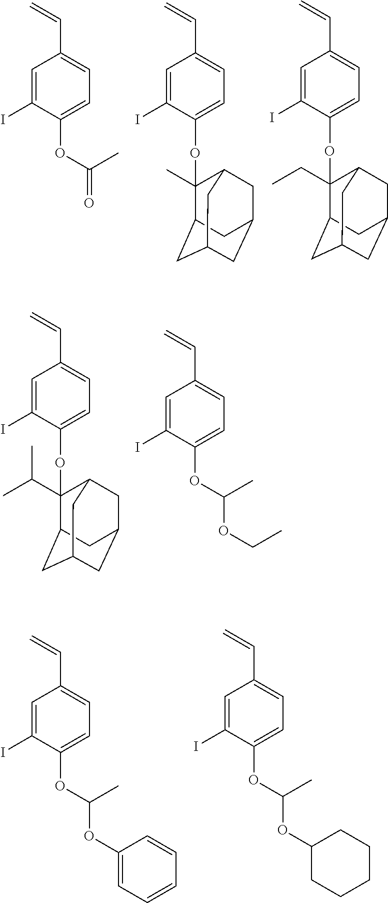

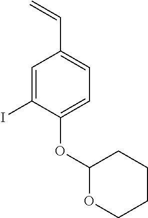 [SMILES: C=Cc1ccc(OC2CCCCO2)c(I)c1]

The compound (A) described above is preferably used in combination with a compound represented by the following formula (1A). That is, the composition according to the third embodiment preferably contains the compound (A) and the compound represented by the formula (1A).

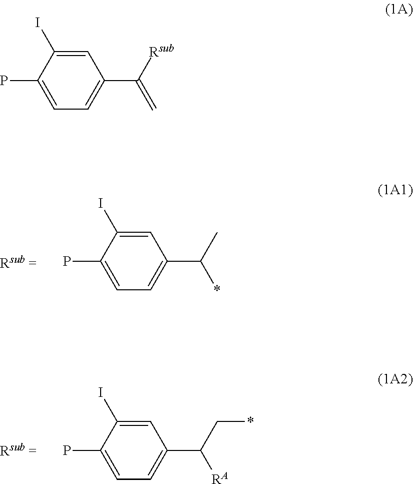

(In the formula (1A), the formula (1A1), and the formula (1A2), R^{A} and P have the same meanings as in the formula (1); R^{sub} represents the formula (1A1) or formula (1A2); and * is a site for binding with an adjacent constitutional unit.)

The composition is preferably prepared such that the compound represented by the formula (1A) is contained within a range of 1 ppm by mass or more and 10% by mass or less, more preferably within a range of 1 ppm by mass or more and 5% by mass or less, further preferably within a range of 1 ppm by mass or more and 3% by mass or less, and particularly preferably within a range of 1 ppm by mass or more and 1% by mass or less, based on the total compound (A), from the viewpoint of improving the exposure sensitivity and reducing the residue defect. In the resin form after the formation of a resin made of starting materials including the composition thus prepared, the presence of a moiety containing iodine and a moiety consisting of P at a high density in the proximity area becomes the starting point for improving the exposure sensitivity. Further, a local increase in the solubility of the resin leads to a reduction in the residue defect after development in a lithography process.

Examples of the compound (1A) according to the third embodiment include compounds having the structures given below.

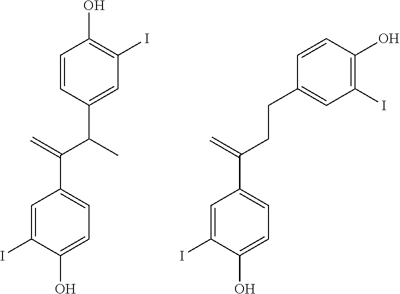 [SMILES: C=C(c1ccc(O)c(I)c1)C(C)c1ccc(O)c(I)c1 ; C=C(CCc1ccc(O)c(I)c1)c1ccc(O)c(I)c1]

The compound (A) described above is preferably used in combination with a compound represented by the following formula (1B). That is, the composition according to the third embodiment preferably contains the compound (A) and the compound represented by the formula (1B).

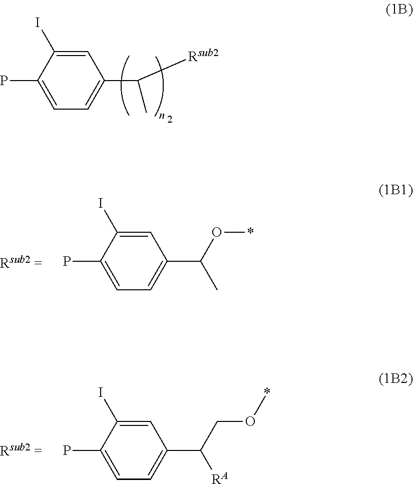

(In the formula (1B), the formula (1B1), or the formula (1B2), R^{A} and P have the same meanings as in the formula (1); n₂ is an integer of 0 to 4; R^{sub2} represents the formula (1B1) or the formula (1B2); and * is a site for binding with an adjacent constitutional unit.)

The composition is preferably prepared such that the compound represented by the formula (1B) is contained within a range of 1 ppm by mass or more and 10% by mass or less, more preferably within a range of 1 ppm by mass or more and 5% by mass or less, further preferably within a range of 1 ppm by mass or more and 3% by mass or less, and particularly preferably within a range of 1 ppm by mass or more and 1% by mass or less, based on the total compound (A), from the viewpoint of improving the exposure sensitivity and reducing the residue defect. In the resin form after the formation of a resin made of starting materials including the composition thus prepared, the presence of a moiety containing iodine and a moiety consisting of P at a high density in the proximity area becomes the starting point for improving the exposure sensitivity. Further, a local increase in the solubility of the resin leads to a reduction in the residue defect after development in a lithography process.

Examples of the compound (1B) according to the third embodiment include compounds having the structures given below.

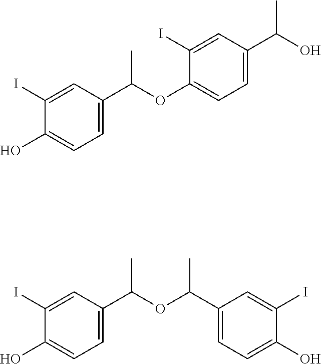 [SMILES: CC(O)c1ccc(OC(C)c2ccc(O)c(I)c2)c(I)c1 ; CC(OC(C)c1ccc(O)c(I)c1)c1ccc(O)c(I)c1]

The compound (A) described above is preferably used in combination with a compound represented by the following formula (1C). That is, the composition according to the third embodiment preferably contains the compound (A) and the compound represented by the formula (1C).

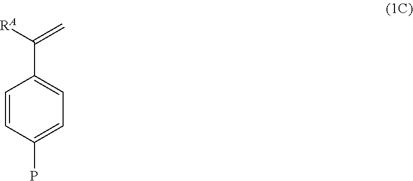

In the formula (1C), R^{A} and P have the same meanings as in the formula (1). Provided that P does not contain I.

The composition preferably contains the compound represented by the formula (1C) within a range of 1 ppm by mass or more and 10% by mass or less, more preferably within a range of 1 ppm by mass or more and 5% by mass or less, further preferably within a range of 1 ppm by mass or more and 3% by mass or less, and particularly preferably within a range of 1 ppm by mass or more and 1% by mass or less, based on the total compound (A), from the viewpoint of stability and reducing the residue defect.

In the composition thus prepared, its stability tends to increase. The reason is not clear, but can be presumed that the equilibrium reaction of iodine atoms occurs between the compound (A) containing iodine and the compound (1C) containing no iodine and thus the composition is stabilized.

In this case, the composition preferably contains, as the compound (1C), a compound having a structure in which iodine atoms are eliminated from a compound exemplified as the compound (A) mentioned above, in combination.

In the composition thus prepared, its stability increases, leading to not only an increase in the storage stability, but also formation of a resin having stable properties, providing stable resist performance, and further, a reduction in the residue defect after development in a lithography process.

Examples of the method for using the compound represented by the formula (1C) in a range of 1 ppm by mass or more and 10% by mass or less based on the total compound (A) in the composition containing the compound (A) include, but are not particularly limited to, a method for adding the compound (1C) to the compound (A) and a method for producing the compound (1C) as a by-product during production of the compound (A).

Examples of the compound (1C) according to the third embodiment include compounds having the structures given below.

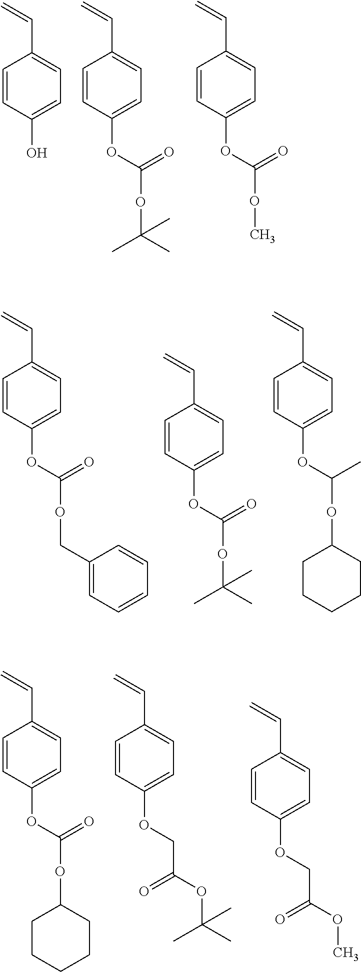

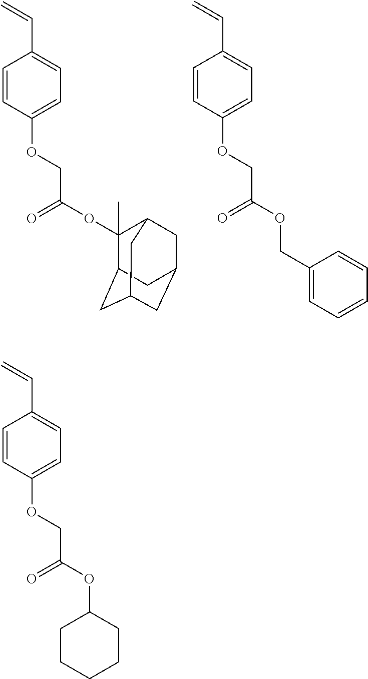 [SMILES: C=Cc1ccc(OCC(=O)OC2(C)C3CC4CC(C3)CC2C4)cc1 ; C=Cc1ccc(OCC(=O)OCc2ccccc2)cc1 ; C=Cc1ccc(OCC(=O)OC2CCCCC2)cc1]

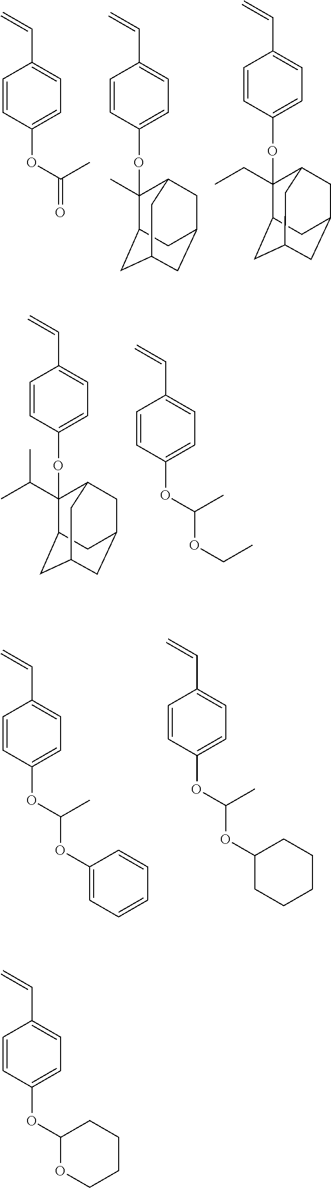

### [Method for Producing Compound (A)]

The compound represented by the formula (1) can be produced by various known synthetic methods.

As an example of the synthetic method for the compound represented by the formula (1) in which P is a hydroxy group, the synthesis can be carried out by introducing a halogen group, I, F, Cl, or Br, into a hydroxy group-containing aromatic aldehyde derivative, and then converting the aldehyde group into a vinyl group, but it is not particularly limited thereto. As another example of the synthetic method, a method for reacting iodine chloride in an organic solvent by carrying out iodination reaction on a hydroxybenzaldehyde derivative (e.g., see Japanese Patent Laid-Open No. 2012-180326), a method for dropping iodine in an aqueous alkaline solution of phenol under alkaline conditions in the presence of βcyclodextrin (Japanese Patent Laid-Open No. 63-101342, Japanese Patent Laid-Open No. 2003-64012), or the like can be arbitrarily selected.

In the third embodiment, the iodination reaction through iodine chloride in an organic solvent is preferably used. The compound (A) of the third embodiment can be synthesized by converting the aldehyde moiety of the synthesized iodine-introduced hydroxybenzaldehyde derivative into a vinyl group. As the method for converting the aldehyde moiety into a vinyl group, a Wittig reaction (e.g., the methods described in Synthetic Communications; Vol. 22; nb4; 1992 p 513, Synthesis; Vol. 49; nb. 23; 2017; p 5217) can be arbitrarily used.

That is, the method for producing the compound (A) represented by the formula (1) (iodine-containing vinyl monomer) comprises:
a) a step of providing an iodine-containing aldehyde substrate or iodine-containing ketone substrate having a general structure represented by the formula (1-5):

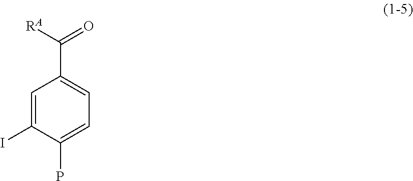

wherein R^{A} is a hydrogen atom, a methyl group, or a trifluoromethyl group; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and
b) a Wittig reaction step of forming an alkene from a carbonyl moiety of the iodine-containing aldehyde substrate or the iodine-containing ketone substrate by a Wittig reaction.

Examples of the iodine-containing aldehyde substrate or iodine-containing ketone substrate having a general structure represented by the formula (1-5) include 4-hydroxy-3-iodobenzaldehyde.

The Wittig reaction step is a step of forming alkene by a Witting reaction, and is a step of forming alkene from a carbonyl moiety having aldehyde or ketone using phosphorus ylide, although it is not limited. As the phosphorus ylide, for example, triphenyl alkyl phosphine bromide such as triphenyl methyl phosphine bromide that can form a stable phosphorus ylide can be used. Also, as the phosphorus ylide, a phosphonium salt can be reacted with a base to form a phosphorus ylide in the reaction system, which can be used in the aforementioned reaction. As the base, a conventionally known one can be used, and for example, an alkali metal salt of alkoxide can be arbitrarily used.

As another method for converting the aldehyde moiety into a vinyl group, a method for reacting malonic acid in the presence of a base (e.g., the methods described in Tetrahedron; Vol. 46; nb. 40; 2005; p 6893, Tetrahedron; Vol. 63; nb. 4; 2007; p 900, US2004/118673), or the like can be arbitrarily used.

In the third embodiment, the method for producing the compound (A) represented by the formula (1) (iodine-containing vinyl monomer) comprises:
a) a step of providing an iodine-containing aldehyde substrate or iodine-containing ketone substrate having a general structure represented by the formula (1-5);
b) a malonic acid addition step of adding malonic acid to the iodine-containing aldehyde substrate or the iodine-containing ketone substrate;
c) a hydrolysis step of subjecting the iodine-containing aldehyde substrate or iodine-containing ketone substrate to which the malonic acid is added to a hydrolysis treatment to produce an iodine-containing carboxylic acid substrate; and
d) a decarbonation step of subjecting the iodine-containing carboxylic acid substrate subjected to the hydrolysis treatment to a decarbonation treatment.

The malonic acid addition step in the third embodiment is a step of forming a malonic acid derivative, and is a reaction of aldehyde with malonic acid, a malonic acid ester, or a malonic acid anhydride, although it is not limited.

The hydrolysis step in the third embodiment is a step of forming a carboxylic acid substrate by hydrolysis, and is a reaction of hydrolyzing an ester by an action of an acid or water, although it is not limited.

The decarbonation step in the third embodiment is a step of carrying out decarboxylation of the carboxylic acid substrate to obtain a vinyl monomer, and is preferably carried out at a low temperature of 100° C. or less, and more preferably uses a fluoride-based catalyst, although it is not limited.

As the synthetic method of the compound (A) of the third embodiment, for example, the method described in the above references can be arbitrarily used, but is not limited thereto.

As an example of the synthetic method for the compound represented by the formula (1) in which P is an alkoxy group, an ester group, an acetal group, a carboxyalkoxy group, or a carbonate ester group, the compound can be obtained by reacting the compound represented by the formula (1) in which P is a hydroxy group with, for example, an active carboxylic acid derivative compound such as acid chloride, acid anhydride, or dicarbonate, an alkyl halide, a vinyl alkyl ether, dihydropyran, or a halocarboxylic acid alkyl ester, but it is not particularly limited thereto.

For example, the compound represented by the formula (1) in which P is a hydroxy group is dissolved or suspended in an aprotic solvent such as acetone, tetrahydrofuran, or propylene glycol monomethyl ether acetate. Subsequently, a vinyl alkyl ether such as ethyl vinyl ether, or dihydropyran is added to the solution or the suspension, and the mixture is reacted at 20 to 60° C. at normal pressure for 6 to 72 hours in the presence of an acid catalyst such as pyridinium p-toluenesulfonate. The reaction solution is neutralized with an alkali compound, added to distilled water to precipitate a white solid, and then, the separated white solid is washed with distilled water and dried, thereby allowing the compound represented by the formula (1) in which P is an alkoxy group, an ester group, an acetal group, a carboxyalkoxy group, or a carbonate ester group to be obtained.

Moreover, the compound represented by the formula (1) in which P is a hydroxy group is dissolved or suspended in an aprotic solvent such as acetone, THF, or propylene glycol monomethyl ether acetate. Subsequently, an alkyl halide such as ethyl chloromethyl ether or a halocarboxylic acid alkyl ester such as methyladamantyl bromoacetate is added to the solution or the suspension, and the mixture is reacted at 20 to 110° C. at normal pressure for 6 to 72 hours in the presence of an alkali catalyst such as potassium carbonate. The reaction solution is neutralized with an acid such as hydrochloric acid, added to distilled water to precipitate a white solid, and then, the separated white solid is washed with distilled water and dried, thereby allowing the compound represented by the formula (1) in which P is an alkoxy group, an ester group, an acetal group, a carboxyalkoxy group, or a carbonate ester group to be obtained.

The synthetic method of the compound (A) of the third embodiment more preferably comprises the synthetic methods given below, from the viewpoint of suppressing the yield and the amount of waste.

### [Iodine-Containing Alcohol Substrate]

The iodine-containing alcohol substrate used in the third embodiment may be, for example, an iodine-containing alcohol substrate having a general structure represented by the following formula (1-1).

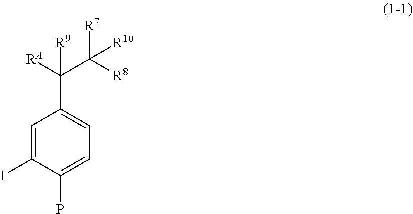

(In the formula (1-1), P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently hydrogen, a hydroxy group, a methoxy group, a halogen, or a cyano group, provided that one of R⁷ to R¹⁰ is a hydroxy group or a methoxy group.)

Examples of suitable iodine-containing alcohol substrates include, but are not limited to, 1-(4-hydroxy-3-iodophenyl)ethanol and 4-(1-hydroxyethyl)-3-iodophenol. At least one iodine atom is introduced, and two or more iodine atoms are preferably introduced.

These iodine-containing alcohol substrates can be obtained by many methods, but are preferably obtained by the method described later, from the viewpoint of the availability and yield of the raw material.

The method for producing the iodine-containing vinyl monomer represented by the formula (1) comprises:
a) a step of providing an iodine-containing alcohol substrate having a general structure represented by the formula (1-1); and
b) a dehydration step of subjecting the iodine-containing alcohol substrate to a dehydration treatment.

### Reaction Conditions

The reaction mixture is formed by adding the iodine-containing alcohol substrate having the formula (1-1), the catalyst, and the organic solvent to a reactor. Any suitable reactor is used.

The reaction may be carried out by arbitrarily selecting a known method such as a batch method, a semi-batch method, or a continuous method.

The reaction temperature is not particularly limited. The preferred range varies according to the concentration of the substrate, the stability of the formed product, the selection of the catalyst, and the desired yield. In general, a temperature of 0° C. to 200° C. is suitable, and from the viewpoint of the yield, a temperature of 0° C. to 100° C. is preferable.

In the reaction using 1-(4-hydroxy-3-iodophenyl)ethanol as the iodine-containing alcohol substrate, the temperature range is preferably 0° C. to 100° C.

The reaction pressure is not particularly limited. The preferred range varies according to the concentration of the substrate, the stability of the formed product, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation.

In the reaction using 1-(4-hydroxy-3-iodophenyl)ethanol as the iodine-containing alcohol substrate, the preferred reaction pressure is reduced pressure to normal pressure, and reduced pressure is preferable.

The reaction time is not particularly limited. The preferred range varies according to the concentration of the substrate, the stability of the formed product, the selection of the catalyst, and the desired yield. However, the majority of the reactions are carried out for less than 6 hours, and the reaction time is typically 15 minutes to 600 minutes.

In the reaction using 1-(4-hydroxy-3-iodophenyl)ethanol as the substrate, the preferred reaction time range is 15 minutes to 600 minutes.

Isolation and purification can be conducted by using a conventionally known suitable method after terminating the reaction. For example, the reaction mixture is poured in ice water and extracted in an organic solvent such as ethyl acetate or diethyl ether. Then, the product is recovered by removing the solvent using evaporation at reduced pressure. The product can be isolated and purified as the desired high purity monomer by a separation and purification method by filtration, concentration, distillation, extraction, crystallization, recrystallization, column chromatography, activated carbon, or the like, which are well-known purification methods in the art, or a combined method thereof.

### [Production Method (I) of Iodine-Containing Alcohol Substrate Represented by Formula (1-1)]

The iodine-containing ketone substrate used in the production of the iodine-containing alcohol substrate represented by the formula (1-1) is, for example, an iodine-containing ketone substrate having a general structure represented by the formula (1-2).

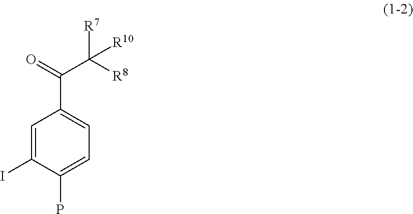

(In the formula (1-2), P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group.)

Examples of suitable iodine-containing ketone substrate include, but are not limited to, 4-hydroxy-3-iodophenylmethylketone.

These iodine-containing ketone substrates can be obtained by many methods, but is preferably obtained by the method described later, from the viewpoint of the availability and yield of the raw material.

The method for producing the iodine-containing alcohol substrate having a general structure represented by the formula (1-1) comprises:
c) a step of providing an iodine-containing ketone substrate having a general structure represented by the formula (1-2); and
d) a reduction step of subjecting the iodine-containing ketone substrate to a reduction treatment.

The method for producing the iodine-containing vinyl monomer having a general structure represented by the formula (1) may comprise the method for producing the iodine-containing alcohol substrate having a general structure represented by the formula (1-1). That is, the method for producing the iodine-containing vinyl monomer having a general structure represented by the formula (1) may comprise:
c) a step of providing an iodine-containing ketone substrate having a general structure represented by the formula (1-2); and
d) a reduction step of subjecting the iodine-containing ketone substrate to a reduction treatment.

### Reaction Conditions

The reaction mixture is formed by adding the iodine-containing ketone substrate having the formula (1-2), the catalyst, and the organic solvent to a reactor. Any suitable reactor is used.

The reaction may be carried out by arbitrarily selecting a known method such as a batch method, a semi-batch method, or a continuous method.

The reaction temperature is not particularly limited. The preferred range varies according to the concentration of the iodine-containing ketone substrate, the stability of the formed product, the selection of the catalyst, and the desired yield. In general, a temperature of 0° C. to 200° C. is suitable, and from the viewpoint of the yield, a temperature of 0° C. to 100° C. is preferable.

In the reaction using 4′-hydroxy-3′-iodoacetophenone as the iodine-containing ketone substrate, the temperature range is preferably 0° C. to 100° C.

The reaction pressure is not particularly limited. The preferred range varies according to the concentration of the iodine-containing ketone substrate, the stability of the formed product, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation.

In the reaction using 4′-hydroxy-3′-iodoacetophenone as the iodine-containing ketone substrate, the preferred reaction pressure is reduced pressure to normal pressure, and reduced pressure is preferable.

The reaction time is not particularly limited. The preferred range varies according to the concentration of the iodine-containing ketone substrate, the stability of the formed product, the selection of the catalyst, and the desired yield. However, the majority of the reactions are carried out for less than 6 hours, and the reaction time is typically 15 minutes to 600 minutes.

In the reaction using 4′-hydroxy-3′-iodoacetophenone as the iodine-containing ketone substrate, the preferred reaction time range is 15 minutes to 600 minutes.

Isolation and purification can be conducted by using a conventionally known suitable method after terminating the reaction. For example, the reaction mixture is poured in ice water and extracted in an organic solvent such as ethyl acetate or diethyl ether. Then, the product is recovered by removing the solvent using evaporation at reduced pressure. The product can be isolated and purified as the desired high purity monomer by a separation and purification method by filtration, concentration, distillation, extraction, crystallization, recrystallization, column chromatography, activated carbon, or the like, which are well-known purification methods in the art, or a combined method thereof.

### [Production Method (II) of Iodine-Containing Alcohol Substrate Represented by Formula (1-1)]

The alcohol substrate used in the iodine-containing alcohol substrate represented by the formula (1-1) is, for example, an alcohol substrate having a general structure represented by the formula (1-3).

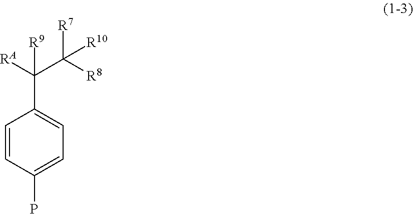

(In the formula (1-3), R^{A} is a hydrogen atom, a methyl group, or a trifluoromethyl group; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group.)

Examples of suitable alcohol substrate include, but are not limited to, 1-(4-hydroxyphenyl)ethanol and 4-(1-hydroxyethyl)phenol.

These alcohol substrates can be obtained by many methods, but are preferably obtained by the method described later, from the viewpoint of the availability and yield of the raw material.

The method for producing the iodine-containing alcohol substrate having a general structure represented by the formula (1-1) comprises:
e) a step of providing an alcohol substrate having a general structure represented by the formula (1-3); and
f) an iodine introduction step of introducing an iodine atom into the alcohol substrate.

The iodine introduction step in the third embodiment is not particularly limited, but a method for reacting an iodinating agent in a solvent (e.g., Japanese Patent Laid-Open No. 2012-180326), a method for dropping iodine in an aqueous alkaline solution of phenol under alkaline conditions in the presence of βcyclodextrin (Japanese Patent Laid-Open No. 63-101342, Japanese Patent Laid-Open No. 2003-64012), or the like can be arbitrarily selected. Examples of the iodinating agent include, but are not particularly limited to, iodinating agents such as iodine chloride, iodine, and N-iodosuccinimide. Among them, iodine chloride is preferable.

In the third embodiment, the iodination reaction through iodine chloride in an organic solvent is preferably used, particularly when introduction of a plurality of iodine atoms is intended. As the synthetic method of the compound (A) of the third embodiment, for example, the method described in the above references can be arbitrarily used, but is not limited thereto.

The method for producing the iodine-containing vinyl monomer having a general structure represented by the formula (1) may comprise the method for producing the iodine-containing alcohol substrate having a general structure represented by the formula (1-1). That is, the method for producing the iodine-containing vinyl monomer having a general structure represented by the formula (1) may comprise:
e) a step of providing an alcohol substrate having a general structure represented by the formula (1-3); and
f) an iodine introduction step.

### Reaction Conditions

The reaction mixture is formed by adding the alcohol substrate having the formula (1-3), the catalyst, and the organic solvent to a reactor. Any suitable reactor is used.

The reaction may be carried out by arbitrarily selecting a known method such as a batch method, a semi-batch method, or a continuous method.

The reaction temperature is not particularly limited. The preferred range varies according to the concentration of the substrate, the stability of the formed product, the selection of the catalyst, and the desired yield. In general, a temperature of 0° C. to 200° C. is suitable, and from the viewpoint of the yield, a temperature of 0° C. to 100° C. is preferable.

In the reaction using 1-(4-hydroxyphenyl)ethanol as the substrate, the temperature range is preferably 0° C. to 100° C.

The reaction pressure is not particularly limited. The preferred range varies according to the concentration of the substrate, the stability of the formed product, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation.

In the reaction using 1-(4-hydroxyphenyl)ethanol as the substrate, the preferred reaction pressure is reduced pressure to normal pressure, and reduced pressure is preferable.

The reaction time is not particularly limited. The preferred range varies according to the concentration of the substrate, the stability of the formed product, the selection of the catalyst, and the desired yield. However, the majority of the reactions are carried out for less than 6 hours, and the reaction time is typically 15 minutes to 600 minutes.

In the reaction using 1-(4-hydroxyphenyl)ethanol as the substrate, the preferred reaction time range is 15 minutes to 600 minutes.

Isolation and purification can be conducted by using a conventionally known suitable method after terminating the reaction. For example, the reaction mixture is poured in ice water and extracted in an organic solvent such as ethyl acetate or diethyl ether. Then, the product is recovered by removing the solvent using evaporation at reduced pressure. The product can be isolated and purified as the desired high purity monomer by a separation and purification method by filtration, concentration, distillation, extraction, crystallization, recrystallization, column chromatography, activated carbon, or the like, which are well-known purification methods in the art, or a combined method thereof.

### [Production Method) of Iodine-Containing Ketone Substrate Represented by Formula (1-2)]

The ketone substrate used in the production of the iodine-containing ketone substrate represented by the formula (1-2) is, for example, a ketone substrate having a general structure represented by the formula (1-4).

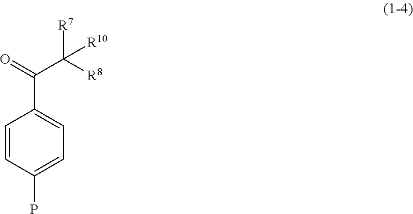

(In the formula (1-4), P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group.)

Examples of suitable ketone substrate include, but are not limited to, 4-hydroxyphenylmethylketone.

These ketone substrates can be obtained by many methods.

The method for producing the iodine-containing ketone substrate having a general structure represented by the formula (1-2) may comprise:
g) a step of providing a ketone substrate having a general structure represented by the formula (1-4); and
h) an iodine introduction step of introducing an iodine atom into the ketone substrate.

The method for producing the iodine-containing vinyl monomer having a general structure represented by the formula (1) may comprise the method for producing the iodine-containing ketone substrate having a general structure represented by the formula (1-2). That is, the method for producing the iodine-containing vinyl monomer having a general structure represented by the formula (1) may comprise:
g) a step of providing a ketone substrate having a general structure represented by the formula (1-4); and
h) an iodine introduction step of introducing an iodine atom into the ketone substrate.

### Reaction Conditions

The reaction mixture is formed by adding the ketone substrate having the formula (1-4), the catalyst, and the organic solvent to a reactor. Any suitable reactor is used.

The reaction may be carried out by arbitrarily selecting a known method such as a batch method, a semi-batch method, or a continuous method.

The reaction temperature is not particularly limited. The preferred range varies according to the concentration of the substrate, the stability of the formed product, the selection of the catalyst, and the desired yield. In general, a temperature of 0° C. to 200° C. is suitable, and from the viewpoint of the yield, a temperature of 0° C. to 100° C. is preferable.

In the reaction using 4′-hydroxy-3′-methoxyacetophenone as the substrate, the temperature range is preferably 0° C. to 100° C.

The reaction pressure is not particularly limited. The preferred range varies according to the concentration of the substrate, the stability of the formed product, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation.

In the reaction using 4′-hydroxyacetophenone as the substrate, the preferred reaction pressure is reduced pressure to normal pressure, and reduced pressure is preferable.

The reaction time is not particularly limited. The preferred range varies according to the concentration of the substrate, the stability of the formed product, the selection of the catalyst, and the desired yield. However, the majority of the reactions are carried out for less than 6 hours, and the reaction time is typically 15 minutes to 600 minutes.

In the reaction using 4′-hydroxyacetophenone as the substrate, the preferred reaction time range is 15 minutes to 600 minutes.

### [Production Method) of Alcohol Substrate Represented by Formula (1-3)]

The ketone substrate used in the production of the alcohol substrate having a general structure represented by the formula (1-3) is, for example, the ketone substrate having a general structure represented by the aforementioned formula (1-4).

The method for producing the alcohol substrate having a general structure represented by the formula (1-3) may comprise:
i) a step of providing a ketone substrate having a general structure represented by the formula (1-4); and
j) a reduction step of subjecting the ketone substrate to a reduction treatment.

The method for producing the iodine-containing vinyl monomer having a general structure represented by the formula (1) may comprise the method for producing the alcohol substrate having a general structure represented by the formula (1-3). That is, the method for producing the iodine-containing vinyl monomer having a general structure represented by the formula (1) may comprise:
i) a step of providing a ketone substrate having a general structure represented by the formula (1-4); and
j) a reduction step of subjecting the ketone substrate to a reduction treatment.

As the organic solvent, a wide variety of organic solvents including a polar aprotic organic solvent and protic polar organic solvent are used. A single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used, and a polar aprotic solvent or a mixture thereof is preferable. The solvent is effective, but is not an essential component. Examples of suitable polar aprotic solvents include, but are not limited to, an alcohol solvent such as methanol and ethanol, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, hexamethylphosphorous triamide; and dimethylsulfoxide. Dimethylsulfoxide is preferable. Examples of suitable protic polar solvents include, but are not limited to, di(propylene glycol) methyl ether, di(ethylene glycol) methyl ether, 2-butoxyethanol, ethylene glycol, 2-methoxyethanol, propyleneglycol methylether, n-hexanol, and n-butanol.

The amount of the solvent used can be arbitrarily set according to, for example, the substrate, catalyst, and reaction conditions to be used, without particular limitation. In general, it is suitably 0 to 10000 parts by mass, and from the viewpoint of the yield, preferably 100 to 2000 parts by mass, based on 100 parts by mass of the reaction raw materials.

### Reaction Conditions

The reaction mixture is formed by adding the ketone substrate having the formula (1-4), the catalyst, and the organic solvent to a reactor. Any suitable reactor is used.

The reaction may be carried out by arbitrarily selecting a known method such as a batch method, a semi-batch method, or a continuous method.

The reaction temperature is not particularly limited. The preferred range varies according to the concentration of the substrate, the stability of the formed product, the selection of the catalyst, and the desired yield. In general, a temperature of 0° C. to 200° C. is suitable, and from the viewpoint of the yield, a temperature of 0° C. to 100° C. is preferable.

In the reaction using 4′-hydroxyacetophenone as the substrate, the temperature range is preferably 0° C. to 100° C.

The reaction pressure is not particularly limited. The preferred range varies according to the concentration of the substrate, the stability of the formed product, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation.

In the reaction using 4′-hydroxyacetophenone as the substrate, the preferred reaction pressure is reduced pressure to normal pressure, and reduced pressure is preferable.

The reaction time is not particularly limited. The preferred range varies according to the concentration of the substrate, the stability of the formed product, the selection of the catalyst, and the desired yield. However, the majority of the reactions are carried out for less than 6 hours, and the reaction time is typically 15 minutes to 600 minutes.

In the reaction using 4′-hydroxyacetophenone as the substrate, the preferred reaction time range is 15 minutes to 600 minutes.

Isolation and purification can be conducted by using a conventionally known suitable method after terminating the reaction. For example, the reaction mixture is poured in ice water and extracted in an organic solvent such as ethyl acetate or diethyl ether. Then, the product is recovered by removing the solvent using evaporation at reduced pressure. The product can be isolated and purified as the desired high purity monomer by a separation and purification method by filtration, concentration, distillation, extraction, crystallization, recrystallization, column chromatography, activated carbon, or the like, which are well-known purification methods in the art, or a combined method thereof.

### [Method for Producing Iodine-Containing Vinyl Monomer Represented by Formula (2)]

The method for producing the iodine-containing vinyl monomer according to the third embodiment may be the method for producing the iodine-containing vinyl monomer represented by the following formula (2), and specifically may be the method for producing an iodine-containing alkoxystyrene.

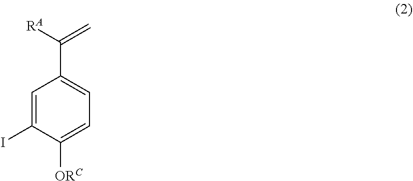

(In the formula (2), R^{A} is a hydrogen atom, a methyl group, or a trifluoromethyl group; and R^{C} is a substituted or unsubstituted acyl group having 1 to 30 carbon atoms.)

Examples of the acetoxystyrene produced by the method of the third embodiment include, but are not limited to, 4-acetoxy-3-iodostyrene.

### [Iodine-Containing Vinyl Monomer]

The iodine-containing vinyl monomer used in the third embodiment is, for example, the iodine-containing vinyl monomer having a general structure represented by the formula (1).

The iodine-containing vinyl monomer having a general structure represented by the formula (2) may comprise:
k) a step of providing an iodine-containing vinyl monomer having a general structure represented by the formula (1); and
l) an acylation step of subjecting the iodine-containing vinyl monomer to an acylation treatment.

As the organic solvent, a wide variety of organic solvents including a polar aprotic organic solvent and protic polar organic solvent are used. A single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used, and a polar aprotic solvent or a mixture thereof is preferable. The solvent is effective, but is not an essential component. Examples of suitable polar aprotic solvents include, but are not limited to, an alcohol solvent such as methanol and ethanol, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, hexamethylphosphorous triamide; and dimethylsulfoxide. Dimethylsulfoxide is preferable. Examples of suitable protic polar solvents include, but are not limited to, di(propylene glycol) methyl ether, di(ethylene glycol) methyl ether, 2-butoxyethanol, ethylene glycol, 2-methoxyethanol, propyleneglycol methylether, n-hexanol, and n-butanol.

The amount of the solvent used can be arbitrarily set according to, for example, the substrate, catalyst, and reaction conditions to be used, without particular limitation. In general, it is suitably 0 to 10000 parts by mass, and from the viewpoint of the yield, preferably 100 to 2000 parts by mass, based on 100 parts by mass of the reaction raw materials.

### Reaction Conditions

The reaction mixture is formed by adding the iodine-containing vinyl monomer having the formula (1), the catalyst, and the organic solvent to a reactor. Any suitable reactor is used.

The reaction may be carried out by arbitrarily selecting a known method such as a batch method, a semi-batch method, or a continuous method.

The reaction temperature is not particularly limited. The preferred range varies according to the concentration of the substrate, the stability of the formed product, the selection of the catalyst, and the desired yield. In general, a temperature of 0° C. to 200° C. is suitable, and from the viewpoint of the yield, a temperature of 0° C. to 100° C. is preferable.

In the reaction using 4-hydroxy-3-iodostyrene as the substrate, the temperature range is preferably 0° C. to 100° C.

The reaction pressure is not particularly limited. The preferred range varies according to the concentration of the substrate, the stability of the formed product, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation.

In the reaction using 4-hydroxy-3-iodostyrene as the substrate, the preferred reaction pressure is reduced pressure to normal pressure, and reduced pressure is preferable.

The reaction time is not particularly limited. The preferred range varies according to the concentration of the substrate, the stability of the formed product, the selection of the catalyst, and the desired yield. However, the majority of the reactions are carried out for less than 6 hours, and the reaction time is typically 15 minutes to 600 minutes.

In the reaction using 4-hydroxy-3-iodostyrene as the substrate, the preferred reaction time range is 15 minutes to 600 minutes.

Isolation and purification can be conducted by using a conventionally known suitable method after terminating the reaction. For example, the reaction mixture is poured in ice water and extracted in an organic solvent such as ethyl acetate or diethyl ether. Then, the product is recovered by removing the solvent using evaporation at reduced pressure. The product can be isolated and purified as the desired high purity monomer by a separation and purification method by filtration, concentration, distillation, extraction, crystallization, recrystallization, column chromatography, activated carbon, or the like, which are well-known purification methods in the art, or a combined method thereof.

It is preferable that the compound in the third embodiment be obtained as a crude by the reaction described above and be then further subjected to purification, thereby removing the residual metal impurities. That is, it is preferable to avoid residual metal impurities derived from contamination by metal components which are used as a reaction aid during the production process of the compound or contaminated from a reaction vessel for production or other production equipment, from the viewpoint of preventing the deterioration of the resin with time, storage stability, and further, production yield due to processability, defects, and the like when the composition is formed into a resin and applied to a semiconductor production process.

The residual amounts of the aforementioned metal impurities are preferably less than 1 ppm, more preferably less than 100 ppb, further preferably less than 50 ppb, still more preferably less than 10 ppb, and most preferably less than 1 ppb, based on the resin. In particular, with respect to metal species such as Fe, Ni, Sb, W, and Al which are classified as transition metals, there is concern that the amount of residual metals of 1 ppm or more may cause the denaturation and deterioration of materials with time due to the interaction with the compound in the third embodiment. Further, there is also concern that the metal balance cannot be sufficiently reduced with the amount of residual metals of 1 ppm or more when a resin for semiconductor process is prepared using the compound prepared, which results in defects derived from residual metals in a semiconductor production process and reduction in yield due to performance deterioration.

The purification method is not particularly limited, but comprises a step of obtaining a solution (S) by dissolving the compound in the third embodiment in a solvent; and a step of extracting impurities in the compound in the third embodiment by bringing the obtained solution (S) into contact with an acidic aqueous solution (a first extraction step), wherein the solvent used in the step of obtaining the solution (S) contains an organic solvent that does not inadvertently mix with water.

According to the purification method, the contents of various metals that may be contained as impurities in the resin can be reduced.

More specifically, the compound in the third embodiment can be dissolved in an organic solvent that does not inadvertently mix with water to obtain the solution (S), and further, an extraction treatment can be carried out by bringing the solution (S) into contact with an acidic aqueous solution. Thereby, after the metals contained in the solution (S) is transferred to the aqueous phase, the organic phase and the aqueous phase can be separated to obtain a resin having a reduced metal content.

The amount of the acidic aqueous solution used in the purification method is not particularly limited, but it is preferable to regulate the amount from the viewpoint of reducing the number of extraction operations for removing metals and from the viewpoint of ensuring operability in consideration of the overall amount of fluid. From the above viewpoints, the amount of the acidic aqueous solution to be used is preferably 10 to 200% by mass, and more preferably 20 to 100% by mass, based on 100% by mass of the solution (S).

In the purification method, by bringing the acidic aqueous solution into contact with the solution (S), metals can be extracted from the compound in the solution (S).

In the purification method, the solution (S) can further contain an organic solvent that inadvertently mixes with water. When the solution (S) contains an organic solvent that inadvertently mixes with water, there is a tendency that the amount of the compound charged can be increased, also the fluid separability is improved, and purification can be carried out at a high reaction vessel efficiency. The method for adding the organic solvent that inadvertently mixes with water is not particularly limited. For example, any of a method involving adding it to the organic solvent-containing solution in advance, a method involving adding it to water or the acidic aqueous solution in advance, and a method involving adding it after bringing the organic solvent-containing solution into contact with water or the acidic aqueous solution may be employed. Among them, the method involving adding it to the organic solvent-containing solution in advance is preferable in terms of the workability of operations and the ease of managing the amount to be charged.

It is preferable that the purification method comprise the step of extracting impurities in the compound by further bringing the solution phase containing the compound into contact with water after the first extraction step (the second extraction step). Specifically, for example, it is preferable that, after the extraction treatment is carried out using an acidic aqueous solution, the solution phase that is extracted and recovered from the aqueous solution and that contains the resin and the solvent be further subjected to an extraction treatment with water. The aforementioned extraction treatment with water is not particularly limited, and can be carried out by, for example, thoroughly mixing the solution phase and water by stirring or the like and then leaving the obtained mixed solution to stand still. The mixed solution after being left to stand still is separated into an aqueous phase and a solution phase containing the compound and the solvent, and thus the solution phase can be recovered by decantation or the like.

Water used herein is preferably water, the metal content of which is small, such as ion exchanged water, according to the purpose of the third embodiment. While the extraction treatment may be carried out only once, it is effective to repeat mixing, leaving-to-stand-still, and separating operations multiple times. The proportions of both used in the extraction treatment, and temperature, time and other conditions are not particularly limited, and may be the same as those of the previous contact treatment with the acidic aqueous solution.

Water that is possibly present in the thus-obtained solution containing the compound and the solvent can be easily removed by performing vacuum distillation or a like operation. Also, if required, the concentration of the compound can be regulated to be any concentration by adding a solvent to the solution.

In the purification method of the compound according to the third embodiment, the compound can be purified by passing a solution obtained by dissolving the resin in a solvent through a filter.

According to the purification method according to the third embodiment, the content of various metals in the resin can be effectively and significantly reduced. The amount of these metal components can be measured by the method described in Examples, which will be mentioned later.

Note that “passing” in the third embodiment means that the solution is passed from the outside of a filter through the inside of the filter, and then transferred to the outside of the filter again, and for example, an aspect in which the solution is brought into contact merely on the surface of a filter and an aspect in which the solution is transferred outside of the ion exchange resin while being brought into contact with the surface of a filter (that is, a mere contact aspect) are eliminated.

### [Filtering Purification Step (Passing Step)]

In the filter-passing step in the third embodiment, the filter used for removing the metals in a solution containing the compound and the solvent can be one which is normally commercially available as a filter for liquid filtration. The filtering precision of the filter is not particularly limited, but the nominal pore size of the filter is preferably 0.2 μm or less, more preferably less than 0.2 μm, further preferably 0.1 μm or less, still more preferably less than 0.1 μm, and even more preferably 0.05 μm or less. The lower limit value of the nominal pore size of the filter is not particularly limited, but is normally 0.005 μm. The nominal pore size herein refers to the nominal pore size indicating the separation performance of a filter and is the pore size determined by test methods defined by the manufacturer of the filter, such as a bubble point test, a mercury intrusion porosimetry test, and a standard particle capture test. In the case of using a commercial product, it is the value described in the catalog data of the manufacturer. By setting the nominal pore size to 0.2 μm or less, the content of the metals after the solution is passed through the filter once can be effectively reduced. In the third embodiment, the filter-passing step may be carried out twice or more to further reduce the content of each metal in the solution.

In the purification method of the compound according to the third embodiment, purification can be carried out by distilling the compound itself. The distillation method is not particularly limited, but a known method such as normal pressure distillation, vacuum distillation, molecular distillation, or steam distillation can be used.

### [Applications of Compound (A)]

The compound (A) according to the third embodiment can increase the sensitivity to an exposure light source by being added to a composition for film formation as it is or as the polymer described below. The compound (A) or a polymer thereof is preferably used for photoresists.

### [Composition]

The composition of the third embodiment contains the compound (A). The content of the compound (A) in the third embodiment is preferably 90% by mass or more, more preferably 95% by mass or more, and further preferably 99% by mass or more.

As other preferred form of the composition of the third embodiment, it is preferable that the compound (A) at least include a compound represented by the formula (1) other than the formula (1C) and a compound represented by the formula (1C). The proportion of the monomer represented by the formula (1C) contained is preferably as little as 1 ppm by mass or more and 10% by mass or less, more preferably 20 ppm by mass or more and 2% by mass or less, and 50 ppm by mass or more and 1% by mass or less, based on the total monomer represented by the formula (1).

By setting the content of the compound represented by the formula (1C) to the described range, the interaction between resins when being formed into a resin can be reduced, and by suppressing the crystalline due to the interaction between the resins after a film is formed using the resin, the locality in the solubility in the developing solution upon developing can be reduced at a molecular level from several to several tens of nanometers, the reduction in pattern quality, such as the line edge roughness and the residue defect, of a pattern which is formed in the pattern formation process in a series of lithography processes of exposure, post exposure bake, and development can be suppressed, and the resolution can be further improved.

The influence of these effects for lithography performance increases in the compound represented by the formula (1C), as a result that the hydrophilicity/hydrophobicity of the compound represented by the formula (1) and the compound represented by the formula (1C) each having the core A into which a halogen element, in particular, iodine, fluorine, or the like is introduced, is shifted to a compound having a hydroxystyrene skeleton in which no iodine and the like are introduced, and the polarization at the polar moieties is thus increased.

The composition of the third embodiment contains the compound (A). In the composition, the content of impurities containing K (potassium) is preferably 1 ppm by mass or less, more preferably 0.5 ppm by mass or less, further preferably 0.1 ppm by mass or less, and still more preferably 0.005 ppm by mass or less, in terms of element, based on the total compound (A).

In the composition of the third embodiment, the content of one or more elemental impurities selected from the group consisting of Mn (manganese), Al (aluminum), Si (silicon), and Li (lithium) (preferably, one or more elemental impurities selected from the group consisting of Mn and Al) is preferably 1 ppm or less, more preferably 0.5 ppm or less, further preferably 0.1 ppm or less, in terms of element, based on the total compound (A).

The amount of K, Mn, Al, Si, Li, and the like is measured by inorganic elemental analysis (IPC-AES/IPC-MS). Examples of the inorganic element analyzer include “AG8900” manufactured by Agilent Technologies, Inc.

In the composition of the third embodiment, the content of the phosphorus-containing compound is preferably 10 ppm or less, more preferably 8 ppm or less, and further preferably 5 ppm or less, based on the total compound (A).

In the composition of the third embodiment, the content of maleic acid is preferably 10 ppm or less, more preferably 8 ppm or less, and further preferably 5 ppm or less, based on the total compound (A).

The amount of the phosphorus-containing compound and maleic acid is calculated from the area fraction of the GC chart and the peak intensity ratio between the target peak and the reference peak by gas chromatography mass spectrometry (GC-MS).

In the composition of the third embodiment, the content of peroxide is preferably 10 ppm or less, more preferably 1 ppm or less, and further preferably 0.1 ppm or less, based on the total compound (A).

The content of peroxide is quantified by an ammonium ferrothiocyanate acid method (hereinafter, AFTA method) by adding trichloroacetic acid in a sample, then adding ammonium iron (II) sulfate and potassium thiocyanate, determining a calibration curve of peroxide which is known as a standard, and measuring the absorbance at a wavelength of 480 μm.

In the composition of the third embodiment, the moisture content is preferably 100,000 ppm or less, more preferably 20,000 ppm or less, further preferably 1,000 ppm or less, still more preferably 500 ppm or less, and still more preferably 100 ppm or less, based on the total compound (A). The moisture content is measured by a Karl Fischer method (Karl Fischer moisture content measuring apparatus).

### [Polymer (A)]

The polymer (A) of the third embodiment contains a constitutional unit derived from the aforementioned compound (A). By containing the constitutional unit derived from the compound (A), the polymer (A) can increase the sensitivity to an exposure light source when being blended in a resist composition. In particular, the polymer (A) can exhibit sufficient sensitivity and can form good thin line patterns having a narrow line width even in the case of using an extreme ultraviolet ray as the exposure light source.

The conventional resist compositions were difficult to expand for actual semiconductor production, since their sensitivity to an exposure light source may be reduced over time due to storage or the like. However, according to the polymer (A) of the third embodiment, the stability of the resist composition is improved, and the reduction in the sensitivity to an exposure light source is suppressed even in the case of long-term storage.

The polymer (A) of the third embodiment contains the constitutional unit derived from the compound (A).

The constitutional unit derived from the compound (A) contained in the polymer (A) includes, for example, a constitutional unit represented by the following formula (1-A).

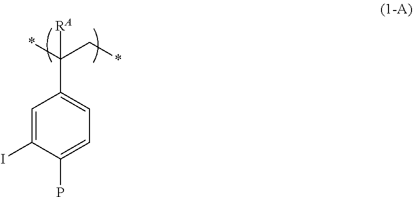

In the formula (1-A), R^{A} and P have the same meanings as in the formula (1); and * is a site for binding with an adjacent constitutional unit.

R^{A} is preferably a hydrogen atom or a methyl group.

Furthermore, P is a hydroxy group, a tertiary ester group, an acetal group, a carbonate ester group, or a carboxyalkoxy group.

The polymer (A) can be obtained by polymerizing the compound (A) of the third embodiment, or by copolymerizing the compound (A) and other monomers. For example, the polymer (A) can be used as a material for forming a film for lithography.

The amount of the constitutional unit derived from the compound (A) is preferably 5 mol % or more, more preferably 8 mol % or more, and further preferably 10 mol % or more, based on the total amount of the monomer components of the polymer (A). The amount of the constitutional unit derived from the compound (A) is 100 mol % or less, preferably 80 mol % or less, more preferably 50 mol % or less, and further preferably 30 mol % or less, based on the total amount of the monomer components of the polymer (A).

As a preferred form of the polymer of the third embodiment, it is preferable that, as the constitutional unit of the polymer (A), the monomer represented by the compound (A) at least include a compound represented by the formula (1) and a compound represented by the formula (1C). The proportion of the monomer represented by the formula (1C) contained is preferably as little as 10 ppm by mass or more and 10% by mass or less, more preferably 20 ppm or more and 2% by mass or less, and 50 ppm or more and 1% by mass or less, based on the total monomer represented by the formula (1).

By setting the proportion of the compound represented by the formula (1C) contained to the aforementioned range, the interaction between resins when being formed into a resin can be reduced. By suppressing the crystalline due to the interaction between the resins after a film is formed using the resin, the locality in the solubility in the developing solution upon developing can be reduced at a molecular level from several to several tens of nanometers. As a result, the reduction in pattern quality, such as the line edge roughness and the residue defect, of a pattern which is formed in the pattern formation process in a series of lithography processes of exposure, post exposure bake, and development can be suppressed, and the resolution can be further improved.

The influence of these effects for lithography performance increases in the monomer represented by the formula (1C), as a result that the hydrophilicity/hydrophobicity of the compound represented by the formula (1) and the compound represented by the formula (1C) each having the core into which a halogen element, in particular, iodine is introduced, is shifted to a compound having a hydroxystyrene skeleton in which no iodine and the like are introduced, and the polarization at the polar moieties is thus increased.

In the polymer (A), as the other monomers to be copolymerized with the compound (A), it is preferable to contain a polymerization unit that has an aromatic compound having an unsaturated double bond as a substituent, as a polymerization unit, and has a functional group for improving solubility in an alkaline developing solution by the action of an acid or a base.

In the polymer (A), examples of other monomers to be copolymerized with the compound (A) include, but are not particularly limited to, those described in International Publication No. WO 2016/125782, International Publication No. WO 2015/115613, Japanese Patent Laid-Open No. 2015/117305, International Publication No. WO 2014/175275, and Japanese Patent Laid-Open No. 2012/162498, or a compound represented by the following formula (C1) or the following formula (C2). Among them, the compound represented by the following formula (C1) or the following formula (C2) is preferable. Other monomers to be copolymerized with the compound (A) in the polymer (A) preferably contains a constitutional unit represented by the following formula (C0).

That is, the polymer (A) preferably further contains the constitutional unit represented by the following formula (C0), a constitutional unit represented by the following formula (C1), or a constitutional unit represented by the following formula (C2), in addition to the constitutional unit represented by the formula (1-A).

From the viewpoint of the quality of the pattern shape after exposure and development in a lithography process, in particular, suppressing the roughness and the pattern collapse, it is preferable that the difference between the dissolution rate Rₘᵢₙ of the resin that becomes protrusions of a pattern during alkali development on unexposed portions during exposure in the alkaline developing solution and the dissolution rate Rₘₐₓ of the resin that becomes recesses of the pattern during alkali development on exposed portions during exposure in the alkaline developing solution be 3 or more orders of magnitude larger, and it is preferable that the difference in the dissolution rate due to the presence or absence of a protective group be large and the elimination rate of the protective group in post-exposure bake (PEB) and development be large. From these viewpoints, other monomers to be copolymerized with the compound (A) in the polymer (A) preferably have a constitutional unit represented by the following formula (C1).

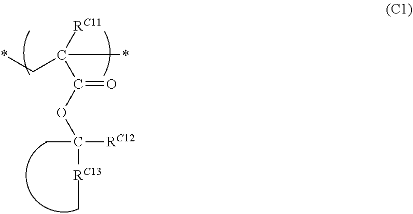

In the formula (C1),
R^{C11} is a hydrogen atom, a methyl group, or a trifluoromethyl group;
R^{C12} is a hydrogen atom, or an alkyl group having 1 to 4 carbon atoms;
R^{C13} is a cycloalkyl group or heterocycloalkyl group having 4 to 20 carbon atoms formed by being taken together with a carbon atom bonded to R^{C13}; and
* is a site for binding with an adjacent constitutional unit.

R^{C12} is preferably a hydrogen atom, or an alkyl group having 1 to 3 carbon atoms. R^{C13} is preferably a cycloalkyl group or heterocycloalkyl group having 4 to 10 carbon atoms formed by being taken together with a carbon atom bonded to R^{C13}. The cycloalkyl group or heterocycloalkyl group of R^{C13} may have a substituent (e.g., an oxo group).

The amount of the constitutional unit represented by the formula (C1) is preferably 5 mol % or more, more preferably 10 mol % or more, and further preferably 20 mol % or more, based on the total amount of the monomer components of the polymer (A). The amount of the constitutional unit represented by the formula (C1) is preferably 90 mol % or less, more preferably 80 mol % or less, further preferably 70 mol % or less, based on the total amount of the monomer components of the polymer (A).

Other monomers to be copolymerized with the compound (A) in the polymer (A) are preferably the constitutional unit represented by the following formula (C2), from the viewpoint of the quality of the pattern shape after exposure and development in a lithography process, in particular, suppressing the roughness and the pattern collapse.

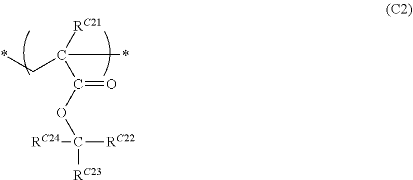

In the formula (C2),
R^{C21} is a hydrogen atom, a methyl group, or a trifluoromethyl group;
each of R^{C22} and R^{C23} is independently an alkyl group having 1 to 4 carbon atoms;
R^{C24} is an alkyl group having 1 to 4 carbon atoms or a cycloalkyl group having 5 to 20 carbon atoms;
two or three selected from R^{C22}, R^{C23}, and R^{C24} are optionally taken together with a carbon atom bonded to the two or three selected from R^{C22}, R^{C23}, and R^{C24} to form an alicyclic structure having 3 to 20 carbon atoms; and
* is a site for binding with an adjacent constitutional unit.

R^{C22} is preferably an alkyl group having 1 to 3 carbon atoms, and R^{C24} is a cycloalkyl group having 5 to 10 carbon atoms. The alicyclic structure formed by R^{C22}, R^{C23}, and R^{C24} may contain a plurality of rings such as an adamantyl group. The alicyclic structure may have a substituent (e.g., a hydroxyl group and an alkyl group).

The amount of the constitutional unit represented by the formula (C2) is preferably 5 mol % or more, more preferably 10 mol % or more, and further preferably 20 mol % or more, based on the total amount of the monomer components of the polymer (A). The amount of the constitutional unit represented by the formula (C2) is preferably 80 mol % or less, more preferably 60 mol % or less, further preferably 40 mol % or less, based on the total amount of the monomer components of the polymer (A).

Examples of the monomer raw material of the constitutional unit represented by the formula (C2) include, but are not limited to, 2-methyl-2-(meth)acryloyloxyadamantane, 2-ethyl-2-(meth)acryloyloxyadamantane, 2-isopropyl-2-(meth)acryloyloxyadamantane, 2-n-propyl-2-(meth)acryloyloxyadamantane, 2-n-butyl-2-(meth)acryloyloxyadamantane, 1-methyl-1-(meth)acryloyloxycyclopentane, 1-ethyl-1-(meth)acryloyloxycyclopentane, 1-methyl-1-(meth)acryloyloxycyclohexane, 1-ethyl-1-(meth)acryloyloxycyclohexane, 1-methyl-1-(meth)acryloyloxycycloheptane, 1-ethyl-1-(meth)acryloyloxycycloheptane, 1-methyl-1-(meth)acryloyloxycyclooctane, 1-ethyl-1-(meth)acryloyloxycyclooctane, 2-ethyl-2-(meth)acryloyloxydecahydro-1,4:5,8-dimethanonaphtalene, and 2-ethyl-2-(meth)acryloyloxynorbornane. Commercial products may be used as these monomer.

Other monomers to be copolymerized with the compound (A) in the polymer (A) are preferably the constitutional unit represented by the following formula (C0), from the viewpoint of the quality of the pattern shape after exposure and development in a lithography process, sensitization, in particular, suppressing the roughness and the pattern collapse.

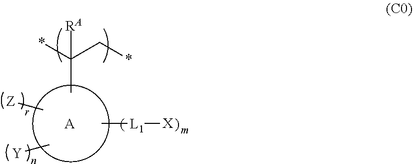

In the formula (C0),
each X is independently I, F, Cl, Br, or an organic group having 1 to 30 carbon atoms and having 1 or more and 5 or less substituents selected from the group consisting of I, F, Cl, and Br;
each L¹ is independently a single bond, an ether group, an ester group, a thioether group, an amino group, a thioester group, an acetal group, a phosphine group, a phosphone group, a urethane group, a urea group, an amide group, an imide group, or a phosphate group, and the ether group, the ester group, the thioether group, the amino group, the thioester group, the acetal group, the phosphine group, the phosphone group, the urethane group, the urea group, the amide group, the imide group, or the phosphate group of L¹ optionally has a substituent;
each Y is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;
R^{A} is as defined in the formula (1);
A is an organic group having 1 to 30 carbon atoms;
each Z is independently an alkoxy group, an ester group, an acetal group, a carboxyalkoxy group, or a carbonate ester group, and the alkoxy group, the ester group, the acetal group, the carboxyalkoxy group, or the carbonate ester group of Z optionally has a substituent; and
m is an integer of 0 or more, n is an integer of 1 or more, and r is an integer of 0 or more.

For example, X may be an aromatic group into which one or more F, Cl, Br, or I are introduced. Examples of such an aromatic group include a group having a benzene ring such as a phenyl group and having 1 to 5 halogens, and a group having a heteroaromatic ring such as furan, thiophene, and pyridine and having 1 to 5 halogens. Examples thereof include a phenyl group having 1 to 5 I, a phenyl group having 1 to 5 F, a phenyl group having 1 to 5 Cl, a phenyl group having 1 to 5 Br, a naphthyl group having 1 to 5 F, a naphthyl group having 1 to 5 Cl, a naphthyl group having 1 to 5 Br, a naphthyl group having 1 to 5 I, a phenol group having 1 to 4 F, a phenol group having 1 to 4 Cl, a phenol group having 1 to 4 Br, a phenol group having 1 to 4 I, a furan group having 1 to 3 F, a furan group having 1 to 3 Cl, a furan group having 1 to 3 Br, a furan group having 1 to 3 I, a thiophene group having 1 to 3 F, a thiophene group having 1 to 3 Cl, a thiophene group having 1 to 3 Br, a thiophene group having 1 to 3 I, a pyridine group having 1 to 4 F, a pyridine group having 1 to 4 Cl, a pyridine group having 1 to 4 Br, a pyridine group having 1 to 4 I, a benzodiazole group having 1 to 5 F, a benzodiazole group having 1 to 5 Cl, a benzodiazole group having 1 to 5 Br, a benzodiazole group having 1 to 5 I, a benzimidazole group having 1 to 4 F, a benzimidazole group having 1 to 4 Cl, a benzimidazole group having 1 to 4 Br, a benzimidazole group having 1 to 4 I, a benzoxazole group having 1 to 4 F, a benzoxazole group having 1 to 4 Cl, a benzoxazole group having 1 to 4 Br, a benzoxazole group having 1 to 4 I, a benzothiophene group having 1 to 4 F, a benzothiophene group having 1 to 4 Cl, a benzothiophene group having 1 to 4 Br, and a benzothiophene group having 1 to 4 I. X may be an alicyclic group into which one or more F, Cl, Br, or I are introduced. Examples of such an alicyclic group include an adamantyl group having 1 to 3 halogens, an adamantyl group having 1 to 3 F, an adamantyl group having 1 to 3 Cl, an adamantyl group having 1 to 3 Br, an adamantyl group having 1 to 3 I, a cyclopentyl group having 1 to 3 F, a cyclopentyl group having 1 to 3 Cl, a cyclopentyl group having 1 to 3 Br, a cyclopentyl group having 1 to 3 I, a bicycloundecyl group having 1 to 3 F, a bicycloundecyl group having 1 to 3 Cl, a bicycloundecyl group having 1 to 3 Br, a bicycloundecyl group having 1 to 3 I, a norbornyl group having 1 to 3 F, a norbornyl group having 1 to 3 Cl, a norbornyl group having 1 to 3 Br, and a norbornyl group having 1 to 3 I.

L¹ is a single bond, an ether group, an ester group, a thioether group, an amino group, a thioester group, an acetal group, a phosphine group, a phosphone group, a urethane group, a urea group, an amide group, an imide group, or a phosphate group. Among them, L¹ is preferably a single bond. The ether group, the ester group, the thioether group, the amino group, the thioester group, the acetal group, the phosphine group, the phosphone group, the urethane group, the urea group, the amide group, the imide group, or the phosphate group of L¹ optionally has a substituent. Examples of such a substituent are as described above.

m is an integer of 0 or more, preferably an integer of 0 or more and 5 or less, more preferably an integer of 0 or more and 2 or less, further preferably 0 or 1, and particularly preferably 0.

Each Y is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent.

Examples of Y include at least one group selected from the group consisting of an alkoxy group [^{*3}—O—R²], an ester group [^{*3}—O—(C═O)—R² or ^{*3}—(C═O)—O—R²], an acetal group [^{*3}—O—(C(R²¹)₂)—O—R² (wherein each R²¹ is independently H or a hydrocarbon group having 1 to 10 carbon atoms)], a carboxyalkoxy group [^{*3}—O—R²²—(C═O)—O—R² (wherein R²² is a divalent hydrocarbon group having 1 to 10 carbon atoms)], and a carbonate ester group [^{*3}—O—(C═O)—O—R²]. The ester group is preferably a tertiary ester group, from the viewpoint of achieving high sensitivity. In the formula, ^{*3} is a site for binding with A.

Among them, Y is preferably a tertiary ester group, an acetal group, a carbonate ester group, or a carboxyalkoxy group, more preferably an acetal group, a carbonate ester group, or a carboxyalkoxy group, and further preferably an acetal group or a carboxyalkoxy group, from the viewpoint of high sensitivity. From the viewpoint of producing a polymer having a stable quality by radical polymerization, an ester group, a carboxyalkoxy group, and a carbonate ester group are preferable.

Each Y is preferably independently a group represented by the following formula (Y-1).

-L²-R² (Y-1)

In the formula (Y-1),

L² is a group which is cleaved by the action of an acid or a base. Examples of the group which is cleaved by the action of an acid or a base include at least one divalent linking group selected from the group consisting of an ester group [^{*1}—O—(C═O)—^{*2} or ^{*1}—(C═O)—O—^{*2}], an acetal group [^{*1}—O—(C(R²¹)₂)—O—^{*2} (each R²¹ is independently H or a hydrocarbon group having 1 to 10 carbon atoms)], a carboxyalkoxy group [^{*1}—O—R²²—(C═O)—O—^{*2} (R²² is a divalent hydrocarbon group having 1 to 10 carbon atoms)], and a carbonate ester group [^{*1}—O—(C═O)—O—^{*2}]. The ester group is preferably a tertiary ester group, from the viewpoint of achieving high sensitivity. In the formula, ^{*1} is a site for binding with A, and ^{*2} is a site for binding with R². Among them, L² is preferably a tertiary ester group, an acetal group, a carbonate ester group, or a carboxyalkoxy group, more preferably an acetal group, a carbonate ester group, or a carboxyalkoxy group, and further preferably an acetal group or a carboxyalkoxy group, from the viewpoint of high sensitivity. From the viewpoint of producing a polymer having a stable quality by radical polymerization, an ester group, a carboxyalkoxy group, and a carbonate ester group are preferable.

As another effect, Y is preferably a group represented by the formula (Y-1) to control the polymerization properties of resin and the degree of polymerization in a desired range, when the compound (A) of the third embodiment is used as a polymerization unit of a copolymer. Since the compound (A) has a large influence on activity species in the polymer formation reaction due to having an X group and thus the desired control is difficult, variation of copolymer formation derived from the hydrophilic group and polymerization inhibition can be suppressed by having a group represented by the formula (Y-1) in the hydrophilic group in the compound (A) as a protecting group.

R² is a linear, branched, or cyclic aliphatic group having 1 to 30 carbon atoms, an aromatic group having 6 to 30 carbon atoms, a linear, branched, or cyclic aliphatic group containing a heteroatom and having 1 to 30 carbon atoms, or a linear, branched, or cyclic aromatic group containing a heteroatom and having 1 to 30 carbon atoms, and the aliphatic group, the aromatic group, the aliphatic group containing a heteroatom, and the aromatic group containing a heteroatom of R² optionally further have a substituent. As the substituent here, the aforementioned substituents are used, but a linear, branched, or cyclic aliphatic group having 1 to 20 carbon atoms or an aromatic group having 6 to 20 carbon atoms are preferable. Among them, R² is preferably an aliphatic group. The aliphatic group in R² is preferably a branched or cyclic aliphatic group. The number of carbon atoms of the aliphatic group is preferably 1 or more and 20 or less, more preferably 3 or more and 10 or less, and further preferably 4 or more and 8 or less. Examples of the aliphatic group include, but are not particularly limited to, a methyl group, an isopropyl group, a sec-butyl group, a tert-butyl group, an isobutyl group, a cyclohexyl group, a methylcyclohexyl group, and an adamantyl group. Among them, a tert-butyl group, a cyclohexyl group, or an adamantyl group is preferable.

L² is preferably ^{*1}—(C═O)—O—^{*2} or a carboxyalkoxy group, because, when L² is cleaved by the action of an acid or a base, a carboxylic acid group is formed and the difference in the solubility and the difference in the dissolution rate between a cleaved portion and an uncleaved portion are increased in the development treatment, so that the resolution is improved and, in particular, residues at the pattern bottom in thin line patterns are suppressed.

Specific examples of Y include the followings. Each Y is independently a group represented by any of the following formulas.

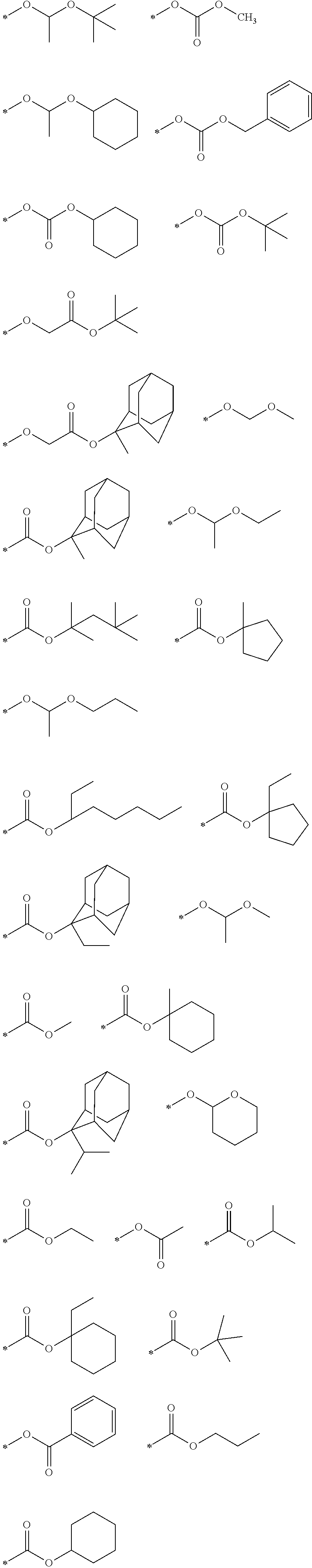

n is an integer of 1 or more, preferably an integer of 1 or more and 5 or less, more preferably an integer of 1 or more and 4 or less, further preferably an integer of 1 or more and 3 or less, still more preferably 1 or 2, and particularly preferably 1.
each R^{A} is independently H, I, F, Cl, Br, or an organic group having 1 to 60 carbon atoms and optionally having a substituent. Examples of the substituent of the organic group having 1 to 60 carbon atoms include, but are not particularly limited to, I, F, Cl, Br, or other substituents. Examples of other substituents include, but are not particularly limited to, a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, and a phosphate group. Among them, the alkoxy group, the ester group, the carbonate ester group, the amino group, the ether group, the thioether group, the phosphine group, the phosphone group, the urethane group, the urea group, the amide group, the imide group, and the phosphate group optionally further have a substituent. Examples of the substituent here include a linear, branched, or cyclic aliphatic group having 1 to 20 carbon atoms and an aromatic group having 6 to 20 carbon atoms.

The number of carbon atoms of the organic group optionally having a substituent in R^{A} is preferably 1 to 30.

Examples of the organic group having 1 to 60 carbon atoms and optionally having a substituent include, but are not particularly limited to, a linear or branched aliphatic hydrocarbon group having 1 to 60 carbon atoms, a cycloaliphatic hydrocarbon group having 4 to 60 carbon atoms, and an aromatic group having 6 to 60 carbon atoms and optionally having a heteroatom.

Examples of the linear or branched aliphatic hydrocarbon group having 1 to 60 carbon atoms include, but are not particularly limited to, a methyl group, an ethyl group, an n-propyl group, an isopropyl group, an n-butyl group, an isobutyl group, a tert-butyl group, an n-pentyl group, an n-hexyl group, an n-dodecyl group, a valeryl group, and a 2-ethylhexyl group.

Examples of the cycloaliphatic hydrocarbon group include, but are not particularly limited to, a cyclohexyl group, a cyclododecyl group, a dicyclopentyl group, a tricyclodecyl group, and an adamantyl group. Further, an aromatic group optionally having a heteroatom such as a benzodiazole group, a benzotriazole group, and a benzothiadiazole group can be arbitrarily selected. A combination of these organic groups can also be selected.

Examples of the aromatic group having 6 to 60 carbon atoms and optionally having a heteroatom include, but are not particularly limited to, a phenyl group, a naphthalene group, a biphenyl group, an anthracyl group, a pyrenyl group, a benzodiazole group, a benzotriazole group, and a benzothiadiazole group.

Among the organic group having 1 to 60 carbon atoms and optionally having a substituent, a methyl group is preferable, from the viewpoint of producing a polymer having a stable quality.

A is an organic group having 1 to 30 carbon atoms. A may be a monocyclic organic group or a polycyclic organic group, or optionally has a substituent. A is preferably an aromatic ring optionally having a substituent. The number of carbon atoms of A is preferably 6 to 14, and more preferably 6 to 10.

A is preferably a group represented by any of the following formulas, more preferably a group represented by the following formulas (A-1) to (A-2), and further preferably a group represented by the following formula (A-1).

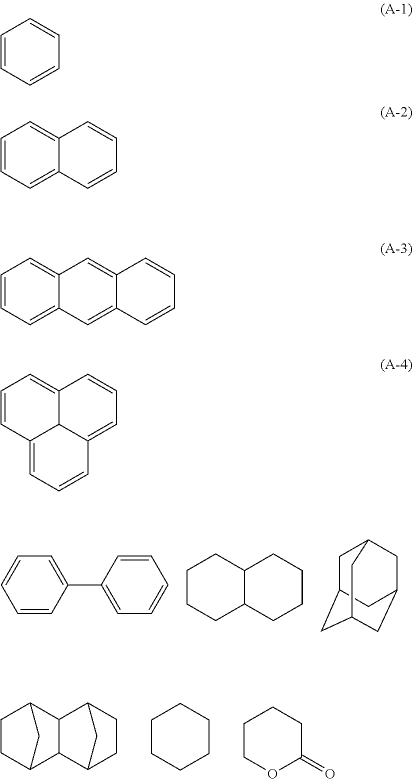

A may be an alicyclic structure optionally having a substituent. Here, the “alicyclic structure” refers to a saturated or unsaturated carbocycle having no aromatic properties. Examples of the alicyclic structure include a saturated or unsaturated carbocycle having 3 to 30 carbon atoms, and a saturated or unsaturated carbocycle having 3 to 20 carbon atoms is preferable. Examples of the alicyclic structure include a group having cyclopropyl, cyclobutyl, cyclopentyl, cyclohexyl, cycloheptyl, cyclooctyl, cyclononyl, cyclodecyl, cycloicosyl, cyclopropenyl, cyclobutenyl, cyclopentenyl, cyclohexenyl, cycloheptenyl, cyclooctenyl, cyclopentadienyl, cyclooctadienyl, adamantyl, bicycloundecyl, decahydronaphthyl, norbornyl, norbornadienyl, cubane, basketane, or housane.

A may also be a heterocyclic structure optionally having a substituent. Examples of the heterocyclic structure include, but are not particularly limited to, a nitrogen-containing cyclic structure such as pyridine, piperidine, piperidone, benzodiazole, and benzotriazole; a cyclic ether such as triazine, a cyclic urethane structure, cyclic urea, cyclic amide, cyclic imide, furan, pyran, and dioxolane; an alicyclic group having a lactone structure such as caprolactone, butyrolactone, nonalactone, decalactone, undecalactone, bicycloundecalactone, and phthalide.

Each Z is independently an alkoxy group, an ester group, an acetal group, a carboxyalkoxy group, or a carbonate ester group. These groups optionally have a substituent, and examples of the substituent include a hydrocarbon group having 1 to 60 carbon atoms and optionally further having a substituent. r is an integer of 0 or more, preferably an integer of 0 or more and 2 or less, more preferably an integer of 0 or more and 1 or less, and further preferably 0.

Examples of Z include at least one group selected from the group consisting of an alkoxy group [^{*3}—O—R²], an ester group [^{*3}—O—(C═O)—R² or ^{*3}—(C═O)—O—R²], an acetal group [^{*3}—O—(C(R²¹)₂)—O—R² (wherein each R²¹ is independently H or a hydrocarbon group having 1 to 10 carbon atoms)], a carboxyalkoxy group [^{*3}—O—R²²—(C═O)—O—R² (wherein R²² is a divalent hydrocarbon group having 1 to 10 carbon atoms)], and a carbonate ester group [^{*3}—O—(C═O)—O—R²]. The ester group is preferably a tertiary ester group, from the viewpoint of achieving high sensitivity. In the formula, ^{*3} is a site for binding with A.

Among them, Z is preferably a tertiary ester group, an acetal group, a carbonate ester group, or a carboxyalkoxy group, more preferably an acetal group, a carbonate ester group, or a carboxyalkoxy group, and further preferably an acetal group or a carboxyalkoxy group, from the viewpoint of high sensitivity. From the viewpoint of producing a polymer having a stable quality by radical polymerization, an ester group, a carboxyalkoxy group, and a carbonate ester group are preferable.

Other monomers to be copolymerized with the compound (A) in the polymer (A) preferably have a constitutional unit represented by the following formula (C3).

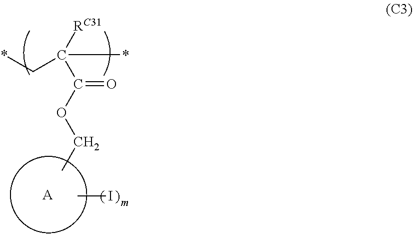

In the formula (C3), R^{C31} is a hydrogen atom, a methyl group, or a trifluoromethyl group; and m, A, and * are as defined in the formula (C0).

Next, the method for producing the polymer (A) will be described. The polymerization reaction is carried out by dissolving a monomer as a constitutional unit in a solvent and adding a polymerization initiator while heating or cooling. The reaction conditions can be inadvertently set depending on the kind of polymerization initiator, the initiation method such as heat and light, the temperature, the pressure, the concentration, the solvent, the additive, and the like. Examples of the polymerization initiator include a radical polymerization initiator such as azoisobutyronitrile and peroxide, and an anion polymerization initiator such as alkyllithium and a Grignard reagent.

As the solvent used in the polymerization reaction, a commonly available product can be used. For example, a wide variety of solvents such as alcohols, ethers, hydrocarbons, and halogenated solvents can be arbitrarily used within a range not inhibiting the reaction. A plurality of solvents may be mixed and used within a range not inhibiting the reaction.

The polymer (A) obtained in the polymerization reaction can be purified by a publicly known method. Specifically, ultrafiltration, crystallization, microfiltration, acid washing, water washing at an electrical conductivity of 10 mS/m or less, and extraction can be used in combination.

### [Composition and Composition for Film Formation]

The composition or composition for film formation of the third embodiment contains the compound (A) or the polymer (A), and is the composition particularly suitable for lithography technology. The composition or the composition for film formation can be used for, without particular limitation, film formation purposes for lithography, for example, resist film formation purposes (that is, a “resist composition”). Furthermore, the composition or the composition for film formation can be used for upper layer film formation purposes (that is, a “composition for upper layer film formation”), intermediate layer formation purposes (that is, a “composition for intermediate layer formation”), underlayer film formation purposes (that is, a “composition for underlayer film formation”), and the like. According to the composition of the third embodiment, not only a film having high sensitivity can be formed, but also a good resist pattern shape can be imparted.

The composition for film formation of the third embodiment can also be used as an optical component forming composition applying lithography technology. The optical component is used in the form of a film or a sheet and is also useful as a plastic lens (a prism lens, a lenticular lens, a microlens, a Fresnel lens, a viewing angle control lens, a contrast improving lens, etc.), a phase difference film, a film for electromagnetic wave shielding, a prism, an optical fiber, a solder resist for flexible printed wiring, a plating resist, an interlayer insulating film for multilayer printed circuit boards, a photosensitive optical waveguide, a liquid crystal display, an organic electroluminescent (EL) display, an optical semiconductor (LED) element, a solid state image sensing element, an organic thin film solar cell, a dye sensitized solar cell, and an organic thin film transistor (TFT). The composition can be suitably utilized as an embedded film and a smoothed film on a photodiode, a smoothed film in front of or behind a color filter, a microlens, and a smoothed film and a conformal film on a microlens, all of which are components of a solid state image sensing element, to which high refractive index is particularly demanded.

The composition for film formation of the third embodiment may contain the compound (A), the composition of the third embodiment, or the polymer (A). The composition for film formation of the third embodiment may further contain an acid generating agent (C), a base generating agent (G), or an acid diffusion controlling agent (E) (basic compound). The composition for film formation of the third embodiment may further contain other components such as a base material (B) and a solvent (S), if required. Since the base material (B), the acid generating agent (C), the base generating agent (G), the acid diffusion controlling agent (E), and other components that may be contained in the composition for film formation of the third embodiment are the same as those of the second embodiment, the description thereof is omitted here.

### [Method for Forming Resist Pattern and Insulating Film]

The resist pattern formation method of the third embodiment comprises:
a step of forming a resist film on a substrate using the composition for film formation of the third embodiment;
a step of exposing a pattern on the resist film; and
a step of subjecting the resist film after exposure to a development treatment.

The insulating film formation method of the third embodiment may comprise the resist pattern formation method of the third embodiment. That is, the insulating film formation method of the third embodiment may comprise:
a step of forming a resist film on a substrate using the composition for film formation of the third embodiment;
a step of exposing a pattern on the resist film; and
a step of subjecting the resist film after exposure to a development treatment.

The composition for film formation of the third embodiment contains, for example, the compound (A), the composition of the third embodiment, or the polymer (A).

Examples of the coating method in the step of forming a resist film include, but are not particularly limited to, spin coating, dip coating, and roll coating. Examples of the substrate include, but are not particularly limited to, a silicon wafer, metal, plastic, glass, and ceramic. After formation of the resist film, a heat treatment may be carried out at a temperature of about 50° C. to 200° C. The film thickness of the resist film is not particularly limited and is for example, 50 nm to 1 μm.

In the exposure step, exposure may be carried out via a predetermined mask pattern, or shot exposure may be carried out without masking. The thickness of the coating film is, for example, 0.1 to 20 μm, and preferably about 0.3 to 2 μm. Lights of various wavelengths such as ultraviolet ray, X-ray can be utilized for exposure, and for example, far ultraviolet ray such as F2 excimer laser (wavelength: 157 nm), ArF excimer laser (wavelength: 193 nm), and KrF excimer laser (wavelength: 248 nm), extreme ultraviolet ray (wavelength: 13 n), X-ray, and electron beam can be arbitrarily selected and used, as a light source. Among them, extreme ultraviolet ray is preferable. The exposure conditions such as the exposure amount are arbitrarily selected depending on the composition of the aforementioned resin and/or compound blended, the kind of each additive, and the like.

In the third embodiment, to stably form a fine pattern with a high degree of accuracy, the resist film is preferably subjected to a heat treatment at a temperature of 50 to 200° C. for 30 seconds or more, after exposure. In this case, variation of sensitivity depending on the kind of substrate may increase at a temperature lower than 50° C. Thereafter, development is carried out with an alkaline developing solution under the conditions of normally at 10 to 50° C. for 10 to 200 seconds, and preferably 20 to 25° C. for 15 to 90 seconds, resulting in formation of a predetermined resist pattern.

A plurality of above solvents may be mixed, or the solvent may be used by mixing the solvent with a solvent other than those described above or water within the range having performance. In order to sufficiently exhibit the effect of the third embodiment, the moisture content as the whole developing solution is preferably less than 70% by mass, and furthermore, preferably less than 50% by mass, more preferably less than 30% by mass, and further preferably less than 10% by mass. Particularly preferably, the developing solution is substantially moisture free. That is, the content of the organic solvent in the developing solution is preferably 30% by mass or more and 100% by mass or less based on the total amount of the developing solution, and furthermore, preferably 50% by mass or more and 100% by mass or less, more preferably 70% by mass or more and 100% by mass or less, further preferably 90% by mass or more and 100% by mass or less, and particularly preferably 95% by mass or more and 100% by mass or less, without particular limitation.

The amount of the surfactant used is usually 0.001 to 5% by mass based on the total amount of the developing solution, preferably 0.005 to 2% by mass, and further preferably 0.01 to 0.5% by mass.

For the development method, for example, a method for dipping a substrate in a bath filled with a developing solution for a fixed time (dipping method), a method for raising a developing solution on a substrate surface by the effect of a surface tension and keeping it still for a fixed time, thereby conducting the development (puddle method), a method for spraying a developing solution on a substrate surface (spraying method), and a method for continuously ejecting a developing solution on a substrate rotating at a constant speed while scanning a developing solution ejecting nozzle at a constant rate (dynamic dispense method), or the like may be applied. The time for carrying out the pattern development is not particularly limited, but is preferably 10 seconds to 90 seconds.

In addition, after the step of conducting the development, a step of stopping the development by the replacement with another solvent may be carried out.

After the development, it is preferable that a step of rinsing the resist film with a rinsing solution containing an organic solvent is included.

The rinsing solution used in the rinsing step after the development is not particularly limited as long as the rinsing solution does not dissolve the resist pattern cured by crosslinking. A solution containing a general organic solvent or water may be used as the rinsing solution. As the foregoing rinsing solution, a rinsing solution containing at least one kind of organic solvent selected from a hydrocarbon-based solvent, a ketone-based solvent, an ester-based solvent, an alcohol-based solvent, an amide-based solvent, and an ether-based solvent is preferably used. More preferably, after development, a step of rinsing the film by using a rinsing solution containing at least one kind of organic solvent selected from the group consisting of a ketone-based solvent, an ester-based solvent, an alcohol-based solvent and an amide-based solvent is conducted. Further preferably, after development, a step of rinsing the film by using a rinsing solution containing an alcohol-based solvent or an ester-based solvent is conducted. Even more preferably, after the development, a step of rinsing the film by using a rinsing solution containing a monohydric alcohol is conducted. Particularly preferably, after the development, a step of rinsing the film by using a rinsing solution containing a monohydric alcohol having 5 or more carbon atoms is carried out. The time for rinsing the pattern is not particularly limited, but is preferably 10 seconds to 90 seconds.

The composition of the third embodiment can also be used as an optical component forming composition applying lithography technology. The optical component is used in the form of a film or a sheet and is also useful as a plastic lens (a prism lens, a lenticular lens, a microlens, a Fresnel lens, a viewing angle control lens, a contrast improving lens, etc.), a phase difference film, a film for electromagnetic wave shielding, a prism, an optical fiber, a solder resist for flexible printed wiring, a plating resist, an interlayer insulating film for multilayer printed circuit boards, a photosensitive optical waveguide, a liquid crystal display, an organic electroluminescent (EL) display, an optical semiconductor (LED) element, a solid state image sensing element, an organic thin film solar cell, a dye sensitized solar cell, and an organic thin film transistor (TFT). The composition can be suitably utilized as an embedded film and a smoothed film on a photodiode, a smoothed film in front of or behind a color filter, a microlens, and a smoothed film and a conformal film on a microlens, all of which are components of a solid state image sensing element, to which high refractive index is particularly demanded.

Also, the composition of the third embodiment can be used as a patterning material for lithography applications. For lithography process applications, the composition can be used for various applications such as a semiconductor, a liquid crystal display panel, a display panel using OLED, a power device, and CCD and other sensors. In particular, for semiconductors and integrated circuits on devices, the composition of the third embodiment can be suitably utilized in the process of forming a device element on a silicon wafer, for the purpose of constructing a semiconductor element and other devices by forming a pattern on an insulating film on the substrate side using etching based on the pattern formed on the upper surface side of the insulating layer such as a silicon oxide film and other oxide films utilizing the composition of the third embodiment, further laminating a metal film or a semiconductor material based on the formed insulating film pattern, and forming a circuit pattern.

The compound, polymer, composition, composition for film formation, pattern formation method, insulating film formation method, and method for producing a compound described above in the third embodiment may be applied to extreme ultraviolet applications.

That is, the compound may be used as the composition irradiated with extreme ultraviolet (composition for extreme ultraviolet). The polymer may be used in the composition for extreme ultraviolet. The composition may be the composition for extreme ultraviolet. The composition for film formation may be the composition for extreme ultraviolet. The pattern formation method may comprise a step of exposing a pattern with extreme ultraviolet on a resist film formed on a substrate using the composition for film formation. The insulating film formation method may comprise a step of exposing a pattern with extreme ultraviolet on a resist film formed on a substrate using the composition for film formation. The method for producing a compound may comprise the method for producing a compound used in the composition irradiated with extreme ultraviolet.

The composition or composition for film formation in which the compound in the third embodiment is used can increase the sensitivity to an exposure light source, and in particular, can exhibit sufficient sensitivity and can form good thin line patterns having a narrow line width even in the case of using an extreme ultraviolet ray as the exposure light source. Therefore, the pattern formation method or the insulating film formation method allows the composition to exhibit sufficient sensitivity and to form good thin line patterns having a narrow line width even in the case of comprising the step of exposing a pattern with extreme ultraviolet.

The description of the third embodiment is as above.

### EXAMPLES

Hereinafter, the present invention will be described in further detail with reference to Examples and Comparative Examples, but the present invention is not limited by these Examples in any way.

The example number imparted to each Example described below is the individual example number for each Example Group. That is, for example, Example 1 of Example Group 1 and Example 1 of Example Group 2 are distinguished as being different from each other.

### [Measurement Method]

### [Nuclear Magnetic Resonance (NMR)]

The structure of the compound was verified by carrying out NMR measurement under the following conditions using a nucleus magnetic resonance apparatus “Advance 600 II spectrometer” (product name, manufactured by Bruker).

### [¹H-NMR Measurement]

Frequency: 400 MHz
Solvent: CDCl₃, or d₆-DMSO
Internal standard: TMS
Measurement temperature: 23° C.

### [¹³C-NMR Measurement]

Frequency: 500 MHz
Solvent: CDCl₃, or d₆-DMSO
Internal standard: TMS
Measurement temperature: 23° C.

### [Inorganic Element Content]

The metal content contained in the compounds prepared in Examples and Comparative Examples was measured using an inorganic element analyzer (ICP-AES/ICP-MS) “AG8900” (product name, manufactured by Agilent Technologies, Inc.).

### [Organic Impurity Content]

The organic impurity content contained in the compounds prepared in Examples and Comparative Examples was calculated from the area fraction of the GC chart and the peak intensity ratio between the target peak and the reference peak by gas chromatography mass spectrometry (GC-MS).

### Example Group 1

### Example Group 1: Example A1

Into a 3 L glass flask as a reaction vessel, 283 g (792 mmol) of triphenylphosphonium methyl bromide, 7 mg of methylhydroquinone, and 1470 mL of dehydrated THF were put and dissolved. While the temperature was regulated to 15° C. or less, 148 g (1320 mmol) of potassium tert-butoxide was added portionwise into a THF solution which was put in an ice bath, and then the mixture was stirred for 30 minutes as it was. While the temperature was further regulated to 25° C. or less, 147 g (529 mmol) of 4-hydroxy-3-iodo-5-methoxybenzenecarbaldehyde was added portionwise, and then the mixture was stirred for 30 minutes as it was. Thereafter, the reaction solution was added to 4000 mL of a 3N aqueous HCl solution, and then the mixture was further washed with 1 L of toluene and 2 L of water in the order presented. 128 g of 4-hydroxy-3-iodo-5-methoxystyrene represented by the formula (M1) as the target substance was isolated using a silica gel column.

As a result of the analysis by liquid chromatography-mass spectrometry (LC-MS), the molecular weight was found to be 276.

The following peaks were found by carrying out ¹H-NMR measurement under the above measurement conditions, and the compound was confirmed to have the chemical structure of the compound A1 represented by the formula (M1).

δ (ppm) (d6-DMSO): 3.8 (3H, —CH3), 7.7 (2H, Ph), 6.7 (1H, —CH═), 5.3 (1H, ═CH2), 5.7 (1H, ═CH2), 9.5 (1H, —OH)

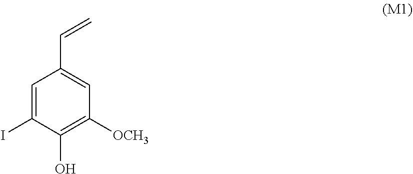

### Example Group 1: Example A1-A

### (Step 1) Malonic Acid Addition Reaction

Using a 200 mL eggplant flask equipped with a Dienstark reflux tube, 10.8 g (38 mmol) of 4-hydroxy-3-iodo-5-methoxybenzaldehyde was mixed with dimethyl malonate (10.6 g, 80 mmol), piperidine (3.4 g, 40 mmol), acetic acid (2.4 g, 40 mmol), and 40 mL of benzene, and allowed to react for 3 hours under reflux conditions. The obtained reaction solution was washed with 20 mL of a 5% by mass aqueous HCl solution, and then washed with a 5% aqueous NaHCO₃ solution. The obtained organic phase was dried over magnesium sulfate, and then concentrated under reduced pressure to obtain 11.8 g of a reaction product (M1-1).

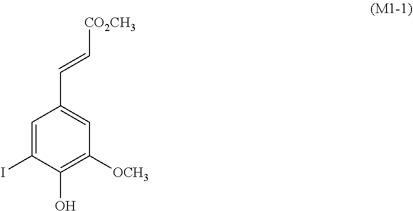 [SMILES: COC(=O)/C=C/c1cc(I)c(O)c(OC)c1]

### (Step 2) Hydrolysis Reaction

Using a 1 L eggplant flask equipped with a reflux tube, hydrochloric acid (6N, 131 mL) and acetic acid (131 mL) were added to 38 mmol of the product (M1-1) obtained above, and the mixture was refluxed for 48 hours. Thereafter, 6M, 500 mL of NaOH aq. was added, and the mixture was extracted with 250 mL of ethyl acetate to recover the organic phase consisting of ethyl acetate. The obtained organic phase was subjected to a dehydration treatment with magnesium sulfate, and the filtrate filtered thereafter was concentrated under reduced pressure to obtain 15.2 g of a cinnamic acid derivative (M1-2).

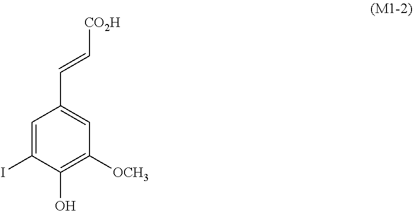 [SMILES: COc1cc(/C=C/C(=O)O)cc(I)c1O]

### (Step 3) Decarboxylation Reaction

Using a 1 L eggplant flask, a solution in which 0.13 g (0.4 mmol) of tetrabutylammonium fluoride trihydrate was dissolved in 20 mL of dimethylsulfoxide was slowly added to a solution in which 40 mmol of the cinnamic acid derivative (M1-2) prepared above was dissolved in 40 mL of dimethylsulfoxide at 10° C. and stirred, and then the mixture was warmed to 40° C. and stirred for 12 hours. The obtained reaction solution was washed three times with 20 mL of pure water, dried over magnesium sulfate, and the filtrate obtained after filtration was concentrated under reduced pressure to obtain 14.4 g of a compound A1 represented by the formula (M1).

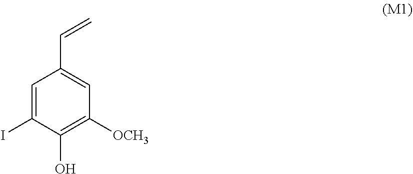

### Example Group 1: Example A1-B

### (Step 1) Synthesis of 4′-hydroxy-3′-iodo-5′-methoxyacetophenone

A reactor was charged with 61.27 g of 4′-hydroxy-3′-methoxyacetophenone, 91.38 g of iodine, 1,620 mL of methanol, and 180 mL of pure water, the reactor was immersed in an ice bath, and stirring was initiated. Subsequently, 44.06 g of an aqueous iodic acid solution with a mass percent concentration of 71.9 was added dropwise thereto for 30 minutes. Subsequently, the reactor was immersed in a water bath at 35° C., and stirring was continued over 3.5 hours. Subsequently, 13.37 g of an aqueous sodium hydrogen sulfite solution with a mass percent concentration of 35 was added to quench the reaction. Subsequently, the content of the reactor was gradually added to 3,600 mL of pure water with vigorous stirring and mixed. Subsequently, the precipitate was filtered off with a suction filter, compressed, and washed with 540 mL of an aqueous methanol solution with a volume percent concentration of 33.3. Subsequently, the precipitate was dried in vacuo at 40° C. to obtain 169.54 g of 4′-hydroxy-3′-iodo-5′-methoxyacetophenone. The yield was 97.1%.

As a result of the analysis by liquid chromatography-mass spectrometry (LC-MS), the molecular weight was found to be 292, and the substance was confirmed to be 4′-hydroxy-3′-iodo-5′-methoxyacetophenone.

The following peaks were found by carrying out ¹H-NMR measurement under the above measurement conditions, and the compound was confirmed to have the chemical structure of 4′-hydroxy-3′-iodo-5′-methoxyacetophenone.

δ (ppm) (d6-DMSO): 10.5 (1H, OH), 8.3 (2H, Ph), 3.8 (3H, —CH3), 2.5 (3H, —CH3)

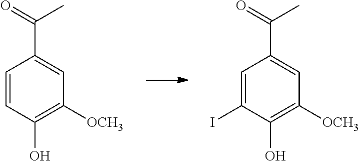

### (Step 2) Synthesis of 1-(4-hydroxy-3-methoxyphenyl)ethanol

The reactor was charged with 8.77 g of sodium borohydride and 180 mL of tetrahydrofuran, the reactor was immersed in an ice bath, and stirring was initiated. Subsequently, a mixed solution consisting of 21.00 g of 4′-hydroxy-3′-methoxyacetophenone, 9.32 g of isopropanol, and 180 mL of tetrahydrofuran were added dropwise thereto over 3 hours. Subsequently, stirring was continued over 8 hours while the reactor was immersed in the ice bath. Subsequently, 59.47 g of methanol was added to quench the reaction. Subsequently, the pressure in the reactor was reduced to 50 hPa, immersed in a water bath at 20° C. to concentrate the reaction solution. Subsequently, the reactor was immersed in an ice bath, and 120 mL of cold methanol was added to dilute the reaction solution. Subsequently, the pressure in the reactor was reduced to 50 hPa, immersed in a water bath at 20° C. to concentrate the reaction solution. Subsequently, the reactor was immersed in an ice bath, and 600 mL of cold methanol was added to dilute the reaction solution. Subsequently, the reaction solution was gradually added to 1,200 g of dilute sulfuric acid with a mass percent concentration of 1 with vigorous stirring and mixed. Subsequently, the precipitate was filtered off with a suction filter, compressed, and washed with 300 mL of an aqueous methanol solution with a volume percent concentration of 33.3. Subsequently, the precipitate was dried in vacuo at 40° C. to obtain 20.3 g of 1-(4-hydroxy-3-methoxyphenyl)ethanol. The yield was 95.2%.

As a result of the analysis by liquid chromatography-mass spectrometry (LC-MS), the molecular weight was found to be 168, and the substance was confirmed to be 1-(4-hydroxy-3-methoxyphenyl)ethanol.

The following peaks were found by carrying out ¹H-NMR measurement under the above measurement conditions, and the compound was confirmed to have the chemical structure of 1-(4-hydroxy-3-methoxyphenyl)ethanol.

δ (ppm) (d6-DMSO): 9.4 (1H, —OH), 7.7 (3H, Ph), 5.2 (1H, —CH—OH), 4.6 (1H, —CH—OH), 3.8 (3H, —CH3), 1.3 (3H, —CH3)

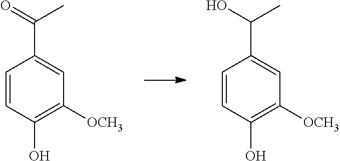

### (Step 3-1) Synthesis of 1-(4-hydroxy-3-iodo-5-methoxyphenyl)ethanol

A reactor was charged with 1.2000 g of 1-(4-hydroxy-3-methoxyphenyl)ethanol, 1.7630 g of iodine, and 17.37 mL of methanol, the reactor was immersed in an ice bath, and stirring was initiated. Subsequently, 0.8736 g of an aqueous iodic acid solution with a mass percent concentration of 70 was added dropwise thereto for 30 minutes. Subsequently, the reactor was immersed in a water bath at 25° C., and stirring was continued over 3.5 hours. Subsequently, 0.174 mL of an aqueous sodium hydrogen sulfite solution with a mass percent concentration of 35 was added to quench the reaction. Subsequently, the reaction solution was gradually added to 34.74 mL of pure water with vigorous stirring and mixed. Subsequently, the precipitate was filtered off with a suction filter, compressed, and washed with an aqueous methanol solution with a volume percent concentration of 33.3. Subsequently, the precipitate was dried in vacuo at 40° C. to obtain 3.0969 g of a mixture of 1-(4-hydroxy-3-iodo-5-methoxyphenyl)ethanol and 2-iodo-6-methoxy-4-(1-methoxyethyl)phenol. As a result of the HPLC analysis using a UV detector at a measurement wavelength of 254 nm, the ratio between 1-(4-hydroxy-3-iodo-5-methoxyphenyl)ethanol and 2-iodo-6-methoxy-4-(1-methoxyethyl)phenol was 50.88:47.15.

As a result of the analysis by liquid chromatography-mass spectrometry (LC-MS), the molecular weights were found to be 294 and 308, and the substance was confirmed to be a mixture of 1-(4-hydroxy-3-iodo-5-methoxyphenyl)ethanol and 2-iodo-6-methoxy-4-(1-methoxyethyl)phenol.

The following peaks were found by carrying out ¹H-NMR measurement under the above measurement conditions, and the compound was confirmed to have the chemical structures of 1-(4-hydroxy-3-iodo-5-methoxyphenyl)ethanol and 2-iodo-6-methoxy-4-(1-methoxyethyl)phenol.

δ (ppm) (d6-DMSO): 9.4 (1H, —OH), 7.7 (2H, Ph), 5.2 (0.5H, —CH—OH), 4.6-4.3 (1H, —CH—OH), 3.8 (3H, —CH3), 3.0 (1.5H, —O—CH3), 1.3 (3H, —CH3)

### (Step 3-2) Synthesis of 1-(4-hydroxy-3-iodo-5-methoxyphenyl)ethanol

A reactor was charged with 1.1881 g of 1-(4-hydroxy-3-methoxyphenyl)ethanol, 1.7472 g of iodine, 15.48 mL of methanol, and 1.72 mL of pure water, the reactor was immersed in an ice bath, and stirring was initiated. Subsequently, 0.8687 g of an aqueous iodic acid solution with a mass percent concentration of 70 was added dropwise thereto for 30 minutes. Subsequently, the reactor was immersed in a water bath at 25° C., and stirring was continued over 3.5 hours. Subsequently, 0.172 mL of an aqueous sodium hydrogen sulfite solution with a mass percent concentration of 35 was added to quench the reaction. Subsequently, the reaction solution was gradually added to 34.40 mL of pure water with vigorous stirring and mixed. Subsequently, the precipitate was filtered off with a suction filter, compressed, and washed with an aqueous methanol solution with a volume percent concentration of 33.3. Subsequently, the precipitate was dried in vacuo at 40° C. to obtain 3.1023 g of a mixture of 1-(4-hydroxy-3-iodo-5-methoxyphenyl)ethanol and 2-iodo-6-methoxy-4-(1-methoxyethyl)phenol. As a result of the HPLC analysis using a UV detector at a measurement wavelength of 254 nm, the ratio between 1-(4-hydroxy-3-iodo-5-methoxyphenyl)ethanol and 2-iodo-6-methoxy-4-(1-methoxyethyl)phenol was 83.16:16.03.

As a result of the analysis by liquid chromatography-mass spectrometry (LC-MS), the molecular weights were found to be 294 and 308, and the substance was confirmed to be a mixture of 1-(4-hydroxy-3-iodo-5-methoxyphenyl)ethanol and 2-iodo-6-methoxy-4-(1-methoxyethyl)phenol.

### (Step 3-3) Synthesis of 1-(4-hydroxy-3-iodo-5-methoxyphenyl)ethanol

A reactor was charged with 1.2086 g of 1-(4-hydroxy-3-methoxyphenyl)ethanol, 1.7787 g of iodine, 14.00 mL of methanol, and 3.50 mL of pure water, the reactor was immersed in an ice bath, and stirring was initiated. Subsequently, 0.8795 g of an aqueous iodic acid solution with a mass percent concentration of 70 was added dropwise thereto for 30 minutes. Subsequently, the reactor was immersed in a water bath at 25° C., and stirring was continued over 3.5 hours. Subsequently, 0.175 mL of an aqueous sodium hydrogen sulfite solution with a mass percent concentration of 35 was added to quench the reaction. Subsequently, the reaction solution was gradually added to 35.00 mL of pure water with vigorous stirring and mixed. Subsequently, the precipitate was filtered off with a suction filter, compressed, and washed with an aqueous methanol solution with a volume percent concentration of 33.3. Subsequently, the precipitate was dried in vacuo at 40° C. to obtain 3.1655 g of a mixture of 1-(4-hydroxy-3-iodo-5-methoxyphenyl)ethanol and 2-iodo-6-methoxy-4-(1-methoxyethyl)phenol. As a result of the HPLC analysis using a UV detector at a measurement wavelength of 254 nm, the ratio between 1-(4-hydroxy-3-iodo-5-methoxyphenyl)ethanol and 2-iodo-6-methoxy-4-(1-methoxyethyl)phenol was 73.88:25.39.

As a result of the analysis by liquid chromatography-mass spectrometry (LC-MS), the molecular weights were found to be 294 and 308, and the substance was confirmed to be a mixture of 1-(4-hydroxy-3-iodo-5-methoxyphenyl)ethanol and 2-iodo-6-methoxy-4-(1-methoxyethyl)phenol.

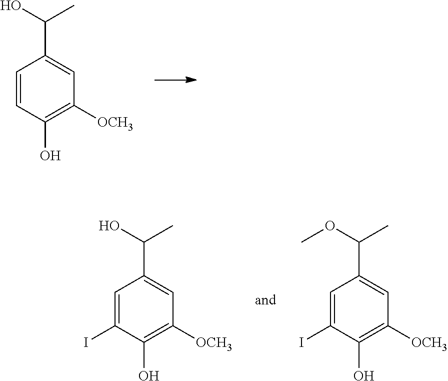

### (Step 4) Synthesis of 1-(4-hydroxy-3-iodo-5-methoxyphenyl)ethanol

The reactor was charged with 8.77 g of sodium borohydride and 180 mL of tetrahydrofuran, the reactor was immersed in an ice bath, and stirring was initiated. Subsequently, a mixed solution consisting of 60.00 g of 4′-hydroxy-3′-iodo-5′-methoxyacetophenone, 9.31 g of isopropanol, and 180 mL of tetrahydrofuran were added dropwise thereto over 3 hours. Subsequently, stirring was continued over 9 hours while the reactor was immersed in the ice bath. Subsequently, 59.47 g of methanol was added to quench the reaction. Subsequently, the pressure in the reactor was reduced to 50 hPa, immersed in a water bath at 20° C. to concentrate the reaction solution. Subsequently, the reactor was immersed in an ice bath, and 120 mL of cold methanol was added to dilute the reaction solution. Subsequently, the pressure in the reactor was reduced to 50 hPa, immersed in a water bath at 20° C. to concentrate the reaction solution. Subsequently, the reactor was immersed in an ice bath, and 600 mL of cold methanol was added to dilute the reaction solution. Subsequently, the reaction solution was gradually added to 1,200 g of dilute sulfuric acid with a mass percent concentration of 1 with vigorous stirring and mixed. Subsequently, the precipitate was filtered off with a suction filter, compressed, and washed with 300 mL of an aqueous methanol solution with a volume percent concentration of 33.3. Subsequently, the precipitate was dried in vacuo at 40° C. to obtain 58.64 g of 1-(4-hydroxy-3-iodo-5-methoxyphenyl)ethanol. The yield was 97.2%. As a result of the analysis by liquid chromatography-mass spectrometry (LC-MS), the molecular weight was found to be 294, and the substance was confirmed to be 1-(4-hydroxy-3-iodo-5-methoxyphenyl)ethanol.

The following peaks were found by carrying out ¹H-NMR measurement under the above measurement conditions, and the compound was confirmed to have the chemical structure of 1-(4-hydroxy-3-iodo-5-methoxyphenyl)ethanol.

δ (ppm) (d6-DMSO): 9.4 (1H, —OH), 7.7 (2H, Ph), 5.2 (1H, —CH—OH), 4.6 (1H, —CH—OH), 3.8 (3H, —CH3), 1.3 (3H, —CH3)

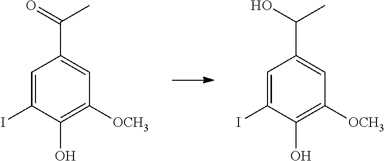

### (Step 5-1) Synthesis of 4-hydroxy-3-iodo-5-methoxystyrene

The reactor was charged with 120.00 g of 1-(4-hydroxy-3-methoxyphenyl)ethanol, 7.94 g of concentrated sulfuric acid, 0.30 g of 4-hydroxy-2,2,6,6-tetramethylpiperidinyl-oxyl free radical, and 1,500 mL of dimethylsulfoxide, and stirring was initiated. Subsequently, the pressure in the reactor was reduced to 30 hPa, and blowing of air into the reaction solution at a flow rate of 9 mL/minute was initiated. Subsequently, the reactor was immersed in a water bath at 90° C., and stirring was continued over 5 hours. Subsequently, the reactor was immersed in a water bath at 25° C. to cool the reaction solution. Subsequently, the reaction solution was gradually added to 3,000 g of an aqueous sodium hydrogen sulfite solution with a mass percent concentration of 0.1 with vigorous stirring and mixed. Subsequently, the precipitate was filtered off with a suction filter, compressed, and washed with 1,500 mL of an aqueous methanol solution with a volume percent concentration of 33.3. Subsequently, the precipitate was dried in vacuo at 40° C. to obtain 109.69 g of 4-hydroxy-3-iodo-5-methoxystyrene. The yield was 95.8%.

As a result of the analysis by liquid chromatography-mass spectrometry (LC-MS), the molecular weight was found to be 372, and the substance was confirmed to be 4-hydroxy-3-iodo-5-methoxystyrene.

The following peaks were found by carrying out ¹H-NMR measurement under the above measurement conditions, and the compound was confirmed to have the chemical structure of 4-hydroxy-3-iodo-5-methoxystyrene.

δ (ppm) (d6-DMSO): 9.5 (1H, —OH), 7.7 (2H, Ph), 6.7 (1H, —CH═), 5.7 (1H, ═CH2), 5.3 (1H, ═CH2), 3.8 (3H, —CH3)

### (Step 5-2) Synthesis of 4-hydroxy-3-iodo-5-methoxystyrene

The reactor was charged with 2.0045 g of a mixture in which the ratio between 1-(4-hydroxy-3-iodo-5-methoxyphenyl)ethanol and 2-iodo-6-methoxy-4-(1-methoxyethyl)phenol is 74.40:24.18, 0.2895 mL of concentrated sulfuric acid, 0.0020 g of 4-hydroxy-2,2,6,6-tetramethylpiperidinyl-oxyl free radical, and 20 mL of dimethylsulfoxide, and stirring was initiated. Subsequently, the pressure in the reactor was reduced to 30 hPa, the reactor was immersed in a water bath at 90° C., and stirring was continued over 3 hours. Subsequently, the reactor was immersed in a water bath at 25° C. to cool the reaction solution. As a result of the HPLC analysis using a UV detector at a measurement wavelength of 254 nm, the ratio among 1-(4-hydroxy-3-iodo-5-methoxyphenyl)ethanol, 2-iodo-6-methoxy-4-(1-methoxyethyl)phenol, and 4-hydroxy-3-iodo-5-methoxystyrene in the reaction solution was 0.08:0.01:98.12.

As a result of the analysis by liquid chromatography-mass spectrometry (LC-MS), the molecular weight was found to be 372, and the substance was confirmed to be 4-hydroxy-3-iodo-5-methoxystyrene.

The compound was confirmed to have a similar chemical structure by carrying out ¹H-NMR measurement under the above measurement conditions.

### (Step 5-3) Synthesis of 4-hydroxy-3-iodo-5-methoxystyrene

The reactor was charged with 2.0045 g of a mixture in which the ratio between 1-(4-hydroxy-3-iodo-5-methoxyphenyl)ethanol and 2-iodo-6-methoxy-4-(1-methoxyethyl)phenol is 74.40:24.18, 0.3 g of paratoluenesulfonic acid, 0.0020 g of tert-butylcatechol, and 20 mL of dimethylsulfoxide, and stirring was initiated. Subsequently, the pressure in the reactor was reduced to 30 hPa, the reactor was immersed in a water bath at 90° C., and stirring was continued over 3 hours. Subsequently, the reactor was immersed in a water bath at 25° C. to cool the reaction solution. As a result of the HPLC analysis using a UV detector at a measurement wavelength of 254 nm, the ratio among 1-(4-hydroxy-3-iodo-5-methoxyphenyl)ethanol, 2-iodo-6-methoxy-4-(1-methoxyethyl)phenol, and 4-hydroxy-3-iodo-5-methoxystyrene in the reaction solution was 0.06:0.01:98.82.

As a result of the analysis by liquid chromatography-mass spectrometry (LC-MS), the molecular weight was found to be 372, and the substance was confirmed to be 4-hydroxy-3-iodo-5-methoxystyrene.

The compound was confirmed to have a similar chemical structure by carrying out ¹H-NMR measurement under the above measurement conditions.

### (Step 5-4) Synthesis of 4-hydroxy-3-iodo-5-methoxystyrene

The reactor was charged with 2.0045 g of a mixture in which the ratio between 1-(4-hydroxy-3-iodo-5-methoxyphenyl)ethanol and 2-iodo-6-methoxy-4-(1-methoxyethyl)phenol is 74.40:24.18, 0.3 g of methanesulfonic acid, 0.0020 g of 4-methoxyquinone, and 20 mL of dimethylsulfoxide, and stirring was initiated. Subsequently, the pressure in the reactor was reduced to 30 hPa, the reactor was immersed in a water bath at 90° C., and stirring was continued over 3 hours. Subsequently, the reactor was immersed in a water bath at 25° C. to cool the reaction solution. As a result of the HPLC analysis using a UV detector at a measurement wavelength of 254 nm, the ratio among 1-(4-hydroxy-3-iodo-5-methoxyphenyl)ethanol, 2-iodo-6-methoxy-4-(1-methoxyethyl)phenol, and 4-hydroxy-3-iodo-5-methoxystyrene in the reaction solution was 0.05:0.02:98.67.

As a result of the analysis by liquid chromatography-mass spectrometry (LC-MS), the molecular weight was found to be 372, and the substance was confirmed to be 4-hydroxy-3-iodo-5-methoxystyrene.

The compound was confirmed to have a similar chemical structure by carrying out ¹H-NMR measurement under the above measurement conditions.

### (Step 5-5) Synthesis of 4-hydroxy-3-iodo-5-methoxystyrene

The reactor was charged with 2.0045 g of a mixture in which the ratio between 1-(4-hydroxy-3-iodo-5-methoxyphenyl)ethanol and 2-iodo-6-methoxy-4-(1-methoxyethyl)phenol is 74.40:24.18, 0.2895 mL of concentrated sulfuric acid, 0.0020 g of Q1300 (manufactured by FUJIFILM Wako Pure Chemical Corporation), and 20 mL of dimethylsulfoxide, and stirring was initiated. Subsequently, the pressure in the reactor was reduced to 30 hPa, the reactor was immersed in a water bath at 90° C., and stirring was continued over 3 hours. Subsequently, the reactor was immersed in a water bath at 25° C. to cool the reaction solution. As a result of the HPLC analysis using a UV detector at a measurement wavelength of 254 nm, the ratio among 1-(4-hydroxy-3-iodo-5-methoxyphenyl)ethanol, 2-iodo-6-methoxy-4-(1-methoxyethyl)phenol, and 4-hydroxy-3-iodo-5-methoxystyrene in the reaction solution was 0.10:0.01:98.43.

As a result of the analysis by liquid chromatography-mass spectrometry (LC-MS), the molecular weight was found to be 372, and the substance was confirmed to be 4-hydroxy-3-iodo-5-methoxystyrene.

The compound was confirmed to have a similar chemical structure by carrying out ¹H-NMR measurement under the above measurement conditions.

### (Step 5-6) Synthesis of 4-hydroxy-3-iodo-5-methoxystyrene

The reactor equipped with a reflux tube and Dienstark was charged with 2.0045 g of a mixture in which the ratio between 1-(4-hydroxy-3-iodo-5-methoxyphenyl)ethanol and 2-iodo-6-methoxy-4-(1-methoxyethyl)phenol is 74.40:24.18, 0.2895 mL of concentrated sulfuric acid, 0.0020 g of 4-hydroxy-2,2,6,6-tetramethylpiperidinyl-oxyl free radical, 20 mL of dimethylsulfoxide, and 20 mL of toluene, and stirring was initiated. Subsequently, the pressure in the reactor was reduced to 30 hPa, the reactor was immersed in a water bath at 90° C., and stirring was continued over 3 hours, while removing the solvent component/water distilled off. Subsequently, the reactor was immersed in a water bath at 25° C. to cool the reaction solution. As a result of the HPLC analysis using a UV detector at a measurement wavelength of 254 nm, the ratio among 1-(4-hydroxy-3-iodo-5-methoxyphenyl)ethanol, 2-iodo-6-methoxy-4-(1-methoxyethyl)phenol, and 4-hydroxy-3-iodo-5-methoxystyrene in the reaction solution was 0.03:0.01:99.11.

As a result of the analysis by liquid chromatography-mass spectrometry (LC-MS), the molecular weight was found to be 372, and the substance was confirmed to be 4-hydroxy-3-iodo-5-methoxystyrene.

The compound was confirmed to have a similar chemical structure by carrying out ¹H-NMR measurement under the above measurement conditions.

### (Step 5-7) Synthesis of 4-hydroxy-3-iodo-5-methoxystyrene

The reactor was charged with 2.0045 g of a mixture in which the ratio between 1-(4-hydroxy-3-iodo-5-methoxyphenyl)ethanol and 2-iodo-6-methoxy-4-(1-methoxyethyl)phenol is 74.40:24.18, 0.2895 mL of concentrated sulfuric acid, 0.0020 g of 4-hydroxy-2,2,6,6-tetramethylpiperidinyl-oxyl free radical, and 20 mL of N-methylpyrrolidone, and stirring was initiated. Subsequently, the pressure in the reactor was reduced to 30 hPa, the reactor was immersed in a water bath at 90° C., and stirring was continued over 3 hours. Subsequently, the reactor was immersed in a water bath at 25° C. to cool the reaction solution. As a result of the HPLC analysis using a UV detector at a measurement wavelength of 254 nm, the ratio among 1-(4-hydroxy-3-iodo-5-methoxyphenyl)ethanol, 2-iodo-6-methoxy-4-(1-methoxyethyl)phenol, and 4-hydroxy-3-iodo-5-methoxystyrene in the reaction solution was 0.12:0.01:98.51.

As a result of the analysis by liquid chromatography-mass spectrometry (LC-MS), the molecular weight was found to be 372, and the substance was confirmed to be 4-hydroxy-3-iodo-5-methoxystyrene.

The compound was confirmed to have a similar chemical structure by carrying out ¹H-NMR measurement under the above measurement conditions.

### (Step 5-8) Synthesis of 4-hydroxy-3-iodo-5-methoxystyrene

The reactor was charged with 2.0045 g of a mixture in which the ratio between 1-(4-hydroxy-3-iodo-5-methoxyphenyl)ethanol and 2-iodo-6-methoxy-4-(1-methoxyethyl)phenol is 74.40:24.18, 0.2895 mL of concentrated sulfuric acid, 0.0020 g of 4-hydroxy-2,2,6,6-tetramethylpiperidinyl-oxyl free radical, and 20 mL of dimethylformamide, and stirring was initiated. Subsequently, the pressure in the reactor was reduced to 30 hPa, the reactor was immersed in a water bath at 90° C., and stirring was continued over 3 hours. Subsequently, the reactor was immersed in a water bath at 25° C. to cool the reaction solution. As a result of the HPLC analysis using a UV detector at a measurement wavelength of 254 nm, the ratio among 1-(4-hydroxy-3-iodo-5-methoxyphenyl)ethanol, 2-iodo-6-methoxy-4-(1-methoxyethyl)phenol, and 4-hydroxy-3-iodo-5-methoxystyrene in the reaction solution was 0.11:0.01:99.01.

As a result of the analysis by liquid chromatography-mass spectrometry (LC-MS), the molecular weight was found to be 372, and the substance was confirmed to be 4-hydroxy-3-iodo-5-methoxystyrene.

The compound was confirmed to have a similar chemical structure by carrying out ¹H-NMR measurement under the above measurement conditions.

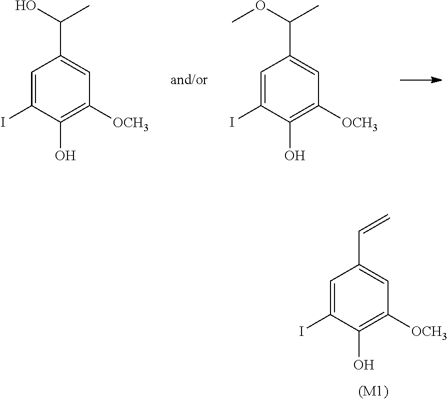

### Example Group 1: Synthetic Example 1: Example A1 a

### Synthesis of 4-acetoxy-3-iodo-5-methoxystyrene

Using a 100 mL glass flask as a reaction vessel, 16.7 g (45 mmol) of 4-hydroxy-3-iodo-5-methoxystyrene was dissolved by using dimethylsulfoxide as a solvent, 2 eq. of acetic anhydride and 1 eq. of sulfuric acid were added thereto, and then the mixture was warmed to 80° C. and stirred for 3 hours. Thereafter, the stirred solution was cooled, and the precipitate was filtered off, washed, and dried to obtain 9.0 g of a white solid. The white solid sample was analyzed by liquid chromatography-mass spectrometry (LC-MS) and, as a result, the molecular weight was found to be 414 and the white solid was confirmed to be 4-acetoxy-3-iodo-5-methoxystyrene.

The following peaks were found by carrying out ¹H-NMR measurement under the above measurement conditions, and the compound was confirmed to have the chemical structure of 4-acetoxy-3-iodo-5-methoxystyrene of the compound Ala represented by the formula (M1a). δ (ppm) (d6-DMSO): 7.9 (2H, Ph), 6.6 (1H, —CH2-), 5.7 (1H, ═CH2), 5.1 (1H, ═CH2), 3.8 (3H, —CH3), 2.3 (3H, —CH3)

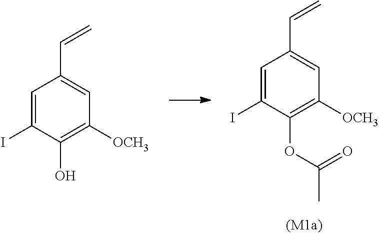

### Example Group 1: Example A2

The reaction was carried out in the same manner as Example A1, except that 4-hydroxy-3-iodo-5-methoxybenzenecarbaldehyde of Example A1 was changed to 3-ethoxy-4-hydroxy-5-iodobenzenecarbaldehyde, thereby isolating 132 g of 3-ethoxy-4-hydroxy-5-iodostyrene represented by the formula (M2) as the target substance.

As a result of the analysis by liquid chromatography-mass spectrometry (LC-MS), the molecular weight was found to be 290.

The following peaks were found by carrying out ¹H-NMR measurement under the above measurement conditions, and the compound was confirmed to have the chemical structure of the compound A2 represented by the formula (M2).

δ (ppm) (d6-DMSO): 9.5 (1H, —OH), 7.7 (2H, Ph), 6.7 (1H, —CH═), 5.7 (1H, ═CH2), 5.3 (1H, ═CH2), 4.1 (2H, —CH2-), 1.4 (3H, —CH3)

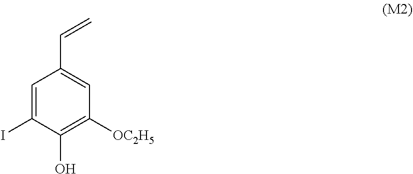

### Example Group 1: Synthetic Example 2: Example A2a

The reaction was carried out in the same manner as Synthetic Example 1: Example Ala except that 4-hydroxy-3-iodo-5-methoxystyrene of Synthetic Example 1: Example Ala was changed to 3-ethoxy-4-hydroxy-5-iodostyrene, thereby isolating 9.1 g of a white solid. The white solid sample was analyzed by liquid chromatography-mass spectrometry (LC-MS) and, as a result, the molecular weight was found to be 332 and the white solid was confirmed to be 4-acetoxy-3-ethoxy-5-iodostyrene.

The following peaks were found by carrying out ¹H-NMR measurement under the above measurement conditions, and the compound was confirmed to have the chemical structure of 4-acetoxy-3-ethoxy-5-iodostyrene of the compound A2a represented by the formula (M2a).

δ (ppm) (d6-DMSO): 7.7 (2H, Ph), 6.7 (1H, —CH═), 5.7 (1H, ═CH2), 5.3 (1H, ═CH2), 4.1 (2H, —CH2-), 2.3 (3H, —CH3) 1.4 (3H, —CH3)

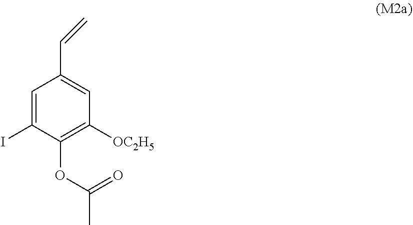 [SMILES: C=Cc1cc(I)c(OC(C)=O)c(OCC)c1]

### Example Group 1: Example A3

Into 400 mL of dichloromethane in a 2 L flask, 41 g of the obtained compound A1, 16.2 g of triethylamine, and 0.7 g of N-(4-pyridyl)dimethylamine (DMAP) were dissolved under a nitrogen flow. 33.6 g of di-tert-butyl dicarbonate was dissolved in 100 mL of dichloromethane, which was added dropwise into the aforementioned 2 L flask with stirring, and the mixture was stirred at room temperature for 3 hours. Thereafter, water washing was conducted three times by a separation operation using 100 mL of water, the solvent was distilled off from the obtained organic phase, original components were removed by silica gel chromatography using dichloromethane/hexane, and the solvent was further distilled off to obtain 4.5 g of a BOC group-substituted compound (a compound represented by the following formula (M3), hereinafter, also referred to as the “compound A3”) of the compound A1, as the target component.

As a result of the analysis by liquid chromatography-mass spectrometry (LC-MS), the molecular weight was found to be 376.

The following peaks were found by carrying out ¹H-NMR measurement under the above measurement conditions, and the compound was confirmed to have the chemical structure of the compound represented by the formula (M3).

δ (ppm) (d6-DMSO): 7.7 (2H, Ph), 6.7 (1H, —CH═), 5.7 (1H, ═CH2), 5.3 (1H, ═CH2), 3.8 (3H, —CH3), 1.4 (9H, —C—(CH3)3)

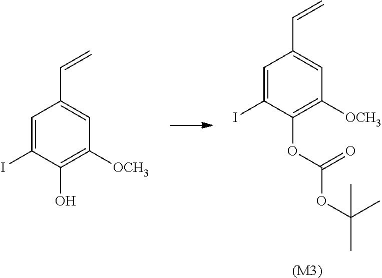

### Example Group 1: Example A4

In a container (internal capacity: 200 mL) equipped with a stirrer, a condenser tube, and a burette, 100 mL of acetone was charged with 4.61 g (12.4 mmol) of the compound A1 obtained in Example A1 and 2.42 g (12.4 mmol) of ethylvinyl ether, 2.5 g of pyridinium p-toluenesulfonate was added, and the contents were reacted by being stirred at room temperature for 24 hours to obtain a reaction solution. Next, the reaction solution was concentrated, and the reaction product was filtered to separate solid matter.

The obtained solid matter was filtered and then dried, and then the solid matter was separated and purified by column chromatography to obtain 3.2 g of the compound A4 (a compound represented by the following formula (M4)).

As a result of the analysis by liquid chromatography-mass spectrometry (LC-MS), the molecular weight was found to be 348. The following peaks were found by carrying out ¹H-NMR measurement under the above measurement conditions, and the compound was confirmed to have the chemical structure of the compound represented by the formula (M4).

δ (ppm) (d6-DMSO): 7.7 (2H, Ph), 6.7 (1H, —CH═), 5.7 (1H, ═CH2), 5.6 (1H, CH3CH—), 5.3 (1H, ═CH2), 3.8 (3H, —CH3), 3.9 (2H, CH3CH2-), 1.6 (3H, CH3CH—), 1.2 (3H, CH3CH2-)

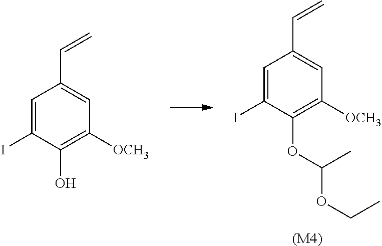

### Example Group 1: Example A5

In a container (internal capacity: 200 mL) equipped with a stirrer, a condenser tube, and a burette, 4.61 g (12.4 mmol) of the compound A1 obtained in Example A1 and 2.42 g (12.4 mmol) of tetrahydropyran were charged to 100 mL of acetone, 2.5 g of pyridinium p-toluenesulfonate was added, and the contents were reacted by being stirred at room temperature for 24 hours to obtain a reaction solution. Next, the reaction solution was concentrated, and the reaction product was filtered to separate solid matter.

The obtained solid matter was filtered and then dried, and then the solid matter was separated and purified by column chromatography to obtain 3.2 g of the compound A4 (a compound represented by the following formula (M4)).

The following peaks were found by carrying out ¹H-NMR measurement under the above measurement conditions, and the compound was confirmed to have the chemical structure of the compound represented by the formula (M5).

As a result of the analysis by liquid chromatography-mass spectrometry (LC-MS), the molecular weight was found to be 360.

δ (ppm) (d6-DMSO): 7.7 (2H, Ph), 6.7 (1H, —CH═), 5.8 (1H, proton of tetrahydropyranyl group ═CH—), 5.7 (1H, ═CH2), 5.3 (1H, ═CH2), 3.8 (3H, —CH3), 1.6-3.7 (8H, protons of tetrahydropyranyl group —CH2-)

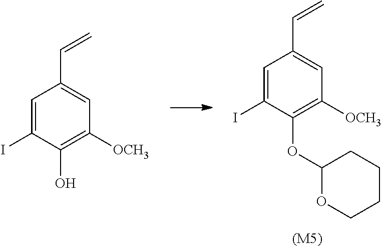

### (Example Group 1: Example A6) Synthesis of Compound A6 Represented by Formula (M6)

In a container (internal capacity: 200 mL) equipped with a stirrer, a condenser tube, and a burette, 4.61 g (12.4 mmol) of the compound A1 obtained in the above Example A1 and 2.42 g (12.4 mmol) of tert-butyl bromoacetate were charged to 100 mL of acetone, 1.71 g (12.4 mmol) of potassium carbonate and 0.4 g of 18-crown-6 (IUPAC name: 1,4,7,10,13,16-hexaoxacyclooctadecane) was added, and the contents were reacted by being stirred under reflux for 3 hours to obtain a reaction liquid. Next, the reaction solution was concentrated, and the reaction product was precipitated by the addition of 100 g of pure water to the concentrate, cooled to room temperature, and then filtered to separate solid matter.

The obtained solid matter was filtered and then dried, and then the solid matter was separated and purified by column chromatography to obtain 3.2 g of the compound A6 (a compound represented by the following formula (M6)).

As a result of the analysis by liquid chromatography-mass spectrometry (LC-MS), the molecular weight was found to be 390.

The following peaks were found by carrying out ¹H-NMR measurement under the above measurement conditions, and the compound was confirmed to have the chemical structure of the compound represented by the formula (M6).

δ (ppm) (d6-DMSO): 7.7 (2H, Ph), 6.7 (1H, —CH═), 5.7 (1H, ═CH2), 5.3 (1H, ═CH2), 5.0 (2H, —CH2-), 3.8 (3H, —CH3), 1.4 (9H, —C—(CH3)3)

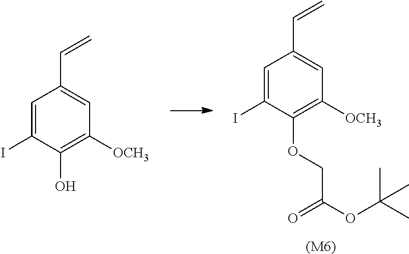

### (Example Group 1: Example A7) Synthesis of Compound A7 Represented by Formula (M7)

In a container (internal capacity: 200 mL) equipped with a stirrer, a condenser tube, and a burette, 4.61 g (12.4 mmol) of the compound A1 obtained in the above Example A1 and 2.42 g (12.4 mmol) of bromoacetic acid 2-methyl-2-adamantyl ester were charged to 100 mL of acetone, 1.71 g (12.4 mmol) of potassium carbonate and 0.4 g of 18-crown-6 (IUPAC name: 1,4,7,10,13,16-hexaoxacyclooctadecane) was added, and the contents were reacted by being stirred under reflux for 3 hours to obtain a reaction liquid. Next, the reaction solution was concentrated, and the reaction product was precipitated by the addition of 100 g of pure water to the concentrate, cooled to room temperature, and then filtered to separate solid matter.

The obtained solid matter was filtered and then dried, and then the solid matter was separated and purified by column chromatography to obtain 3.2 g of the compound A7 (a compound represented by the following formula (M7)).

The following peaks were found by carrying out ¹H-NMR measurement under the above measurement conditions, and the compound was confirmed to have the chemical structure of the compound represented by the formula (M7).

As a result of the analysis by liquid chromatography-mass spectrometry (LC-MS), the molecular weight was found to be 482.

δ (ppm) (d6-DMSO): 7.7 (2H, Ph), 6.7 (1H, —CH═), 5.7 (1H, ═CH2), 5.3 (1H, ═CH2), 5.0 (2H, —CH2-), 3.8 (3H, —CH3), 0.8-2.4 (17H, protons of 2-methyl-2-adamantyl group)

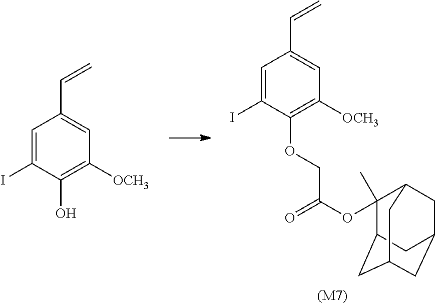

### (Example Group 1: Example A8) Synthesis of Compound A8 Represented by Formula (M8)

In a container (internal capacity: 200 mL) equipped with a stirrer, a condenser tube, and a burette, 4.61 g (12.4 mmol) of the compound A1 obtained in the above Example A1 and 1.70 g (12.4 mmol) of t-butyl bromide were charged to 100 mL of acetone, 1.71 g (12.4 mmol) of potassium carbonate and 0.4 g of 18-crown-6 (IUPAC name: 1,4,7,10,13,16-hexaoxacyclooctadecane) was added, and the contents were reacted by being stirred under reflux for 3 hours to obtain a reaction liquid. Next, the reaction solution was concentrated, and the reaction product was precipitated by the addition of 100 g of pure water to the concentrate, cooled to room temperature, and then filtered to separate solid matter.

The obtained solid matter was filtered and then dried, and then the solid matter was separated and purified by column chromatography to obtain 0.5 g of the compound A8 (a compound represented by the following formula (M8)).

The following peaks were found by carrying out ¹H-NMR measurement under the above measurement conditions, and the compound was confirmed to have the chemical structure of the compound represented by the formula (M8).

As a result of the analysis by liquid chromatography-mass spectrometry (LC-MS), the molecular weight was found to be 332.

δ (ppm) (d6-DMSO): 7.7 (2H, Ph), 6.7 (1H, —CH═), 5.7 (1H, ═CH2), 5.3 (1H, ═CH2), 3.8 (3H, —CH3), 1.4 (9H, —C—(CH3)3)

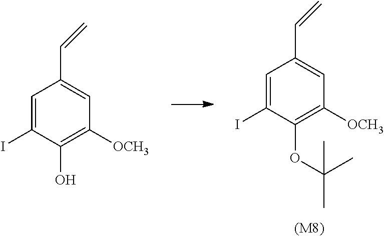

### Example Group 1: Example A9

The reaction was carried out in the same manner as Example A3, except that 4-hydroxy-3-iodo-5-methoxystyrene of Example A3 was changed to 3-ethoxy-4-hydroxy-5-iodostyrene, thereby obtaining 4.6 g of a BOC group-substituted compound of the compound A2 represented by the formula (M9) as the target substance (a compound represented by the following formula (M9), hereinafter, also referred to as the “compound A9”).

As a result of the analysis by liquid chromatography-mass spectrometry (LC-MS), the molecular weight was found to be 390.

The following peaks were found by carrying out ¹H-NMR measurement under the above measurement conditions, and the compound was confirmed to have the chemical structure of the compound represented by the formula (M9).

δ (ppm) (d6-DMSO): 7.7 (2H, Ph), 6.7 (1H, —CH═), 5.7 (1H, ═CH2), 5.3 (1H, ═CH2), 4.1 (2H, —CH2-), 1.4 (3H, —CH3), 1.3 (H, —C—(CH3)3)

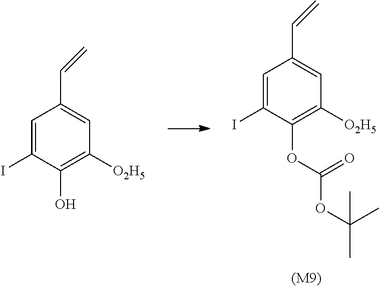

### Example Group 1: Example A10

The reaction was carried out in the same manner as Example A4, except that 4-hydroxy-3-iodo-5-methoxystyrene of Example A4 was changed to 3-ethoxy-4-hydroxy-5-iodostyrene, thereby obtaining 3.5 g of a compound represented by the formula (M10) as the target substance (hereinafter, also referred to as the “compound A10”).

As a result of the analysis by liquid chromatography-mass spectrometry (LC-MS), the molecular weight was found to be 362. The following peaks were found by carrying out ¹H-NMR measurement under the above measurement conditions, and the compound was confirmed to have the chemical structure of the compound represented by the formula (M10).

δ (ppm) (d6-DMSO): 7.7 (2H, Ph), 6.7 (1H, —CH═), 5.7 (1H, ═CH2), 5.6 (1H, CH3CH—), 5.3 (1H, ═CH2), 4.1 (2H, —CH2-), 3.9 (2H, CH3CH2-), 1.6 (3H, CH3CH—), 1.4 (3H, —CH3), 1.2 (3H, CH3CH2-)

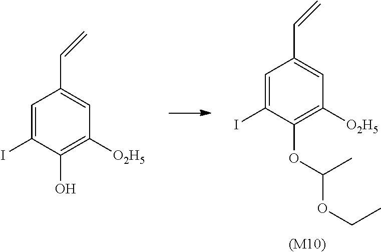

### Example Group 1: Example A11

The reaction was carried out in the same manner as Example A5, except that 4-hydroxy-3-iodo-5-methoxystyrene of Example A5 was changed to 3-ethoxy-4-hydroxy-5-iodostyrene, thereby obtaining 3.6 g of a compound represented by the formula (M11) as the target substance, hereinafter, also referred to as the “compound A11”).

As a result of the analysis by liquid chromatography-mass spectrometry (LC-MS), the molecular weight was found to be 374.

The following peaks were found by carrying out ¹H-NMR measurement under the above measurement conditions, and the compound was confirmed to have the chemical structure of the compound represented by the formula (M10).

δ (ppm) (d6-DMSO): 7.7 (2H, Ph), 6.7 (1H, —CH═), 5.8 (1H, proton of tetrahydropyranyl group ═CH—), 5.7 (1H, ═CH2), 5.3 (1H, ═CH2), 4.1 (2H, —CH2-), 1.6-3.7 (8H, protons of tetrahydropyranyl group —CH2-), 1.4 (3H, —CH3)

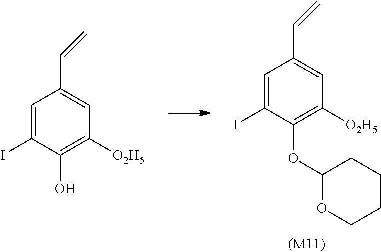

### Example Group 1: Example A12

The reaction was carried out in the same manner as Example A6, except that 4-hydroxy-3-iodo-5-methoxystyrene of Example A6 was changed to 3-ethoxy-4-hydroxy-5-iodostyrene, thereby obtaining 3.8 g of a compound represented by the formula (M12) as the target substance, hereinafter, also referred to as the “compound A12”).

As a result of the analysis by liquid chromatography-mass spectrometry (LC-MS), the molecular weight was found to be 404. The following peaks were found by carrying out ¹H-NMR measurement under the above measurement conditions, and the compound was confirmed to have the chemical structure of the compound represented by the formula (M12).

δ (ppm) (d6-DMSO): 7.7 (2H, Ph), 6.7 (1H, —CH═), 5.7 (1H, ═CH2), 5.3 (1H, ═CH2), 5.0 (2H, —CH2-), 4.1 (2H, —CH2-), 1.4 (9H, —C—(CH3)3), 1.3 (3H, —CH3)

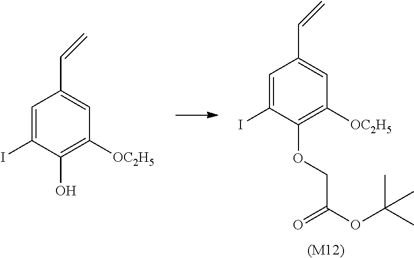

### Example Group 1: Example A13

The reaction was carried out in the same manner as Example A7, except that 4-hydroxy-3-iodo-5-methoxystyrene of Example A7 was changed to 3-ethoxy-4-hydroxy-5-iodostyrene, thereby obtaining 4.1 g of a compound represented by the formula (M13) as the target substance, hereinafter, also referred to as the “compound A13”).

As a result of the analysis by liquid chromatography-mass spectrometry (LC-MS), the molecular weight was found to be 496.

The following peaks were found by carrying out ¹H-NMR measurement under the above measurement conditions, and the compound was confirmed to have the chemical structure of the compound represented by the formula (M12).

δ (ppm) (d6-DMSO): 7.7 (2H, Ph), 6.7 (1H, —CH═), 5.7 (1H, ═CH2), 5.3 (1H, ═CH2), 5.0 (2H, —CH2-), 4.1 (2H, —CH2-), 0.8-2.4 (17H, protons of 2-methyl-2-adamantyl group+3H, —CH3)

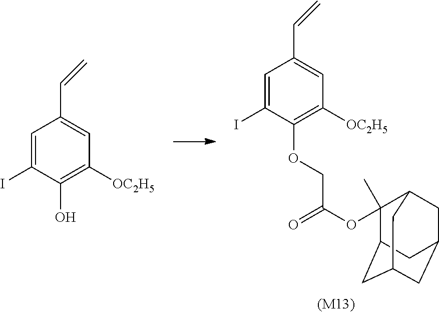

### Example Group 1: Example A14

The reaction was carried out in the same manner as Example A8, except that 4-hydroxy-3-iodo-5-methoxystyrene of Example A8 was changed to 3-ethoxy-4-hydroxy-5-iodostyrene, thereby obtaining 3.5 g of a compound represented by the formula (M14) as the target substance, hereinafter, also referred to as the “compound A14”).

As a result of the analysis by liquid chromatography-mass spectrometry (LC-MS), the molecular weight was found to be 346.

The following peaks were found by carrying out ¹H-NMR measurement under the above measurement conditions, and the compound was confirmed to have the chemical structure of the compound represented by the formula (M14).

δ (ppm) (d6-DMSO): 7.7 (2H, Ph), 6.7 (1H, —CH═), 5.7 (1H, ═CH2), 5.3 (1H, ═CH2), 4.1 (2H, —CH2-), 1.4 (9H, —C—(CH3)3), 1.3 (3H, —CH3)

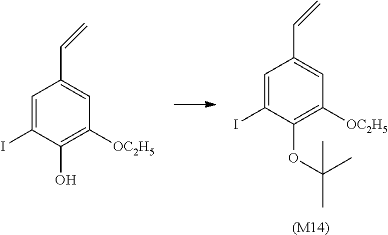

### (Example Group 1: Synthetic Example AD1a) Synthesis of Compound AD1a Represented by Formula (AD1a)

The compound AD1a represented by the formula (AD1a) was synthesized by the method described below.

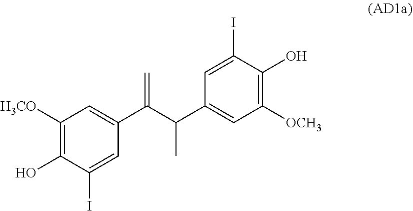 [SMILES: C=C(c1cc(I)c(O)c(OC)c1)C(C)c1cc(I)c(O)c(OC)c1]

The reactor was charged with 11.6 g of 1-(4-hydroxy-3-methoxyphenyl)ethanol, 0.12 g of concentrated sulfuric acid, 0.04 g of 4-hydroxy-2,2,6,6-tetramethylpiperidine 1-oxyl free radical, and 1.60 mL of DMSO, and stirring was initiated. Subsequently, reduced pressure conditions were regulated to reflux at 120° C. using a Dienstark and a condenser, and blowing of air into the reaction solution at a flow rate of 1 mL/minute was initiated. The moisture recovered in the Dienstark was arbitrarily discharged to the outside of the system. Subsequently, the reactor was immersed in a water bath at 90° C., and stirring was continued over 30 hours. Subsequently, the reactor was immersed in a water bath at 25° C. to cool the reaction solution. Subsequently, the reaction solution was gradually added to 400 g of an aqueous sodium hydrogen sulfite solution with a mass percent concentration of 0.1 with vigorous stirring and mixed. Subsequently, the precipitate was filtered off with a suction filter, compressed, and washed with 200 mL of an aqueous methanol solution with a volume percent concentration of 33.3. After only main components were isolated from the obtained precipitate by column purification, the solvent was distilled off by evaporation, and the obtained solid was dried in vacuo at 40° C. to obtain 7.0 g of a white solid.

As a result of the analysis by liquid chromatography-mass spectrometry (LC-MS), the molecular weight was found to be 552, and the white solid was confirmed to be the compound AD1a represented by the formula (AD1a).

The following peaks were found by carrying out ¹H-NMR measurement under the above measurement conditions, and the compound was confirmed to have the chemical structure of the compound AD1a.

δ (ppm) (d6-DMSO): 9.6 (2H, OH), 7.5 (2H, Ph), 7.9 (2H, Ph), 5.3 (1H, ═CH2), 4.9 (1H, ═CH2), 3.5 (1H, —CH—), 1.4 (6H, —CH3), 1.3 (3H, —CH3)

### (Example Group 1: Synthetic Example AD2a) Synthesis of Compound AD2a Represented by Formula (AD2a)

The reaction was carried out in the same manner as Synthetic Example AD1a, except that 1-(4-hydroxy-3-methoxyphenyl)ethanol was changed to 1-(3-ethoxy-4-hydroxyphenyl)ethanol, thereby synthesizing the compound AD2a represented by the formula (AD2a).

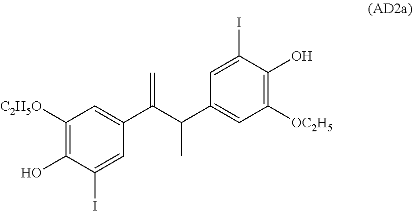

δ (ppm) (d6-DMSO): 9.6 (2H, OH), 7.5 (2H, Ph), 7.9 (2H, Ph), 5.3 (1H, ═CH2), 4.9 (1H, ═CH2), 4.1 (4H, —CH2-), 3.5 (1H, —CH—), 1.4 (6H, —CH3), 1.3 (3H, —CH3)

### (Example Group 1: Synthetic Example AD1b) Synthesis of Compound AD1b Represented by Formula (AD1b)

The compound AD1b represented by the formula (AD1b) was synthesized by the method described below.

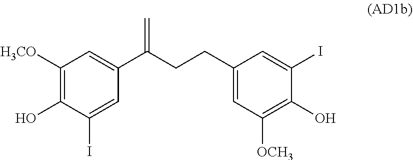 [SMILES: C=C(CCc1cc(I)c(O)c(OC)c1)c1cc(I)c(O)c(OC)c1]

The reactor was charged with 11.6 g of 1-(4-hydroxy-3-methoxyphenyl)ethanol, 0.12 g of concentrated sulfuric acid, 0.2 g of 4-methoxyphenol, and 150 mL of toluene, and stirring was initiated. Subsequently, blowing of air into the reaction solution at a flow rate of 1 mL/minute was initiated under reflux conditions at 113° C. using a Dienstark and a condenser. The moisture recovered in the Dienstark was arbitrarily discharged to the outside of the system. Subsequently, the reactor was immersed in a water bath at 90° C., and stirring was continued over 30 hours. Subsequently, the reactor was immersed in a water bath at 25° C. to cool the reaction solution. Subsequently, the reaction solution was gradually added to 400 g of an aqueous sodium hydrogen sulfite solution with a mass percent concentration of 0.1 with vigorous stirring and mixed. Subsequently, the precipitate was filtered off with a suction filter, compressed, and washed with 200 mL of an aqueous methanol solution with a volume percent concentration of 33.3. After only main components were isolated from the obtained precipitate by column purification, the solvent was distilled off by evaporation, and the obtained solid was dried in vacuo at 40° C. to obtain 2.9 g of a white solid.

As a result of the analysis by liquid chromatography-mass spectrometry (LC-MS), the molecular weight was found to be 552, and the white solid was confirmed to be the compound AD1b represented by the formula (AD1b).

The following peaks were found by carrying out ¹H-NMR measurement under the above measurement conditions, and the compound was confirmed to have the chemical structure of the compound AD1b.

δ (ppm) (d6-DMSO): 9.6 (2H, OH), 7.6 (2H, Ph), 7.5 (2H, Ph), 5.3 (1H, ═CH2), 4.9 (1H, ═CH2), 2.6 (2H, —CH2-), 2.3 (2H, —CH2-), 1.4 (6H, —CH3)

### (Example Group 1: Synthetic Example AD2b) Synthesis of Compound AD2b Represented by Formula (AD2b)

The reaction was carried out in the same manner as Synthetic Example AD1b, except that 1-(4-hydroxy-3-methoxyphenyl)ethanol was changed to 1-(3-ethoxy-4-hydroxyphenyl)ethanol, thereby synthesizing the compound AD2b represented by the formula (AD2b).

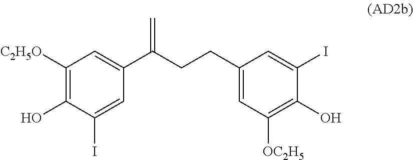

δ (ppm) (d6-DMSO): 9.6 (2H, OH), 7.6 (2H, Ph), 7.5 (2H, Ph), 2.6 (2H, —CH2-), 5.3 (1H, ═CH2), 4.9 (1H, ═CH2), 4.1 (4H, —CH2-), 2.3 (2H, —CH2-), 1.4 (6H, —CH3)

### Example Group 1: Comparative Example A1

The reaction was carried out in the same manner as Example A1, except that 4-hydroxy-3-iodo-5-methoxybenzenecarbaldehyde was changed to 4-hydroxybenzenecarbaldehyde, thereby isolating 90 g of the compound AR1 (4-hydroxystyrene) represented by the formula (MR1) as the target substance.

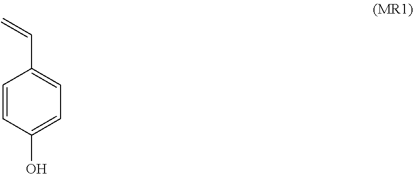

### (Example Group 1: Reference Example AX1) Synthesis of Compound AX1 Represented by Formula (MX1)

Using a 200 mL glass flask as a reaction vessel, 4.96 g (40 mmol) of 4-hydroxybenzyl alcohol was dissolved using butanol as a solvent, a 20% by mass aqueous iodine chloride solution (81.2 g, 100 mmol) was added dropwise thereto at 50° C. over 60 minutes, and then the mixture was stirred at 50° C. for 2 hours to allow 4-hydroxybenzyl alcohol to react with iodine chloride. An aqueous sodium thiosulfate solution was added to the reaction solution after the reaction, the mixture was stirred for 1 hour, and then the liquid temperature was cooled to 10° C. The precipitate precipitated by cooling was filtered off, washed, and dried to obtain 12.1 g of a white solid. The white solid sample was analyzed by liquid chromatography-mass spectrometry (LC-MS) and, as a result, confirmed to be 4-hydroxy-3,5-diiodobenzyl alcohol.

MnO₂ (3.4 g, 40 mmol) was added to a methylene chloride solvent and stirred, which was then stirred for 1 hour while a 50% by mass solution in which the total amount of the synthesized 4-hydroxy-3,5-diiodobenzyl alcohol was dissolved in methylene chloride was added dropwise and stirred at room temperature for 4 hours, and then the reaction solution was filtered off and the solvent was distilled off to obtain 4-hydroxy-3,5-diiodobenzaldehyde.

A solution in which dimethyl malonate (5.3 g, 40 mmol) and the total amount of 4-hydroxy-3,5-diiodobenzaldehyde synthesized above was dissolved in a DMF solvent was prepared and then stirred for 1 hour while a solution in which ethylenediamine (0.3 g) was dissolved in DMF was added dropwise, and the mixture was allowed to react for 6 hours with stirring while controlling the liquid temperature at 150° C. in an oil bath. Thereafter, ethyl acetate and water was added, a 2 mol/L aqueous HCl solution was added to control pH to 4 or less, and then a separation operation was carried out to collect the organic phase. Further, the obtained organic phase was washed by being subjected to a separation operation with a 2 mol/L aqueous sodium carbonate solution, water, and brine in the order presented, and then purified by a filter, and the solvent was distilled off from the organic phase to obtain 8.1 g of the compound AX1 (4-hydroxy-3,5-diiodostyrene (a compound represented by the following formula (MX1))). The inorganic element content and the organic impurity content were measured by the aforementioned method, and the results are shown in Table 1.

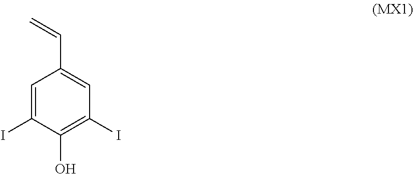

### Example Group 1: Comparative Example A2

The reaction was carried out in the same manner as Example A1, except that 4-hydroxy-3-iodo-5-methoxybenzenecarbaldehyde was changed to 3,4-dihydroxybenzenecarbaldehyde, thereby isolating 90 g of 3,4-dihydroxystyrene represented by the formula (MR2) as the target substance.

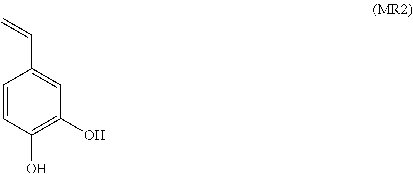

For the impurity content of the compounds synthesized in the aforementioned Examples and Comparative Examples, the inorganic element content and the organic impurity content were measured by the aforementioned method, and the results are shown in Table 1.

TABLE 1

| | | | Monomer physical properties | | | | |
|---|---|---|---|---|---|---|---|
| | | | Impurity content (ppm) | | | | |
| | | | | | | | Phosphorus- |
| | Compound | Structural | | | | Maleic | containing |
| | No. | formula | Mn | A1 | K | acid | compound |
| Example A1 | A1 | M1 | 2 | 2 | 11 | DL | 30 |
| Example A2 | A2 | M2 | 2 | 2 | 11 | DL | 28 |
| Example A3 | A3 | M3 | 1 | 1 | 6 | DL | 10 |
| Example A4 | A4 | M4 | 1 | 1 | 6 | DL | 10 |
| Example A5 | A5 | M5 | 1 | 1 | 6 | DL | 10 |
| Example A6 | A6 | M6 | 1 | 1 | 6 | DL | 10 |
| Example A7 | A7 | M7 | 1 | 1 | 6 | DL | 10 |
| Comparative | R1 | R1 | 2 | 2 | 11 | DL | 35 |
| Example A1 | | | | | | | |
| Comparative | R2 | R2 | 2 | 2 | 11 | DL | 40 |
| Example A2 | | | | | | | |
| Reference | AX1 | MX1 | 2 | 2 | 11 | DL | 10 |
| Example AX1 | | | | | | | |

DL: below detection limit (<0.1 ppm)

TABLE 1-2

| | | | Monomer physical properties | | | | |
|---|---|---|---|---|---|---|---|
| | | | Impurity content (ppm) | | | | |
| | | | | | | | Phosphorus- |
| | Compound | Structural | | | | Maleic | containing |
| | No. | formula | Mn | A1 | K | acid | compound |
| Example A8 | A8 | M8 | 1 | 1 | 6 | DL | 30 |
| Example A9 | A9 | M9 | 1 | 1 | 6 | DL | 28 |
| Example A10 | A10 | M10 | 1 | 1 | 6 | DL | 10 |
| Example A11 | A11 | M11 | 1 | 1 | 6 | DL | 10 |
| Example A12 | A12 | M12 | 1 | 1 | 6 | DL | 10 |
| Example A13 | A13 | M13 | 1 | 1 | 6 | DL | 10 |
| Example A14 | A14 | M14 | 1 | 1 | 6 | DL | 10 |
| Example A1a | A1a | M1a | 1 | 1 | 6 | DL | 10 |
| Example A2a | A2a | M2a | 1 | 1 | 6 | DL | 10 |

DL: below detection limit (<0.1 ppm)

### (Evaluation of Aging Stability of Composition Containing Compound)

The stability of the composition containing the compound obtained in Examples or Comparative Examples described above was evaluated using an index of the amount of change of the purity before and after the aging test in a solution state in which a single or a plurality of compounds are combined.

As the sample for evaluation, a solution in which the compounds of Examples or Comparative Examples (the compounds shown as the compound a1, the compound a2, or the compound a3) and a solvent described in Table A and Table A-2 were mixed was prepared, which was filled in an inactivation-treated 100 mL brown glass container until 90 mL and capped to prepare a sample. As aging conditions, the sample was subjected to an aging treatment in a light-shielded constant temperature tester at 45° C. for 30 days.

For the prepared sample, the purity before and after aging treatment was measured by HPLC analysis.

The amount of change of the HPLC purity before and after aging was determined as follows and used as an index of the evaluation.

The obtained results were described in Table A and Table A-2.

Amount of change of purity due to aging=area % of target component before aging−area % of target component after aging

### (Evaluation Criteria)

A: Amount of change of purity due to aging≤0.2%
B: 0.2%<Amount of change of purity due to aging≤0.5%
C: 0.5%<Amount of change of purity due to aging≤1.0%
D: 1.0%<Amount of change of purity due to aging≤3.0%
E: 3.0%<Amount of change of purity due to aging

TABLE 1-3
Table A

| | Composition | | | | | | | |
|---|---|---|---|---|---|---|---|---|
| | Compound a1 | | Compound a2 | | Compound a3 | | Solvent | Evaluation |
| | | Amount | | Amount | | Amount | Toluene | due to |
| | No. | (g) | No. | (g) | No. | (g) | (g) | aging |
| Example SL1 | A1 | 1.3875 | AR1 | 0.1125 | — | — | 13.5 | A |
| Example SL2 | A2 | 1.3875 | AR1 | 0.1125 | — | — | 13.5 | A |
| Example SL3 | A1 | 1.4985 | AR1 | 0.0015 | — | — | 13.5 | B |
| Example SL4 | A2 | 1.4985 | AR1 | 0.0015 | — | — | 13.5 | B |
| Example SL5 | A1 | 1.3875 | AR2 | 0.1125 | — | — | 13.5 | A |
| Example SL6 | A2 | 1.3875 | AR2 | 0.1125 | — | — | 13.5 | A |
| Example SL7 | A1 | 1.3875 | AD1a | 0.1125 | — | — | 13.5 | A |
| Example SL8 | A1 | 1.3875 | AD2a | 0.1125 | — | — | 13.5 | A |
| Example SL9 | A1 | 1.3875 | AD1b | 0.1125 | — | — | 13.5 | A |
| Example SL10 | A1 | 1.3875 | AD2b | 0.1125 | — | — | 13.5 | A |
| Example SL11 | A1 | 1.4985 | AD1a | 0.0015 | — | — | 13.5 | B |
| Example SL12 | A1 | 1.4985 | AD2a | 0.0015 | — | — | 13.5 | B |
| Example SL13 | A1 | 1.4985 | AD1b | 0.0015 | — | — | 13.5 | B |
| Example SL14 | A1 | 1.4985 | AD2b | 0.0015 | — | — | 13.5 | B |
| Example SL15 | A1 | 1.3803 | AR1 | 0.0825 | AD1a | 0.1125 | 13.5 | A |
| Example SL16 | A1 | 1.491 | AR1 | 0.0045 | AD1a | 0.0045 | 13.5 | A |
| Example SL17 | A1 | 1.3803 | AR1 | 0.0825 | AD2a | 0.1125 | 13.5 | B |
| Example SL18 | A1 | 1.491 | AR1 | 0.0045 | AD2a | 0.0045 | 13.5 | B |
| Example SL19 | A1 | 1.5 | — | — | — | — | 13.5 | C |
| Example SL20 | A2 | 1.5 | — | — | — | — | 13.5 | C |
| Example SL21 | A3 | 1.5 | — | — | — | — | 13.5 | B |
| Example SL22 | A4 | 1.5 | — | — | — | — | 13.5 | B |
| Example SL23 | A5 | 1.5 | — | — | — | — | 13.5 | B |
| Example SL24 | A6 | 1.5 | — | — | — | — | 13.5 | B |
| Example SL25 | A7 | 1.5 | — | — | — | — | 13.5 | B |

TABLE A-2

| | Composition | | | | | | | |
|---|---|---|---|---|---|---|---|---|
| | Compound a1 | | Compound a2 | | Compound a3 | | Solvent | Evaluation |
| | | Amount | | Amount | | Amount | Toluene | due to |
| | No. | (g) | No. | (g) | No. | (g) | (g) | aging |
| Example SL26 | A2 | 1.1391 | AR1 | 0.0681 | AD2a | 0.0928 | 13.5 | A |
| Example SL27 | A2 | 1.2922 | AR1 | 0.0039 | AD2a | 0.0039 | 13.5 | A |
| Example SL28 | A2 | 1.1391 | AR1 | 0.0681 | AD2a | 0.0464 | 13.5 | B |
| Example SL29 | A2 | 1.2922 | AR1 | 0.0039 | AD2a | 0.0020 | 13.5 | B |
| Example SL30 | A8 | 1.3 | — | — | — | — | 13.5 | C |
| Example SL31 | A9 | 1.3 | — | — | — | — | 13.5 | C |
| Example SL32 | A10 | 1.3 | — | — | — | — | 13.5 | B |
| Example SL33 | A11 | 1.3 | — | — | — | — | 13.5 | B |
| Example SL34 | A12 | 1.3 | — | — | — | — | 13.5 | B |
| Example SL35 | A13 | 1.3 | — | — | — | — | 13.5 | B |
| Example SL36 | A14 | 1.3 | — | — | — | — | 13.5 | B |

The results were obtained from Table A that determines that the compound (A) according to the embodiment can improve the stability in a solution state by containing a trace amount of the compound of the formula (1A) or the compound of the formula (1C).

### (Example Group 1: Example B1) Synthesis of Polymer

4.7 g of the compound A1, 3.0 g of 2-methyl-2-adamantyl methacrylate, 2.0 g of γ-butyrolactone methacrylate, and 1.5 g of hydroxyadamantyl methacrylate were dissolved in 45 mL of tetrahydrofuran, and 0.20 g of azobisisobutyronitrile was added thereto. After the mixture was refluxed for 12 hours, the reaction solution was added dropwise to 2 L of n-heptane. The polymer precipitated was filtered off and dried under reduced-pressure to obtain a white powdery polymer B1 represented by the following formula (MA1). This polymer had a weight average molecular weight (Mw) of 12,000 and a dispersity (Mw/Mn) of 1.90. As a result of the measurement of ¹³C-NMR, the composition ratio (molar ratio) in the following formula (MA1) was a:b:c:d=40:30:15:15. Although the following formula (MA1) is illustrated in a simplified form to show the ratio of respective constitutional units, the order of arrangement of respective constitutional units is random and this polymer is not a block copolymer in which respective constitutional units form an independent block.

The inorganic element content and organic impurity content of the synthesized polymer were measured by the aforementioned method, and the obtained measurement results are shown in Table 3. The molar ratio was determined based on the integral ratio of each of the carbon on the bottom of a benzene ring for the polystyrene monomer (the compound A1) and the carbonyl carbon of an ester bond for methacrylate monomer (2-methyl-2-adamantyl methacrylate, γ-butyrolactone methacrylate, and hydroxyadamantyl methacrylate). The kind of respective monomers, the ratio thereof, and the composition ratio in the polymer obtained in Example B1 are shown in Table 2. The kind of respective monomers, the ratio thereof, and the composition ratio in the polymers obtained in Examples described below are also shown in Table 2.

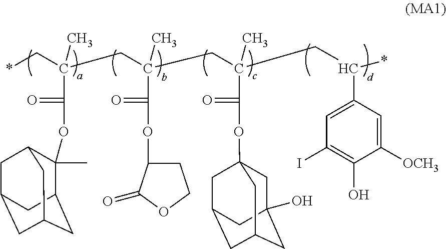

### Example B2 and Comparative Example BR1: Synthesis of Polymer B2 and Polymer BR1

The synthesis was carried out in the same manner as the method described in Example B1, except that 1.5 g of the compound A1 was changed to the monomer compounds of the kind and the amount shown in Table 2, thereby obtaining the polymers B2 and BR1 represented by the formula (MA2) and the formula (MAR1). The inorganic element content and organic impurity content of the polymers were measured by the aforementioned method, and the obtained measurement results are shown in Table 3.

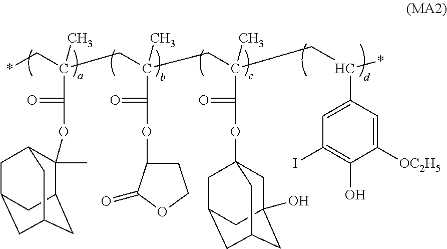

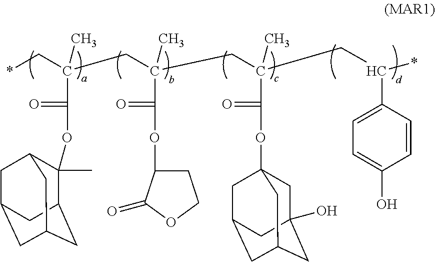

### (Example Group 1: Example B1P) Synthesis of Polymer B1P

For the synthesized compound A1, purification treatments of respective raw materials were additionally conducted before synthesis of the polymer. Using ethyl acetate (PrimePure manufactured by Kanto Chemical Co., Inc.) as a solvent, a 10% by mass ethyl acetate solution of the compound A1 in which the compound A1 was dissolved was prepared. To remove metal impurities, an ion exchange resin “AMBERLYST MSPS2-1·DRY” (product name, manufactured by ORGANO CORPORATION) was immersed in ethyl acetate (PrimePure manufactured by Kanto Chemical Co., Inc.), stirred for 1 hour, and then the solvent was removed. Washing of the ion exchange resin was carried out by repeating washing in this manner 10 times. In the aforementioned ethyl acetate solution of the compound A1, the same mass of the washed ion exchange resin as the resin solid content was put and stirred at room temperature for a day, and then the ion exchange resin was filtered off. Washing in which an ion exchange treatment was carried out in this manner was repeated three times to prepare an ion-exchanged ethyl acetate solution of the compound A1. Furthermore, the same treatment was carried out for other monomers, thereby preparing an ion-exchanged monomer-containing ethyl acetate solution. Synthesis was carried out according to the same scheme as the synthesis of the polymer B1 in Example B1 by using the obtained ion exchange treated monomer-containing ethyl acetate solution, using Pruimepure manufactured by Kanto Chemical Co., Inc., which is electronic grade as the solvent such as n-heptane and tetrahydrofuran, and further using instruments, all of which were immersed in nitric acid for a day and then washed with ultrapure water, as the reaction vessel such as a flask. Further in the treatment after synthesis, a purification treatment was carried out by using a 5 nm Nylon filter (manufactured by Pall) and a 15 nm PTFE filter (manufactured by Entegris) in the order presented, and the purified product was dried under reduced pressure to obtain a white powdery polymer B1P (a polymer whose chemical structure is represented by the formula (MA1)). The inorganic element content and organic impurity content of the monomer compounds used in the synthesis of the obtained polymers after the purification treatment were measured by the aforementioned method, and the obtained measurement results are shown in Table 3.

### (Example Group 1: Examples B2P to B7P) Synthesis of Polymers B2P to B7P

The polymers B2P to B7P (polymers whose chemical structures are represented by the formulas (MA2 to MA7) and BX1) were obtained in the same manner as Example B1P except for using the compounds M2 to M7 and MX1 instead of the compound M1. The inorganic element content and organic impurity content of the monomer compounds used in the synthesis of the obtained polymers after the purification treatment were measured by the aforementioned method, and the obtained measurement results are shown in Table 3.

TABLE 2

| | Monomer | | | | | | | | | | |
|---|---|---|---|---|---|---|---|---|---|---|---|
| | Compound | | MAMA | BLMA | HAMA | Polymer | | | | | |
| | | Amount | Amount | Amount | Amount | | Structural | | | | |
| | No. | g | g | g | g | No. | formula | a | b | c | d |
| Example B1 | A1 | 4.7 | 3 | 2 | 1.5 | B1 | MA1 | 40 | 30 | 15 | 15 |
| Example B1P | A1 | 4.7 | 3 | 2 | 1.5 | B1P | MA1 | 40 | 30 | 15 | 15 |
| Example B2 | A2 | 4.7 | 3 | 2 | 1.5 | B2 | MA2 | 40 | 30 | 15 | 15 |
| Example B2P | A2 | 4.7 | 3 | 2 | 1.5 | B2P | MA3 | 40 | 30 | 15 | 15 |
| Example B3P | A3 | 4.7 | 3 | 2 | 1.5 | B3P | MA4 | 40 | 30 | 15 | 15 |
| Example B4P | A4 | 4.7 | 3 | 2 | 1.5 | B4P | MA5 | 40 | 30 | 15 | 15 |
| Example B5P | A5 | 4.7 | 3 | 2 | 1.5 | B5P | MA6 | 40 | 30 | 15 | 15 |
| Example B6P | A6 | 4.7 | 3 | 2 | 1.5 | B6P | MA7 | 40 | 30 | 15 | 15 |
| Example B7P | A7 | 4.7 | 3 | 2 | 1.5 | B7P | MA2 | 40 | 30 | 15 | 15 |
| Comparative | AR1 | 2.1 | 3 | 2 | 1.5 | BR1 | MAR1 | 40 | 30 | 15 | 15 |
| Example BR1 | | | | | | | | | | | |

The meanings of the abbreviations and symbols in the Tables are as follows.

MAMA: 2-methyl-2-adamantyl methacrylate
BLMA: γ-butyrolactone methacrylate
HAMA: hydroxyadamantyl methacrylate
a, b, c, and d of the polymer are molar ratios.

TABLE 3

| | | | Monomer physical properties | | | | |
|---|---|---|---|---|---|---|---|
| | | | Impurity content (ppm) | | | | |
| | | | | | | | Phosphorus- |
| | | Structural | | | | Maleic | containing |
| | Polymer | formula | Mn | A1 | K | acid | compound |
| Example B1 | B1 | MA1 | 0.01 | 0.01 | 0.01 | DL | 0.1 |
| Example B1P | B1P | MA1 | 0.0001 | 0.0001 | 0.0001 | DL | DL |
| Example B2 | B2 | MA2 | 0.01 | 0.01 | 0.01 | DL | 0.1 |
| Example B2P | B2P | MA2 | 0.0001 | 0.0001 | 0.0001 | DL | DL |
| Example B3P | B3P | MA4 | 0.0001 | 0.0001 | 0.0001 | DL | DL |
| Example B4P | B4P | MA5 | 0.0001 | 0.0001 | 0.0001 | DL | DL |
| Example B5P | B5P | MA6 | 0.0001 | 0.0001 | 0.0001 | DL | DL |
| Example B6P | B6P | MA7 | 0.0001 | 0.0001 | 0.0001 | DL | DL |
| Example B7P | B7P | MA2 | 0.0001 | 0.0001 | 0.0001 | DL | DL |
| Comparative | BR1 | MAR1 | 0.1 | 0.1 | 0.1 | DL | DL |
| Example BR1 | | | | | | | |
| Reference | BX1 | MX1 | 0.0001 | 0.0001 | 0.0001 | DL | DL |
| Example BX1 | | | | | | | |

DL: below detection limit (<0.1 ppm)

### (Example Group 1: Examples BD1 to BD30) Synthesis of Polymers PMD1 to PMD30

The polymers BD1 to BD30 (polymers whose chemical structures are represented by the formulas (PMD1 to PMD30)) were obtained in the same manner as Example B1P except for using the compound a1, the compound a2, and the compound a3 described in Table 2-2 and Table 2-3 instead of the compound M1, in the described ratio. The inorganic element content and organic impurity content of the monomer compounds used in the synthesis of the obtained polymers were measured by the aforementioned method, and the obtained measurement results are shown in Table 3-2 and Table 3-3.

TABLE 2-2

| | Composition | | | | | | | | |
|---|---|---|---|---|---|---|---|---|---|
| | Compound a1 | | Compound a2 | | Compound a3 | | | | |
| | | Amount | | Amount | | Amount | | | |
| | No. | (g) | No. | (g) | No. | (g) | MMA | BLMA | HAMA |
| Example BD1 | A1 | 4.35 | AR1 | 0.154 | — | — | 3 | 2 | 1.5 |
| Example BD2 | A1 | 4.65 | AR1 | 0.026 | — | — | 3 | 2 | 1.5 |
| Example BD3 | A1 | 4.68 | AR1 | 0.013 | — | — | 3 | 2 | 1.5 |
| Example BD4 | A1 | 4.71 | AR1 | 0.002 | — | — | 3 | 2 | 1.5 |
| Example BD5 | A1 | 4.35 | AD1a | 0.952 | — | — | 3 | 2 | 1.5 |
| Example BD6 | A1 | 4.65 | AD1a | 0.159 | — | — | 3 | 2 | 1.5 |
| Example BD7 | A1 | 4.68 | AD1a | 0.079 | — | — | 3 | 2 | 1.5 |
| Example BD8 | A1 | 4.71 | AD1a | 0.012 | — | — | 3 | 2 | 1.5 |
| Example BD9 | A1 | 4.35 | AD1b | 0.952 | — | — | 3 | 2 | 1.5 |
| Example BD10 | A1 | 4.65 | AD1b | 0.159 | — | — | 3 | 2 | 1.5 |
| Example BD11 | A1 | 4.68 | AD1b | 0.079 | — | — | 3 | 2 | 1.5 |
| Example BD12 | A1 | 4.71 | AD1b | 0.012 | — | — | 3 | 2 | 1.5 |
| Example BD13 | A1 | 4.10 | AR1 | 0.154 | AD1a | 0.698 | 3 | 2 | 1.5 |
| Example BD14 | A1 | 4.59 | AR1 | 0.030 | AD1a | 0.127 | 3 | 2 | 1.5 |
| Example BD15 | A1 | 4.70 | AR1 | 0.002 | AD1a | 0.013 | 3 | 2 | 1.5 |

| | | | a1 | a2 | a3 | b | c | d |
|---|---|---|---|---|---|---|---|---|
| | | Structural | Molar | Molar | Molar | Molar | Molar | Molar |
| | No. | formula | ratio | ratio | ratio | ratio | ratio | ratio |
| Example BD1 | BD16 | PME16 | 37 | 3 | | 30 | 30 | 15 |
| Example BD2 | BD17 | PME17 | 39.5 | 0.5 | | 30 | 30 | 15 |
| Example BD3 | BD18 | PME18 | 39.75 | 0.25 | | 30 | 30 | 15 |
| Example BD4 | BD19 | PME19 | 39.96 | 0.04 | | 30 | 30 | 15 |
| Example BD5 | BD20 | PME20 | 37 | 3 | | 30 | 30 | 15 |
| Example BD6 | BD21 | PME21 | 39.5 | 0.5 | | 30 | 30 | 15 |
| Example BD7 | BD22 | PME22 | 39.75 | 0.25 | | 30 | 30 | 15 |
| Example BD8 | BD23 | PME23 | 39.96 | 0.04 | | 30 | 30 | 15 |
| Example BD9 | BD24 | PME24 | 37 | 3 | | 30 | 30 | 15 |
| Example BD10 | BD25 | PME25 | 39.5 | 0.5 | | 30 | 30 | 15 |
| Example BD11 | BD26 | PME26 | 39.75 | 0.25 | | 30 | 30 | 15 |
| Example BD12 | BD27 | PME27 | 39.96 | 0.04 | | 30 | 30 | 15 |
| Example BD13 | BD28 | PME28 | 34.2 | 3 | 2.2 | 30 | 30 | 15 |
| Example BD14 | BD29 | PME29 | 39.0 | 0.6 | 0.4 | 30 | 30 | 15 |
| Example BD15 | BD30 | PME30 | 39.92 | 0.04 | 0.04 | 30 | 30 | 15 |

TABLE 2-3

| | Composition | | | | | | | | |
|---|---|---|---|---|---|---|---|---|---|
| | Compound a1 | | Compound a2 | | Compound a3 | | | | |
| | | Amount | | Amount | | Amount | | | |
| | No. | (g) | No. | (g) | No. | (g) | MMA | BLMA | HAMA |
| Example BD16 | A2 | 4.35 | AR1 | 0.154 | — | — | 3 | 2 | 1.5 |
| Example BD17 | A2 | 4.65 | AR1 | 0.026 | — | — | 3 | 2 | 1.5 |
| Example BD18 | A2 | 4.68 | AR1 | 0.013 | — | — | 3 | 2 | 1.5 |
| Example BD19 | A2 | 4.71 | AR1 | 0.002 | — | — | 3 | 2 | 1.5 |
| Example BD20 | A2 | 4.35 | AD2a | 0.952 | — | — | 3 | 2 | 1.5 |
| Example BD21 | A2 | 4.65 | AD2a | 0.159 | — | — | 3 | 2 | 1.5 |
| Example BD22 | A2 | 4.68 | AD2a | 0.079 | — | — | 3 | 2 | 1.5 |
| Example BD23 | A2 | 4.71 | AD2a | 0.012 | — | — | 3 | 2 | 1.5 |
| Example BD24 | A2 | 4.35 | AD2b | 0.952 | — | — | 3 | 2 | 1.5 |
| Example BD25 | A2 | 4.65 | AD2b | 0.159 | — | — | 3 | 2 | 1.5 |
| Example BD26 | A2 | 4.68 | AD2b | 0.079 | — | — | 3 | 2 | 1.5 |
| Example BD27 | A2 | 4.71 | AD2b | 0.012 | — | — | 3 | 2 | 1.5 |
| Example BD27 | A2 | 4.10 | AR1 | 0.154 | AD2a | 0.698 | 3 | 2 | 1.5 |
| Example BD28 | A2 | 4.59 | AR1 | 0.030 | AD2a | 0.127 | 3 | 2 | 1.5 |
| Example BD30 | A2 | 4.70 | AR1 | 0.002 | AD2a | 0.013 | 3 | 2 | 1.5 |

| | | | a1 | a2 | a3 | b | c | d |
|---|---|---|---|---|---|---|---|---|
| | | Structural | Molar | Molar | Molar | Molar | Molar | Molar |
| | No. | formula | ratio | ratio | ratio | ratio | ratio | ratio |
| Example BD16 | BD31 | PME16 | 37 | 3 | | 30 | 30 | 15 |
| Example BD17 | BD32 | PME17 | 39.5 | 0.5 | | 30 | 30 | 15 |
| Example BD18 | BD33 | PME18 | 39.75 | 0.25 | | 30 | 30 | 15 |
| Example BD19 | BD34 | PME19 | 39.96 | 0.04 | | 30 | 30 | 15 |
| Example BD20 | BD35 | PME20 | 37 | 3 | | 30 | 30 | 15 |
| Example BD21 | BD36 | PME21 | 39.5 | 0.5 | | 30 | 30 | 15 |
| Example BD22 | BD37 | PME22 | 39.75 | 0.25 | | 30 | 30 | 15 |
| Example BD23 | BD38 | PME23 | 39.96 | 0.04 | | 30 | 30 | 15 |
| Example BD24 | BD39 | PME24 | 37 | 3 | | 30 | 30 | 15 |
| Example BD25 | BD40 | PME25 | 39.5 | 0.5 | | 30 | 30 | 15 |
| Example BD26 | BD41 | PME26 | 39.75 | 0.25 | | 30 | 30 | 15 |
| Example BD27 | BD42 | PME27 | 39.96 | 0.04 | | 30 | 30 | 15 |
| Example BD27 | BD43 | PME28 | 34.2 | 3 | 2.2 | 30 | 30 | 15 |
| Example BD28 | BD44 | PME29 | 39.0 | 0.6 | 0.4 | 30 | 30 | 15 |
| Example BD30 | BD45 | PME30 | 39.92 | 0.04 | 0.04 | 30 | 30 | 15 |

TABLE 3-2

| | | | Monomer physical properties | | | | |
|---|---|---|---|---|---|---|---|
| | | | Impurity content (ppm) | | | | |
| | | | | | | | Phosphorus- |
| | | Structural | | | | Maleic | containing |
| | Polymer | formula | Mn | A1 | K | acid | compound |
| Example BD1 | BD1 | PMD1 | 0.0001 | 0.0001 | 0.0001 | DL | DL |
| Example BD2 | BD2 | PMD2 | 0.0001 | 0.0001 | 0.0001 | DL | DL |
| Example BD3 | BD3 | PMD3 | 0.0001 | 0.0001 | 0.0001 | DL | DL |
| Example BD4 | BD4 | PMD4 | 0.0001 | 0.0001 | 0.0001 | DL | DL |
| Example BD5 | BD5 | PMD5 | 0.0001 | 0.0001 | 0.0001 | DL | DL |
| Example BD6 | BD6 | PMD6 | 0.0001 | 0.0001 | 0.0001 | DL | DL |
| Example BD7 | BD7 | PMD7 | 0.0001 | 0.0001 | 0.0001 | DL | DL |
| Example BD8 | BD8 | PMD8 | 0.0001 | 0.0001 | 0.0001 | DL | DL |
| Example BD9 | BD9 | PMD9 | 0.0001 | 0.0001 | 0.0001 | DL | DL |
| Example BD10 | BD10 | PMD10 | 0.0001 | 0.0001 | 0.0001 | DL | DL |
| Example BD11 | BD11 | PMD11 | 0.0001 | 0.0001 | 0.0001 | DL | DL |
| Example BD12 | BD12 | PMD12 | 0.0001 | 0.0001 | 0.0001 | DL | DL |
| Example BD13 | BD13 | PMD13 | 0.0001 | 0.0001 | 0.0001 | DL | DL |
| Example BD14 | BD14 | PMD14 | 0.0001 | 0.0001 | 0.0001 | DL | DL |
| Example BD15 | BD15 | PMD15 | 0.0001 | 0.0001 | 0.0001 | DL | DL |

TABLE 3-3

| | | | Monomer physical properties | | | | |
|---|---|---|---|---|---|---|---|
| | | | Impurity content (ppm) | | | | |
| | | | | | | | Phosphorus- |
| | | Structural | | | | Maleic | containing |
| | Polymer | formula | Mn | A1 | K | acid | compound |
| Example BD16 | BD16 | PMD16 | 0.0001 | 0.0001 | 0.0001 | DL | DL |
| Example BD17 | BD17 | PMD17 | 0.0001 | 0.0001 | 0.0001 | DL | DL |
| Example BD18 | BD18 | PMD18 | 0.0001 | 0.0001 | 0.0001 | DL | DL |
| Example BD19 | BD19 | PMD19 | 0.0001 | 0.0001 | 0.0001 | DL | DL |
| Example BD20 | BD20 | PMD20 | 0.0001 | 0.0001 | 0.0001 | DL | DL |
| Example BD21 | BD21 | PMD21 | 0.0001 | 0.0001 | 0.0001 | DL | DL |
| Example BD22 | BD22 | PMD22 | 0.0001 | 0.0001 | 0.0001 | DL | DL |
| Example BD23 | BD23 | PMD23 | 0.0001 | 0.0001 | 0.0001 | DL | DL |
| Example BD24 | BD24 | PMD24 | 0.0001 | 0.0001 | 0.0001 | DL | DL |
| Example BD25 | BD25 | PMD25 | 0.0001 | 0.0001 | 0.0001 | DL | DL |
| Example BD26 | BD26 | PMD26 | 0.0001 | 0.0001 | 0.0001 | DL | DL |
| Example BD27 | BD27 | PMD27 | 0.0001 | 0.0001 | 0.0001 | DL | DL |
| Example BD28 | BD28 | PMD28 | 0.0001 | 0.0001 | 0.0001 | DL | DL |
| Example BD29 | BD29 | PMD29 | 0.0001 | 0.0001 | 0.0001 | DL | DL |
| Example BD30 | BD30 | PMD30 | 0.0001 | 0.0001 | 0.0001 | DL | DL |

### [Evaluation]

The aforementioned polymers obtained in Examples and Comparative Examples were evaluated as follows. The results are shown in Table 4, Table 4-2, Table 4-3, Table 4-4, Table 5, and Table 5-2.

### (EUV Sensitivity-Aqueous TMAH Solution Development)

A solution was prepared by blending 5 parts by mass of the compound or the polymer obtained in Examples or Comparative Examples, 1 part by mass of triphenylsulfonium nonafluoromethanesulfonate, 0.2 parts by mass of tributylamine, 80 parts by mass of PGMEA, and 12 parts by mass of PGME.

The solution was applied on a silicon wafer, which was baked at 110° C. for 60 seconds to form a photoresist layer having a film thickness of 100 nm.

Then, using an extreme ultraviolet (EUV) exposure apparatus “EUVES-7000” (product name, manufactured by Litho Tech Japan Corporation), the photoresist layer was subjected to shot exposure without masking by incrementing the exposure amount by 1 mJ/cm² from 1 mJ/cm² to 80 mJ/cm², baked (PEB) at 110° C. for 90 seconds, and developed with a 2.38% by mass aqueous tetramethylammonium hydroxide (TMAH) solution for 60 seconds to obtain a wafer on which 80 shots of shot exposure was carried out. For each obtained shot exposure area, the film thickness was measured by using an optical interference film thickness meter “VM3200” (product name, manufactured by SCREEN Semiconductor Solutions Co., Ltd.), profile data about the film thickness relative to the exposure amount was obtained, and the exposure amount at which the slope of film thickness variation relative to an exposure amount was the largest was calculated as a sensitivity value (mJ/cm²), which was used as an index of the EUV sensitivity of the resist.

### (Change of Sensitivity Due to Aging)

The solution prepared in the aforementioned EUV sensitivity evaluation was subjected to a forced aging treatment under light-shielding conditions at 40° C./240 hours, and the solution after the aging treatment was evaluated for EUV sensitivity in the same manner. Evaluations were conducted based on the amount of change of sensitivity. As the specific evaluation method, in a film thickness-sensitivity curve after development when the abscissa was set to sensitivity and the ordinate was set to film thickness in the EUV sensitivity evaluation, the sensitivity value at which the slope value was maximum was measured as the standard sensitivity. The standard sensitivities of the solutions before and after being subjected to a forced aging treatment were determined, and sensitivity variations due to the aging treatment were evaluated from the numerical value obtained by the following calculation expression. The evaluation criteria are as follows.

[Sensitivity variation]=1−([standard sensitivity of solution after aging]÷[standard sensitivity of solution before aging])

### (Evaluation Criteria)

S: [sensitivity variation]≤0.0025
A: 0.0025<[sensitivity variation]≤0.005
B: 0.005<[sensitivity variation]≤0.02
C: 0.02<[sensitivity variation]≤0.05
D: 0.05<[sensitivity variation]

### (EB Pattern-Aqueous TMAH Solution Development)

A solution was prepared by blending 5 parts by mass of the compound or the polymer obtained in Examples or Comparative Examples, 1 part by mass of triphenylsulfonium nonafluoromethanesulfonate, 0.1 parts by mass of tributylamine, and 92 parts by mass of PGMEA.

The solution was applied on a silicon wafer, which was baked at 110 to 130° C. for 60 seconds to form a resist film having a film thickness of 100 nm.

Then, the resist film was exposed using an electron beam lithography system “ELS-7500” (product name, manufactured by ELIONIX INC., 50 keV), baked (PEB) at 115° C. for 90 seconds, and developed with a 2.38 mass % tetramethylammonium hydroxide (TMAH) aqueous solution for 60 seconds to obtain a positive type pattern. The exposure amount was regulated to have a line and space pattern with a half pitch of 50 nm.

For the obtained resist pattern, 80 pattern images were obtained with a scanning electron microscope “S-4800” (product name, manufactured by Hitachi, Ltd.) at 100000 times magnification, the number of residues on the space portion between resist patterns were counted, and evaluations were carried out from the total amount of the residues. The evaluation criteria are as follows.

### (Evaluation Criteria)

A: Number of residues≤less than 10
B: 10<number of residues≤80
C: 80<number of residues≤400
D: 400<number of residues

### (Evaluation of Etching Defect)

A solution was prepared by blending 5 parts by mass of the compound or the polymer obtained in Examples or Comparative Examples, 1 part by mass of triphenylsulfonium nonafluoromethanesulfonate, 0.2 parts by mass of tributylamine, 80 parts by mass of PGMEA, and 12 parts by mass of PGME.

The solution was applied on a 8 inch silicon wafer, on the outermost layer of which an oxide film having a film thickness of 100 nm was formed, and this was baked at 110° C. for 60 seconds to form a photoresist layer having a film thickness of 100 nm.

Then, using an extreme ultraviolet (EUV) exposure apparatus “EUVES-7000” (product name, manufactured by Litho Tech Japan Corporation), the photoresist layer was subjected to shot exposure with an exposure amount that is 10% less than the EUV sensitivity value obtained in the aforementioned EUV sensitivity evaluation on the entire wafer surface, further baked (PEB) at 110° C. for 90 seconds, and developed with a 2.38% by mass aqueous tetramethylammonium hydroxide (TMAH) solution for 60 seconds to obtain a wafer on the entire surface of which 80 shots of shot exposure was carried out.

The prepared exposed wafer was subjected to an etching treatment with an etching apparatus “Telius SCCM” (product name, manufactured by Tokyo Electron Ltd.), using CF₄/Ar gas until the oxide film was etched by 50 nm. Evaluation of defects were carried out for the wafer prepared by etching with a defect inspection apparatus “Surfscan SP5” (product name, manufactured by KLA), and the number of cone defects with a size of 19 nm or more was determined as an index of the etching defect.

### (Evaluation Criteria)

A: Number of cone defects≤less than 10
B: 10<number of cone defects≤80
C: 80<number of cone defects≤400
D: 400<number of cone defects

TABLE 4

| | | | Evaluation | | | |
|---|---|---|---|---|---|---|
| | | | EUV | | | |
| | | | sensitivity - | Change | EB pattern - | |
| | | | aqueous | of | aqueous | |
| | | | TMAH | sensitivity | TMAH | |
| | | Structural | solution | due to | solution | Etching |
| | Polymer | formula | development | aging | development | defect |
| Example B1 | B1 | MA1 | 20 | B | B | B |
| Example B1P | B1P | MA1 | 18 | A | A | A |
| Example B2 | B2 | MA2 | 22 | B | B | B |
| Example B2P | B2P | MA2 | 19 | A | A | A |
| Example B3P | B3P | MA4 | 20 | A | A | A |
| Example B4P | B4P | MA5 | 20 | A | A | A |
| Example B5P | B5P | MA6 | 20 | A | A | A |
| Example B6P | B6P | MA7 | 20 | A | A | A |
| Example B7P | B7P | MA2 | 20 | A | A | A |
| Comparative | BR1 | MAR1 | 49 | C | C | C |
| Example BR1 | | | | | | |
| Reference | BX1 | MX1 | 26 | A | A | A |
| Example BX1 | | | | | | |

TABLE 4-2

| | | | Evaluation | | | |
|---|---|---|---|---|---|---|
| | | | EUV | | | |
| | | | sensitivity - | Change | EB pattern - | |
| | | | aqueous | of | aqueous | |
| | | | TMAH | sensitivity | TMAH | |
| | | Structural | solution | due to | solution | Etching |
| | Polymer | formula | development | aging | development | defect |
| Example BD1 | BD1 | PMD1 | 17 | A | A | A |
| Example BD2 | BD2 | PMD2 | 17 | A | A | A |
| Example BD3 | BD3 | PMD3 | 17 | A | A | A |
| Example BD4 | BD4 | PMD4 | 17 | A | A | A |
| Example BD5 | BD5 | PMD5 | 16 | A | A | A |
| Example BD6 | BD6 | PMD6 | 16 | A | A | A |
| Example BD7 | BD7 | PMD7 | 16 | A | A | A |
| Example BD8 | BD8 | PMD8 | 16 | A | A | A |
| Example BD9 | BD9 | PMD9 | 16 | A | A | A |
| Example BD10 | BD10 | PMD10 | 16 | A | A | A |
| Example BD11 | BD11 | PMD11 | 16 | A | A | A |
| Example BD12 | BD12 | PMD12 | 16 | A | A | A |
| Example BD13 | BD13 | PMD13 | 15 | A | A | A |
| Example BD14 | BD14 | PMD14 | 15 | A | A | A |
| Example BD15 | BD15 | PMD15 | 15 | A | A | A |

TABLE 4-3

| | | | Evaluation | | | |
|---|---|---|---|---|---|---|
| | | | EUV | | | |
| | | | sensitivity - | Change | EB pattern - | |
| | | | aqueous | of | aqueous | |
| | | | TMAH | sensitivity | TMAH | |
| | | Structural | solution | due to | solution | Etching |
| | Polymer | formula | development | aging | development | defect |
| Example BD16 | BD16 | PMD16 | 16 | A | A | A |
| Example BD17 | BD17 | PMD17 | 15 | A | A | A |
| Example BD18 | BD18 | PMD18 | 16 | A | A | A |
| Example BD19 | BD19 | PMD19 | 15 | A | A | A |
| Example BD20 | BD20 | PMD20 | 16 | A | A | A |
| Example BD21 | BD21 | PMD21 | 15 | A | A | A |
| Example BD22 | BD22 | PMD22 | 16 | A | A | A |
| Example BD23 | BD23 | PMD23 | 16 | A | A | A |
| Example BD24 | BD24 | PMD24 | 15 | A | A | A |
| Example BD25 | BD25 | PMD25 | 17 | A | A | A |
| Example BD26 | BD26 | PMD26 | 17 | A | A | A |
| Example BD27 | BD27 | PMD27 | 16 | A | A | A |
| Example BD28 | BD28 | PMD28 | 16 | A | A | A |
| Example BD29 | BD29 | PMD29 | 17 | A | A | A |
| Example BD30 | BD30 | PMD30 | 13 | A | A | A |

### (EB Pattern 2-Aqueous TMAH Solution Development)

A solution was prepared by blending 8 parts by mass of the compound or the polymer obtained in Examples or Comparative Examples, 1 part by mass of triphenylsulfonium nonafluoromethanesulfonate, 1 part by mass of triphenylsulfonium trifluoromethanesulfonate, 0.2 parts by mass of tributylamine, and 92 parts by mass of PGMEA.

The solution was applied on a silicon wafer, which was baked at 120° C. for 60 seconds to form a resist film having a film thickness of 80 nm.

Then, the resist film was exposed using an electron beam lithography system “ELS-7500” (product name, manufactured by ELIONIX INC., 50 keV), baked (PEB) at 110° C. for 90 seconds, and developed with a 2.38 mass % tetramethylammonium hydroxide (TMAH) aqueous solution for 60 seconds to obtain a positive type pattern. The exposure amount was regulated to have a line and space pattern with a half pitch of 30 nm.

For the obtained resist pattern, 80 pattern images were obtained with a scanning electron microscope “S-4800” (product name, manufactured by Hitachi, Ltd.) at 100000 times magnification, the number of residues on the space portion between resist patterns were counted, and evaluations were carried out from the total amount of the residues. The evaluation criteria are as follows.

### (Evaluation Criteria)

A: Number of residues≤less than 10
B: 10<number of residues≤80
C: 80<number of residues≤400
D: 400<number of residues

Furthermore, sampling for the line width of the obtained pattern image was carried out at 100 arbitrary points, the standard deviation of the variation value of the line width, the line width cy, was determined, and evaluations were carried out under the following evaluation criteria.

### (Evaluation Criteria)

A: Line width σ≤less than 2 nm
B: 2 nm<line width σ≤4 nm
C: 4 nm<line width σ≤7 nm
D: 7 nm<line width σ

TABLE 4-4

| | | | Evaluation EB pattern 2 - aqueous | |
|---|---|---|---|---|
| | | | TMAH solution development | |
| | | Structural | Number of | Line |
| | Polymer | formula | residues | width σ |
| Example B1P | B1P | MA1 | A | A |
| Example B2P | B2P | MA2 | A | A |
| Example B3P | B3P | MA4 | A | A |
| Example B4P | B4P | MA5 | A | A |
| Example B5P | B5P | MA6 | A | A |
| Example B6P | B6P | MA7 | A | A |
| Example B7P | B7P | MA2 | A | A |
| Reference | BX1 | MX1 | B | B |
| Example BX1 | | | | |

The above results show that excellent resolution of a line and space pattern, in particular, with a thin line is obtained by introducing the compound of the present invention in the second embodiment.

### (EUV Sensitivity—Organic Solvent Development)

A solution containing the compound or the polymer obtained in Examples or Comparative Examples was prepared in the same manner as the EUV sensitivity-aqueous TMAH solution development, which was applied on a silicon wafer and baked at 110° C. for 60 seconds to form a photoresist layer having a film thickness of 100 nm.

Then, using an extreme ultraviolet (EUV) exposure apparatus “EUVES-7000” (product name, manufactured by Litho Tech Japan Corporation), the photoresist layer was subjected to shot exposure without masking by incrementing the exposure amount by 1 mJ/cm² from 1 mJ/cm² to 80 mJ/cm², baked (PEB) at 110° C. for 90 seconds, and developed with butyl acetate for 30 seconds to obtain a wafer on which 80 shots of shot exposure was carried out. For each obtained shot exposure area, the film thickness was measured by using an optical interference film thickness meter “VM3200” (product name, manufactured by SCREEN Semiconductor Solutions Co., Ltd.), profile data about the film thickness relative to the exposure amount was obtained, and the exposure amount at which the slope of film thickness variation relative to an exposure amount was the largest was calculated as a sensitivity value (mJ/cm²), which was used as an index of the EUV sensitivity of the resist.

### (EB Pattern-Organic Solvent Development)

A solution containing the compound or the polymer obtained in Examples or Comparative Examples was prepared in the same manner as the EB pattern-aqueous TMAH solution development, which was applied on a silicon wafer and baked at 110 to 130° C. for 60 seconds to form a resist film having a film thickness of 100 nm.

Then, the resist film was exposed using an electron beam lithography system “ELS-7500” (product name, manufactured by ELIONIX INC., 50 keV), baked (PEB) at 115° C. for 90 seconds, and developed with butyl acetate for 30 seconds to obtain a negative type pattern. The exposure amount was regulated to have a line and space pattern with a half pitch of 50 nm.

For the obtained resist pattern, 80 pattern images were obtained with a scanning electron microscope “S-4800” (product name, manufactured by Hitachi, Ltd.) at 100000 times magnification, the number of residues on the space portion between resist patterns were counted, and evaluations were carried out from the total amount of the residues. The evaluation criteria are as follows.

### (Evaluation Criteria)

A: Number of residues≤less than 10
B: 10<number of residues≤80
C: 80<number of residues≤400
D: 400<number of residues

TABLE 5

| | | | EUV sensitivity - | EB pattern - |
|---|---|---|---|---|
| | | Structural | organic solvent | organic solvent |
| | Polymer | formula | development | development |
| Example B1 | B1 | MA1 | 22 | B |
| Example B1P | B1P | MA1 | 20 | A |
| Example B2 | B2 | MA2 | 20 | B |
| Example B2P | B2P | MA2 | 18 | A |
| Example B3P | B3P | MA4 | 22 | A |
| Example B4P | B4P | MA5 | 22 | A |
| Example B5P | B5P | MA6 | 22 | A |
| Example B6P | B6P | MA7 | 22 | A |
| Example B7P | B7P | MA2 | 22 | A |
| Comparative | BR1 | MAR1 | 55 | C |
| Example BR1 | | | | |
| Reference | BX1 | MX1 | 29 | A |
| Example BX1 | | | | |

TABLE 5-2

| | | | EUV | |
|---|---|---|---|---|
| | | | sensitivity - | EB pattern - |
| | | | organic | organic |
| | | Structural | solvent | solvent |
| | Polymer | formula | development | development |
| Example BD1 | BD1 | PMD1 | 17 | |
| Example BD2 | BD2 | PMD2 | 17 | A |
| Example BD3 | BD3 | PMD3 | 17 | A |
| Example BD4 | BD4 | PMD4 | 17 | A |
| Example BD5 | BD5 | PMD5 | 16 | A |
| Example BD6 | BD6 | PMD6 | 16 | A |
| Example BD7 | BD7 | PMD7 | 16 | A |
| Example BD8 | BD8 | PMD8 | 16 | A |
| Example BD9 | BD9 | PMD9 | 16 | A |
| Example BD10 | BD10 | PMD10 | 16 | A |
| Example BD11 | BD11 | PMD11 | 16 | A |
| Example BD12 | BD12 | PMD12 | 16 | A |
| Example BD13 | BD13 | PMD13 | 17 | A |
| Example BD14 | BD14 | PMD14 | 17 | |
| Example BD15 | BD15 | PMD15 | 17 | A |

The description of Example Group 1 is as above.

### Example Group 2

### Example Group 2: Example A1

Into a 3 L glass flask as a reaction vessel, 283 g (792 mmol) of triphenylphosphonium methyl bromide, 7 mg of methylhydroquinone, and 1470 mL of dehydrated THF were put and dissolved. While the temperature was regulated to 15° C. or less, 148 g (1320 mmol) of potassium tert-butoxide was added portionwise into a THF solution which was put in an ice bath, and then the mixture was stirred for 30 minutes as it was. While the temperature was further regulated to 25° C. or less, 131 g (529 mmol) of 4-hydroxy-3-iodobenzenecarbaldehyde was added portionwise, and then the mixture was stirred for 30 minutes as it was. Thereafter, the reaction solution was added to 4000 mL of a 3N aqueous HCl solution, and then the mixture was further washed with 1 L of toluene and 2 L of water in the order presented. 104 g of 4-hydroxy-3-iodostyrene represented by the formula (M1) as the target substance was isolated using a silica gel column.

As a result of the analysis by liquid chromatography-mass spectrometry (LC-MS), the molecular weight was found to be 246.

The following peaks were found by carrying out ¹H-NMR measurement under the above measurement conditions, and the compound was confirmed to have the chemical structure of the compound A1 represented by the formula (M1).

δ (ppm) (d6-DMSO): 9.4 (1H, —OH), 7.7 (3H, Ph), 6.7 (1H, —CH═), 5.6 (1H, ═CH2), 5.3 (1H, ═CH2)

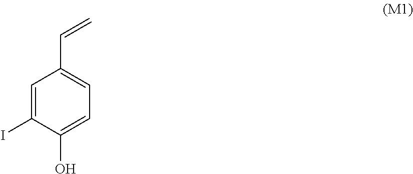

### Example Group 2: Example A1-A

### (Step 1) Malonic Acid Addition Reaction

Using a 200 mL eggplant flask equipped with a Dienstark reflux tube, 9.4 g (38 mmol) of 4-hydroxy-3-iodobenzaldehyde was mixed with dimethyl malonate (10.6 g, 80 mmol), piperidine (3.4 g, 40 mmol), acetic acid (2.4 g, 40 mmol), and 40 mL of benzene, and allowed to react for 3 hours under reflux conditions. The obtained reaction solution was washed with 20 mL of a 5% by mass aqueous HCl solution, and then washed with a 5% aqueous NaHCO₃ solution. The obtained organic phase was dried over magnesium sulfate, and then concentrated under reduced pressure to obtain 10.5 g of a reaction product (M1-1).

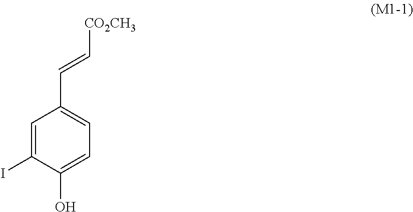 [SMILES: COC(=O)/C=C/c1ccc(O)c(I)c1]

### (Step 2) Hydrolysis Reaction

Using a 1 L eggplant flask equipped with a reflux tube, hydrochloric acid (6N, 131 mL) and acetic acid (131 mL) were added to 38 mmol of the product (M1-1) obtained above, and the mixture was refluxed for 48 hours. Thereafter, 6M, 500 mL of NaOH aq. was added, and the mixture was extracted with 250 mL of ethyl acetate to recover the organic phase consisting of ethyl acetate. The obtained organic phase was subjected to a dehydration treatment with magnesium sulfate, and the filtrate filtered thereafter was concentrated under reduced pressure to obtain 10.1 g of a cinnamic acid derivative (M1-2).

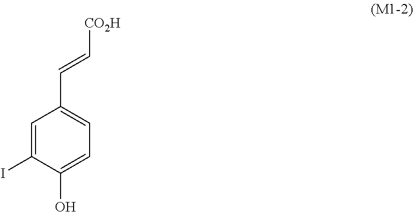 [SMILES: O=C(O)/C=C/c1ccc(O)c(I)c1]

### (Step 3) Decarboxylation Reaction

Using a 1 L eggplant flask, a solution in which 0.13 g (0.4 mmol) of tetrabutylammonium fluoride trihydrate was dissolved in 20 mL of dimethylsulfoxide was slowly added to a solution in which 40 mmol of the cinnamic acid derivative (M1-2) prepared above was dissolved in 40 mL of dimethylsulfoxide at 10° C. and stirred, and then the mixture was warmed to 40° C. and stirred for 12 hours. The obtained reaction solution was washed three times with 20 mL of pure water, dried over magnesium sulfate, and the filtrate obtained after filtration was concentrated under reduced pressure to obtain 9.2 g of a compound A1 represented by the formula (M1).

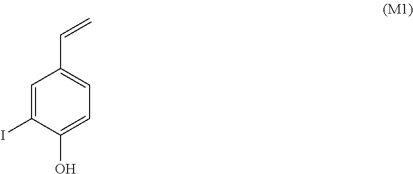

### Example Group 2: Example A2

The reaction was carried out in the same manner as Example A1, except that 4-hydroxy-3-iodobenzenecarbaldehyde was changed to 4-methoxy-3-iodobenzenecarbaldehyde, thereby isolating 129 g of 3-iodo-4-methoxystyrene represented by the formula (M2) as the target substance.

As a result of the analysis by liquid chromatography-mass spectrometry (LC-MS), the molecular weight was found to be 260.

The following peaks were found by carrying out ¹H-NMR measurement under the above measurement conditions, and the compound was confirmed to have the chemical structure of the compound represented by the formula (M2).

δ (ppm) (d6-DMSO): 3.7 (3H, —CH3), 4.1 (2H, —CH2-), 7.7 (2H, Ph), 6.7 (1H, —CH═), 5.3 (1H, ═CH2), 5.7 (1H, ═CH2)

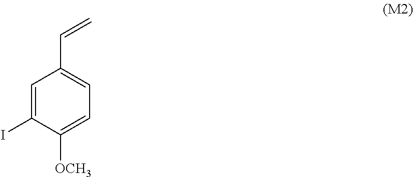

### Example Group 2: Example A1-B

### (Step 1) Synthesis of 4′-hydroxy-3′-iodoacetophenone

A reactor was charged with 50.20 g of 4′-hydroxy-acetophenone, 91.38 g of iodine, 1,620 mL of methanol, and 180 mL of pure water, the reactor was immersed in an ice bath, and stirring was initiated. Subsequently, 44.06 g of an aqueous iodic acid solution with a mass percent concentration of 71.9 was added dropwise thereto for 30 minutes. Subsequently, the reactor was immersed in a water bath at 35° C., and stirring was continued over 3.5 hours. Subsequently, 13.37 g of an aqueous sodium hydrogen sulfite solution with a mass percent concentration of 35 was added to quench the reaction. Subsequently, the content of the reactor was gradually added to 3,600 mL of pure water with vigorous stirring and mixed. Subsequently, the precipitate was filtered off with a suction filter, compressed, and washed with 540 mL of an aqueous methanol solution with a volume percent concentration of 33.3. Subsequently, the precipitate was dried in vacuo at 40° C. to obtain 141.1 g of 4′-hydroxy-3′-iodoacetophenone. The yield was 90.1%.

As a result of the analysis by liquid chromatography-mass spectrometry (LC-MS), the molecular weight was found to be 262, and the substance was confirmed to be 4′-hydroxy-3′-iodoacetophenone.

The following peaks were found by carrying out ¹H-NMR measurement under the above measurement conditions, and the compound was confirmed to have the chemical structure of 4′-hydroxy-3′-iodoacetophenone.

δ (ppm) (d6-DMSO): 10.5 (1H, OH), 8.3 (3H, Ph), 2.5 (3H, —CH3)

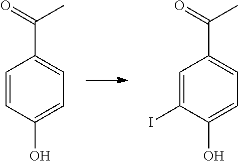

### (Step 2) Synthesis of 1-(4-hydroxy-3-methoxyphenyl)ethanol

The reactor was charged with 8.77 g of sodium borohydride and 180 mL of tetrahydrofuran, the reactor was immersed in an ice bath, and stirring was initiated. Subsequently, a mixed solution consisting of 17.20 g of 4′-hydroxyacetophenone, 9.32 g of isopropanol, and 180 mL of tetrahydrofuran were added dropwise thereto over 3 hours. Subsequently, stirring was continued over 8 hours while the reactor was immersed in the ice bath. Subsequently, 59.47 g of methanol was added to quench the reaction. Subsequently, the pressure in the reactor was reduced to 50 hPa, immersed in a water bath at 20° C. to concentrate the reaction solution. Subsequently, the reactor was immersed in an ice bath, and 120 mL of cold methanol was added to dilute the reaction solution. Subsequently, the pressure in the reactor was reduced to 50 hPa, immersed in a water bath at 20° C. to concentrate the reaction solution. Subsequently, the reactor was immersed in an ice bath, and 600 mL of cold methanol was added to dilute the reaction solution. Subsequently, the reaction solution was gradually added to 1,200 g of dilute sulfuric acid with a mass percent concentration of 1 with vigorous stirring and mixed. Subsequently, the precipitate was filtered off with a suction filter, compressed, and washed with 300 mL of an aqueous methanol solution with a volume percent concentration of 33.3. Subsequently, the precipitate was dried in vacuo at 40° C. to obtain 16.4 g of 1-(4-hydroxyphenyl)ethanol. The yield was 93.8%.

As a result of the analysis by liquid chromatography-mass spectrometry (LC-MS), the molecular weight was found to be 138, and the substance was confirmed to be 1-(4-hydroxyphenyl)ethanol.

The following peaks were found by carrying out ¹H-NMR measurement under the above measurement conditions, and the compound was confirmed to have the chemical structure of 1-(4-hydroxyphenyl)ethanol.

δ (ppm) (d6-DMSO): 9.4 (1H, —OH), 7.7 (4H, Ph), 5.2 (1H, —CH—OH), 4.6 (1H, —CH—OH), 1.3 (3H, —CH3)

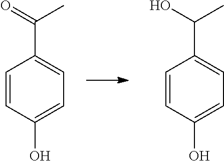

### (Step 3-1) Synthesis of 1-(4-hydroxy-3-iodophenyl)ethanol

A reactor was charged with 0.9800 g of 1-(4-hydroxyphenyl)ethanol, 1.7630 g of iodine, and 17.37 mL of methanol, the reactor was immersed in an ice bath, and stirring was initiated. Subsequently, 0.8736 g of an aqueous iodic acid solution with a mass percent concentration of 70 was added dropwise thereto for 30 minutes. Subsequently, the reactor was immersed in a water bath at 25° C., and stirring was continued over 3.5 hours. Subsequently, 0.174 mL of an aqueous sodium hydrogen sulfite solution with a mass percent concentration of 35 was added to quench the reaction. Subsequently, the reaction solution was gradually added to 34.74 mL of pure water with vigorous stirring and mixed. Subsequently, the precipitate was filtered off with a suction filter, compressed, and washed with an aqueous methanol solution with a volume percent concentration of 33.3. Subsequently, the precipitate was dried in vacuo at 40° C. to obtain 2.7808 g of a mixture of 1-(4-hydroxy-3-iodophenyl)ethanol and 2-iodo-4-(1-methoxyethyl)phenol. As a result of the HPLC analysis using a UV detector at a measurement wavelength of 254 nm, the ratio between 1-(4-hydroxy-3-iodophenyl)ethanol and 2-iodo-4-(1-methoxyethyl)phenol was 50.66:47.14.

As a result of the analysis by liquid chromatography-mass spectrometry (LC-MS), the molecular weights were found to be 264 and 278, and the substance was confirmed to be a mixture of 1-(4-hydroxy-3-iodophenyl)ethanol and 2-iodo-4-(1-methoxyethyl)phenol.

The following peaks were found by carrying out ¹H-NMR measurement under the above measurement conditions, and the compound was confirmed to have the chemical structures of 1-(4-hydroxy-3-iodophenyl)ethanol and 2-iodo-4-(1-methoxyethyl)phenol.

δ (ppm) (d6-DMSO): 9.4 (1H, —OH), 7.7 (3H, Ph), 5.2 (0.5H, —CH—OH), 4.6-4.3 (1H, —CH—OH), 3.0 (1.5H, —O—CH3), 1.3 (3H, —CH3)

### (Step 3-2) Synthesis of 1-(4-hydroxy-3-iodo-5-methoxyphenyl)ethanol

A reactor was charged with 0.9759 g of 1-(4-hydroxyphenyl)ethanol, 1.7472 g of iodine, 15.48 mL of methanol, and 1.72 mL of pure water, the reactor was immersed in an ice bath, and stirring was initiated. Subsequently, 0.8687 g of an aqueous iodic acid solution with a mass percent concentration of 70 was added dropwise thereto for 30 minutes. Subsequently, the reactor was immersed in a water bath at 25° C., and stirring was continued over 3.5 hours. Subsequently, 0.172 mL of an aqueous sodium hydrogen sulfite solution with a mass percent concentration of 35 was added to quench the reaction. Subsequently, the reaction solution was gradually added to 34.40 mL of pure water with vigorous stirring and mixed. Subsequently, the precipitate was filtered off with a suction filter, compressed, and washed with an aqueous methanol solution with a volume percent concentration of 33.3. Subsequently, the precipitate was dried in vacuo at 40° C. to obtain 2.7857 g of a mixture of 1-(4-hydroxy-3-iodophenyl)ethanol and 2-iodo-4-(1-methoxyethyl)phenol. As a result of the HPLC analysis using a UV detector at a measurement wavelength of 254 nm, the ratio between 1-(4-hydroxy-3-iodophenyl)ethanol and 2-iodo-4-(1-methoxyethyl)phenol was 83.11:16.00.

As a result of the analysis by liquid chromatography-mass spectrometry (LC-MS), the molecular weights were found to be 264 and 278, and the substance was confirmed to be a mixture of 1-(4-hydroxy-3-iodophenyl)ethanol and 2-iodo-4-(1-methoxyethyl)phenol.

### (Step 3-3) Synthesis of 1-(4-hydroxy-3-iodophenyl)ethanol

A reactor was charged with 0.9928 g of 1-(4-hydroxyphenyl)ethanol, 1.7787 g of iodine, 14.00 mL of methanol, and 3.50 mL of pure water, the reactor was immersed in an ice bath, and stirring was initiated. Subsequently, 0.8795 g of an aqueous iodic acid solution with a mass percent concentration of 70 was added dropwise thereto for 30 minutes. Subsequently, the reactor was immersed in a water bath at 25° C., and stirring was continued over 3.5 hours. Subsequently, 0.175 mL of an aqueous sodium hydrogen sulfite solution with a mass percent concentration of 35 was added to quench the reaction. Subsequently, the reaction solution was gradually added to 35.00 mL of pure water with vigorous stirring and mixed. Subsequently, the precipitate was filtered off with a suction filter, compressed, and washed with an aqueous methanol solution with a volume percent concentration of 33.3. Subsequently, the precipitate was dried in vacuo at 40° C. to obtain 2.8425 g of a mixture of 1-(4-hydroxy-3-iodophenyl)ethanol and 2-iodo-4-(1-methoxyethyl)phenol. As a result of the HPLC analysis using a UV detector at a measurement wavelength of 254 nm, the ratio between 1-(4-hydroxy-3-iodophenyl)ethanol and 2-iodo-4-(1-methoxyethyl)phenol was 73.82:25.28.

As a result of the analysis by liquid chromatography-mass spectrometry (LC-MS), the molecular weights were found to be 264 and 278, and the substance was confirmed to be a mixture of 1-(4-hydroxy-3-iodophenyl)ethanol and 2-iodo-4-(1-methoxyethyl)phenol.

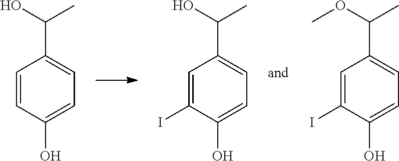

### (Step 4) Synthesis of 1-(4-hydroxy-3-iodophenyl)ethanol

The reactor was charged with 8.77 g of sodium borohydride and 180 mL of tetrahydrofuran, the reactor was immersed in an ice bath, and stirring was initiated. Subsequently, a mixed solution consisting of 53.84 g of 4′-hydroxy-3′-iodoacetophenone, 9.31 g of isopropanol, and 180 mL of tetrahydrofuran were added dropwise thereto over 3 hours. Subsequently, stirring was continued over 9 hours while the reactor was immersed in the ice bath. Subsequently, 59.47 g of methanol was added to quench the reaction. Subsequently, the pressure in the reactor was reduced to 50 hPa, immersed in a water bath at 20° C. to concentrate the reaction solution. Subsequently, the reactor was immersed in an ice bath, and 120 mL of cold methanol was added to dilute the reaction solution. Subsequently, the pressure in the reactor was reduced to 50 hPa, immersed in a water bath at 20° C. to concentrate the reaction solution. Subsequently, the reactor was immersed in an ice bath, and 600 mL of cold methanol was added to dilute the reaction solution. Subsequently, the reaction solution was gradually added to 1,200 g of dilute sulfuric acid with a mass percent concentration of 1 with vigorous stirring and mixed. Subsequently, the precipitate was filtered off with a suction filter, compressed, and washed with 300 mL of an aqueous methanol solution with a volume percent concentration of 33.3. Subsequently, the precipitate was dried in vacuo at 40° C. to obtain 48.27 g of 1-(4-hydroxy-3-iodophenyl)ethanol. The yield was 89.1%. As a result of the analysis by liquid chromatography-mass spectrometry (LC-MS), the molecular weight was found to be 264, and the substance was confirmed to be 1-(4-hydroxy-3-iodophenyl)ethanol.

The following peaks were found by carrying out ¹H-NMR measurement under the above measurement conditions, and the compound was confirmed to have the chemical structure of 1-(4-hydroxy-3-iodophenyl)ethanol.

δ (ppm) (d6-DMSO): 9.4 (1H, —OH), 7.7 (3H, Ph), 5.2 (1H, —CH—OH), 4.6 (1H, —CH—OH), 1.3 (3H, —CH3)

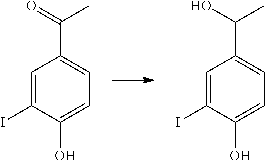

### (Step 5-1) Synthesis of 4-hydroxy-3-iodostyrene

The reactor was charged with 98.57 g of 1-(4-hydroxyphenyl)ethanol, 7.94 g of concentrated sulfuric acid, 0.30 g of 4-hydroxy-2,2,6,6-tetramethylpiperidinyl-oxyl free radical, and 1,500 mL of dimethylsulfoxide, and stirring was initiated. Subsequently, the pressure in the reactor was reduced to 30 hPa, and blowing of air into the reaction solution at a flow rate of 9 mL/minute was initiated. Subsequently, the reactor was immersed in a water bath at 90° C., and stirring was continued over 5 hours. Subsequently, the reactor was immersed in a water bath at 25° C. to cool the reaction solution. Subsequently, the reaction solution was gradually added to 3,000 g of an aqueous sodium hydrogen sulfite solution with a mass percent concentration of 0.1 with vigorous stirring and mixed. Subsequently, the precipitate was filtered off with a suction filter, compressed, and washed with 1,500 mL of an aqueous methanol solution with a volume percent concentration of 33.3. Subsequently, the precipitate was dried in vacuo at 40° C. to obtain 97.76 g of 4-hydroxy-3-iodostyrene. The yield was 95.7%.

As a result of the analysis by liquid chromatography-mass spectrometry (LC-MS), the molecular weight was found to be 246, and the substance was confirmed to be 4-hydroxy-3-iodostyrene.

The following peaks were found by carrying out ¹H-NMR measurement under the above measurement conditions, and the compound was confirmed to have the chemical structure of 4-hydroxy-3-iodostyrene.

δ (ppm) (d6-DMSO): 9.5 (1H, —OH), 7.7 (3H, Ph), 6.7 (1H, —CH═), 5.7 (1H, ═CH2), 5.3 (1H, ═CH2)

### (Step 5-2) Synthesis of 4-hydroxy-3-iodostyrene

The reactor was charged with 1.8000 g of a mixture in which the ratio between 1-(4-hydroxy-3-iodophenyl)ethanol and 2-iodo-4-(1-methoxyethyl)phenol is 73.82:23.28, 0.2895 mL of concentrated sulfuric acid, 0.0020 g of 4-hydroxy-2,2,6,6-tetramethylpiperidine1-oxyl free radical, and 20 mL of dimethylsulfoxide, and stirring was initiated. Subsequently, the pressure in the reactor was reduced to 30 hPa, the reactor was immersed in a water bath at 90° C., and stirring was continued over 3 hours. Subsequently, the reactor was immersed in a water bath at 25° C. to cool the reaction solution. As a result of the HPLC analysis using a UV detector at a measurement wavelength of 254 nm, the ratio among 1-(4-hydroxy-3-iodophenyl)ethanol, 2-iodo-4-(1-methoxyethyl)phenol, and 4-hydroxy-3-iodostyrene in the reaction solution was 0.08:0.01:98.12.

As a result of the analysis by liquid chromatography-mass spectrometry (LC-MS), the molecular weight was found to be 246, and the substance was confirmed to be 4-hydroxy-3-iodostyrene.

The compound was confirmed to have a similar chemical structure by carrying out ¹H-NMR measurement under the above measurement conditions.

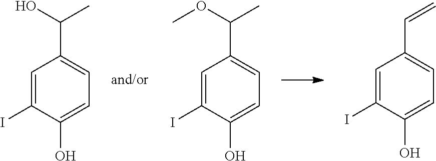

### Example Group 2: Synthetic Example 1

### Synthesis of 4-acetoxy-3-iodostyrene

Using a 100 mL glass flask as a reaction vessel, 14.9 g (45 mmol) of 4-hydroxy-3-iodostyrene was dissolved by using dimethylsulfoxide as a solvent, 2 eq. of acetic anhydride and 1 eq. of sulfuric acid were added thereto, and then the mixture was warmed to 80° C. and stirred for 3 hours. Thereafter, the stirred solution was cooled, and the precipitate was filtered off, washed, and dried to obtain 9.0 g of a white solid. The white solid sample was analyzed by liquid chromatography-mass spectrometry (LC-MS) and, as a result, the molecular weight was found to be 414 and the white solid was confirmed to be 4-acetoxy-3-iodostyrene.

The following peaks were found by carrying out ¹H-NMR measurement under the above measurement conditions, and the compound was confirmed to have the chemical structure of 4-acetoxy-3-iodostyrene.

δ (ppm) (d6-DMSO): 7.9 (3H, Ph), 6.6 (1H, —CH2-), 5.7 (1H, ═CH2), 5.1 (1H, ═CH2), 2.3 (3H, —CH3)

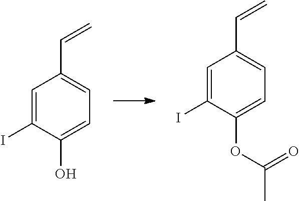

### Example Group 2: Example A3

In a 2 L flask, 400 mL of dichloromethane, 36 g of the obtained compound A1, 16.2 g of triethylamine, and 0.7 g of N-(4-pyridyl)dimethylamine (DMAP) was dissolved in a nitrogen flow. 33.6 g of di-tert-butyl dicarbonate was dissolved in 100 mL of dichloromethane, which was added dropwise into the aforementioned 2 L flask with stirring, and the mixture was stirred at room temperature for 3 hours. Thereafter, water washing was conducted three times by a separation operation using 100 mL of water, the solvent was distilled off from the obtained organic phase, original components were removed by silica gel chromatography using dichloromethane/hexane, and the solvent was further distilled off to obtain 4.0 g of a BOC group-substituted compound (a compound represented by the following formula (M3), hereinafter, also referred to as the “compound A3”) of the compound A1, as the target component.

As a result of the analysis by liquid chromatography-mass spectrometry (LC-MS), the molecular weight was found to be 346.

The following peaks were found by carrying out ¹H-NMR measurement under the above measurement conditions, and the compound was confirmed to have the chemical structure of the compound represented by the formula (M3).

δ (ppm) (d6-DMSO): 7.7 (2H, Ph), 6.7 (1H, —CH═), 5.7 (1H, ═CH2), 5.3 (1H, ═CH2), 1.4 (9H, —C—(CH3)3)

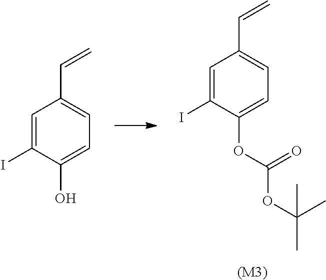

### Example Group 2: Example A4

In a container (internal capacity: 200 mL) equipped with a stirrer, a condenser tube, and a burette, 100 mL of acetone was charged with 3.05 g (12.4 mmol) of the compound A1 obtained in Example A1 and 2.42 g (12.4 mmol) of ethylvinyl ether, 2.5 g of pyridinium p-toluenesulfonate was added, and the contents were reacted by being stirred at room temperature for 24 hours to obtain a reaction solution. Next, the reaction solution was concentrated, and the reaction product was filtered to separate solid matter.

The obtained solid matter was filtered and then dried, and then the solid matter was separated and purified by column chromatography to obtain 1.9 g of the compound A4 (a compound represented by the following formula (M4)).

As a result of the analysis by liquid chromatography-mass spectrometry (LC-MS), the molecular weight was found to be 318. The following peaks were found by carrying out ¹H-NMR measurement under the above measurement conditions, and the compound was confirmed to have the chemical structure of the compound represented by the formula (M4).

δ (ppm) (d6-DMSO): 7.7 (3H, Ph), 6.7 (1H, —CH═), 5.7 (1H, ═CH2), 5.6 (1H, CH3CH—), 5.3 (1H, ═CH2), 3.9 (2H, CH3CH2-), 1.6 (3H, CH3CH—), 1.2 (3H, CH3CH2-)

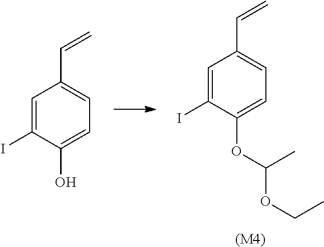

### Example Group 2: Example A5

In a container (internal capacity: 200 mL) equipped with a stirrer, a condenser tube, and a burette, 3.05 g (12.4 mmol) of the compound A1 obtained in Example A1 and 2.42 g (12.4 mmol) of tetrahydropyran were charged to 100 mL of acetone, 2.5 g of pyridinium p-toluenesulfonate was added, and the contents were reacted by being stirred at room temperature for 24 hours to obtain a reaction solution. Next, the reaction solution was concentrated, and the reaction product was filtered to separate solid matter.

The obtained solid matter was filtered and then dried, and then the solid matter was separated and purified by column chromatography to obtain 2.1 g of the compound A4 (a compound represented by the following formula (M4)).

The following peaks were found by carrying out ¹H-NMR measurement under the above measurement conditions, and the compound was confirmed to have the chemical structure of the compound represented by the formula (M5).

As a result of the analysis by liquid chromatography-mass spectrometry (LC-MS), the molecular weight was found to be 318.

δ (ppm) (d6-DMSO): 7.7 (2H, Ph), 6.7 (1H, —CH═), 5.8 (1H, proton of tetrahydropyranyl group ═CH—), 5.7 (1H, ═CH2), 5.3 (1H, ═CH2), 1.6-3.7 (8H, protons of tetrahydropyranyl group —CH2-)

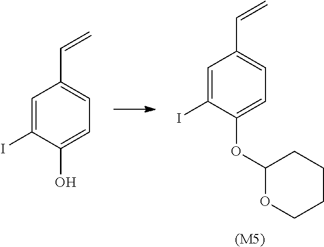

### (Example Group 2: Example A6) Synthesis of Compound A6 Represented by Formula (M6)

In a container (internal capacity: 200 mL) equipped with a stirrer, a condenser tube, and a burette, 3.05 g (12.4 mmol) of the compound A1 obtained in the above Example A1 and 2.42 g (12.4 mmol) of tert-butyl bromoacetate were charged to 100 mL of acetone, 1.71 g (12.4 mmol) of potassium carbonate and 0.4 g of 18-crown-6 (IUPAC name: 1,4,7,10,13,16-hexaoxacyclooctadecane) was added, and the contents were reacted by being stirred under reflux for 3 hours to obtain a reaction liquid. Next, the reaction solution was concentrated, and the reaction product was precipitated by the addition of 100 g of pure water to the concentrate, cooled to room temperature, and then filtered to separate solid matter.

The obtained solid matter was filtered and then dried, and then the solid matter was separated and purified by column chromatography to obtain 2.0 g of the compound A6 (a compound represented by the following formula (M6)).

As a result of the analysis by liquid chromatography-mass spectrometry (LC-MS), the molecular weight was found to be 330.

The following peaks were found by carrying out ¹H-NMR measurement under the above measurement conditions, and the compound was confirmed to have the chemical structure of the compound represented by the formula (M6).

δ (ppm) (d6-DMSO): 7.7 (3H, Ph), 6.7 (1H, —CH═), 5.7 (1H, ═CH2), 5.3 (1H, ═CH2), 5.0 (2H, —CH2-), 1.4 (9H, —C—(CH3)3)

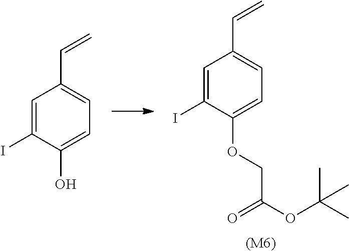

### (Example Group 2: Example A7) Synthesis of Compound A7 Represented by Formula (M7)

In a container (internal capacity: 200 mL) equipped with a stirrer, a condenser tube, and a burette, 3.05 g (12.4 mmol) of the compound A1 obtained in the above Example A1 and 2.42 g (12.4 mmol) of bromoacetic acid 2-methyl-2-adamantyl ester were charged to 100 mL of acetone, 1.71 g (12.4 mmol) of potassium carbonate and 0.4 g of 18-crown-6 (IUPAC name: 1,4,7,10,13,16-hexaoxacyclooctadecane) was added, and the contents were reacted by being stirred under reflux for 3 hours to obtain a reaction liquid. Next, the reaction solution was concentrated, and the reaction product was precipitated by the addition of 100 g of pure water to the concentrate, cooled to room temperature, and then filtered to separate solid matter.

The obtained solid matter was filtered and then dried, and then the solid matter was separated and purified by column chromatography to obtain 2.1 g of the compound A7 (a compound represented by the following formula (M7)).

The following peaks were found by carrying out ¹H-NMR measurement under the above measurement conditions, and the compound was confirmed to have the chemical structure of the compound represented by the formula (M7).

As a result of the analysis by liquid chromatography-mass spectrometry (LC-MS), the molecular weight was found to be 452.

δ (ppm) (d6-DMSO): 7.7 (2H, Ph), 6.7 (1H, —CH═), 5.7 (1H, ═CH2), 5.3 (1H, ═CH2), 5.0 (2H, —CH2-), 0.8-2.4 (17H, protons of 2-methyl-2-adamantyl group)

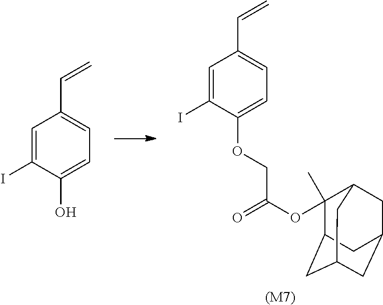

### (Example Group 2: Example A8) Synthesis of Compound A8 Represented by Formula (M8)

In a container (internal capacity: 200 mL) equipped with a stirrer, a condenser tube, and a burette, 4.61 g (12.4 mmol) of the compound A1 obtained in the above Example A1 and 1.70 g (12.4 mmol) of t-butyl bromide were charged to 100 mL of acetone, 1.71 g (12.4 mmol) of potassium carbonate and 0.4 g of 18-crown-6 (IUPAC name: 1,4,7,10,13,16-hexaoxacyclooctadecane) was added, and the contents were reacted by being stirred under reflux for 3 hours to obtain a reaction liquid. Next, the reaction solution was concentrated, and the reaction product was precipitated by the addition of 100 g of pure water to the concentrate, cooled to room temperature, and then filtered to separate solid matter.

The obtained solid matter was filtered and then dried, and then the solid matter was separated and purified by column chromatography to obtain 0.3 g of the compound A8 (a compound represented by the following formula (M8)).

The following peaks were found by carrying out ¹H-NMR measurement under the above measurement conditions, and the compound was confirmed to have the chemical structure of the compound represented by the formula (M8).

As a result of the analysis by liquid chromatography-mass spectrometry (LC-MS), the molecular weight was found to be 302.

δ (ppm) (d6-DMSO): 7.7 (2H, Ph), 6.7 (1H, —CH═), 5.7 (1H, ═CH2), 5.3 (1H, ═CH2), 1.4 (9H, —C—(CH3)3)

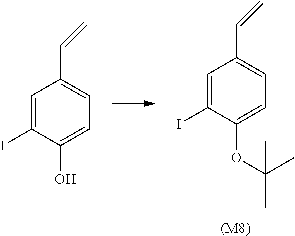

### (Example Group 2: Synthetic Example AD1a) Synthesis of Compound AD1a Represented by Formula (AD1a)

The compound AD1a represented by the formula (AD1a) was synthesized by the method described below.

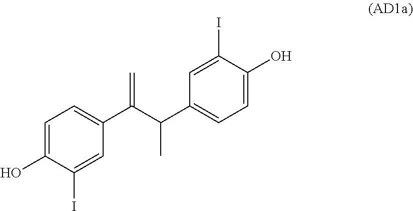 [SMILES: C=C(c1ccc(O)c(I)c1)C(C)c1ccc(O)c(I)c1]

The reactor was charged with 9.5 g of 1-(4-hydroxyphenyl)ethanol, 0.12 g of concentrated sulfuric acid, 0.04 g of 4-hydroxy-2,2,6,6-tetramethylpiperidine 1-oxyl free radical, and 1.60 mL of DMSO, and stirring was initiated. Subsequently, reduced pressure conditions were regulated to reflux at 120° C. using a Dienstark and a condenser, and blowing of air into the reaction solution at a flow rate of 1 mL/minute was initiated. The moisture recovered in the Dienstark was arbitrarily discharged to the outside of the system. Subsequently, the reactor was immersed in a water bath at 90° C., and stirring was continued over 30 hours. Subsequently, the reactor was immersed in a water bath at 25° C. to cool the reaction solution. Subsequently, the reaction solution was gradually added to 400 g of an aqueous sodium hydrogen sulfite solution with a mass percent concentration of 0.1 with vigorous stirring and mixed. Subsequently, the precipitate was filtered off with a suction filter, compressed, and washed with 200 mL of an aqueous methanol solution with a volume percent concentration of 33.3. After only main components were isolated from the obtained precipitate by column purification, the solvent was distilled off by evaporation, and the obtained solid was dried in vacuo at 40° C. to obtain 3.8 g of a white solid.

As a result of the analysis by liquid chromatography-mass spectrometry (LC-MS), the molecular weight was found to be 492, and the white solid was confirmed to be the compound AD1a represented by the formula (AD1a).

The following peaks were found by carrying out ¹H-NMR measurement under the above measurement conditions, and the compound was confirmed to have the chemical structure of the compound AD1a.

δ (ppm) (d6-DMSO): 9.6 (2H, OH), 7.5 (3H, Ph), 7.9 (3H, Ph), 5.3 (1H, ═CH2), 4.9 (1H, ═CH2), 3.5 (1H, —CH—), 1.3 (3H, —CH3)

### (Example Group 2: Synthetic Example AD1b) Synthesis of Compound AD1b Represented by Formula (AD1b)

The compound AD1b represented by the formula (AD1b) was synthesized by the method described below.

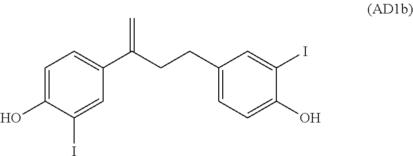 [SMILES: C=C(CCc1ccc(O)c(I)c1)c1ccc(O)c(I)c1]

The reactor was charged with 9.5 g of 1-(4-hydroxyphenyl)ethanol, 0.12 g of concentrated sulfuric acid, 0.2 g of 4-methoxyphenol, and 150 mL of toluene, and stirring was initiated. Subsequently, blowing of air into the reaction solution at a flow rate of 1 mL/minute was initiated under reflux conditions at 113° C. using a Dienstark and a condenser. The moisture recovered in the Dienstark was arbitrarily discharged to the outside of the system. Subsequently, the reactor was immersed in a water bath at 90° C., and stirring was continued over 30 hours. Subsequently, the reactor was immersed in a water bath at 25° C. to cool the reaction solution. Subsequently, the reaction solution was gradually added to 400 g of an aqueous sodium hydrogen sulfite solution with a mass percent concentration of 0.1 with vigorous stirring and mixed. Subsequently, the precipitate was filtered off with a suction filter, compressed, and washed with 200 mL of an aqueous methanol solution with a volume percent concentration of 33.3. After only main components were isolated from the obtained precipitate by column purification, the solvent was distilled off by evaporation, and the obtained solid was dried in vacuo at 40° C. to obtain 2.0 g of a white solid.

As a result of the analysis by liquid chromatography-mass spectrometry (LC-MS), the molecular weight was found to be 492, and the white solid was confirmed to be the compound AD1b represented by the formula (AD1b).

The following peaks were found by carrying out ¹H-NMR measurement under the above measurement conditions, and the compound was confirmed to have the chemical structure of the compound AD1b.

δ (ppm) (d6-DMSO): 9.6 (2H, OH), 7.6 (3H, Ph), 7.5 (3H, Ph), 5.3 (1H, ═CH2), 4.9 (1H, ═CH2), 2.6 (2H, —CH2-), 2.3 (2H, —CH2-)

### Example Group 2: Comparative Example A1

The reaction was carried out in the same manner as Example A1, except that 4-hydroxy-3-iodobenzenecarbaldehyde was changed to 4-hydroxybenzenecarbaldehyde, thereby isolating 90 g of the compound AR1 (4-hydroxystyrene) represented by the formula (MR1) as the target substance.

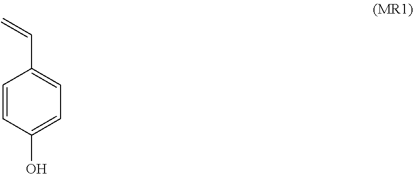

### Example Group 2: Comparative Example A2

The reaction was carried out in the same manner as Example A1, except that 4-hydroxy-3-iodobenzenecarbaldehyde was changed to 3,4-dihydroxybenzenecarbaldehyde, thereby isolating 90 g of the compound AR2 (3,4-dihydroxystyrene) represented by the formula (MR2) as the target substance.

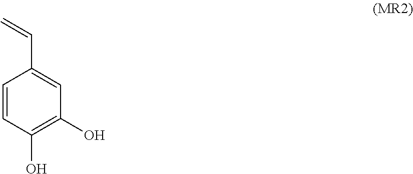

For the impurity content of the compounds synthesized in the aforementioned Examples and Comparative Examples, the inorganic element content and the organic impurity content were measured by the aforementioned method, and the results are shown in Table 1A.

TABLE 1A

| | | | Monomer physical properties | | | | |
|---|---|---|---|---|---|---|---|
| | | | Impurity content (ppm) | | | | |
| | | | | | | | Phosphorus- |
| | Compound | Structural | | | | Maleic | containing |
| | No | formula | Mn | A1 | K | acid | compound |
| Example A1 | A1 | M1 | 2 | 2 | 11 | DL | 30 |
| Example A2 | A2 | M2 | 2 | 2 | 11 | DL | 28 |
| Example A3 | A3 | M3 | 1 | 1 | 6 | DL | 10 |
| Example A4 | A4 | M4 | 1 | 1 | 6 | DL | 10 |
| Example A5 | A5 | M5 | 1 | 1 | 6 | DL | 10 |
| Example A6 | A6 | M6 | 1 | 1 | 6 | DL | 10 |
| Example A7 | A7 | M7 | 1 | 1 | 6 | DL | 10 |
| Example A8 | A8 | M8 | 2 | 2 | 6 | DL | 30 |
| Comparative | AR1 | MR1 | 2 | 2 | 11 | DL | 35 |
| Example A1 | | | | | | | |
| Comparative | AR2 | MR2 | 2 | 2 | 11 | DL | 40 |
| Example A2 | | | | | | | |

DL: below detection limit (<0.1 ppm)

### (Evaluation of Aging Stability of Composition Containing Compound)

The stability of the composition containing the compound obtained in Examples or Comparative Examples described above was evaluated using an index of the amount of change of the purity before and after the aging test in a solution state in which a single or a plurality of compounds are combined.

As the sample for evaluation, a solution in which the compounds of Examples or Comparative Examples (the compounds shown as the compound a1, the compound a2, or the compound a3) and a solvent described in Table A were mixed was prepared, which was filled in an inactivation-treated 100 mL brown glass container until 90 mL and capped to prepare a sample. As aging conditions, the sample was subjected to an aging treatment in a light-shielded constant temperature tester at 45° C. for 30 days.

For the prepared sample, the purity before and after aging treatment was measured by HPLC analysis.

The amount of change of the HPLC purity before and after aging was determined as follows and used as an index of the evaluation.

The obtained results were described in Table A.

Amount of change of purity due to aging=area % of target component before aging−area % of target component after aging

### (Evaluation Criteria)

A: Amount of change of purity due to aging≤0.2%
B: 0.2%<Amount of change of purity due to aging≤0.5%
C: 0.5%<Amount of change of purity due to aging≤1.0%
D: 1.0%<Amount of change of purity due to aging≤3.0%
E: 3.0%<Amount of change of purity due to aging

TABLE A

| | Composition | | | | | | | |
|---|---|---|---|---|---|---|---|---|
| | Compound a1 | | Compound a2 | | Compound a3 | | Solvent | Evaluation |
| | | Amount | | Amount | | Amount | Toluene | due to |
| | No. | (g) | No. | (g) | No. | (g) | (g) | aging |
| Example SL1 | A1 | 1.2025 | AR1 | 0.0975 | — | — | 13.5 | A |
| Example SL2 | A2 | 1.2025 | AR1 | 0.0975 | — | — | 13.5 | A |
| Example SL3 | A1 | 1.2987 | AR1 | 0.0013 | — | — | 13.5 | B |
| Example SL4 | A2 | 1.2987 | AR1 | 0.0013 | — | — | 13.5 | B |
| Example SL5 | A1 | 1.2025 | AR2 | 0.0975 | — | — | 13.5 | A |
| Example SL6 | A2 | 1.2025 | AR2 | 0.0975 | — | — | 13.5 | A |
| Example SL7 | A1 | 1.2025 | AD1a | 0.0975 | — | — | 13.5 | A |
| Example SL8 | A1 | 1.2025 | AD1a | 0.0975 | — | — | 13.5 | A |
| Example SL9 | A1 | 1.2025 | AD1b | 0.0975 | — | — | 13.5 | A |
| Example SL10 | A1 | 1.2025 | AD1b | 0.0480 | — | — | 13.5 | A |
| Example SL11 | A1 | 1.2987 | AD1a | 0.0007 | — | — | 13.5 | B |
| Example SL12 | A1 | 1.2987 | AD1a | 0.0013 | — | — | 13.5 | B |
| Example SL13 | A1 | 1.2987 | AD1b | 0.0007 | — | — | 13.5 | B |
| Example SL14 | A1 | 1.2987 | AD1b | 0.0007 | — | — | 13.5 | B |
| Example SL15 | A1 | 1.1391 | AR1 | 0.0681 | AD1a | 0.0928 | 13.5 | A |
| Example SL16 | A1 | 1.2922 | AR1 | 0.0039 | AD1a | 0.0039 | 13.5 | A |
| Example SL17 | A1 | 1.1391 | AR1 | 0.0681 | AD1a | 0.0464 | 13.5 | B |
| Example SL18 | A1 | 1.2922 | AR1 | 0.0039 | AD1a | 0.0020 | 13.5 | B |
| Example SL19 | A1 | 1.3 | — | — | — | — | 13.5 | C |
| Example SL20 | A2 | 1.3 | — | — | — | — | 13.5 | C |
| Example SL21 | A3 | 1.3 | — | — | — | — | 13.5 | B |
| Example SL22 | A4 | 1.3 | — | — | — | — | 13.5 | B |
| Example SL23 | A5 | 1.3 | — | — | — | — | 13.5 | B |
| Example SL24 | A6 | 1.3 | — | — | — | — | 13.5 | B |
| Example SL25 | A7 | 1.3 | — | — | — | — | 13.5 | B |
| Example SL26 | A8 | 1.2025 | AR1 | 0.0975 | — | — | 13.5 | A |

The results were obtained from Table A that determines that the compound (A) according to the embodiment can improve the stability in a solution state by containing a trace amount of the compound of the formula (1A) or the compound of the formula (1C).

### (Example Group 2: Example B1) Synthesis of Polymer

4.2 g of the compound A1, 3.0 g of 2-methyl-2-adamantyl methacrylate, 2.0 g of γ-butyrolactone methacrylate, and 1.5 g of hydroxyadamantyl methacrylate were dissolved in 45 mL of tetrahydrofuran, and 0.20 g of azobisisobutyronitrile was added thereto. After the mixture was refluxed for 12 hours, the reaction solution was added dropwise to 2 L of n-heptane. The polymer precipitated was filtered off and dried under reduced-pressure to obtain a white powdery polymer B1 represented by the following formula (MA1). This polymer had a weight average molecular weight (Mw) of 12,000 and a dispersity (Mw/Mn) of 1.90. As a result of the measurement of ¹³C-NMR, the composition ratio (molar ratio) in the following formula (MA1) was a:b:c:d=40:30:15:15. Although the following formula (MA1) is illustrated in a simplified form to show the ratio of respective constitutional units, the order of arrangement of respective constitutional units is random and this polymer is not a block copolymer in which respective constitutional units form an independent block.

The inorganic element content and organic impurity content of the synthesized polymer were measured by the aforementioned method, and the obtained measurement results are shown in Table 3A. The molar ratio was determined based on the integral ratio of each of the carbon on the bottom of a benzene ring for the polystyrene monomer (the compound A1) and the carbonyl carbon of an ester bond for methacrylate monomer (2-methyl-2-adamantyl methacrylate, γ-butyrolactone methacrylate, and hydroxyadamantyl methacrylate). The kind of respective monomers, the ratio thereof, and the composition ratio in the polymer obtained in Example B1 are shown in Table 2A and Table 2-1A. The kind of respective monomers, the ratio thereof, and the composition ratio in the polymers obtained in Examples described below are also shown in Table 2A.

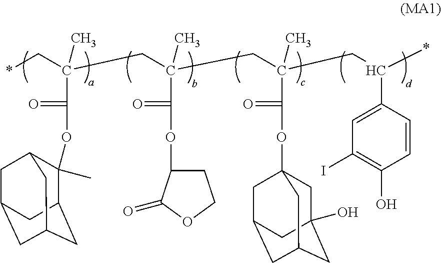

### Example B2 and Comparative Example BR1: Synthesis of Polymer B2 and Polymer BR1

The synthesis was carried out in the same manner as the method described in Example B1, except that 1.5 g of the compound A1 was changed to the monomer compounds of the kind and the amount shown in Table 2A, thereby obtaining the polymers B2 and BR1 represented by the formula (MA2), the formula (MAR1), and the formula (MAR3). The inorganic element content and organic impurity content of the polymers were measured by the aforementioned method, and the obtained measurement results are shown in Table 3A.

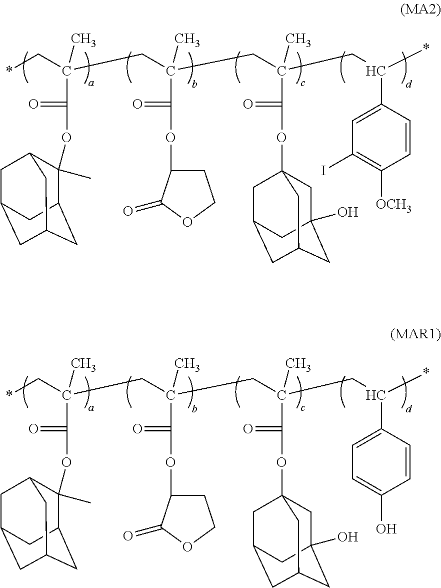

### (Example Group 2: Example B1P) Synthesis of Polymer B1P

For the synthesized compound A1, purification treatments of respective raw materials were additionally conducted before synthesis of the polymer. Using ethyl acetate (PrimePure manufactured by Kanto Chemical Co., Inc.) as a solvent, a 10% by mass ethyl acetate solution of the compound A1 in which the compound A1 was dissolved was prepared. To remove metal impurities, an ion exchange resin “AMBERLYST MSPS2-1·DRY” (product name, manufactured by ORGANO CORPORATION) was immersed in ethyl acetate (PrimePure manufactured by Kanto Chemical Co., Inc.), stirred for 1 hour, and then the solvent was removed. Washing of the ion exchange resin was carried out by repeating washing in this manner 10 times. In the aforementioned ethyl acetate solution of the compound A1, the same mass of the washed ion exchange resin as the resin solid content was put and stirred at room temperature for a day, and then the ion exchange resin was filtered off. Washing in which an ion exchange treatment was carried out in this manner was repeated three times to prepare an ion-exchanged ethyl acetate solution of the compound A1. Furthermore, the same treatment was carried out for other monomers, thereby preparing an ion-exchanged monomer-containing ethyl acetate solution. Synthesis was carried out according to the same scheme as the synthesis of the polymer B1 in Example B1 by using the obtained ion exchange treated monomer-containing ethyl acetate solution, using Pruimepure manufactured by Kanto Chemical Co., Inc., which is electronic grade as the solvent such as n-heptane and tetrahydrofuran, and further using instruments, all of which were immersed in nitric acid for a day and then washed with ultrapure water, as the reaction vessel such as a flask. Further in the treatment after synthesis, a purification treatment was carried out by using a 5 nm Nylon filter (manufactured by Pall) and a 15 nm PTFE filter (manufactured by Entegris) in the order presented, and the purified product was dried under reduced pressure to obtain a white powdery polymer B1P (a polymer whose chemical structure is represented by the formula (MA1)). The inorganic element content and organic impurity content of the monomer compounds used in the synthesis of the obtained polymers after the purification treatment were measured by the aforementioned method, and the obtained measurement results are shown in Table 3A.

### (Example Group 2: Examples B2P to B8P) Synthesis of Polymers B2P to B8P

The polymers B2P to B8P (polymers whose chemical structures are represented by the formulas (MA2 to MA8) were obtained in the same manner as Example B1P except for using the compounds M2 to M8 instead of the compound M1. The inorganic element content and organic impurity content of the monomer compounds used in the synthesis of the obtained polymers after the purification treatment were measured by the aforementioned method, and the obtained measurement results are shown in Table 3A.

TABLE 2A

| | Monomer | | | | | | | | | | |
|---|---|---|---|---|---|---|---|---|---|---|---|
| | Compound | | MAMA | BLMA | HAMA | Polymer | | | | | |
| | | Amount | Amount | Amount | Amount | | Structural | | | | |
| | No. | g | g | g | g | No. | formula | a | b | c | d |
| Example B1 | A1 | 4.2 | 3 | 2 | 1.5 | B1 | MA1 | 40 | 30 | 30 | 15 |
| Example B1P | A1 | 4.2 | 3 | 2 | 1.5 | B1P | MA1 | 40 | 30 | 30 | 15 |
| Example B2 | A2 | 4.2 | 3 | 2 | 1.5 | B2 | MA2 | 40 | 30 | 30 | 15 |
| Example B2P | A2 | 4.2 | 3 | 2 | 1.5 | B2P | MA3 | 40 | 30 | 30 | 15 |
| Example B3P | A3 | 4.2 | 3 | 2 | 1.5 | B3P | MA4 | 40 | 30 | 30 | 15 |
| Example B4P | A4 | 4.2 | 3 | 2 | 1.5 | B4P | MA5 | 40 | 30 | 30 | 15 |
| Example B5P | A5 | 4.2 | 3 | 2 | 1.5 | B5P | MA6 | 40 | 30 | 30 | 15 |
| Example B6P | A6 | 4.2 | 3 | 2 | 1.5 | B6P | MA7 | 40 | 30 | 30 | 15 |
| Example B7P | A7 | 4.2 | 3 | 2 | 1.5 | B7P | MA2 | 40 | 30 | 30 | 15 |
| Example B8P | A8 | 4.2 | 3 | 2 | 1.5 | B6P | MA7 | 40 | 30 | 30 | 15 |
| Comparative | AR1 | 2.1 | 3 | 2 | 1.5 | BR1 | MAR1 | 40 | 30 | 30 | 15 |
| Example BR1 | | | | | | | | | | | |

The meanings of the abbreviations and symbols in the Tables are as follows.
MAMA: 2-methyl-2-adamantyl methacrylate
BLMA: γ-butyrolactone methacrylate
HAMA: hydroxyadamantyl methacrylate
a, b, c and d of the polymer are molar ratios.

TABLE 3A

| | | | Monomer physical properties | | | | |
|---|---|---|---|---|---|---|---|
| | | | Impurity content (ppm) | | | | |
| | | | | | | | Phosphorus- |
| | | Structural | | | | Maleic | containing |
| | Polymer | formula | Mn | A1 | K | acid | compound |
| Example B1 | B1 | MA1 | 0.01 | 0.01 | 0.01 | DL | 0.1 |
| Example B1P | B1P | MA1 | 0.0001 | 0.0001 | 0.0001 | DL | DL |
| Example B2 | B2 | MA2 | 0.01 | 0.01 | 0.01 | DL | 0.1 |
| Example B2P | B2P | MA2 | 0.0001 | 0.0001 | 0.0001 | DL | DL |
| Example B3P | B3P | MA4 | 0.0001 | 0.0001 | 0.0001 | DL | DL |
| Example B4P | B4P | MA5 | 0.0001 | 0.0001 | 0.0001 | DL | DL |
| Example B5P | B5P | MA6 | 0.0001 | 0.0001 | 0.0001 | DL | DL |
| Example B6P | B6P | MA7 | 0.0001 | 0.0001 | 0.0001 | DL | DL |
| Example B7P | B7P | MA2 | 0.0001 | 0.0001 | 0.0001 | DL | DL |
| Example B8P | B8P | MA8 | 0.0001 | 0.0001 | 0.0001 | DL | DL |
| Comparative | BR1 | MAR1 | 0.1 | 0.1 | 0.1 | DL | DL |
| Example BR1 | | | | | | | |

DL: below detection limit (<0.1 ppm)

### (Example Group 2: Examples BD1 to BD15) Synthesis of Polymers PMD1 to PMD15

The polymers BD1 to BD15 (polymers whose chemical structures are represented by the formulas (PMD1 to PMD15)) were obtained in the same manner as Example B1P except for using the compound a1, the compound a2, and the compound a3 described in Table 2-2A instead of the compound M1, in the described ratio. The inorganic element content and organic impurity content of the monomer compounds used in the synthesis of the obtained polymers were measured by the aforementioned method, and the obtained measurement results are shown in Table 3-2A.

TABLE 2-2A

| | Composition | | | | | | | | |
|---|---|---|---|---|---|---|---|---|---|
| | Compound a1 | | Compound a2 | | Compound a3 | | | | |
| | | Amount | | Amount | | Amount | | | |
| | No. | (g) | No. | (g) | No. | (g) | MMA | BLMA | HAMA |
| Example BD1 | A1 | 3.89 | AR1 | 0.153 | — | — | 3 | 2 | 1.5 |
| Example BD2 | A1 | 4.15 | AR1 | 0.026 | — | — | 3 | 2 | 1.5 |
| Example BD3 | A1 | 4.18 | AR1 | 0.012 | — | — | 3 | 2 | 1.5 |
| Example BD4 | A1 | 4.20 | AR1 | 0.002 | — | — | 3 | 2 | 1.5 |
| Example BD5 | A1 | 3.89 | AD1a | 0.630 | — | — | 3 | 2 | 1.5 |
| Example BD6 | A1 | 4.15 | AD1a | 0.105 | — | — | 3 | 2 | 1.5 |
| Example BD7 | A1 | 4.18 | AD1a | 0.052 | — | — | 3 | 2 | 1.5 |
| Example BD8 | A1 | 4.20 | AD1a | 0.008 | — | — | 3 | 2 | 1.5 |
| Example BD9 | A1 | 3.89 | AD1b | 0.630 | — | — | 3 | 2 | 1.5 |
| Example BD10 | A1 | 4.15 | AD1b | 0.105 | — | — | 3 | 2 | 1.5 |
| Example BD11 | A1 | 4.18 | AD1b | 0.052 | — | — | 3 | 2 | 1.5 |
| Example BD12 | A1 | 4.20 | AD1b | 0.008 | — | — | 3 | 2 | 1.5 |
| Example BD13 | A1 | 3.66 | AR1 | 0.154 | AD1a | 0.462 | 3 | 2 | 1.5 |
| Example BD14 | A1 | 4.10 | AR1 | 0.030 | AD1a | 0.084 | 3 | 2 | 1.5 |
| Example BD15 | A1 | 4.20 | AR1 | 0.002 | AD1a | 0.008 | 3 | 2 | 1.5 |

| | | | a1 | a2 | a3 | b | c | d |
|---|---|---|---|---|---|---|---|---|
| | | Structural | Molar | Molar | Molar | Molar | Molar | Molar |
| | No. | formula | ratio | ratio | ratio | ratio | ratio | ratio |
| Example BD1 | BD16 | PME16 | 37 | 3 | | 30 | 30 | 15 |
| Example BD2 | BD17 | PME17 | 39.5 | 0.5 | | 30 | 30 | 15 |
| Example BD3 | BD18 | PME18 | 39.75 | 0.25 | | 30 | 30 | 15 |
| Example BD4 | BD19 | PME19 | 39.96 | 0.04 | | 30 | 30 | 15 |
| Example BD5 | BD20 | PME20 | 37 | 3 | | 30 | 30 | 15 |
| Example BD6 | BD21 | PME21 | 39.5 | 0.5 | | 30 | 30 | 15 |
| Example BD7 | BD22 | PME22 | 39.75 | 0.25 | | 30 | 30 | 15 |
| Example BD8 | BD23 | PME23 | 39.96 | 0.04 | | 30 | 30 | 15 |
| Example BD9 | BD24 | PME24 | 37 | 3 | | 30 | 30 | 15 |
| Example BD10 | BD25 | PME25 | 39.5 | 0.5 | | 30 | 30 | 15 |
| Example BD11 | BD26 | PME26 | 39.75 | 0.25 | | 30 | 30 | 15 |
| Example BD12 | BD27 | PME27 | 39.96 | 0.04 | | 30 | 30 | 15 |
| Example BD13 | BD28 | PME28 | 34.2 | 3 | 2.2 | 30 | 30 | 15 |
| Example BD14 | BD29 | PME29 | 39.0 | 0.6 | 0.4 | 30 | 30 | 15 |
| Example BD15 | BD30 | PME30 | 39.92 | 0.04 | 0.04 | 30 | 30 | 15 |

TABLE 3-2A

| | | | Monomer physical properties | | | | |
|---|---|---|---|---|---|---|---|
| | | | Impurity content (ppm) | | | | |
| | | | | | | | Phosphorus- |
| | | Structural | | | | Maleic | containing |
| | Polymer | formula | Mn | A1 | K | acid | compound |
| Example BD1 | BD1 | PMD1 | 0.0001 | 0.0001 | 0.0001 | DL | DL |
| Example BD2 | BD2 | PMD2 | 0.0001 | 0.0001 | 0.0001 | DL | DL |
| Example BD3 | BD3 | PMD3 | 0.0001 | 0.0001 | 0.0001 | DL | DL |
| Example BD4 | BD4 | PMD4 | 0.0001 | 0.0001 | 0.0001 | DL | DL |
| Example BD5 | BD5 | PMD5 | 0.0001 | 0.0001 | 0.0001 | DL | DL |
| Example BD6 | BD6 | PMD6 | 0.0001 | 0.0001 | 0.0001 | DL | DL |
| Example BD7 | BD7 | PMD7 | 0.0001 | 0.0001 | 0.0001 | DL | DL |
| Example BD8 | BD8 | PMD8 | 0.0001 | 0.0001 | 0.0001 | DL | DL |
| Example BD9 | BD9 | PMD9 | 0.0001 | 0.0001 | 0.0001 | DL | DL |
| Example BD10 | BD10 | PMD10 | 0.0001 | 0.0001 | 0.0001 | DL | DL |
| Example BD11 | BD11 | PMD11 | 0.0001 | 0.0001 | 0.0001 | DL | DL |
| Example BD12 | BD12 | PMD12 | 0.0001 | 0.0001 | 0.0001 | DL | DL |
| Example BD13 | BD13 | PMD13 | 0.0001 | 0.0001 | 0.0001 | DL | DL |
| Example BD14 | BD14 | PMD14 | 0.0001 | 0.0001 | 0.0001 | DL | DL |
| Example BD15 | BD15 | PMD15 | 0.0001 | 0.0001 | 0.0001 | DL | DL |

### [Evaluation]

The aforementioned polymers B1 to B8P, and BR1 obtained in Examples and Comparative Examples were evaluated as follows. The results are shown in Table 4A, Table 4-2A, Table 4-2A, Table 5A, and Table 5-2A.

### (EUV Sensitivity-Aqueous TMAH Solution Development)

A solution was prepared by blending 5 parts by mass of the compound or the polymer obtained in Examples or Comparative Examples, 1 part by mass of triphenylsulfonium nonafluoromethanesulfonate, 0.2 parts by mass of tributylamine, 80 parts by mass of PGMEA, and 12 parts by mass of PGME.

The solution was applied on a silicon wafer, which was baked at 110° C. for 60 seconds to form a photoresist layer having a film thickness of 100 nm.

Then, using an extreme ultraviolet (EUV) exposure apparatus “EUVES-7000” (product name, manufactured by Litho Tech Japan Corporation), the photoresist layer was subjected to shot exposure without masking by incrementing the exposure amount by 1 mJ/cm² from 1 mJ/cm² to 80 mJ/cm², baked (PEB) at 110° C. for 90 seconds, and developed with a 2.38% by mass aqueous tetramethylammonium hydroxide (TMAH) solution for 60 seconds to obtain a wafer on which 80 shots of shot exposure was carried out. For each obtained shot exposure area, the film thickness was measured by using an optical interference film thickness meter “VM3200” (product name, manufactured by SCREEN Semiconductor Solutions Co., Ltd.), profile data about the film thickness relative to the exposure amount was obtained, and the exposure amount at which the slope of film thickness variation relative to an exposure amount was the largest was calculated as a sensitivity value (mJ/cm²), which was used as an index of the EUV sensitivity of the resist.

### (Change of Sensitivity Due to Aging)

The solution prepared in the aforementioned EUV sensitivity evaluation was subjected to a forced aging treatment under light-shielding conditions at 40° C./240 hours, and the solution after the aging treatment was evaluated for EUV sensitivity in the same manner. Evaluations were conducted based on the amount of change of sensitivity. As the specific evaluation method, in a film thickness-sensitivity curve after development when the abscissa was set to sensitivity and the ordinate was set to film thickness in the EUV sensitivity evaluation, the sensitivity value at which the slope value was maximum was measured as the standard sensitivity. The standard sensitivities of the solutions before and after being subjected to a forced aging treatment were determined, and sensitivity variations due to the aging treatment were evaluated from the numerical value obtained by the following calculation expression. The evaluation criteria are as follows.

[Sensitivity variation]=1−([standard sensitivity of solution after aging]÷[standard sensitivity of solution before aging])

### (Evaluation Criteria)

S: [sensitivity variation]≤0.0025
A: 0.0025<[sensitivity variation]≤0.005
B: 0.005<[sensitivity variation]≤0.02
C: 0.02<[sensitivity variation]≤0.05
D: 0.05<[sensitivity variation]

### (EB Pattern-Aqueous TMAH Solution Development)

A solution was prepared by blending 5 parts by mass of the compound or the polymer obtained in Examples or Comparative Examples, 1 part by mass of triphenylsulfonium nonafluoromethanesulfonate, 0.1 parts by mass of tributylamine, and 92 parts by mass of PGMEA.

The solution was applied on a silicon wafer, which was baked at 110 to 130° C. for 60 seconds to form a resist film having a film thickness of 100 nm.

Then, the resist film was exposed using an electron beam lithography system “ELS-7500” (product name, manufactured by ELIONIX INC., 50 keV), baked (PEB) at 115° C. for 90 seconds, and developed with a 2.38 mass % tetramethylammonium hydroxide (TMAH) aqueous solution for 60 seconds to obtain a positive type pattern. The exposure amount was regulated to have a line and space pattern with a half pitch of 50 nm.

For the obtained resist pattern, 80 pattern images were obtained with a scanning electron microscope “S-4800” (product name, manufactured by Hitachi, Ltd.) at 100000 times magnification, the number of residues on the space portion between resist patterns were counted, and evaluations were carried out from the total amount of the residues. The evaluation criteria are as follows.

### (Evaluation Criteria)

A: Number of residues≤less than 10
B: 10<number of residues≤80
C: 80<number of residues≤400
D: 400<number of residues

### (Evaluation of Etching Defect)

A solution was prepared by blending 5 parts by mass of the compound or the polymer obtained in Examples or Comparative Examples, 1 part by mass of triphenylsulfonium nonafluoromethanesulfonate, 0.2 parts by mass of tributylamine, 80 parts by mass of PGMEA, and 12 parts by mass of PGME.

The solution was applied on a 8 inch silicon wafer, on the outermost layer of which an oxide film having a film thickness of 100 nm was formed, and this was baked at 110° C. for 60 seconds to form a photoresist layer having a film thickness of 100 nm.

Then, using an extreme ultraviolet (EUV) exposure apparatus “EUVES-7000” (product name, manufactured by Litho Tech Japan Corporation), the photoresist layer was subjected to shot exposure with an exposure amount that is 10% less than the EUV sensitivity value obtained in the aforementioned EUV sensitivity evaluation on the entire wafer surface, further baked (PEB) at 110° C. for 90 seconds, and developed with a 2.38% by mass aqueous tetramethylammonium hydroxide (TMAH) solution for 60 seconds to obtain a wafer on the entire surface of which 80 shots of shot exposure was carried out.

The prepared exposed wafer was subjected to an etching treatment with an etching apparatus “Telius SCCM” (product name, manufactured by Tokyo Electron Ltd.), using CF₄/Ar gas until the oxide film was etched by 50 nm. Evaluation of defects were carried out for the wafer prepared by etching with a defect inspection apparatus “Surfscan SP5” (product name, manufactured by KLA), and the number of cone defects with a size of 19 nm or more was determined as an index of the etching defect.

### (Evaluation Criteria)

A: Number of cone defects≤less than 10
B: 10<number of cone defects≤80
C: 80<number of cone defects≤400
D: 400<number of cone defects

TABLE 4A

| | | | Evaluation | | | |
|---|---|---|---|---|---|---|
| | | | EUV | | | |
| | | | sensitivity - | Change | EB pattern - | |
| | | | aqueous | of | aqueous | |
| | | | TMAH | sensitivity | TMAH | |
| | | Structural | solution | due to | solution | Etching |
| | Polymer | formula | development | aging | development | defect |
| Example B1 | B1 | MA1 | 17 | B | B | B |
| Example B1P | B1P | MA1 | 15 | A | A | A |
| Example B2 | B2 | MA2 | 19 | B | B | B |
| Example B2P | B2P | MA2 | 16 | A | A | A |
| Example B3P | B3P | MA4 | 17 | A | A | A |
| Example B4P | B4P | MA5 | 17 | A | A | A |
| Example B5P | B5P | MA6 | 17 | A | A | A |
| Example B6P | B6P | MA7 | 17 | A | A | A |
| Example B7P | B7P | MA2 | 17 | A | A | A |
| Example B8P | B8P | MA8 | 17 | A | A | A |
| Comparative | BR1 | MAR1 | 49 | C | C | C |
| Example BR1 | | | | | | |
| Reference | BX1 | MX1 | 26 | A | A | A |
| Example BX1 | | | | | | |

TABLE 4-2A

| | | | Evaluation | | | |
|---|---|---|---|---|---|---|
| | | | EUV | | | |
| | | | sensitivity - | Change | EB pattern - | |
| | | | aqueous | of | aqueous | |
| | | | TMAH | sensitivity | TMAH | |
| | | Structural | solution | due to | solution | Etching |
| | Polymer | formula | development | aging | development | defect |
| Example BD1 | BD1 | PMD1 | 15 | A | A | A |
| Example BD2 | BD2 | PMD2 | 15 | A | A | A |
| Example BD3 | BD3 | PMD3 | 15 | A | A | A |
| Example BD4 | BD4 | PMD4 | 15 | A | A | A |
| Example BD5 | BD5 | PMD5 | 14 | A | A | A |
| Example BD6 | BD6 | PMD6 | 14 | A | A | A |
| Example BD7 | BD7 | PMD7 | 14 | A | A | A |
| Example BD8 | BD8 | PMD8 | 14 | A | A | A |
| Example BD9 | BD9 | PMD9 | 14 | A | A | A |
| Example BD10 | BD10 | PMD10 | 14 | A | A | A |
| Example BD11 | BD11 | PMD11 | 14 | A | A | A |
| Example BD12 | BD12 | PMD12 | 14 | A | A | A |
| Example BD13 | BD13 | PMD13 | 13 | A | A | A |
| Example BD14 | BD14 | PMD14 | 13 | A | A | A |
| Example BD15 | BD15 | PMD15 | 13 | A | A | A |

### (EB Pattern 2-Aqueous TMAH Solution Development)

A solution was prepared by blending 8 parts by mass of the compound or the polymer obtained in Examples or Comparative Examples, 1 part by mass of triphenylsulfonium nonafluoromethanesulfonate, 1 part by mass of triphenylsulfonium trifluoromethanesulfonate, 0.2 parts by mass of tributylamine, and 92 parts by mass of PGMEA.

The solution was applied on a silicon wafer, which was baked at 120° C. for 60 seconds to form a resist film having a film thickness of 80 nm.

Then, the resist film was exposed using an electron beam lithography system “ELS-7500” (product name, manufactured by ELIONIX INC., 50 keV), baked (PEB) at 110° C. for 90 seconds, and developed with a 2.38 mass % tetramethylammonium hydroxide (TMAH) aqueous solution for 60 seconds to obtain a positive type pattern. The exposure amount was regulated to have a hole pattern with a diameter of 30 nm, which was arranged such that the half pitch is 50 nm in X axis and 50 nm in Y axis.

For the obtained resist pattern, 80 pattern images were obtained with a scanning electron microscope “S-4800” (product name, manufactured by Hitachi, Ltd.) at 100000 times magnification, the number of residues on the space portion between resist patterns were counted, and evaluations were carried out from the total amount of the residues. The evaluation criteria are as follows.

### (Evaluation Criteria)

A: Number of residues≤less than 10
B: 10<number of residues≤80
C: 80<number of residues≤400
D: 400<number of residues

Furthermore, sampling for the hole diameter of the obtained pattern image was carried out at 100 arbitrary points, the standard deviation of the variation value of the hole diameter, the line width 6, was determined, and evaluations were carried out under the following evaluation criteria.

### (Evaluation Criteria)

A: Line width σ≤less than 2 nm
B: 2 nm<line width σ≤4 nm
C: 4 nm<line width σ≤7 nm
D: 7 nm<line width σ

TABLE 4-3A

| | | | Evaluation EB pattern 2 - aqueous | |
|---|---|---|---|---|
| | | | TMAH solution development | |
| | | Structural | Number of | Line |
| | Polymer | formula | residues | width σ |
| Example B1P | B1P | MA1 | A | A |
| Example B2P | B2P | MA2 | A | A |
| Example B3P | B3P | MA4 | A | A |
| Example B4P | B4P | MA5 | A | A |
| Example B5P | B5P | MA6 | A | A |
| Example B6P | B6P | MA7 | A | A |
| Example B7P | B7P | MA2 | A | A |
| Example B8P | B8P | MA2 | A | A |
| Reference | BX1 | MX1 | B | B |
| Example BX1 | | | | |

The above results show that, in particular, excellent resolution of a fine hole pattern is obtained by introducing the compound of the present invention in the second embodiment.

### (EUV Sensitivity—Organic Solvent Development)

A solution containing the compound or the polymer obtained in Examples or Comparative Examples was prepared in the same manner as the EUV sensitivity-aqueous TMAH solution development, which was applied on a silicon wafer and baked at 110° C. for 60 seconds to form a photoresist layer having a film thickness of 100 nm.

Then, using an extreme ultraviolet (EUV) exposure apparatus “EUVES-7000” (product name, manufactured by Litho Tech Japan Corporation), the photoresist layer was subjected to shot exposure without masking by incrementing the exposure amount by 1 mJ/cm² from 1 mJ/cm² to 80 mJ/cm², baked (PEB) at 110° C. for 90 seconds, and developed with butyl acetate for 30 seconds to obtain a wafer on which 80 shots of shot exposure was carried out. For each obtained shot exposure area, the film thickness was measured by using an optical interference film thickness meter “VM3200” (product name, manufactured by SCREEN Semiconductor Solutions Co., Ltd.), profile data about the film thickness relative to the exposure amount was obtained, and the exposure amount at which the slope of film thickness variation relative to an exposure amount was the largest was calculated as a sensitivity value (mJ/cm²), which was used as an index of the EUV sensitivity of the resist.

### (EB Pattern-Organic Solvent Development)

A solution containing the compound or the polymer obtained in Examples or Comparative Examples was prepared in the same manner as the EB pattern-aqueous TMAH solution development, which was applied on a silicon wafer and baked at 110 to 130° C. for 60 seconds to form a resist film having a film thickness of 100 nm.

Then, the resist film was exposed using an electron beam lithography system “ELS-7500” (product name, manufactured by ELIONIX INC., 50 keV), baked (PEB) at 115° C. for 90 seconds, and developed with butyl acetate for 30 seconds to obtain a negative type pattern. The exposure amount was regulated to have a line and space pattern with a half pitch of 50 nm.

For the obtained resist pattern, 80 pattern images were obtained with a scanning electron microscope “S-4800” (product name, manufactured by Hitachi, Ltd.) at 100000 times magnification, the number of residues on the space portion between resist patterns were counted, and evaluations were carried out from the total amount of the residues. The evaluation criteria are as follows.

### (Evaluation Criteria)

A: Number of residues≤less than 10
B: 10<number of residues≤80
C: 80<number of residues≤400
D: 400<number of residues

TABLE 5A

| | | | EUV | |
|---|---|---|---|---|
| | | | sensitivity - | EB pattern - |
| | | | organic | organic |
| | | Structural | solvent | solvent |
| | Polymer | formula | development | development |
| Example B1 | B1 | MA1 | 19 | B |
| Example B1P | B1P | MA1 | 17 | A |
| Example B2 | B2 | MA2 | 17 | B |
| Example B2P | B2P | MA2 | 15 | A |
| Example B3P | B3P | MA4 | 19 | A |
| Example B4P | B4P | MA5 | 19 | A |
| Example B5P | B5P | MA6 | 19 | A |
| Example B6P | B6P | MA7 | 19 | A |
| Example B7P | B7P | MA2 | 19 | A |
| Example B8P | B8P | MA2 | 19 | A |
| Comparative | BR1 | MAR1 | 55 | C |
| Example BR1 | | | | |
| Comparative | BR3 | MAR3 | 22 | B |
| Example BR3 | | | | |
| Reference | BX1 | MX1 | 29 | A |
| Example BX1 | | | | |

TABLE 5-2A

| | | | EUV | |
|---|---|---|---|---|
| | | | sensitivity - | EB pattern - |
| | | | organic | solvent |
| | | Structural | solvent | organic |
| | Polymer | formula | development | development |
| Example BD1 | BD1 | PMD1 | 15 | A |
| Example BD2 | BD2 | PMD2 | 15 | A |
| Example BD3 | BD3 | PMD3 | 15 | A |
| Example BD4 | BD4 | PMD4 | 15 | A |
| Example BD5 | BD5 | PMD5 | 14 | A |
| Example BD6 | BD6 | PMD6 | 14 | A |
| Example BD7 | BD7 | PMD7 | 14 | A |
| Example BD8 | BD8 | PMD8 | 14 | A |
| Example BD9 | BD9 | PMD9 | 14 | A |
| Example BD10 | BD10 | PMD10 | 14 | A |
| Example BD11 | BD11 | PMD11 | 14 | A |
| Example BD12 | BD12 | PMD12 | 14 | A |
| Example BD13 | BD13 | PMD13 | 15 | A |
| Example BD14 | BD14 | PMD14 | 15 | A |
| Example BD15 | BD15 | PMD15 | 15 | A |

1. A compound represented by the following formula (1): 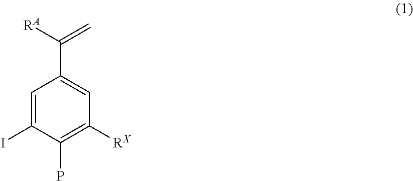
wherein RA is a hydrogen atom, a methyl group, or a trifluoromethyl group; RX is ORB or a hydrogen atom; RB is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group.

2. (canceled)

3. (canceled)

4. (canceled)

5. (canceled)

6. A composition comprising a compound represented by the following formula (1A) in an amount of 1 ppm by mass or more and 10% by mass or less, based on the total compound according to claim 1: 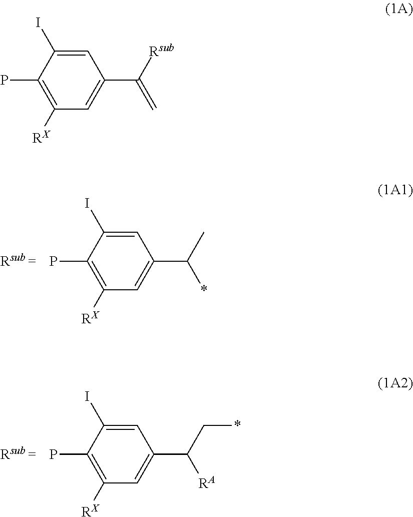
wherein R^{A}, R^{X}, R^{B}, and P have the same meanings as in the formula (1); R^{sub} represents the formula (1A1) or formula (1A2); and * is a site for binding with an adjacent constitutional unit.

7. A composition comprising: the compound according to claim 1; and based on the total compound, 1 ppm by mass or more and 10% by mass or less of a compound represented by the following formula (1B): 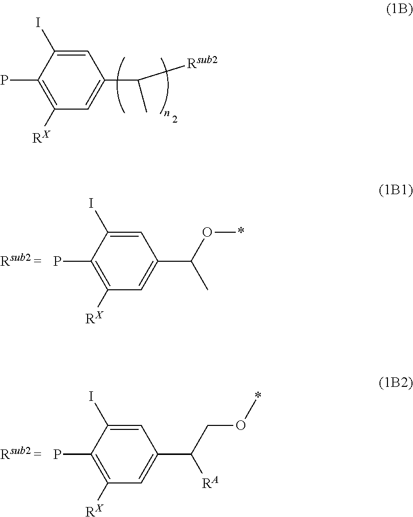
wherein R^{A}, R^{X}, R^{B}, and P have the same meanings as in the formula (1); n₂ is an integer of 0 or more and 4 or less; R^{sub2} represents the formula (1B1) or the formula (1B2); and * is a site for binding with an adjacent constitutional unit.

8. A composition comprising a compound represented by the following formula (1C) in an amount of 1 ppm by mass or more and 10% by mass or less, based on the total compound according to claim 1: 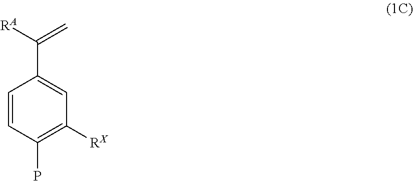
wherein R^{A}, R^{X}, R^{B}, and P have the same meanings as in the formula (1); provided that neither R^{B} nor P contains I.

9. A composition comprising the compound according to claim 1,
wherein a content of impurities containing K is 1 ppm by mass or less, in terms of element, based on the total compound.

10. The composition according to claim 9, wherein a content of peroxide is 10 ppm by mass or less, based on the total compound.

11. The composition according to claim 9, wherein a content of impurities containing one or more elements selected from the group consisting of Mn, Al, Si, and Li is 1 ppm by mass or less, in terms of element, based on the total compound.

12. The composition according to claim 9, wherein a content of a phosphorus-containing compound is 10 ppm by mass or less, based on the total compound.

13. The composition according to claim 9, wherein a content of maleic acid is 10 ppm by mass or less, based on the total compound.

14. A polymer comprising a constitutional unit represented by the following formula (1-A), comprising a constitutional unit derived from the compound according to claim 1: 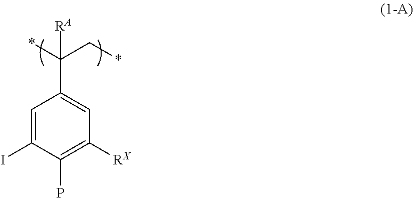
wherein R^{A}, R^{X}, R^{B}, and P have the same meanings as in the formula (1); and * is a site for binding with an adjacent constitutional unit.

15. The polymer according to claim 14, further comprising a constitutional unit represented by the following formula (C0), the following formula (C1), or the following formula (C2): 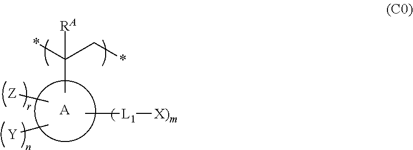
wherein
each X is independently I, F, Cl, Br, or an organic group having 1 to 30 carbon atoms and having 1 or more and 5 or less substituents selected from the group consisting of I, F, Cl, and Br;
each L¹ is independently a single bond, an ether group, an ester group, a thioether group, an amino group, a thioester group, an acetal group, a phosphine group, a phosphone group, a urethane group, a urea group, an amide group, an imide group, or a phosphate group, and the ether group, the ester group, the thioether group, the amino group, the thioester group, the acetal group, the phosphine group, the phosphone group, the urethane group, the urea group, the amide group, the imide group, or the phosphate group of L¹ optionally has a substituent;
each Y is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;
R^{A} is as defined in the formula (1);
A is an organic group having 1 to 30 carbon atoms;
each Z is independently an alkoxy group, an ester group, an acetal group, a carboxyalkoxy group, or a carbonate ester group, and the alkoxy group, the ester group, the acetal group, the carboxyalkoxy group, or the carbonate ester group of Z optionally has a substituent; and
m is an integer of 0 or more, n is an integer of 1 or more, and r is an integer of 0 or more; 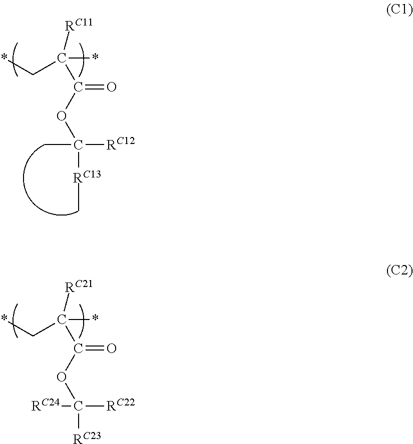
wherein, in the formula (C1),
R^{C11} is a hydrogen atom, a methyl group, or a trifluoromethyl group;
R^{C12} is a hydrogen atom, or an alkyl group having 1 to 4 carbon atoms;
R^{C13} is a cycloalkyl group or heterocycloalkyl group having 4 to 20 carbon atoms formed by taking R^{C13} together with a carbon atom bonded thereto; and
* is a site for binding with an adjacent constitutional unit.
wherein, in the formula (C2),
R^{C21} is a hydrogen atom, a methyl group, or a trifluoromethyl group;
each of R^{C22} and R^{C23} is independently an alkyl group having 1 to 4 carbon atoms;
R^{C24} is an alkyl group having 1 to 4 carbon atoms or a cycloalkyl group having 5 to 20 carbon atoms;
two or three selected from R^{C22}, R^{C23}, and R^{C24} are optionally taken together with a carbon atom bonded to the two or three selected from R^{C22}, R^{C23}, and R^{C24} to form an alicyclic structure having 3 to 20 carbon atoms; and
* is a site for binding with an adjacent constitutional unit.

16. A composition for film formation comprising the compound according to claim 1, the composition according to any one of claims 6 to 13, or the polymer according to claim 14 or 15.

17. (canceled)

18. A resist pattern formation method comprising:
a step of forming a resist film on a substrate using the composition for film formation according to claim 16;
a step of exposing a pattern on the resist film; and
a step of subjecting the resist film after exposure to a development treatment.

19. An insulating film formation method comprising:
a step of forming a resist film on a substrate using the composition for film formation according to claim 16;
a step of exposing a pattern on the resist film; and
a step of subjecting the resist film after exposure to a development treatment.

20. A method for producing an iodine-containing vinyl monomer represented by the following formula (1), comprising:
a) a step of providing an iodine-containing alcohol substrate having a general structure represented by the following formula (1-1): 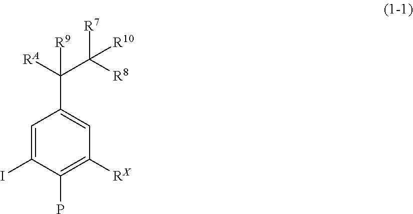
wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
b) a dehydration step of subjecting the iodine-containing alcohol substrate to a dehydration treatment: 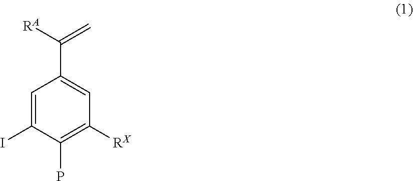
wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group.

21. The method for producing the iodine-containing vinyl monomer represented by the formula (1) according to claim 20, further comprising:
c) a step of providing an iodine-containing ketone substrate having a general structure represented by the following formula (1-2): 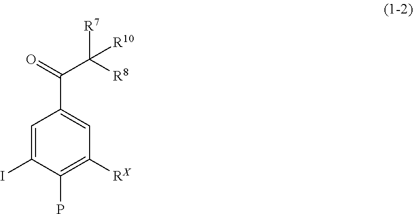
wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
d) a reduction step of subjecting the iodine-containing ketone substrate to a reduction treatment.

22. The method for producing the iodine-containing vinyl monomer represented by the formula (1) according to claim 20, further comprising:
e) a step of providing an alcohol substrate having a general structure represented by the following formula (1-3): 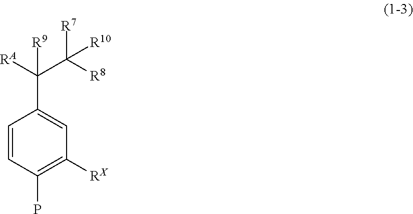
wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
f) an iodine introduction step of introducing an iodine atom into the alcohol substrate.

23. The method for producing the iodine-containing vinyl monomer represented by the formula (1) according to claim 20, further comprising:
g) a step of providing a ketone substrate having a general structure represented by the following formula (1-4): 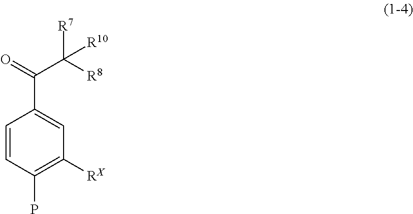
wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
h) an iodine introduction step of introducing an iodine atom into the ketone substrate.

24. The method for producing the iodine-containing vinyl monomer represented by the formula (1) according to claim 20, further comprising:
i) a step of providing a ketone substrate having a general structure represented by the following formula (1-4): 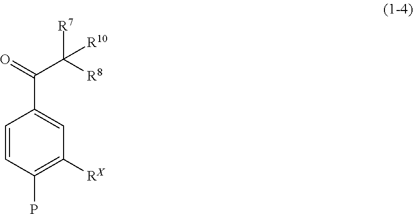
wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
j) a reduction step of subjecting the ketone substrate to a reduction treatment.

25. A method for producing an iodine-containing vinyl monomer represented by the following formula (2), comprising:
k) a step of providing an iodine-containing vinyl monomer having a general structure represented by the following formula (1): 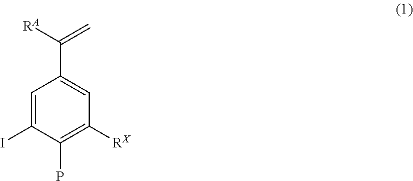
wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and
l) an acylation step of subjecting the iodine-containing vinyl monomer to an acylation treatment; 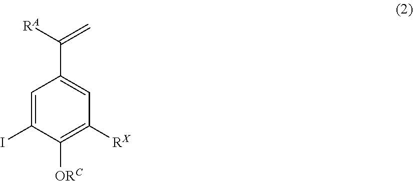
wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and R^{C} is a substituted or unsubstituted acyl group having 1 to 30 carbon atoms.

26. A method for producing an iodine-containing alcohol compound represented by the following formula (1-1), comprising:
c) a step of providing an iodine-containing ketone substrate having a general structure represented by the following formula (1-2): 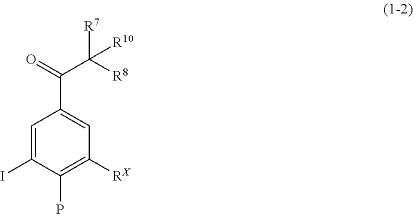
wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
d) a reduction step of subjecting the iodine-containing ketone substrate to a reduction treatment; 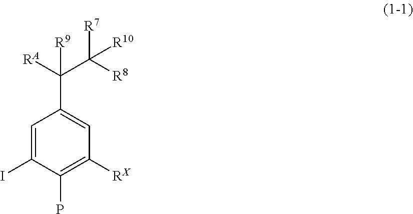
wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group.

27. A method for producing an iodine-containing alcohol compound represented by the following formula (1-1), comprising:
e) a step of providing an alcohol substrate having a general structure represented by the following formula (1-3): 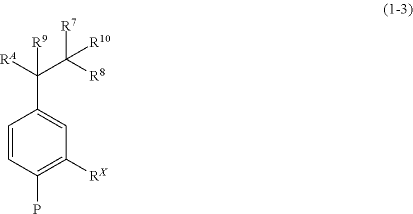
wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
f) an iodine introduction step of introducing an iodine atom into the alcohol substrate; 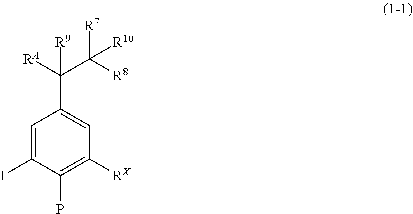
wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group.

28. A method for producing an iodine-containing ketone compound represented by the following formula (1-2), comprising:
g) a step of providing a ketone substrate having a general structure represented by the following formula (1-4): 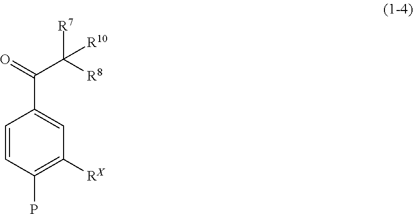
wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
h) an iodine introduction step of introducing an iodine atom into the ketone substrate; 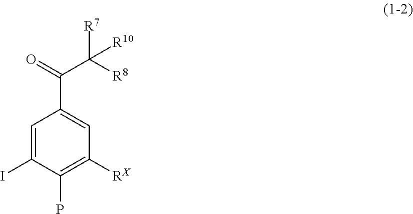
wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group.

29. A method for producing an alcohol compound represented by the following formula (1-3), comprising:
i) a step of providing a ketone substrate having a general structure represented by the following formula (1-4): 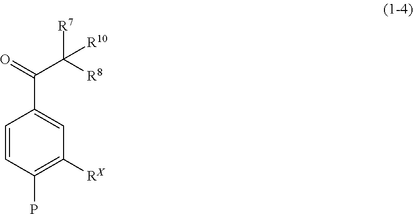
wherein R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group; and
j) a reduction step of subjecting the ketone substrate to a reduction treatment; 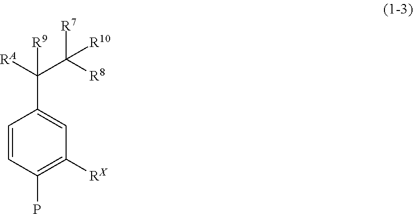
wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and each of R⁷ to R¹⁰ is independently a hydrogen atom, a hydroxy group, a methoxy group, a halogen, or a cyano group, provided that one of R⁷ to R¹⁰ is a hydroxy group or a methoxy group.

30. A method for producing an iodine-containing vinyl monomer represented by the following formula (1), comprising:
a) a step of providing an iodine-containing aldehyde substrate or iodine-containing ketone substrate having a general structure represented by the following formula (1-5): 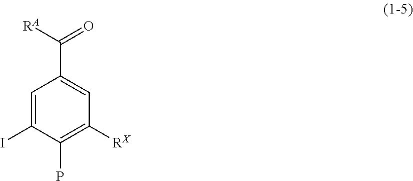
wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group; and
b) a Wittig reaction step of forming an alkene from a carbonyl moiety of the iodine-containing aldehyde substrate or the iodine-containing ketone substrate by a Wittig reaction; 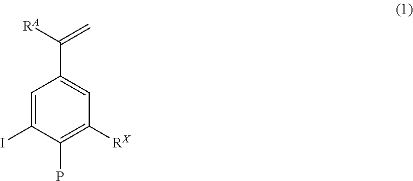
wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group.

31. A method for producing an iodine-containing vinyl monomer represented by the following formula (1), comprising:
a) a step of providing an iodine-containing aldehyde substrate or iodine-containing ketone substrate having a general structure represented by the following formula (1-5): 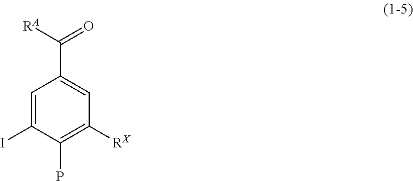
wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group;
b) a malonic acid addition step of adding malonic acid to the iodine-containing aldehyde substrate or the iodine-containing ketone substrate;
c) a hydrolysis step of subjecting the iodine-containing aldehyde substrate or iodine-containing ketone substrate to which the malonic acid is added to a hydrolysis treatment to produce an iodine-containing carboxylic acid substrate; and
d) a decarbonation step of subjecting the iodine-containing carboxylic acid substrate to a decarbonation treatment; 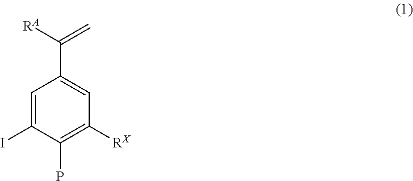
wherein R^{A} is a hydrogen atom, a methyl group, or a trifluoromethyl group; R^{X} is OR^{B} or a hydrogen atom; R^{B} is a substituted or unsubstituted alkyl group having 1 to 30 carbon atoms; and P is a hydroxy group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group.

32. The compound, polymer, composition, composition for film formation, pattern formation method, insulating film formation method, and method for producing a compound according to any one of claim 1, 6-15 or 20-31 applied to extreme ultraviolet applications.
